(19) United States
(12) Patent Application Publication
(10) Pub. No.: US 2025/0388913 A1
(43) Pub. Date: Dec. 25, 2025

(54) DNA ASSEMBLY

(71) Applicant: Oxford University Innovation Limited, Oxford (GB)

(72) Inventors: Da LIN, Oxford (Oxfordshire) (GB); Chris O'CALLAGHAN, Oxford (Oxfordshire) (GB)

(21) Appl. No.: 16/610,121

(22) PCT Filed: May 2, 2018
(86) PCT No.: PCT/GB2018/051174

§ 371 Date: Nov. 1, 2019

(30) Foreign Application Priority Data
May 3, 2017 (GB) 1707049.1
Aug. 23, 2017 (GB) 1713546.8

(51) Int. Cl. C12N 15/64; C12N 9/00; C12N 9/16; C12N 15/10; C12N 15/113; C12N 15/66; C12N 15/74

(52) U.S. Cl. CPC C12N 15/64; C12N 9/16; C12N 9/93; C12N 15/10; C12N 15/113; C12N 15/66; C12N 15/74; C12Y 301/21004; C12Y 605/01001

(57) ABSTRACT

The invention relates to a nucleic acid for use in DNA assembly, wherein the nucleic acid comprises at least one methylation-protectable restriction element, the methylation-protectable restriction element comprising: a restriction enzyme recognition sequence that is recognised by a restriction enzyme that cleaves outside of the recognition sequence; and a DNA methylase recognition sequence, wherein the restriction enzyme recognition sequence and the DNA methylase recognition sequence overlap such that the base modified by the DNA methylase lies within the restriction enzyme recognition sequence, wherein the DNA methylase recognition sequence is not identical to or enclosed by the restriction enzyme recognition sequence, and wherein the DNA methylase recognition sequence does not overlap with the sequence that would form the overhang end sequence generated by the restriction enzyme. The invention further relates to asscociated methods and kits.

### CROSS-REFERENCE TO RELATED APPLICATIONS

This application is the National Stage of International Application No. PCT/GB2018/051174, filed May 2, 2018, which claims the priority to GB 1707049.1, filed May 3, 2017, and GB 1713546.8, filed Aug. 23, 2017, which are entirely incorporated herein by reference.

### SEQUENCE LISTING

This application contains a sequence listing filed in electronic form as an ASCII.txt file entitled “JDM87541P.WOP_ST25.TXT”, created on May 5, 2017, and having a size of 4.2 Mb. The content of the sequence listing is incorporated herein in its entirety.

### FIELD OF THE DISCLOSURE

This invention relates to DNA assembly methods, and related nucleic acid material.

### BACKGROUND

Advances in biology and biotechnology have led to development of a variety of methods for assembly of large DNA constructs. Existing methods could be largely divided into two groups: homology-based methods that utilize long overlapping sequence between fragments to specify the order of assembly, such as Gibson assembly and yeast- or B. subtilis-based in vivo homologous recombination approaches, and restriction enzyme/recombinase-based methods that utilize defined enzyme-specific sequence to specify the order of assembly, such as Moclo (Reviewed in Nat Rev Mol Cell Biol. 2015 September; 16(9):568-76. doi: 10.1038/nrm4014 (PMID 2608161)2 and Crit Rev Biotechnol. 2017 May; 37(3):277-286. doi: 10.3109/07388551.2016.1141394 (PMID 26863154). An ongoing struggle is to achieve a balance between reducing sequence design constraints such as forbidden sequence in assembled DNA and unwanted scar sequence introduced during the assembly process, and improving assembly modularity and part reusability.

The initial type IIS restriction enzyme-based DNA assembly system was named Golden Gate assembly. In Golden Gate assembly system, insert DNA fragments to be assembled within insert plasmids are flanked by recognition sites for a type IIS restriction enzyme that cuts outside the recognition sequence and generates arbitrary adhesive ends with sequences independent of the enzyme recognition sequence. The insert plasmids for Golden Gate assembly contain inserts flanked by type IIS restriction site within the insert vector backbone facing the inserts, so that once the inserts are released from the plasmid by the type IIS restriction enzyme, they do not contain the restriction site. The assembly vector for Golden Gate assembly contains a negative selection marker flanked by type IIS sites located in the selection marker facing the vector, so that once the selection marker is released from the vector, the vector backbone does not contain the type IIS restriction site. These specially designed plasmids allow simple one-pot assembly of multiple DNA fragments by mixing insert plasmids and vectors together with a type IIS restriction enzyme and DNA ligase. In the Golden Gate assembly reaction, restriction digest of inserts and vector, and ligation between inserts and vector occurs in the same tube. A mixture of ligation products will be generated during the one-pot reaction when inserts and assembly vector backbone have compatible adhesive ends. This includes favorable ligation products among inserts and assembly vector backbone, as well as unfavorable ligation products such as between the negative selection marker and assembly vector backbone, between the negative selection marker and insert vector backbone, and between insert and insert vector backbone. Because the type IIS restriction sites are located within the negative selection marker and the insert vector backbone, all the unfavorable ligation products are susceptible to digestion by the type IIS restriction enzyme, while the favorable ligation products are refractory to digestion. As a result the reaction favors generation of correctly assembled ligation products among inserts and assembly vector backbone in the long term. Followed by selection using positive antibiotic selection markers within the assembly vector backbone and negative selection marker within the assembly vector insert, plasmids containing correctly assembled inserts could be selected with high efficiency.

Moclo-based systems were developed from the Golden Gate assembly system by adding a second pair type IIS restriction sites different from the enzyme used for assembly to the assembly vector in order to release the assembled DNA for next stage assembly. In Golden Gate assembly, the assembled DNA could not be released by the same restriction enzyme used for assembly, because the inserts and assembly plasmid vector backbone lack recognition sequence of the type IIS restriction enzyme used. By adding a different pair of type IIS restriction sites to release the assembled fragment, the process of Golden Gate assembly could be repeated using this second restriction enzyme and a different vector carrying a different antibiotic selection marker. Alternative sequential uses of two different type IIS restriction enzymes and antibiotic selection markers enable hierarchical assembly of large DNA fragments by one-pot restriction/ligation reaction.

A major drawback of type IIS enzyme-based DNA assembly is the necessity to remove internal type IIS restriction sites within the DNA parts to be used for assembly. A minimum of two enzymes are required for basic hierarchical assembly, and three enzymes are required for other schemes such as multi-step linear addition of DNA parts. Because most of the known type IIS restriction enzymes recognize <=6 bp sequence, most Moclo-based systems use 6 bp cutters. This requires that the DNA part must be free of two 6 bp asymmetric sequences, which occur at a frequency of ˜ 1 in 1 kb for a random DNA sequence with 50% GC content. A potential improvement of the current Moclo system would be to use type IIS restriction enzymes with 7 bp or more specificity. However, this option is limited by the availability of commercial type IIS restriction enzymes. Only two types of 7 bp type IIS restriction enzymes are currently commercially available: LguI/SapI which recognizes GCTCTTC and leaves 3 bp sticky end, and AarI which recognizes CACCTGC and leaves 4 bp sticky end. AarI also requires addition of oligonucleotides for complete digestion, which is undesirable for DNA assembly.

Existing type IIS restriction enzyme-based DNA assembly systems are also designed for modular DNA assembly which leave unwanted scar sequences in the final assembled DNA, and cannot be used to assemble arbitrary DNA sequence without making custom assembly vectors.

US20160002644 describes a system of DNA assembly. It uses two restriction enzymes LguI and Earl, and a strain with inducible expression of M.TaqI to block overlapping restriction sites in the assembly vector. The M.TaqI site overlaps with the 3 bp adhesive end sequence generated by LguI/Earl, which leaves severe constraints on adaptor sequence design.

What is required is an improved DNA assembly method which provides one or more of a greater freedom to choose and design adapter sequences, assembly with minimal scarring, less reaction steps or complexity, and the ability to assemble larger sequences of DNA. Therefore, an aim of the present invention is to provide an improved DNA assembly method and associated material.

### SUMMARY OF THE DISCLOSURE

According to a first aspect of the invention, there is provided a nucleic acid for use in DNA assembly, wherein the nucleic acid comprises at least one methylation-protectable restriction element, the methylation-protectable restriction element comprising:
a restriction enzyme recognition sequence that is recognised by a restriction enzyme that cleaves outside of the recognition sequence; and
a DNA methylase recognition sequence,
wherein the restriction enzyme recognition sequence and the DNA methylase recognition sequence overlap such that the base modified by the DNA methylase lies within the restriction enzyme recognition sequence,
wherein the DNA methylase recognition sequence is not identical to, or enclosed by, the restriction enzyme recognition sequence, and
wherein the DNA methylase recognition sequence does not overlap with the sequence that would form the overhang end sequence generated by the restriction enzyme.

The invention herein can advantageously allow for the use of a single restriction enzyme throughout different stages of DNA assembly. This reduces the cost and complexity of preparing a starting part of DNA fragment to be free of the restriction enzyme used. As the methylase recognition sequence does not overlap with the sequence that would form the overhang end sequence generated by the restriction enzyme, the invention further provides more freedom to design adapters (i.e. overhangs) for DNA fragment assembly. The invention also provides the first hierarchical scarless DNA assembly system that uses restriction enzyme-based assembly method. Unlike existing modular DNA assembly methods, which leave unwanted scar sequences in the final assembled DNA, the present invention can be used to assemble large and arbitrary DNA sequences without scarring. Existing scarless assembly systems, all of which are based on homology-based assembly method, such as Gibson assembly and yeast homologous recombination, are unable to assemble large DNA constructs with repetitive DNA sequences, in contrast to the present invention. The method of the invention also allows a simple design for hierarchical scarless assembly of DNA using a universal set of 6 plasmids.

The methylation of the cut control element may block/impair the overlapping restriction enzyme recognition site.

In one embodiment, the restriction enzyme that cleaves outside its recognition sequence is a type IIS restriction enzyme.

In one embodiment, the nucleic acid comprises at least two methylation-protectable restriction elements. In another embodiment, the nucleic acid has two methylation-protectable restriction elements. In an embodiment comprising two methylation-protectable restriction elements, the nucleic acid sequence between the cut sites of the two methylation-protectable restriction elements may be a discard sequence (i.e. an unwanted fragment that will be removed from the nucleic acid during a restriction reaction). In an alternative embodiment, the discard sequence may be previously removed. For example, the nucleic acid may be a previously-cut linearized vector having a methylation-protectable restriction element at each end.

The discard sequence may comprise a selectable marker, reporter gene, or label. The skilled person will be familiar with typical markers and reporter genes used in DNA assembly and cloning. For example, a selectable marker may comprise a gene encoding an antibiotic resistance, an enzymatic activity, a luciferase, or the like. The marker could be a label, such as a radiolabel. In one embodiment, the discard sequence encodes lacZ as a reporter. The skilled person will recognise that discard sequence, that may be replaced by the assembled sequence in a vector, may not contain any functional element at all, as the process of the invention favours generating assembled DNA.

Providing a selectable marker, reporter gene, or label on the discard sequence advantageously allows clones to be selected that have had the discard sequence successfully removed or replaced by a fragment/sequence of interest.

In one embodiment, an opposing non-protectable restriction enzyme recognition sequence is provided on the opposing side of the cut site of the methylation-protectable restriction element. For example, in an embodiment comprising a discard sequence, the opposing non-protectable restriction enzyme recognition sequence would be located in the discard sequence. The opposing non-protectable restriction enzyme recognition sequence may also be recognized by a restriction enzyme that cleaves outside its recognition sequence, such as a type IIS restriction enzyme. The opposing non-protectable restriction enzyme recognition sequence may not be protected by (or arranged to be protected by) methylation, or at least may not be protected by methylation from a methylase that recognizes the methylase recognition sequence of the methylation-protectable restriction element. Such an opposing non-protectable restriction enzyme recognition sequence in the discard sequence may otherwise be referred to as an “inner restriction enzyme recognition sequence”, whereas the restriction enzyme recognition sequence of the methylation-protectable restriction element may be referred to as an “outer restriction enzyme recognition sequence”.

The restriction enzyme recognition sequence of the methylation-protectable restriction element may be recognised by the same restriction enzyme species as the opposing non-protectable restriction enzyme recognition sequence. The restriction enzyme recognition sequence of the methylation-protectable restriction element may comprise the same sequence as the opposing non-protectable restriction enzyme recognition sequence.

In one embodiment, the nucleic acid may comprise a “maintained composite element”, wherein the opposing non-protectable restriction enzyme recognition sequence may be arranged to direct the restriction enzyme to cut the nucleic acid at the same site as the restriction enzyme recognition sequence of the methylation-protectable restriction element, such that the same overhang/sticky end would be produced at the same position. This may be achieved by providing the restriction enzyme recognition sequence and opposing non-protectable restriction enzyme recognition sequence at a specific distance apart, depending on the cut site provided by the restriction enzyme(s) selected. In this embodiment, the sequence of the overhang produced by the restriction enzyme cutting the nucleic acid according to the restriction enzyme recognition sequence of the methylation-protectable restriction element is maintained after cutting the nucleic acid with the restriction enzyme that recognizes the opposing non-protectable restriction enzyme recognition sequence of the discard sequence and a subsequent ligation with a DNA fragment insert. As such, this embodiment may be referred to as the “maintained composite element”. i.e. the maintained composite element may comprise two restriction sequences arranged in head-to-head direction (one of which is part of the methylation protectable element, and the other the opposing non-protectable restriction enzyme recognition sequence). The overhang produced from a maintained composite element may be pre-determined by the sequence between the opposing restriction recognition sequences.

In an alternative embodiment to the maintained composite element, there is provided a “truncating composite element”, wherein the distance between the methylation-protectable restriction element and the opposing non-protectable restriction enzyme recognition sequence may be reduced such that the opposing non-protectable restriction enzyme recognition sequence may be arranged to direct the restriction enzyme to cut the nucleic acid at a site that does not overlap the cut site of the restriction enzyme that recognizes the restriction enzyme recognition sequence of the methylation-protectable restriction element (i.e. such that the position of the subsequent overhangs created by the opposing cut sites do not overlap, and the subsequent overhangs may be different in sequence). The cut site of the opposing non-protectable restriction enzyme recognition sequence in a truncating composite element may be within the recognition sequence of the restriction enzyme recognition sequence of the methylation-protectable restriction element, and/or within the nucleotides between the cut site and the recognition sequence of the restriction enzyme recognition sequence of the methylation-protectable restriction element. In one embodiment, the cleavage point between base pairs at the end of one cut site and the cleavage point between base pairs at the start of the opposing cut site may be in adjacent/neighbouring base pairs (i.e. the cut sites may be immediately adjacent to each other, but do not overlap). This may be achieved by providing the restriction enzyme recognition sequence of the methylation-protectable restriction element and opposing non-protectable restriction enzyme recognition sequence at a specific distance apart, depending on the cut site provided by the restriction enzyme(s) selected. The distance will be close enough to cause cutting in the opposing non-protectable restriction enzyme recognition sequence and/or nucleotides between the recognition sequence and cut site, or vice versa (i.e. closer than the opposing restriction enzyme recognition sequences in the “maintained composite element” for an equivalent/same selected restriction enzyme(s)). In this truncating composite element embodiment, the sequence of the overhang produced by the restriction enzyme cutting the nucleic acid according to the restriction enzyme recognition sequence of the methylation-protectable restriction element may or may not be maintained after cutting the nucleic acid with the restriction enzyme that recognizes the opposing non-protectable restriction enzyme recognition sequence of the discard sequence and a subsequent ligation with a DNA fragment insert, whereby the sequence of the DNA fragment insert would dictate the sequence of the overhang (e.g. the overhang produced from a truncating composite element may not be pre-determined by the sequence between the opposing restriction enzyme recognition sites).

In an alternative embodiment to the maintained composite element and truncated composite element, there is provided an “insertional composite element”, wherein a sequence insert is provided between the methylation-protectable restriction element and the opposing non-protectable restriction enzyme recognition sequence. The sequence insert may comprise a functional sequence such that it provides a function. For example, the sequence insert may comprise a promoter sequence or a terminator sequence. In an embodiment comprising an insertional composite element the methylation-protectable restriction element and the opposing non-protectable restriction enzyme recognition sequence may not overlap and may be distanced apart by the sequence insert. The sequence insert may be any suitable length. For example, the distance between the methylation-protectable restriction element and the opposing non-protectable restriction enzyme recognition sequence may be any number of nucleotides as required for the sequence insert, such as a functional sequence insert. The distance between the restriction enzyme recognition sequence within the methylation-protectable restriction element and the opposing non-protectable restriction enzyme recognition sequence may range between 6 bp to 300 kb.

Advantageously, following DNA assembly, the functional sequence will be added into the 5′ or 3′ end of the assembled DNA. This design is convenient for adding common elements to an assembled plasmid by using specialized vectors (for example adding promoter and terminators in a multi-part coding region gene assembly reaction).

In one embodiment, for the “maintained composite element”, the opposing non-protectable restriction enzyme recognition sequence (such as a BsaI sequence) may be distanced apart from the restriction enzyme recognition sequence (such as a BsaI sequence) of the methylation-protectable restriction element by 6 base pairs (i.e. 6 bp in the gap between the end of one recognition sequence and the start of the opposing recognition sequence). The skilled person will recognise that such distance depends on the property of the restriction enzyme(s) selected. For example, assuming the enzyme cuts x bases from the recognition sequence and generates y bases of adhesive end/overhang (e.g. for BsaI x=1 and y=4), then the distance (d) for the number of bases between opposing restriction enzyme recognition sequences of the maintained composite element may be provided by the following equation: d=(2*x)+y (e.g. d=6 bp for BsaI). The distance (d) for the maintained composite element may range from 5 bp (e.g. when using Earl) to 24 bp (e.g. when using BtgZI). In one embodiment, the difference between the opposing restriction enzyme recognition sequences of the maintained composite element is d, wherein d=(2*x)+y, and wherein x is the number of base pairs the cut site of a given restriction enzyme is from its recognition sequence, and y is the length of the overhang that would be produced by the restriction enzyme.

In the alternative truncating composite element embodiment, for example where the overhang produced by the restriction enzyme recognition sequence of the methylation-protectable restriction element may or may not be maintained, the opposing non-protectable restriction enzyme recognition sequence (such as for BsaI) may be distanced apart from the restriction enzyme recognition sequence (such as for BsaI) of the methylation-protectable restriction element by 2 base pairs. As above, the skilled person will recognise that such distance depends on the property of the restriction enzyme(s) selected. For example, assuming the enzyme cuts x bases from the recognition sequence and generates y bases adhesive end (for BsaI x=1 and y=4), the distance (d) for the number of bases between opposing restriction enzyme recognition sequences of the truncating composite element may be provided by the following equation: d=2*x (e.g. d=2 bp for BsaI). The distance (d) for the truncating composite element may range from 2 bp (e.g. when using BsaI) to 20 bp (e.g. when using BtgZI). In one embodiment, the difference between the opposing restriction enzyme recognition sequences of the truncating composite element is d, wherein d=2*x, and wherein x is the number of base pairs the cut site of a given restriction enzyme is from its recognition sequence.

Advantageously, the provision of a maintained composite element facilitates one-pot DNA assembly, whereby multiple fragments of DNA may be assembled in a single one-pot reaction with a single restriction enzyme species. Advantageously, the provision of a truncating composite element facilitates scarless DNA assembly, whereby the overhang ends of a particular DNA fragment insert can be pre-determined to be adapted for ligation into a destination vector or provided in accordance with the native sequence of the DNA fragment of interest for joining with neighbouring fragments in the sequence.

In one embodiment, the nucleic acid may comprise a circular nucleic acid, such as a vector, comprising two maintained composite elements with a discard sequence therebetween, or a linearized version thereof with the discard sequence cut out. In another embodiment, the nucleic acid may comprise a circular nucleic acid, such as a vector, comprising a maintained composite element and a truncating composite element with a discard sequence therebetween, or a linearized version thereof with the discard sequence cut out. In another embodiment, the nucleic acid may comprise a circular nucleic acid, such as a vector, comprising two truncating composite elements with a discard sequence therebetween, or a linearized version thereof with the discard sequence cut out. In another embodiment, the nucleic acid may comprise a circular nucleic acid, such as a vector, comprising an insertional composite element and a truncating composite element with a discard sequence therebetween, or a linearized version thereof with the discard sequence cut out. In another embodiment, the nucleic acid may comprise a circular nucleic acid, such as a vector, comprising an insertional composite element and a maintained composite element with a discard sequence therebetween, or a linearized version thereof with the discard sequence cut out. In another embodiment, the nucleic acid may comprise a circular nucleic acid, such as a vector, comprising two insertional composite elements with a discard sequence therebetween, or a linearized version thereof with the discard sequence cut out. In linearized nucleic acid embodiments, the discard sequence may have been cut out by restriction with the restriction enzyme that recognises the opposing non-protectable restriction enzyme sequence present on the discard sequence (i.e. the inner restriction enzyme recognition sequence).

In embodiments comprising two maintained composite elements, the restriction enzyme recognition sequences of the two maintained composite elements may be the same and/or the methylase recognition sequences of the two maintained composite elements may be the same. In embodiments comprising two truncated composite elements, the restriction enzyme recognition sequences of the two truncated composite elements may be the same and/or the methylase recognition sequences of the two truncated composite elements may be the same. In embodiments comprising two insertional composite elements, the restriction enzyme recognition sequences of the two insertional composite elements may be the same and/or the methylase recognition sequences of the two insertional composite elements may be the same. In embodiments comprising a maintained composite element and a truncated composite element, the restriction enzyme recognition sequences of the maintained and truncated composite elements may be the same and/or the methylase recognition sequences of the maintained and truncated composite elements may be the same. In embodiments comprising a maintained composite element and an insertional composite element, the restriction enzyme recognition sequences of the maintained and insertional composite elements may be the same and/or the methylase recognition sequences of the maintained and insertional composite elements may be the same. In embodiments comprising a truncated composite element and an insertional composite element, the restriction enzyme recognition sequences of the truncated and insertional composite elements may be the same and/or the methylase recognition sequences of the truncated and insertional composite elements may be the same. For example, digestion of the nucleic acid to remove a discard sequence or DNA fragment of interest may be carried out by the same restriction enzyme. Additionally the methylation of the nucleic acid to protect all the methylase-protectable restriction enzyme recognition sites in the nucleic acid may be carried out by the same methylase.

In one embodiment, the methylase comprises a type II DNA methylase. In another embodiment, the methylase comprises a type I DNA methylase. In one embodiment, the methylase comprises a single domain methylase without restriction enzyme activity, or a modified methylase having a non-functional restriction enzyme activity. For example, the modification may comprise modifying the N6 position of methyladenine. In one embodiment, the DNA methylase recognition sequence comprises at least 4 base pairs. The DNA methylase may be active, such as optimally active, at about 37° C.

In one embodiment, the opposing non-protectable restriction enzyme recognition sequence is not capable of being blocked by methylation with the methylase that recognises the DNA methylase recognition sequence of the methylation-protectable restriction element. In one embodiment, the methylase that recognises the DNA methylase recognition sequence of the methylation-protectable restriction element may not recognise a sequence within or overlapping with the opposing non-protectable restriction enzyme recognition sequence. For example, the non-protectable opposing restriction enzyme recognition sequence may not overlap with or comprise a sequence that is recognised by the methylase that recognises DNA methylase recognition sequence of the methylation-protectable restriction element. The restriction enzyme recognition sequence of the methylation-protectable restriction element may be methylated, for example by the methylase that recognises the DNA methylase recognition sequence of the methylation-protectable restriction element. The methylase that recognises the DNA methylase recognition sequence of the methylation-protectable restriction element may be arranged to methylate a nucleotide within the sequence of the restriction enzyme recognition sequence of the methylation-protectable restriction element. The methylation may be capable of blocking the cutting of the DNA by the restriction enzyme that recognises the restriction enzyme recognition sequence of the methylation-protectable restriction element. In one embodiment, the restriction enzyme recognition sequence of the methylation-protectable restriction element may become non-functional as a result of methylation of at least one of the nucleotides in the sequence. In one embodiment, the methylated nucleotide may be an adenine, or the nucleotide arranged to be methylated may be an adenine. In another embodiment, the methylated nucleotide may be a cytosine, or the nucleotide arranged to be methylated may be a cytosine. The methylation may be any one of methylation types selected from N4-methylcytosine (m4C), C5-methyylcytosine (m5C) or N6-methyladenine (m6A). The skilled person will recognize that the type of methylation provided should block/impair the overlapping restriction site function. Additionally, for in vivo methylation, the strain used to express the methylase may be deficient for modification-dependent restriction enzymes that may recognize the methylated bases, such as Mcr/Mrr family restriction enzymes. In one embodiment, the methylation type may comprise N6-methyladenine (m6A).

In one embodiment, the methylase may or may not methylate the outer-most bases of the restriction enzyme recognition sequence. For example in a restriction enzyme recognition sequence of 6 bp, the bases 1 or 6 may not be methylated. In one embodiment, the methylase may or may not methylate the 2^{nd} base of the restriction enzyme recognition sequence. In one embodiment, the methylase may methylate position 3, 4, or 5 for 6 bp restriction enzyme recognition sites. In one embodiment, the methylase may methylate position 3, 4, 5, or 6 for restriction enzyme recognition sites.

In an embodiment comprising two methylation-protectable restriction elements, the DNA methylase recognition sequences of each methylation-protectable restriction element may be recognised by the same methylase. The DNA methylase recognition sequence of the methylation-protectable restriction elements may comprise or consist of the same sequence.

In an embodiment comprising two methylation-protectable restriction elements, the restriction enzyme recognition sequences of each methylation-protectable restriction element may be recognised by the same restriction enzyme species. Each restriction enzyme recognition sequence of the methylation-protectable restriction elements may comprise or consist of the same sequence. The two methylation-protectable restriction elements may comprise or consist of the same sequence.

The use of the same restriction enzyme recognition sequence advantageously provides that a single restriction enzyme species can be used in a DNA assembly reaction. The use of a single restriction enzyme species opens the possibility of less complex DNA assembly, because the presence of further restriction enzyme species in a reaction significantly increases the probability the more that the sequence will randomly/inadvertently contain a recognition and restriction cut site, causing a failure in the cloning procedure.

In one embodiment, the methylation-protectable restriction element comprises or consists of a sequence according to any one of the overlapping methylation/restriction sites identified in Table 3 herein. In an alternative embodiment, the methylation-protectable restriction element comprises or consists of a sequence according to any one of the overlapping methylation/restriction sites identified in Table 3 herein, excluding those which provide methylation of the first or last base in the restriction sequence. In one embodiment, the methylation-protectable restriction element comprises or consists of the sequence GACNNGGTCTCNNNNN (BsaI/M.Osp807II—SEQ ID NO: 1). In another embodiment, the methylation-protectable restriction element comprises or consists of the sequence GAAGACGCNNNNNN (BpiI/M2.NmeMC58II—SEQ ID NO: 2). In another embodiment, the methylation-protectable restriction element comprises or consists of the sequence GAAGCTCTTCNNNN (LguI/M.XmnI—SEQ ID NO: 3).

In one embodiment, the methylation-protectable and/or non-protectable restriction enzyme recognition sequence comprises or consists of a sequence according to any one of the restriction enzyme recognition sequences identified in Table 3 herein. In an alternative embodiment, the methylation-protectable and/or non-protectable restriction enzyme recognition sequence comprises or consists of a sequence according to any one of the restriction enzyme recognition sequences identified in Table 3 herein, excluding those which provide for methylation of the first or last base in the restriction sequence. In one embodiment, the methylation-protectable and/or non-protectable restriction enzyme recognition sequence comprises or consists of the sequence GGTCTC (BsaI-SEQ ID NO: 4). In another embodiment, the methylation-protectable and/or non-protectable restriction enzyme recognition sequence comprises or consists of a sequence GAAGAC (BpiI—SEQ ID NO: 5). In another embodiment, the methylation-protectable and/or non-protectable restriction enzyme recognition sequence comprises or consists of a sequence GCTCTTC (LguI—SEQ ID NO: 6).

In one embodiment, the DNA methylase recognition sequence comprises or consists of a sequence according to any one of the type II DNA methylase recognition sequences identified in Table 3 herein. In an alternative embodiment, the DNA methylase recognition sequence comprises or consists of a sequence according to any one of the type II DNA methylase recognition sequences identified in Table 3 herein, excluding those which provide methylation of the first or last base in the restriction sequence. In one embodiment, the DNA methylase recognition sequence comprises or consists of the sequence GACNNNGTC (M.Osp807II—SEQ ID NO: 7). In another embodiment, the DNA methylase recognition sequence comprises or consists of the sequence GACGC (M2.NmeMC58II—SEQ ID NO: 8). In another embodiment, the DNA methylase recognition sequence comprises or consists of the sequence GAANNNNTTC (M.XmnI—SEQ ID NO: 9).

The restriction enzyme that recognizes the restriction enzyme recognition sequence of the methylation-protectable restriction element, and/or the opposing restriction enzyme recognition sequence, may be capable of cutting nucleic acid to leave at least a 2 bp overhang/sticky end. Alternatively, the restriction enzyme that recognizes the restriction enzyme recognition sequence of the methylation-protectable restriction element, and/or the opposing restriction enzyme recognition sequence, may be capable of cutting nucleic acid to leave at least a 3 bp overhang/sticky end. In another embodiment, the restriction enzyme that recognizes the restriction enzyme recognition sequence of the methylation-protectable restriction element, and/or the opposing restriction enzyme recognition sequence, may be capable of cutting nucleic acid to leave a 3 bp overhang/sticky end. In another embodiment, the restriction enzyme that recognizes the restriction enzyme recognition sequence of the methylation-protectable restriction element, and/or the opposing restriction enzyme recognition sequence, may be capable of cutting nucleic acid to leave a 4 bp overhang/sticky end. In another embodiment, the restriction enzyme that recognizes the restriction enzyme recognition sequence of the methylation-protectable restriction element, and/or the opposing restriction enzyme recognition sequence, may be capable of cutting nucleic acid to leave a 2 bp to 6 bp overhang/sticky end. In another embodiment, the restriction enzyme that recognizes the restriction enzyme recognition sequence of the methylation-protectable restriction element, and/or the opposing restriction enzyme recognition sequence, may be capable of cutting nucleic acid to leave a 2 bp to 5 bp overhang/sticky end. In another embodiment, the restriction enzyme that recognizes the restriction enzyme recognition sequence of the methylation-protectable restriction element, and/or the opposing restriction enzyme recognition sequence, may be capable of cutting nucleic acid to leave a 3 bp to 5 bp overhang/sticky end. In another embodiment, the restriction enzyme that recognizes the restriction enzyme recognition sequence of the methylation-protectable restriction element, and/or the opposing restriction enzyme recognition sequence, may be capable of cutting nucleic acid to leave a 4 bp to 5 bp overhang/sticky end.

In one embodiment, the restriction enzyme comprises or consists of any one type IIS restriction enzyme identified in Table 3 herein. In another embodiment, the type IIS restriction enzyme comprises or consists of BsaI, BpiI, or LguI.

In one embodiment, the DNA methylase comprises or consists of any one type II DNA methylase identified in Table 3 herein. In an alternative embodiment, the DNA methylase comprises or consists of any one type II DNA methylase identified in Table 3 herein, excluding those which provide methylation of the first or last base in the restriction sequence. In another embodiment, the DNA methylase comprises or consists of M.Osp807II, M2. NmeMC58II, or M.XmnI.

In one embodiment, the restriction enzyme and the DNA methylase comprise or consist of any one pairing of type IIS restriction enzymes and the type II DNA methylases identified in Table 3 herein. In one embodiment, the restriction enzyme and the DNA methylase comprise or consist of the pairings BsaI/M.Osp807II, BpiI/M2.NmeMC58II, or LguI/M.XmnI.

In one embodiment, the restriction enzyme recognition sequence and the DNA methylase recognition sequence comprise or consist of any one pairing of type IIS restriction enzyme recognition sequences and the type II DNA methylase recognition sequences identified in Table 3 herein. In an alternative embodiment, the restriction enzyme recognition sequence and the DNA methylase recognition sequence comprise or consist of any one pairing of type IIS restriction enzyme recognition sequences and the type II DNA methylase recognition sequences identified in Table 3 herein, excluding those which provide methylation of the first or last base in the restriction sequence.

In an embodiment comprising two methylation-protectable restriction elements (for example with a discard sequence therebetween), the overhang/sticky end produced by cutting with the restriction enzyme at the first methylation-protectable restriction element may be different in sequence than the overhang/sticky end produced by cutting with the restriction enzyme at the second methylation-protectable restriction element. The skilled person will be familiar with providing different overhangs for directional DNA assembly/cloning or controlling which end of the nucleic acid is ligated to an insert/fragment of interest.

In one embodiment involving a maintained composite element, the nucleic acid may comprise or consist of the sequence of GACNNGGTCTCNNNNNNGAGACC (SEQ ID NO: 10). In another embodiment involving a maintained composite element, the nucleic acid may comprise or consist of the sequence of GAAGACGCNNNNNNGTCTTC (SEQ ID NO: 11). In another embodiment involving a maintained composite element, the nucleic acid may comprise or consist of the sequence of GAAGCTCTTCNNNNNGAAGAGC (SEQ ID NO: 12). N may mean A, C, T or G. Other sequences may be provided according to the combined/overlapping restriction enzyme and methylase recognition sequences provided in table 3, wherein following the series of N bases, the sequence further comprises the palindromic/reverse-complementary sequence of the restriction enzyme recognition sequence.

In one embodiment involving two maintained composite elements, the nucleic acid may comprise or consist of the sequence of GACNNGGTCTCNNNNNNGAGACC(N^{x})GGTCTCNNNNNNGAGACCNNGTC (SEQ ID NO: 13). In another embodiment involving two maintained composite elements, the nucleic acid may comprise or consist of the sequence of GAAGACGCNNNNNNGTCTTC(N^{x})GAAGACNNNNNNGCGTCTTC (SEQ ID NO: 14). In another embodiment involving two maintained composite elements, the nucleic acid consist of may comprise or the sequence of GAAGCTCTTCNNNNNGAAGAGC(N^{x})GCTCTTCNNNNNGAAGAGCTTC (SEQ ID NO: 15). N may mean A, C, T or G. (N^{x}) may mean any number of A, C, T or G, or between 0 bp and 300 kbp of A, C, T, or G.

The skilled person will understand that for some restriction enzyme recognition sequences, such as for LguI, the number of nucleotides (N^{x}) between two maintained composite elements could be negative with the two inner nonprotectable restriction enzyme recognition sites overlapping. Therefore, in another embodiment involving two maintained composite elements, the nucleic acid may comprise or consist of the sequence of GAAGCTCTTCNNNNNGAAGAGCTCTTCNNNNNGAAGAGCTTC (SEQ ID NO: 16) (the overlapping nucleotides are shown in bold and underlined).

In one embodiment involving a truncated composite element, the nucleic acid may comprise or consist of the sequence of GACNNGGTCTCNNGAGACC (SEQ ID NO: 17). In one embodiment involving a truncated composite element, the nucleic acid may comprise or consist of the sequence of GAAGCTCTTCNNGAAGAGC (SEQ ID NO: 18). N may mean A, C, T or G. N may mean A, C, T or G. Other sequences may be provided according to the combined/overlapping restriction enzyme and methylase recognition sequences provided in table 3, wherein following the series of N bases, the sequence further comprises the palindromic/reverse-complementary sequence of the restriction enzyme recognition sequence, and wherein the number of N bases between the restriction recognition sequences is reduced by the number overhang bases produced by the selected restriction enzyme.

In one embodiment involving a maintained and truncated composite element, the nucleic acid may comprise or consist of the sequence of GACNNGGTCTCNNNNNNGAGACC(N^{x})GGTCTCNNGAGACCNNGTC (SEQ ID NO: 19). In one embodiment involving a maintained and truncated composite element, the nucleic acid comprise or consist of may the sequence of GAAGCTCTTCNNNNNGAAGAGC(N^{x})GCTCTTCNNGAAGAGCTTC (SEQ ID NO: 20). N may mean A, C, T or G. (N^{x}) may mean any number of A, C, T or G, or between 0 bp and 300 kbp of A, C, T, or G.

The skilled person will understand that for some restriction enzyme recognition sequences, such as for LguI, the number of nucleotides (N^{x}) between a maintained and truncated composite element could be negative with the two inner nonprotectable restriction enzyme recognition sites overlapping. Therefore, in another embodiment involving a maintained and truncated composite element, the nucleic acid may comprise or consist of the sequence of GAAGCTCTTCNNNNNGAAGAGCTCTTCNNGAAGAGCTTC (SEQ ID NO: 21) (the overlapping nucleotides are shown in bold and underlined).

In one embodiment involving a maintained and truncated composite element, the nucleic acid may comprise or consist of the sequence of GACNNGGTCTCNNGAGACC(N^{x})GGTCTCNNNNNNGAGACCNNGTC (SEQ ID NO: 22). In one embodiment involving a maintained and truncated composite element, the nucleic acid may comprise or consist of the sequence of GAAGCTCTTCNNGAAGAGC(N^{x})GCTCTTCNNNNNGAAGAGCTTC (SEQ ID NO: 23). N may mean A, C, T or G. (N^{x}) may mean any number of A, C, T or G, or between 0 bp and 300 kbp of A, C, T, or G. In another embodiment involving a maintained and truncated composite element, the nucleic acid may comprise or consist of the sequence of GAAGCTCTTCNNGAAGAGCTCTTCNNNNNGAAGAGCTTC (SEQ ID NO: 24) (the overlapping nucleotides are shown in bold and underlined).

In one embodiment involving two truncated composite elements, the nucleic acid may comprise or consist of the sequence of GACNNGGTCTCNNGAGACC(N^{x})GGTCTCNNGAGACCNNGTC (SEQ ID NO: 25). In one embodiment involving two truncated composite elements, the nucleic acid may comprise or consist of the sequence of GAAGCTCTTCNNGAAGAGC(N)GCTCTTCNNGAAGAGCTTC (SEQ ID NO: 26). N may mean A, C, T or G. (N^{x}) may mean any number of A, C, T or G, or between 0 bp and 300 kbp of A, C, T, or G.

The skilled person will understand that for some restriction enzyme recognition sequences, such as for LguI, the number of nucleotides (N^{x}) between two truncated composite elements could be negative with the two inner nonprotectable restriction enzyme recognition sites overlapping. Therefore, in another embodiment involving two truncated composite elements, the nucleic acid may comprise or consist of the sequence of GAAGCTCTTCNNGAAGAGCTCTTCNNGAAGAGCTTC (SEQ ID NO: 27) (the overlapping nucleotides are shown in bold and underlined).

The nucleic acid may be isolated nucleic acid. In one embodiment, the nucleic acid may be synthetic (i.e. not found in nature). In one embodiment, the nucleic acid may be isolated or derived from a bacterial strain, such as E. coli, that expresses a DNA methylase that recognises (and methylates) the methylase recognition sequence of the methylation-protectable restriction element. For example, a bacterial strain, such as E. coli, that expresses any one type II DNA methylase identified in Table 3 herein. In one embodiment the nucleic acid may be isolated or derived from a bacterial strain, such as E. coli, that expresses M.Osp807II, M2.NmeMC58II, or M.XmnI. The expressed DNA methylase may be functional. The bacterial strain may be genetically modified/engineered to express such DNA methylase.

The nucleic acid may be a vector, such as a cloning vector. The skilled person will be familiar with various cloning vectors, which may include, for example, a replication origin, a selectable marker, a reporter gene, expression elements, or combinations thereof. The vector may comprise nucleic acid that comprises a DNA element that supports the replication of DNA that contains it (e.g. a replication origin) and a selection marker (e.g. an antibiotic selection marker). The vector may be a high or low copy number vector. In one embodiment, the vector is a high copy number vector. The skilled person will be familiar with cloning methods and materials/vectors, for example as provided in Green, M. R., Sambrook, J. Molecular Cloning: A Laboratory Manual. 4th. Cold Spring Harbor Laboratory Press, Cold Spring Harbor, NY; 2012, which is herein incorporated by reference.

In linearized embodiments of the nucleic acid, the nucleic acid may not comprise more than two copies/sites of the restriction enzyme recognition sequence.

According to another aspect of the invention, there is provided a method of assembling DNA comprising:
providing a linearised methylated nucleic acid according to the invention, wherein the methylation-protectable restriction element is methylated with the DNA methylase;
providing two or more DNA fragments of interest for assembly with the linearised methylated nucleic acid, wherein a first DNA fragment comprises a complementary overhang for ligation with a first end of the linearised methylated nucleic acid, and a second DNA fragment comprises complementary overhangs for ligation with the other/second end of the linearised methylated nucleic acid; and
(i) the first and second DNA fragments further comprise complementary overhangs for ligation with each other; or
(ii) the first and second DNA fragments further comprise complementary overhangs for ligation with one or more further DNA fragments having complementary overhangs, such that ligating the DNA fragments and the linearised methylated nucleic acid with a DNA ligase would result in a single assembled DNA molecule;
ligating the DNA fragments and linearised methylated nucleic acid with a ligase to form a single assembled DNA molecule comprising the sequence of the assembled DNA fragments flanked by the restriction enzyme recognition sequences of the methylation-protectable restriction elements.

The single assembled DNA molecule may be circular, such as a circular DNA vector.

Advantageously, further cutting of the single assembled DNA molecule by the restriction enzyme that recognises the restriction enzyme recognition sequences of the methylation-protectable restriction elements is blocked by the methylation of the restriction enzyme recognition sequences.

The linearised nucleic acid may be provided by providing a nucleic acid according to the invention in the form of a circular destination vector comprising two methylated methylation-protectable restriction elements and a discard sequence therebetween, wherein the method comprises the step of cutting the circular destination vector with restriction enzymes that recognise the opposing non-protectable restriction enzyme recognition sequences in the discard sequence, thereby leaving a linearised nucleic acid having overhangs defined by the restriction enzymes. The restriction enzymes may be the same species. The restriction enzyme recognition sequences of the methylated methylation-protectable restriction elements may comprise the same sequence. In one embodiment, the restriction enzyme recognition sequences of the methylated methylation-protectable restriction elements and the opposing non-protectable restriction enzyme recognition sequences may comprise the same sequence.

The restriction enzyme and methylase (or recognition sequences thereof) used in the method of the invention may be selected in accordance with the restriction enzyme and methylases (or recognition sequences thereof) provided for the nucleic acid aspect of the present invention herein.

The circular destination vector may be purified/isolated from a bacterial strain that expresses the DNA methylase that recognises the DNA methylase recognition sequence of the methylation-protectable restriction element. In another embodiment, the circular or linearised destination vector may be methylated by the DNA methylase in vitro.

The two or more DNA fragments of interest for assembly with the nucleic acid may be provided in circular donation vectors, wherein the method comprises the step of cutting the circular donation vectors to release the DNA fragments of interest. The cutting of the donor vectors may be with a restriction enzyme that leaves complementary overhangs for ligation with the linearised methylated nucleic acid. The cutting of the donor vectors may be with a restriction enzyme that leaves complementary overhangs for ligation with the linearised methylated nucleic acid and with another DNA fragment of interest for insertion. The cutting of the donor vectors may be with a restriction enzyme that leaves complementary overhangs for ligation with two other DNA fragments of interest for insertion.

The restriction enzyme for cutting the circular donation vectors may comprise a type IIS restriction enzyme. The restriction enzyme for cutting the circular donation vectors may comprise the same restriction enzyme, or a restriction enzyme that recognises the same sequence, as the restriction enzyme used to cut the circular destination vector. The complementary overhangs of the DNA fragments of interest for ligation with the linearised nucleic acid may be defined by the restriction enzyme.

Advantageously, the provision of the same restriction enzyme recognition sequence for the methylation-protectable restriction elements, the opposing restriction enzyme recognition sequence on the discard sequence, and the restriction enzyme recognition sequences on the donor vectors provides that a single type of restriction enzyme can be used. This allows for reduced cost/complexity in removing internal type IIS restriction site from DNA parts, because there are less restriction sites to remove. This makes assembly of longer sequence easier and less costly. Furthermore, the invention allows for a so-called “one-pot” reaction, whereby the restriction and ligation can be carried out in one pot in a single step.

Therefore, the method may combine the steps of restricting and ligating. In particular, the circular destination vector, the donor vectors, the restriction enzyme and the ligase may be provided in the same composition.

Following assembly of the single assembled DNA molecule, the method may further comprise the step of transforming the single assembled DNA molecule into a bacterial strain that does not express the DNA methylase that recognises the DNA methylase recognition sequence of the methylation-protectable restriction element.

Advantageously, transforming the single assembled DNA molecule into a bacterial strain that does not express the DNA methylase that recognises the DNA methylase recognition sequence of the methylation-protectable restriction element results in the cloning and production of a nucleic acid sequence, such as a vector, that comprises a non-methylated (i.e. non-blocked) restriction enzyme recognition sequence to allow subsequent restriction reactions.

The method may further comprise a multistage assembly process in order to form assembled DNA fragments or vectors of greater sequence length. For example the assembled DNA, which is the product of the method of the invention, may be used further for a higher order assembly. For example, the method may comprise a second round of DNA assembly according to the invention wherein the DNA fragments of interest for assembly with the linearised methylated nucleic acid may comprise assembled DNA from a first/earlier DNA assembly round according to the invention.

Providing multiple rounds of DNA assembly to form larger fragments of DNA advantageously reduces the number of adapter/overhang sequences that are necessary. The success rate of DNA assembly is also increased with fewer DNA fragments to be assembled. Additionally, it can be easier to screen for the successfully assembled DNA with fewer fragments assembled in a single reaction.

### Scarless DNA Assembly Method

According to another aspect of the present invention, there is provided a method of scarless DNA assembly of DNA fragments comprising the steps of:
(A) providing a first linearised methylated nucleic acid according to the invention, having a maintained composite element at one end and a truncating composite element at the other end, wherein the maintained and truncating composite elements are methylated with the DNA methylase;
providing a first DNA fragment for assembly having overhang ends that are adapted to ligate to the overhang ends of the first linearised methylated nucleic acid;
ligating the first DNA fragment for assembly and first linearised methylated nucleic acid with a ligase to form a first methylated intermediate vector;
transforming the first methylated intermediate vector into a bacterial strain that does not express the DNA methylase(s) that recognises either of the DNA methylase recognition sequence(s) of the maintained and truncating composite elements, thereby forming a first intermediate vector that is not methylated;
isolating the first intermediate vector;
(B) providing a second linearised methylated nucleic acid according to the invention, having a maintained composite element at one end and a truncating composite element at the other end, wherein the maintained and truncating composite elements are methylated with the DNA methylase, and wherein the overhang provided by the maintained composite element of the first linearised methylated nucleic acid is different in sequence to the overhang provided by the methylation-protectable restriction element of the second linearised methylated nucleic acid;
providing a second DNA fragment for assembly having overhang ends that are adapted to ligate to the overhang ends of the second linearised methylated nucleic acid;
ligating the second DNA fragment for assembly and second linearised methylated nucleic acid with a ligase to form a second methylated intermediate vector;
transforming the second methylated intermediate vector into a bacterial strain that does not express the DNA methylase(s) that recognises either of the DNA methylase recognition sequence(s) of the maintained and truncating composite elements, thereby forming a second intermediate vector that is not methylated;
isolating the second intermediate vector;
(C) cutting the first intermediate vector with a restriction enzyme that recognises the restriction enzyme recognition sequence of the maintained composite element and a restriction enzyme that recognises the restriction enzyme recognition sequence of the truncating composite element, thereby forming a first adapted DNA fragment insert that comprises a maintained-overhang sequence that is determined by the maintained composite element and an opposing native-overhang sequence that is determined by the native sequence of the first DNA fragment for assembly;
(D) cutting the second intermediate vector with a restriction enzyme that recognises the restriction enzyme recognition sequence of the maintained composite element and a restriction enzyme that recognises the restriction enzyme recognition sequence of the truncating composite element, thereby forming a second adapted DNA fragment insert that comprises a maintained-overhang sequence that is determined by the maintained composite element and an opposing native-overhang sequence that is determined by the native sequence of the second DNA fragment for assembly;
wherein
(i) the first and second adapted DNA fragments are end fragments wherein their native-overhang sequences are complementary, such that they are arranged to ligate together; or
(ii) one or more middle DNA fragments for assembly are provided wherein the first and second adapted DNA fragments are respective end fragments in the assembly, and the one or more middle DNA fragments are arranged to be ligated between the first and second adapted DNA fragments via complementary native-overhang sequences;
further comprising the step of ligating together, with a ligase, the first and second adapted DNA fragments, or the first and second adapted DNA fragments and one or more middle DNA fragments, to form an assembled DNA fragment having maintained-overhangs at each end.

The one or more middle DNA fragments may comprise native-overhang sequences at both ends that are determined by their native sequence. In an embodiment comprising a single middle DNA fragment for assembly, the middle DNA fragment may comprise a first native-overhang sequence that is complementary to the native-overhang of the first adapted DNA fragment and a second native-overhang sequence that is complementary to the native-overhang of the second adapted DNA fragment.

In an embodiment comprising a two or more middle DNA fragments for assembly, the middle DNA fragments may each comprise native-overhang sequences that are complementary to the native-overhang of a neighbouring middle DNA fragment, such that they can be ligated together in a pre-determined order. The first and last middle DNA fragments in a sequence will be arranged to ligate to the respective first adapted DNA fragment and second adapted DNA fragment via complementary native-overhang sequences.

In one embodiment, a middle DNA fragment for assembly may be provided by
providing a further linearised methylated nucleic acid according to the invention, wherein the linearised methylated nucleic acid comprises a truncating composite element at each end, wherein the truncating composite elements are methylated with the DNA methylase;
providing an adapted middle DNA fragment for assembly having overhang ends that are adapted to ligate to the overhang ends of the linearised methylated nucleic acid;
ligating the adapted middle DNA fragment for assembly and linearised methylated nucleic acid with a ligase to form a methylated intermediate vector;
transforming the methylated intermediate vector into a bacterial strain that does not express the DNA methylase(s) that recognises either of the DNA methylase recognition sequence(s) of the truncating composite elements, thereby forming an intermediate vector that is not methylated;
isolating the intermediate vector;
cutting the intermediate vector with restriction enzymes that recognise the restriction enzyme recognition sequence of the truncating composite elements, thereby forming a middle DNA fragment insert that comprises native-overhang sequences at each end that are determined by the native sequence of the middle DNA fragment for assembly.

The DNA fragments for assembly may comprise double stranded DNA comprising the overhangs/stick ends at each end. A DNA fragment for assembly may be prepared by PCR from a template, by gene synthesis, or by annealing of two oligonucleotides. Such processes, e.g. PCR or gene synthesis, may generate blunt ended double stranded DNA. For assembly starting with PCR product, an appropriate adaptor sequence containing the restriction site may be included in the PCR primer to trim the DNA to generate the overhangs for cloning into the intermediate vectors. For embodiments comprising the use of annealed oligonucleotides there are more choices. The sequence design could be the same as for PCR/gene synthesis with restriction sites at both ends for trimming with a restriction enzyme to generate the overhangs. Alternatively, two annealed oligos may form the overhangs themselves.

In one embodiment, the DNA fragments for assembly, such as the first DNA fragment for assembly, the second DNA fragment for assembly, and the one or more adapted middle DNA fragments for assembly, may be provided by PCR from a template sequence (i.e. the DNA fragments for assembly may be PCR products). The template sequence may provide the full sequence that is desired to be cloned as the assembled DNA fragment. The DNA fragments for assembly may be replicated, for example by PCR, from different sections of the template, and generated neighbouring PCR products may partially overlap in sequence (i.e. the sequence near the end of one PCR product may comprise the same sequence near the start of the next PCR product for assembly). The overlap may be partial, but sufficient in length to provide a complementary sticky end. In one embodiment, the overlap sequence within one PCR product (DNA fragment for assembly) and the overlap in the next neighbouring PCR product (DNA fragment for assembly) may be at least 2 base pairs in length. In another embodiment, the overlap sequence within one PCR product (DNA fragment for assembly) and the overlap in the next neighbouring PCR product (DNA fragment for assembly) may be at least 3 base pairs in length. In another embodiment, the overlap sequence within one PCR product (DNA fragment for assembly) and the overlap in the next neighbouring PCR product (DNA fragment for assembly) may be 3-6 base pairs in length. In another embodiment, the overlap sequence within one PCR product (DNA fragment for assembly) and the overlap in the next neighbouring PCR product (DNA fragment for assembly) may be 3-5 base pairs in length. In another embodiment, the overlap sequence within one PCR product (DNA fragment for assembly) and the overlap in the next neighbouring PCR product (DNA fragment for assembly) may be 4-5 base pairs in length. In another embodiment, the overlap sequence within one PCR product (DNA fragment for assembly) and the overlap in the next neighbouring PCR product (DNA fragment for assembly) may be 3-4 base pairs in length. In another embodiment, the overlap sequence within one PCR product (DNA fragment for assembly) and the overlap in the next neighbouring PCR product (DNA fragment for assembly) may be 4 base pairs in length.

PCR primers may be arranged to amplify the partially overlapping sections of template DNA in order to form the DNA fragments for assembly that would comprise complementary overhangs. The PCR reaction may add adaptor sequences containing the restriction recognition sequence (i.e. the universal adapter sequence, for example the restriction enzyme recognition sequence may comprise the same sequence as the restriction enzyme recognition sequence of the maintained composite element and/or truncating composite element) plus the sequence for the overhang that would be complementary to the overhang of the neighbouring/consecutive fragment for assembly. The overhang comprising the sequence that overlaps, for example by 4 bp, between consecutive/neighbouring fragments may be formed following digestion with the restriction enzyme (i.e. the overlap among PCR product is referring to the sequence, for example of 4 bp, that inside the whole adaptor sequence comprising the restriction enzyme recognition sequence).

The PCR primers may further provide the adapter sequences for the DNA fragments, which form the complementary overhang for ligation with the linearised methylated nucleic acids (such as the first linearised methylated nucleic acid, second linearised methylated nucleic acid or further linearised methylated nucleic acid) and formation of the intermediate vectors. The PCR primers may further provide a restriction enzyme recognition sequence that is arranged to direct the restriction enzyme to cut the PCR product to form the adapter sequences. The restriction enzyme recognition sequence provided by the PCR primers may be a restriction enzyme recognition sequence. The restriction enzyme recognition sequence may comprise the same sequence as the restriction enzyme recognition sequence of the maintained composite element and/or truncating composite element.

In one embodiment, the adapted middle DNA fragment comprises a region of overlapping native sequence that is the same sequence as a native overhang sequence provided in a consecutive/neighbouring DNA fragment intended for ligation. For example, where neighbouring/consecutive DNA fragments for assembly with each other are encoded from a template, they may each comprise an overlapping/same sequence region from the template, which would form the complementary native overhangs between the two neighbouring/consecutive sequences in a subsequent ligation. In one embodiment, the adapted middle DNA fragment comprises two regions of overlapping native sequence, that are arranged to form the native-overhangs of the middle DNA fragment once cut by the recognising restriction enzyme.

The method may further comprise the step of providing a linearised destination vector for insertion of the assembled DNA fragments. The linearised destination vector may comprise respective overhang sequences that are complementary to the maintained overhangs of the assembled DNA fragment. The overhang sequences of the linearised destination vector may comprise a first overhang that is complementary to the maintained-overhang sequence of the first adapted DNA fragment and a second overhang that is complementary to the second the maintained-overhang sequence of the second adapted DNA fragment.

The linearised destination vector may be provided by cutting a circular destination vector with the restriction enzyme(s) that are arranged to provide the same complementary overhangs as the assembled DNA fragments. For example, the linearised destination vector may be provided by cutting a circular destination vector with the restriction enzyme(s) that recognise the restriction enzyme recognition sequences of the maintained and/or truncating composite element. The linearised destination vector may be a cloning vector.

In one embodiment, the maintained composite element comprises the same sequence in all intermediate vectors that comprise the maintained composite element. In one embodiment, the truncated composite element comprises the same sequence in all intermediate vectors. In one embodiment, the maintained composite element comprises the same restriction enzyme recognition sequence as the truncated composite element. In one embodiment, the restriction enzyme recognition sequences of the maintained composite elements and/or the truncated composite elements of the intermediate vectors are the same. In particular the same restriction enzyme species may be used for restriction of the intermediate vectors, and optionally the destination vector.

The vectors used in the scarless assembly method of the invention may be high copy number vectors. For example the first and/or second linearised methylated nucleic acid may comprise a linearised methylated high copy number vector.

In one embodiment, the DNA assembly method of the invention is carried out in a single composition (i.e. so called “one-pot” process). For example, the DNA assembly process (including digestion with restriction enzyme and ligation) may be carried out in a single composition containing the restriction enzyme and DNA ligase using circular insert plasmids or linearized insert fragments and circular intermediate and/or destination vectors, or linearized versions thereof.

In one embodiment the methylation of the nucleic acid is carried out in a bacterial strain that is modified to expresses the methylase that recognises the methylase recognition sequence. In one embodiment the methylation of the nucleic acid is carried out in vitro.

According to another aspect of the invention, there is provided a modified bacterial strain that is modified to express a DNA methylase described herein.

The modified bacterial strain that is modified to express a DNA methylase may be an E. coli strain. The modification may comprise insertion of the methylase gene in the arsB locus of E. coli genome. The modified bacterial strain that is modified to express a DNA methylase may be formed by assembly of a vector with a methylase expression cassette followed by an antibiotic selection cassette, such as a zeocin antibiotic selection cassette, and flanked by homologous sequence from the arsb gene of E coli, followed by PCR using this as a template to generate linear DNA containing the above element, and recombineering with this fragment followed by selection with the antibiotic, such as zeocin, to generate bacteria strains with the methylase expression cassette and selection marker stably inserted into the arsB locus of E. coli genome.

According to another aspect of the invention, there is provided the use of a modified bacterial strain that is modified to express a DNA methylase described herein, for manufacturing a nucleic acid molecule according to the invention.

The modified bacterial strain may be E. coli.

According to another aspect of the inventions, there is provided a composition comprising the nucleic acid according to the invention. The composition may comprise buffer.

According to another aspect of the inventions, there is provided a kit comprising one or more nucleic acids according to the invention herein.

The kit may further comprise a restriction enzyme and/or a ligase, such as T4 DNA ligase. Additionally or alternatively, the kit may comprise a modified bacterial strain that is modified to express a DNA methylase according to the invention herein and/or a DNA methylase.

Additionally or alternatively, the kit may comprise one or more buffer solutions.

Additionally or alternatively, the kit may comprise antibiotics for clone selection.

One or more, or all, of the nucleic acid, buffer, bacterial strain, restriction enzyme, ligase, and methylase may be provided in a container, such as an Eppendorf tube.

According to another aspect of the invention, there is provided a host cell comprising nucleic acid according to the invention.

According to another aspect of the invention, there is provided the use of the nucleic acid according to the invention for assembling DNA fragments of interest, optionally wherein the use is in vitro.

Reference to the term “scarless” or “scarless assembly” herein is intended to refer to a sequence of DNA that has been assembled from multiple sequences, wherein the joint between the sequences does not comprise artefacts, such as unwanted base pairs, of the restriction and ligation of the DNA assembly process. For example, scarless assembly of DNA does not introduce additional sequence into the assembled DNA fragment. In some literatures it is also called seamless DNA assembly. In one example, an adapter sequence designed to provide complementary sticky ends to facilitate the joining of DNA fragments may be considered a “scar” between the joined sequences.

Reference to the term “adapter” herein is intended to refer to a sequence designed to provide complementary sticky ends to facilitate the joining of DNA fragments.

Reference to the term “overlap” or “overlapping” in the context of overlapping recognition sequences is intended to refer to at least part of the recognition sequence of one enzyme (for example any nucleotide other than N in a recognition sequence) being in a position of the nucleotide sequence that specifies the recognition sequence of another enzyme. (i.e. overlapping sequences share at least some common nucleotides).

Reference to the term “native” in the context of an overhang/sticky end sequence is intended to refer to the natural, non-adapted, sequence of the DNA fragment of interest to be inserted or joined to the nucleic acid. For example, the sequence will not be designed or amended relative to the sequence of the template DNA of interest. This is in contrast with overhangs determined by the vector sequence to facilitate DNA assembly, which might be introduced as a scar into the assembled sequence.

Reference to a “single assembled DNA molecule” is intended to refer to a single type of molecule, and is not intended to refer to the number of copies of the same molecule. In particular, in any given reaction or composition, there may be a plurality of copies of the single assembled DNA molecule.

Reference to “high copy number” plasmids/vectors may refer to those with a copy number of 100 or more copies per cell, including but not limiting to plasmids with replication of origins such as 1. mutated pMB1 replication origin from plasmid pUC plasmids (Vieira and Messing 1982. Gene 19:259-268; Vieira and Messing 1987. Methods Enzymol. 153:3-11; Messing 1983. Methods Enzymol. 101:20-78; Lin-Chao et al. 1992. Mol. Microbiol. 6:3385-3393, each of which are incorporated by reference herein); and 2. ColE1-derived replication origin from pBluescript plasmids—pBluescript II Phagemid Vectors, Instruction Manual, Catalog #212205, #212206. #212207 and #212208, Revision A.01, which is incorporated by reference herein). The replication origin carried by pbluescript is from pUC with a single nucleotide mutation. Particular guides on low and high copy plasmid/vector regulation can be found in Molecular Cloning A Laboratory Manual 3rd edition (2001), Joseph Sambrook and David W Russell, volume 1 page 1.4 Table 1-1 and volume 1 page 4.2 Table 4-1.

Reference to “low copy number” plasmids/vectors may refer to those with a copy number of less than 100 copies per cell, including but not limiting to plasmids with replication of origin such as 1. p15A replication origin from pACYC plasmids (Chang and Cohen 1978. J. Bacteriol. 134:1141-1156, which is incorporated by reference herein); 2. pSC101 replication origin from pSC101 plasmids (Stoker et al. 1982. Gene 18:335-341, which is incorporated by reference herein); 3. F replication origin from F plasmids (Kim et al. 1996. Genomics 34:213-218; Asakawa et al. 1997. Gene 191:69-79, each of which are incorporated by reference herein); 4. pMB1 replication origin from pBR322 plasmids (Bolivar et al. 1977. Gene 2:95-113, which is incorporated by reference herein); and 5. ColE1 replication origin from ColE1 plasmid (Kahn et al. 1979. Methods Enzymol. 68:268-280, which is incorporated by reference herein)

The skilled person will understand that optional features of one embodiment or aspect of the invention may be applicable, where appropriate, to other embodiments or aspects of the invention.

### BRIEF DESCRIPTION OF THE DRAWINGS

Embodiments of the invention will now be described in more detail, by way of example only, with reference to the accompanying drawings.

FIG. 1. Identification of methylases that block overlapping methylation/restriction sites

A. Diagram of overlapping methylation/restriction sites for the initial screening. Restriction enzyme recognition sites are boxed in solid lines. The adhesive end generated by the restriction enzyme is shown by a solid line. Methylation enzyme recognition sites are boxed in dashed lines, and methylated bases are in bold font. All the methylases listed modify N6-adenine, except M.SacI and M.AspJHL3I, which modify C5-cytosine and N4-cytosine respectively.

B. Experimental designs to test blocking of overlapping methylation/restriction sites by in vivo methylation. For each methylase/restriction enzyme combination tested, the test contains plasmid a head-to-head overlapping methylation/restriction site and a nonmethylatable restriction site. Restriction digest of test plasmid prepared from normal strain would result in restriction at both sites, and releases a ˜600 bp fragment from the ˜4.3 kb vector backbone. Restriction digest of test plasmid prepared from methylase-expressing strain would result in a single ˜4.9 kb band, due blocking of overlapping methylation/restriction sites by in vivo methylation. The restriction sites of test plasmid for each restriction enzyme are shown, with the restriction site boxed in solid line, methylation sequence boxed in dashed line, and methylated bases in bold. The head-to-head arrangement of overlapping methylation/restriction site allows assaying of nickase activity in the same assay.

C. Agarose gel electrophoresis analysis of test plasmid for each overlapping methylase/restriction enzyme combination prepared in normal strain or strain expressing DNA methylase and digested with corresponding type IIS restriction enzymes. The combinations tested are BsaI with M.Osp807II (M.Osp) using test plasmid pMOP_BsaINC, BpiI with M2.NmeMC58II (M2.Nme) using test plasmid pMOP_BpiINC, and LguI with M.XmnI using test plasmid pMOP_LguINC. Test conditions are 60 fmol test plasmid digested using 5U BsaI or BpiI, or 2.5U LguI in 10 μl reaction at 37° C. for 1h. Result shows that each of the methylases tested results in successful blocking through in vivo methylation of a restriction site for the corresponding type IIS restriction enzyme when the methylase recognition site overlaps the restriction enzyme site. The data shown represents results from three independent experiments.

FIG. 2. BsaI-M.Osp807II based Metclo system

The donor plasmids contain DNA fragments to be assembled (Frag A-D) flanked by BsaI sites that generate compatible adhesive ends (labelled aaaa-eeee). The BsaI sites overlap with the M.Osp807II methylase recognition sequence. Donor plasmids were prepared from a normal strain that lacks M.Osp807II methylase activity. As a result the BsaI sites are not methylated and so the insert DNA fragments can be released by BsaI digestion. The assembly vector contains a LacZ selection marker flanked by head-to-head BsaI sites. The outside pair of BsaI sites closer to the vector backbone overlap with M.Osp807II methylation sequence, whereas the inside pair do not. Preparation of the assembly vector in M.Osp807II-expressing DH10B strain results in blocking of the outside pair of BsaI sites specifically. The LacZ fragment could still be released by BsaI through the action on the inside pair of BsaI sites. Following a one-pot reaction using BsaI and T4 DNA ligase, ligation among compatible adhesive ends results in ordered assembly of DNA fragments to the assembly vector backbone. The assembled fragment in the assembled plasmid is flanked by blocked BsaI sites refractory to BsaI digestion. Following transformation into normal strain that lacks M.Osp807II activity, methylation of the flanking restriction sites is lost, and the assembled fragment could be released by BsaI for next stage assembly.

FIG. 3. DNA assembly using Metclo vector containing functional DNA elements between the methylation-protectable restriction site and non-protectable restriction site.

A. Diagram showing assembly of a transcription unit for expression of a protein with three different domains (D1, D2, D3) using Metclo vector with the promoter element (Prom) and polyA signal (polyA) pre-embedded into the vector. Methylation-protected BsaI sites are boxed in solid line, functional BsaI sites in dotted line, and methylated bases in bold. XXXX and YYYY represents the adaptor sequence at 5′ and 3′end of the assembled DNA fragment (transcription unit) that could be released for next round of DNA assembly. They may or may not be different from the internal adaptor sequences (AATG, ACTA, ATCT and GCTT), because they are not involved in the assembly reaction.

B. Diagram showing assembly of the same transcription unit using Metclo with the promoter element and polyA signal provided in separate insert plasmids. XXXX and YYYY represents the adaptor sequence at 5′ and 3′end of the assembled DNA fragment (transcription unit) that could be released for next round of DNA assembly. They are different from the internal adaptor sequences.

FIG. 4. Principle of adhesive end switching using universal cloning vectors

A. Diagram of three types of universal assembly vectors.

B. Details of adhesive end switching process of universal cloning vectors through Metclo, using vector VL as an example. Functional type IIS restriction sites are boxed in solid line, blocked restriction sites boxed in dashed line, and methylated bases in bold.

C. Symbolic system for representation of the adhesive end switching process. u and v represents the universal adhesive ends CTCC and CGAG respectively. x and y represents the 4 bp sequence inside the universal adhesive ends defined by the sequence. Assembly using different vector types results in switching of different adhesive ends.

D. Adhesive end switching process as applied to multiple fragments assembly.

FIG. 5. Scarless DNA assembly using universal cloning vectors

A. The DNA fragment to be assembled carries no internal BsaI restriction sites. The aim of the assembly process is to generate a single assembled fragment that carries adhesive ends x and y defined by the ends of the fragment.

B. The sequence is first broken up digitally into 9 different fragments (fragments A-I) with 4 bp overlaps (a-h).

C. Adaptor sequences were then added to the fragments digitally, resulting in DNA fragments that could be trimmed by BsaI to generate fragments flanked by the universal adhesive ends u and v.

D. Synthesized double stranded DNA fragments were first cloned into specific universal assembly vectors to switch the adhesive ends depending on the position of the fragment in the next stage assembly.

E. Cloned fragments were assembled 3 per group into specific universal assembly vectors, with the vector type depending on the position of the assembled fragment in the next stage assembly.

F Final stage of assembly generates the fully assembled fragment with sequence-specified adhesive ends x and y.

FIG. 6. Advantage of Metclo based scarless universal assembly compared to Moclo based universal assembly

For multistage assembly using universal assembly vectors, the Moclo based system uses two different restriction enzymes (BsaI and BpiI) for consecutive stages, which requires different universal adaptors for a given block (CTCC for BsaI and AGAA for BpiI). In order to clone the fragments from different stages into universal assembly vectors, the 5′ end of the first block and the 3′ end of the last block in each subassembly needs to switch between different adaptor sequences depending on the enzyme used for the assembly. The illustration shows the change of the 5′ adaptor for the first block (block A) during 3 consecutive stages of assembly reaction, with BsaI sites boxed in solid line, and BpiI sites boxed in dashed lines. A very long adaptor needs to be added to the 5′ end of block A to cope with the switch of adaptors during the different stages of assembly. The adaptor is “chewed back” 4 bp after each stage of assembly, so the length of the adaptor sequence depends on the number of the stages. The design based on Metclo is much simpler, because it uses a single enzyme BsaI for different stages of assembly. Here nonmethylatable BsaI sites are boxed in solid line, and BsaI sites blocked by methylation boxed in dashed line. The universal adaptor sequence CTCC at the 5′ end of the first block (block A) can be used for different stages of assembly. There is no need to switch adaptor sequence for the outer blocks. As a result the adaptor design for the initial sequence is much simpler.

FIG. 7. DNA assembly using Metclo. The figure illustrates the assembly of four DNA fragments (Fragments A, B, C and D) into a single DNA fragment (Fragment ABCD) through two cycles of Metclo using a single type IIS restriction enzyme. Stage 1 and Stage 2 are effectively the same process with progressively larger inserts. In Stage 1, the insert donor plasmids contain insert fragments (Fragments A, B, C or D) flanked by methylatable type IIS restriction sites that generate compatible adhesive ends. Preparation of insert plasmids in normal E. coli strains, which lack the appropriate site-specific methylase, results in insert fragments releasable by the type IIS restriction enzyme. The assembly vector contains the negative selection marker LacZα flanked by a purposely designed head-to-head configuration of type IIS restriction sites. The inside pair of the restriction sites are designed to be nonmethylatable, but the outside pair are methylatable by a site-specific methylase because the restriction enzyme recognition sequence overlaps with the methylase recognition sequence. Preparation of the assembly vector in E. coli strains expressing the corresponding methylase leads to selective methylation of the outside pair of methylatable restriction sites, and digestion with the type IIS restriction enzyme generates a vector backbone that retains the outside pair of methylated type IIS restriction sites. Ligation of the insert fragments with the vector backbone generates correctly assembled plasmid refractory to restriction by the type IIS restriction enzyme. All of the other unwanted fragments including the vector backbone of the insert plasmid and the LacZα fragment from the assembly vector contain active type IIS restriction sites, which are sensitive to restriction by the enzyme. This scheme enables a one-pot assembly reaction that combines the restriction and ligation step because the reaction favors generation of the correctly assembled plasmid. Transformation of the assembly reaction into a normal E. coli strain, which lacks the appropriate site-specific methylase, effectively results in demethylation of the methylated type IIS restriction sites flanking the assembled fragment in the assembled plasmid. In Stage 2, the assembled inserts (Fragments AB and CD) can then be used for a subsequent round of Metclo assembly into a larger fragment (Fragment ABCD).

FIG. 8. Level jumping: scheme for modification of DNA using components from different levels of assembly. The diagram shows the scheme for assembly of two DNA fragments (Fragments A and B) by the standard Metclo scheme and subsequent modification of the assembled fragment (Fragment AB) using a third fragment (Fragment C). The initial fragments used (Fragments A, B and C) are of the same level in the assembly hierarchy, in the sense that they carry the same antibiotic selection marker. The modification step modifies an existing fragment (Fragment AB) using a fragment (Fragment C) from a different level of assembly, hence “level jumping”. The modified fragment (Fragment ABC) carries the same adhesive ends as the starting fragment (Fragment AB), and an antibiotic selection marker (M3) different from both level 1 and level 2 blocks (M1 and M2). Therefore, the new fragment ABC can be used for the next level of DNA assembly using selection marker M1 in place of fragment AB. The maintenance of the adhesive ends during the modification step is achieved through the use of a special arrangement of head-to-head type IIS restriction sites (to the right of the LacZα fragment during the modification step), in which the inside nonmethylatable type IIS restriction site generates an adhesive end that is compatible with the modification fragment to be inserted (Fragment C), and the outside methylatable type IIS restriction site generates an adhesive end identical to the end of the DNA fragment to be modified (the right-hand end of Fragment AB in the diagram). The modification step is not possible with the standard Moclo approach using two type IIS restriction enzymes, because DNA fragments from consecutive levels of assembly would then need to be released by two different restriction enzymes, each of which needs to be different from the enzyme used for next level of DNA assembly after the modification step. The level-jumping modification scheme is useful for insertion of functional genetic elements such as transcription units into an existing assembly pipeline without changing the higher level assembly design, to maximize the reusability of existing sequence-verified DNA parts.

FIG. 9. Level insertion: scheme for assembly of DNA through an intermediate assembly stage. The diagram shows the scheme for assembly of four DNA fragments (Fragments A, B, C and D) into a single fragment (Fragment ABCD) through an intermediate stage of DNA assembly (Level insertion). In contrast to the standard Metclo assembly scheme (FIG. 3) that assembles the four fragment in a single reaction, this scheme involves an intermediate assembly stage by sub-assembly of fragments B and C into an intermediate assembly vector that carries a selection marker (M3) different from the one for level 1 DNA inserts (M1 carried by fragments A, B, C and D) and for level2 assembled DNA fragment (M2 carried by fragments ABCD). The level-insertion sub-assembly scheme offers more choices in the routes of DNA assembly, and increases the reusablity of intermediate assembled DNA fragments.

FIG. 10. Linear addition: scheme for assembly vector construction using BsaI-based Metclo. The diagram shows the scheme for linear addition of DNA parts for construction of assembly vectors carrying existing functional genetic elements (Fragments A and D). Starting with an assembly vector that carries a nonselectable stuffer DNA fragment flanked by nonmethylatable type IIS restriction sites, two DNA fragments (Fragments A and D) are first assembled with a LacZα selection marker into this vector. The LacZα selection marker is flanked by a specially designed head-to-head arrangement of type IIS restriction sites, such that the inside sites are methylatable and the outside sites are not. Preparation of the LacZα insert plasmid in an E. coli strain expressing the corresponding methylase results in selective blocking of the inside pairs of methylatable type IIS restriction sites. One-pot assembly of the LacZα insert with other inserts prepared from the normal E. coli strain (Fragments A and D) results in an assembled DNA fragment refractory to restriction by the type IIS restriction enzyme used for DNA assembly. Transformation of the assembled DNA into a normal E. coli strain that lacks the corresponding methylase activity results in demethylation of the methylatable type IIS restriction sites flanking the LacZα fragment. As a result, the assembled DNA can then be used as an assembly vector for addition of new DNA fragments (Fragments B and C) in place of the LacZα selection marker in the next round of DNA assembly. The net outcome is linear addition of DNA fragments into the assembly vector (Fragments A and D, then fragments B and C). The scheme is useful for construction of final stage assembly vectors that carry common functional genetic elements, such as mammalian selection markers.

### EXAMPLE 1—METHYLATION ASSISTED MODULAR ASSEMBLY USING A SINGLE RESTRICTION ENZYME (METCLO)

### Introduction

### Overview of Metclo

Here we developed an improved type IIS restriction enzyme-based hierarchical assembly method named Metclo, which reduces the number of restriction enzymes used to one. We achieved this by adding a pair of specific type IIS restriction sites for the same enzyme used for assembly into the assembly vector, which are selectively blocked by DNA methylation. Transformation of assembled DNA into normal strains that lack the specific methylase unblocks the flanking restriction sites, resulting in plasmids carrying the assembled fragment which can be released using the same type IIS restriction enzyme and then used for a subsequent round of DNA assembly. We tested a number of methylases for their abilities to block overlapping methylation/restriction sites for three commonly used commercially available type IIS enzymes BsaI, BpiI and LguI, and identified suitable methylases for each of them, namely M.Osp807II (for BsaI with overlapping sequence GACNNGGTCTCN\hat{ }NNNN), M2.NmeMC58II (for BpiI with overlapping sequence GAAGACGC\hat{ }NNNN), and M.XmnI (for LguI with overlapping sequence GAAGCTCTTCN\hat{ }NNN, methylated base or opposite base is underlined, additional bases required to specify methylase activity in bold, restriction enzyme recognition sequence in italic; all three modifications are m6A N6-methyladenine). We developed E. coli strains carrying chromosomal copy of expression cassettes for constitutive expression of these methylases, and showed that DNA propagated in these strains is refractory to digestion by the restriction enzyme when the restriction site overlaps with the corresponding methylase recognition sequence. We then built proof-of-principle Metclo systems for each pair of methylase/restriction enzyme for assembly of four fragments into a single fragment, which could be released by the same restriction enzyme. Finally, we demonstrated the utility of the M.Osp807II/BsaI-based Metclo system for hierarchical assembly a 218 kb DNA from 28 ×7.7 kb DNA in 2 stages using BsaI.

### Materials and Methods

### Plasmid Construction

Plasmids were constructed by standard restriction enzyme- and ligation-based cloning techniques. DNA fragments for cloning were generated by PCR or by gene synthesis (IDT or Invitrogen). For proof of principle modular assembly of ˜1 kb DNA from 4 fragments, the modular assembly acceptor vector backbone is based on ampicillin-resistant Moclo vector pICH47732 (available from Addgene, and described in A modular cloning system for standardized assembly of multigene constructs. Weber E, Engler C, Gruetzner R, Werner S, Marillonnet S. PLOS One. 2011 Feb. 18; 6(2):e16765. doi: 10.1371/journal.pone.0016765) and the donor plasmid backbone based on a kanamycin-resistant vector built by replacing the ampicillin-resistant gene in pICH47732 with a kanamycin-resistant gene. For hierarchical assembly of ˜200 kb DNA from 28 fragments, the modular assembly acceptor vectors are based on gentamicin or kanamycin-resistant low copy BAC vector backbone with F replication origin, and the donor plasmid backbone is based on kanamycin-resistant low copy vector backbones with p15A or F replication origin. Plasmids for methylase overexpression were built based on a kanamycin-resistant low copy BAC vector backbone with F replication origin. The methylase genes were under the control of J23100 synthetic promoter, and flanked by a zeocin selection cassette as well as homologous sequences of the arsB locus to facilitate stable integration into the E coli genome by recombineering. Template plasmids for methylase assay are based on amplicillin-resistant pICH47732.

### Generation of Methylase Expressing Strains

E. coli strains with stable expression of specific methylases were generated by recombineering. Briefly, DNA fragments containing the methylase gene, zeocin selection marker and flanking arsB homologous sequences were generated by PCR using the corresponding methylase expressing BAC plasmid as template. The fragments were used for lambda red recombineering in DH10B cells followed by zeocin selection to insert the methylase expression cassette into the arsB locus. Clones with correct chromosomal insertions were verified by PCR and sequencing. The methylase expressing strains can be stably maintained without antibiotic selection.

### Modular Assembly

For proof of principle modular assembly of ˜1 kb DNA from 4 fragments, assembly reactions were set up using 60 fmol each of donor plasmids prepared from DH5alpha cells, 60 fmol assembly vector prepared from DH10B strains expressing compatible methylase, 1,000U T4 DNA ligase (NEB), and 5U BsaI (NEB) or BpiI (Fermentas) or 2.5U LguI (Fermentas) in 20 μl 1×T4 ligase buffer (NEB). The reaction condition was: 37° C. 15 min, followed by 45 cycles of 37° C. 2 min plus 16° C. 5 min, then 37° C. 20 min, and 80° C. 5 min. Assembled samples were transformed into DH5alpha chemical competent cells, and plated on LB plates with AIX selection (ampicillin 100 μg/ml, IPTG 100 μM, X-Gal 50 μg/ml) at 37° C. overnight. White colonies were expanded and screened by DNA sequencing.

For hierarchical assembly of ˜200 kb DNA from 28 fragments, the assembly was carried out in two stages. At stage 1, the fragments were assembled 7 per group using 15 fmol each of donor plasmids prepared from DH10B cells, 15 fmol assembly vector prepared from DH10B-M.Osp807II cells, 1,000U T4 ligase (NEB), and 5U BsaI (NEB) in 20 μl 1×T4 ligase buffer (NEB). The reaction condition was the same as described above. The assembled samples were then drop-dialyzed against 50 ml dH2O using 0.05 um cellulose filter (Millipore) at room temperature for 1h. 5 μl of dialyzed sample was transformed into 25 μl NEB-10beta eletrocompetent cells by electroporation at 0.9 kV 100Ω 25 μF, using Gene Pulser and 1 mm electroporation cuvettes (Biorad). Following electroporation, cells were recovered by adding 1 ml LB medium and incubated at 37° C. 220 rpm for 1h. Cells were plated on LB plates with GIX selection (gentamicin 2.5 μg/ml, IPTG 100 μM, X-gal 50 μg/ml) at 37° C. overnight. White colonies were expanded and screened by restriction digestion using XhoI.

At stage 2, the ˜200 kb DNA was assembled using protocols similar to stage 1 with the following modifications: 30 fmol each of donor plasmids and 15 fmol assembly vector was used in the assembly reaction; following modular assembly and before the drop dialysis step, 10U BsaI was added to the assembly reaction for incubation at 37° C. for 45 min followed by heat inactivation at 80° C. for 5 min. Transformed cells were plated on LB plates with KIX selection (kanamycin 30 μg/ml, IPTG 100 μM, X-gal 50 μg/ml). White colonies were screened by PCR using primers detecting the junction between the second and the third 54 kb fragments (TACCCACGTGATTCACGCTG and ATCCTACAGACTCGCTGTGG). Selected PCR positive clones were screened by restriction digest using XhoI or BsaI with pulsed-field electrophoresis.

### Result

The Metclo system is based on the standard type IIS restriction enzyme-based one-pot DNA assembly system, and added an additional pair of type IIS restriction sites in the assembly vector backbone for release of the assembled DNA for next stage assembly. Unlike the Moclo system which uses restriction sites different from the type IIS enzyme used in the assembly process, Metclo uses the restriction sites for the same enzyme. To prevent the assembled plasmid from being cut by this enzyme in the one-pot assembly reaction, Metclo uses specific methylase to methylate the type IIS restriction sites overlapping with the methylase recognition sequences, in order to block these type IIS restriction sites in the vector backbone. The methylation is lost when the assembled plasmid is transformed into E. coli strains lacking the specific methylase, thus enabling the release of assembled fragment from the assembled plasmid by the same type IIS restriction enzyme used in the assembly reaction. The resulting Metclo system therefore allows hierarchical assembly of DNA fragments using one-pot reaction with a single type IIS restriction enzyme, among various applications.

The key to the Metclo system is the identification of suitable methylase to block type IIS restriction sites that overlap with the methylase recognition sequence in the vector backbone. There are several criteria for selection of suitable methylase. First of all, methylation of the overlapping methylation/restriction site must result in blocking the action of the type IIS restriction enzyme. The methylation process could occur by in vitro methylation of purified vector plasmid, or by propagating vector plasmid in strains that express the methylase in vivo. In vivo methylation is preferred due to the reduced cost and time in the vector preparation process. Secondly, the methylase recognition sequence must not be identical to or entirely enclosed by the type IIS restriction site; additional base pairs must be required to specify the action of the methylase on the overlapping methylation/restriction site. The reason for this requirement is because there are two pairs of type IIS restriction sites for the same enzyme in the assembly vector. One pair within the vector backbone need to be blocked by the methylase, whereas the other pair within the negative selection marker insert must not be blocked. If the methylase recognition sequence is identical to, or enclosed by the type IIS restriction site, all the restriction sites in the assembly vector for this particular type II restriction enzyme will be blocked by methylation, including the pair within the negative selection marker insert; the resulting methylated assembly vector won't be cut by the type IIS enzyme at all, therefore cannot be used for DNA assembly. Thirdly, additional base pairs to specify methylase activity should not overlap with the adhesive end sequence generated by the type IIS restriction enzyme. This will maintain maximum flexibility in design of adaptor sequence for DNA assembly.

We searched for methylases with predicted ability to block restriction sites for type IIS restriction enzymes BsaI, BpiI and LguI. These are common type IIS restriction enzymes used in type IIS-based assembly systems. We used a few extra criteria in addition to the ones listed above to choose candidate methylases for testing. We aimed to generate methylated plasmids by in vivo methylation, so the methylase should ideally be active at the growth temperature of E coli. We also preferred methylases that recognize longer sequences (5-6 bp), because with longer recognition sequences, fewer bases in the E coli host genome and assembly vector backbone would serve as methylation substrate by chance. This potentially increases the efficiency of methylation, and reduces the chance of adverse effect of heterologous DNA methylation on host gene function. In addition, we preferred single subunit methylases from type II restriction/modification systems that lack restriction activity towards unmethylated sequence to avoid potential problem of restriction during the vector construction step. Lastly, to increase the chance of methylation blocking the overlapping methylation/restriction sites, we preferred methylases that modify bases close to the middle of the restriction site.

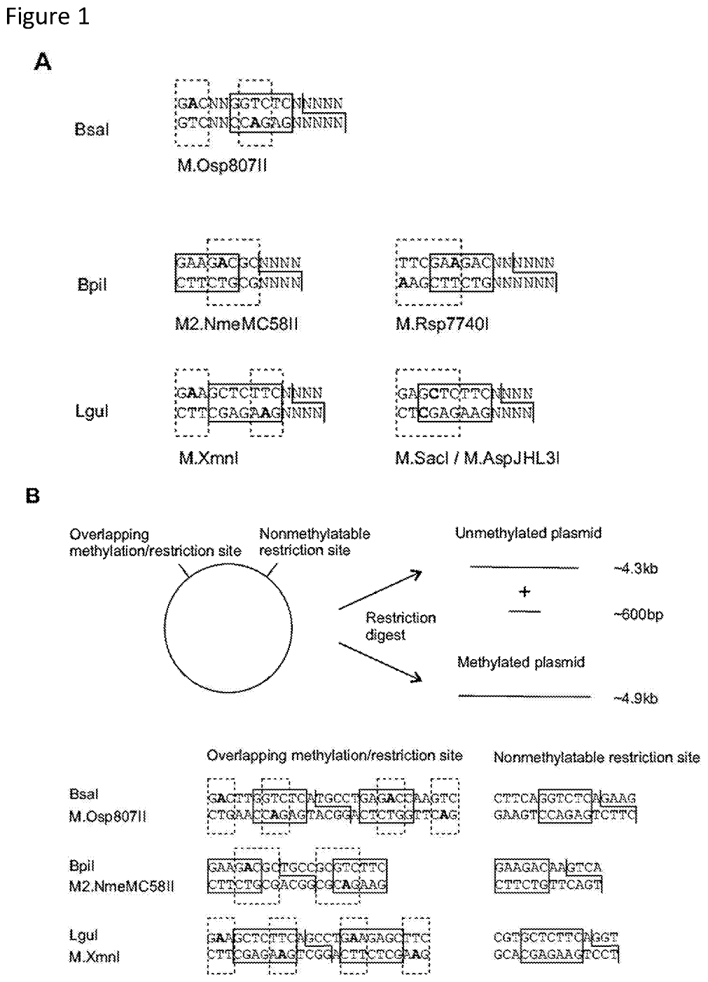

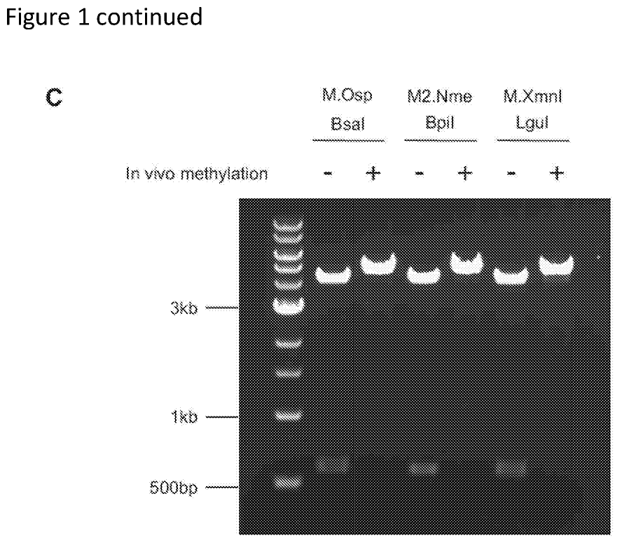

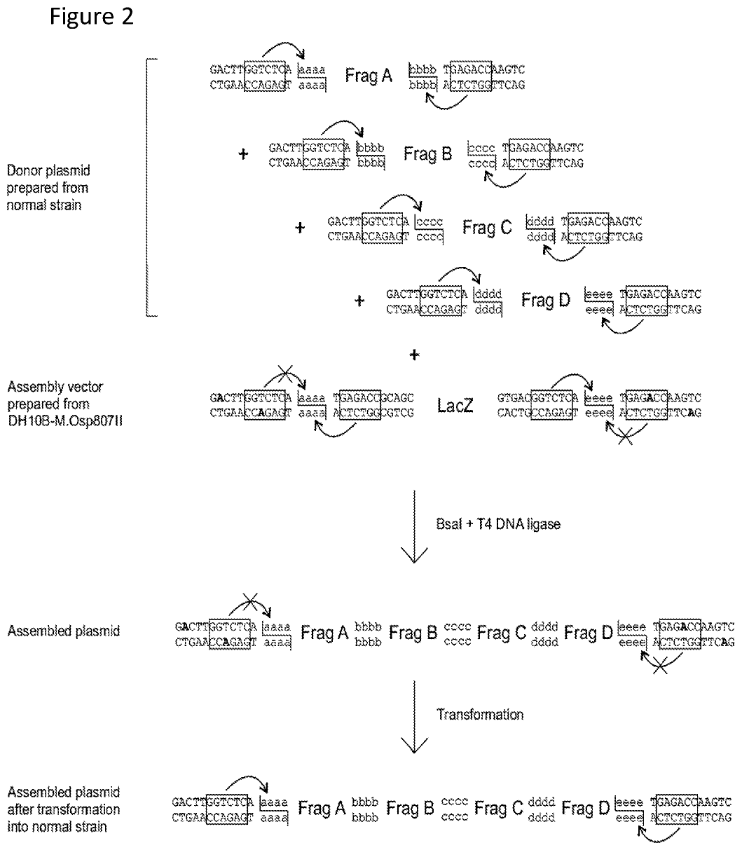

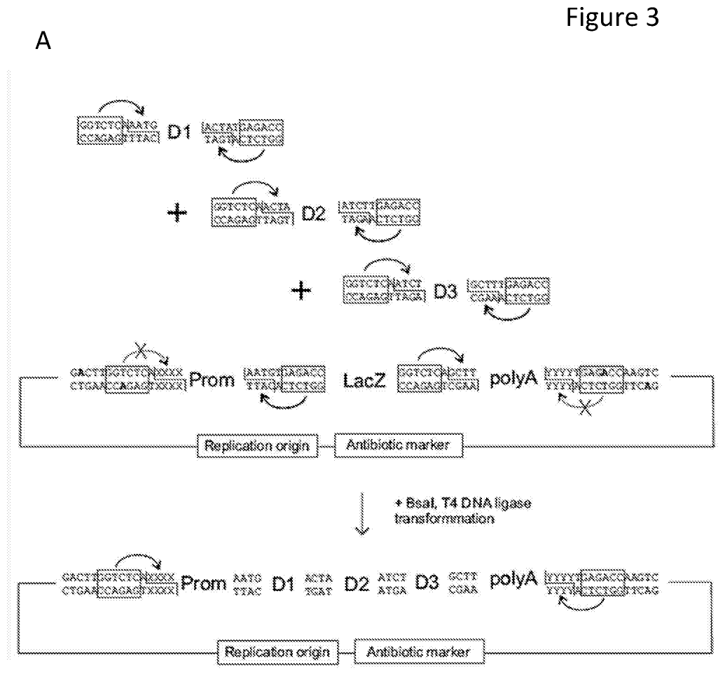

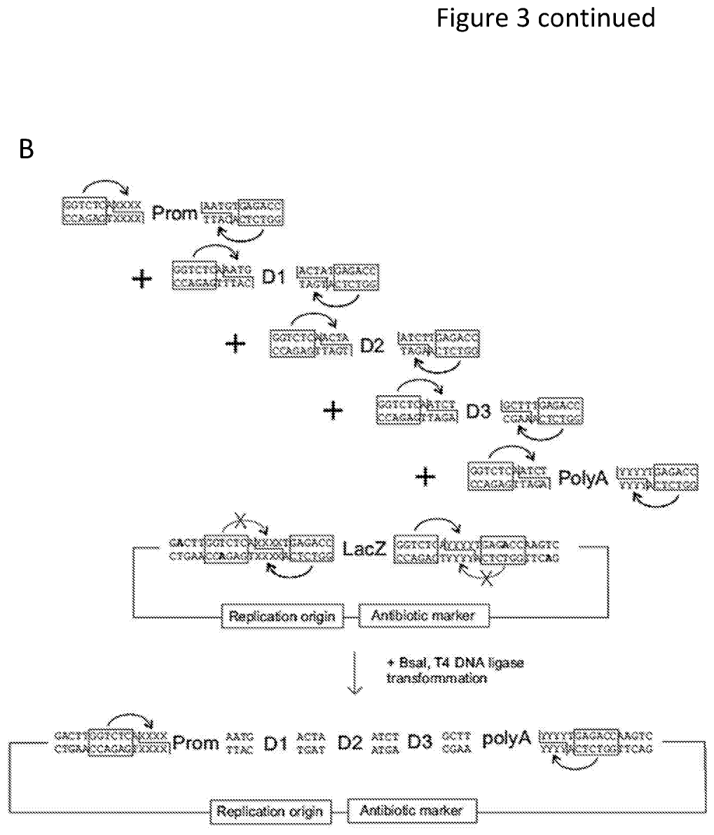

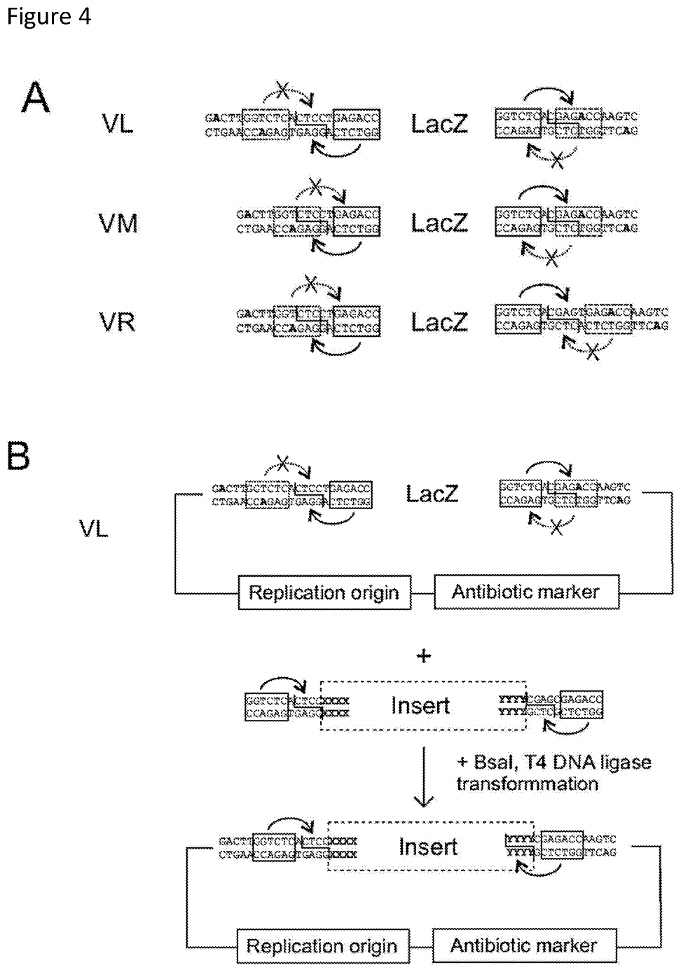

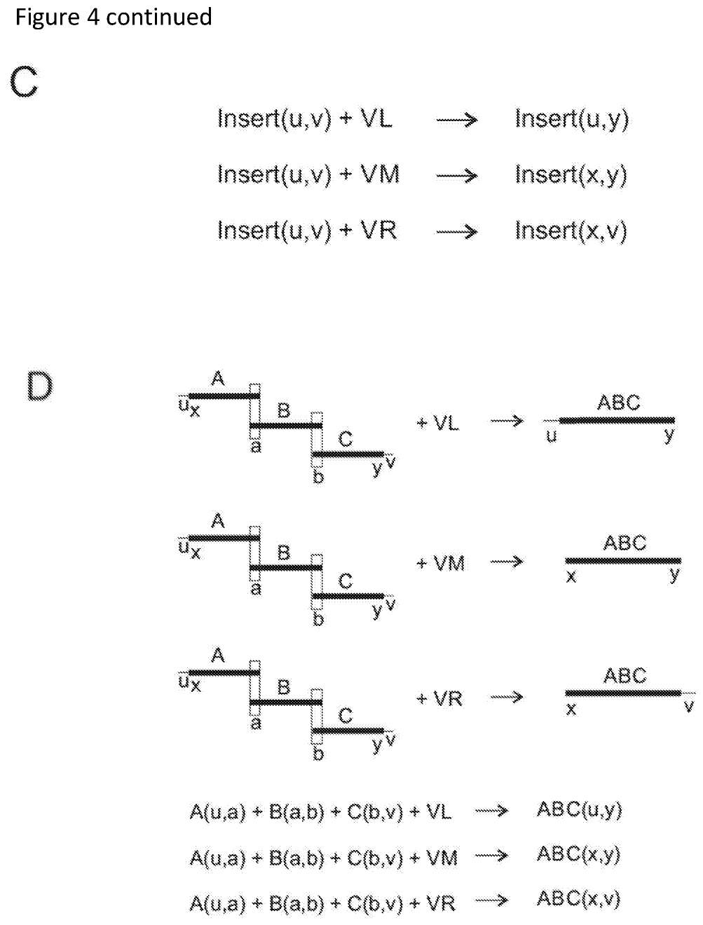

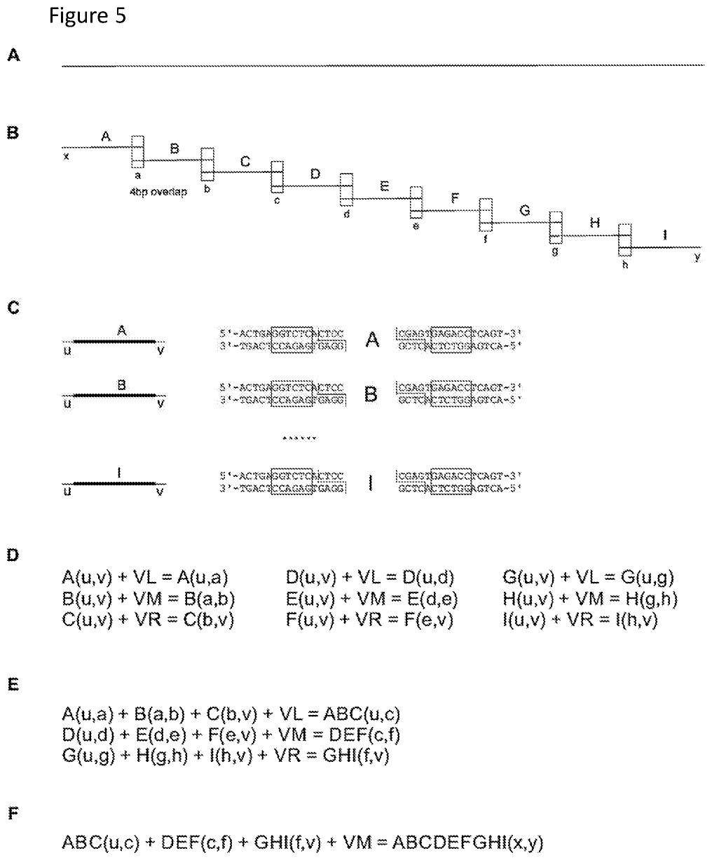

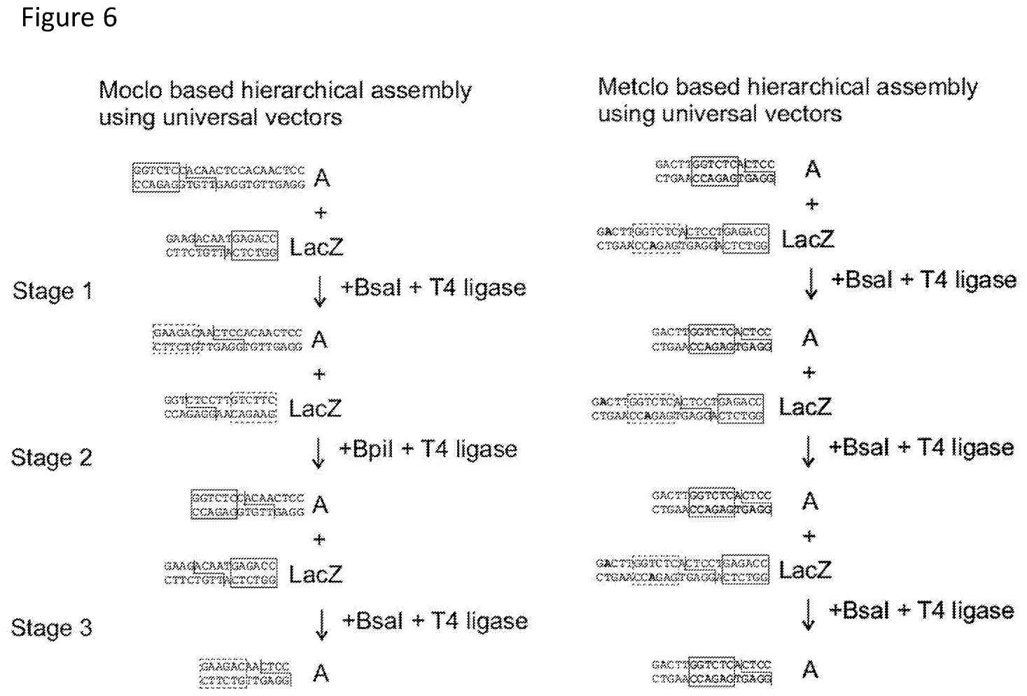

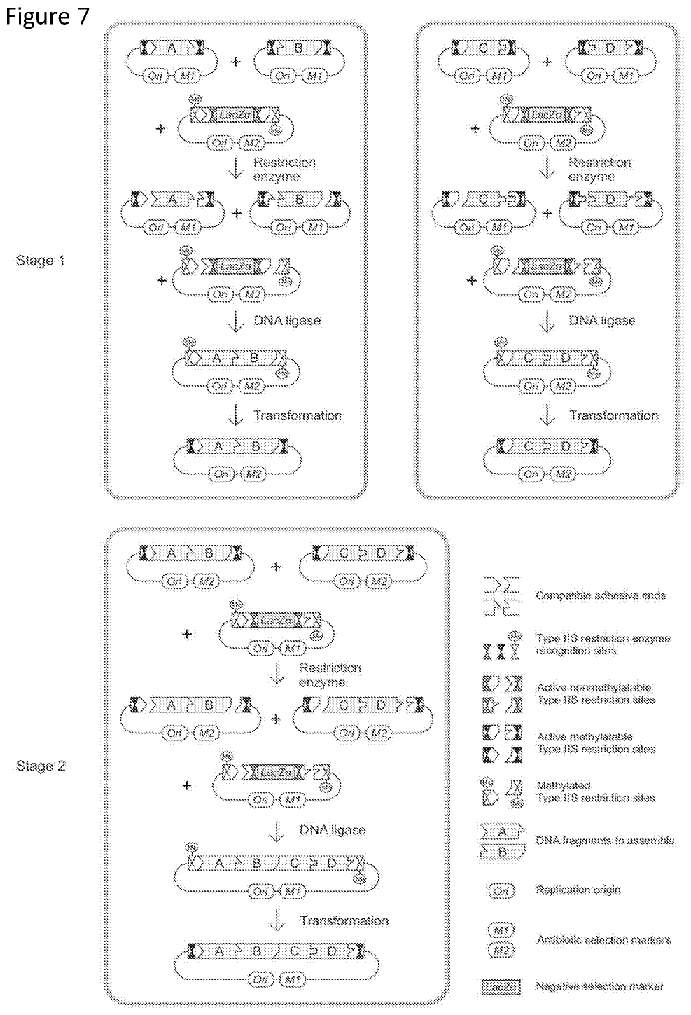

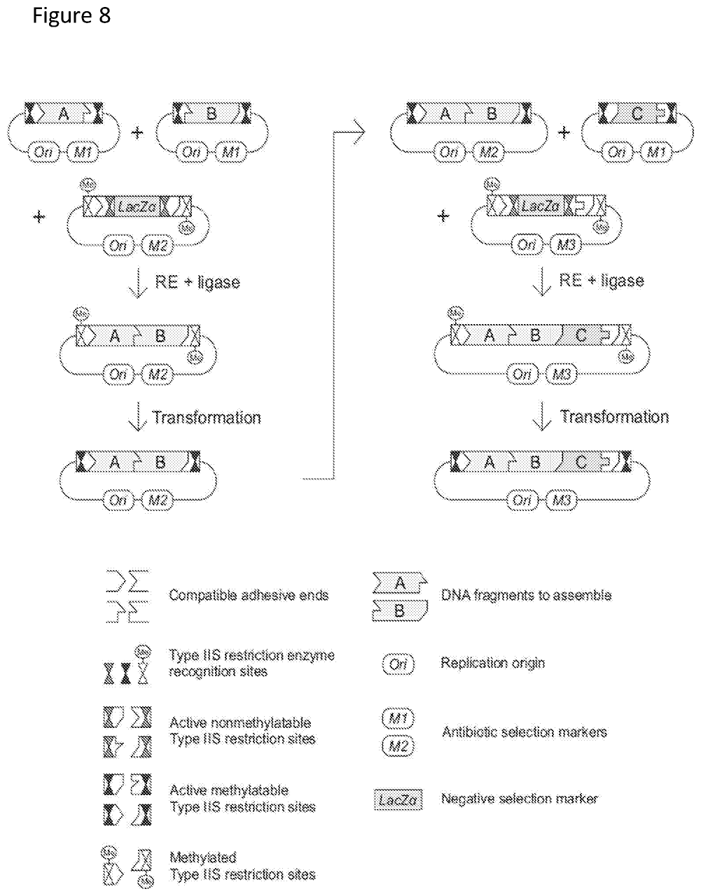

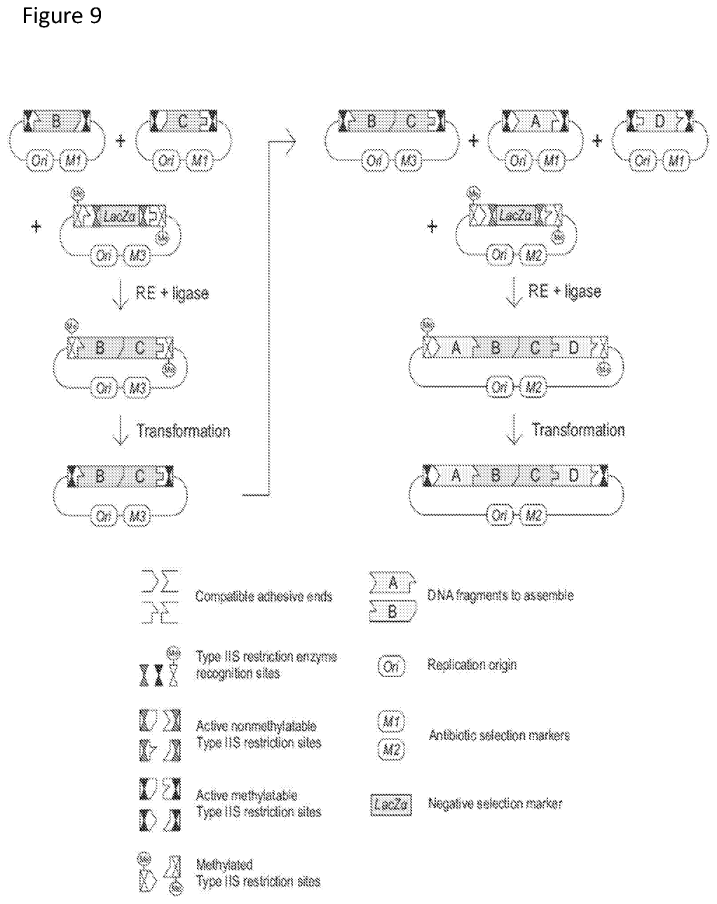

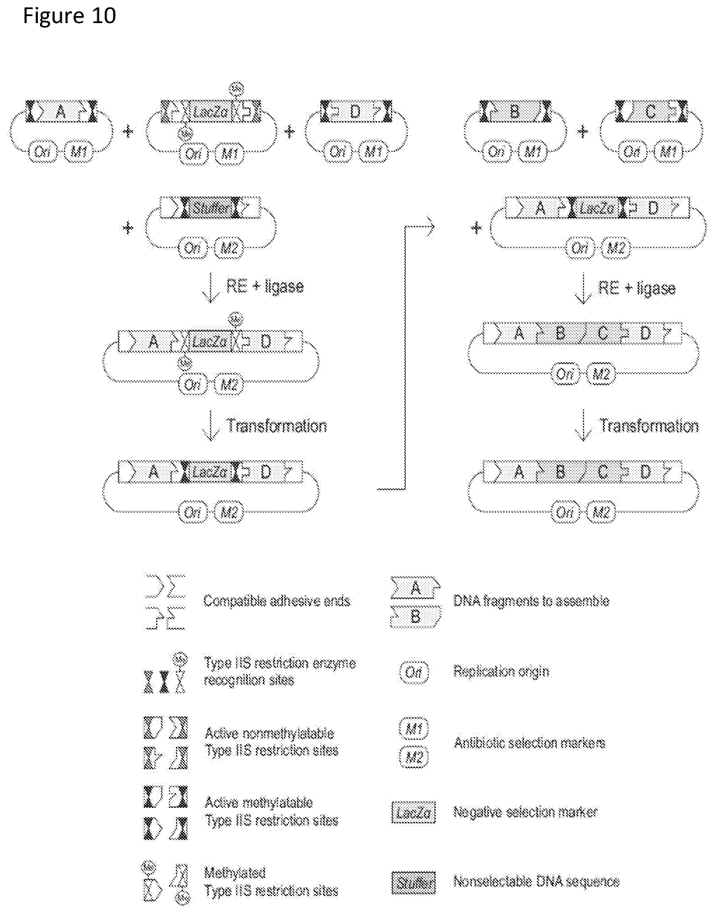

Based on these criteria, we selected the following methylases for testing: M.Osp807II (GACNNGGTCTC m6A) for BsaI, M.Rsp7740I (TTCGAAGAC m6A) and M2.NmeMC58II (GAAGACGC m6A) for BpiI, and M.SacI (GAGCTCTTC m5C), M.AspJHL3I (GAGCTCTTC m4C) and M.XmnI (GAAGCTCTTC m6A) for LguI (methylated base or complementary base underlined). We screened methylase activity by preparing ColE-based (i.e. ColE-derived) high copy template plasmid carrying overlapping methylation/restriction sites in DH10B cells expressing the corresponding methylase under the J23100 constitutive synthetic promoter from a low copy BAC vector (low copy BAC vector backbone with F replication origin). This identified M.Osp807II (for BsaI), M2.NmeMC58II (for BpiI) and M.XmnI (for LguI) as suitable candidates for further analysis (FIG. 1A). We then integrated the methylase expression cassette with a zeocin selection marker into the arsB locus using lambda-red recombineering to generate stable strains overexpressing the methylase from a chromosomal copy (Microb Cell Fact. 2013 Jun. 24; 12:60. doi: 10.1186/1475-2859-12-60 (PMID 23799955)), and tested the ability of these methylases to block overlapping methylation/restriction sites in vivo. The test vector carries one restriction site that does not overlap with methylation sequence, thus permissible for restriction in both wild-type and methylase-expressing strain, and one site with two head-to-head type IIS restriction sites, both of which overlap with the corresponding methylation sequence and would, therefore, be blocked in the strain expressing the methylase. The head-to-head arrangement allows for assessment of nickase activity as well as double strand restriction activity in the same assay (FIG. 1B). Following 1h digestion of 60 fmol test plasmid (pMOP_BsaINC, pMOP_BpiINC and pMOP_LguINC) in 10 μl reaction using 5U (BsaI, BpiI) or 2.5U (LguI) enzyme at 37° C., DNA were analyzed by agarose gel electrophoresis with EtBr staining. The result shows that under test condition, the three methylases block restriction of the overlapping sites, as demonstrated by the lack of generation of ˜600 bp fragment (FIG. 1C).

We then developed proof-of-principle Metclo DNA assembly systems using these methylases for one-pot DNA assembly (FIG. 2 shows BsaI-MOsp807II as an example). In these systems, the donor DNA inserts to be assembled are flanked by overlapping methylation/restriction site. Because the donor plasmids containing the DNA inserts to be assembled are prepared in a normal E coli strain that lacks the corresponding methylase, the restriction site is not blocked and the donor insert could be released by the enzyme in the assembly reaction. The assembly vector carries a different selection marker from the donor plasmid, and a LacZ selection marker flanked by head-to-head typeIIS restriction sites. For each head-to-head site, the two type IIS restriction sites generate the same adhesive end. The outside site close to the vector backbone overlaps with the methylation sequence, and the inside site close to the LacZ insert does not. The destination vectors are prepared from an E coli strain that expresses the corresponding methylase. As a result, only the inside typeIIS sites are functional in the assembly reaction. During the reaction, the LacZ insert is released from the vector backbone due to action of enzyme on the inside typeIIS site; ligation of the donor insert with the destination vector results in typeIIS restriction sites within from the destination vector backbone, which is blocked by methylation; religation of the freed LacZ insert with the destination vector backbone results in regeneration of the head-to-head typeIIS site, which is sensitive to restriction due to the inside site not blocked. As a result, the reaction favors ligation of donor fragments to destination vector backbone. The moclo reaction is then transformed into normal E coli strain that lacks the specific methylase activity for screening for LacZ expression using LB plates containing IPTG and X-Gal. The white colonies that lack LacZ activity should contain successfully assembled plasmids. The assembled DNA fragment have flanking type IIS restriction sites that overlap with methylase recognition sequences, which are identical to the donor vector. As a result, this allows a further round of modular assembly at the next level using the same restriction enzyme and so on for multiple rounds as required.

We tested the systems for the three enzymes for assembly from four fragments (Table 1). The donor vectors are kanamycin-resistant, and destination vectors ampicillin-resistant. Destination vectors were prepared from corresponding methylase-expressing strains. Following selection, 8 white colonies were picked for miniprep and analyzed by Sanger sequencing. Sequencing result shows that 100% (8/8) of clones are correct for each of the three Metclo systems (Table 1). This confirms the utility of Metclo for modular assembly using a single restriction enzyme such as BsaI, BpiI or LguI.

To further explore the utility of Metclo for hierarchical assembly of large DNA constructs, we assembled a 218 kb DNA fragment from 28×7.7 kb fragments using BsaI restriction enzyme in two stages. In stage one, 28 DNA fragments of 7.7 kb which were free of BsaI sites were assembled 7 per group into 4 pieces of 54 kb fragments using BsaI-based Metclo system. In stage two, the 4 pieces of 54 kb fragments were assembled into a single 218 kb fragment. The final 218 kb fragment is composed of non-repetitive sequence with ˜48% GC content. Restriction digest shows a ˜50% success rate for stage 1 assembly (2/4 to 3/4 clones correct), and ˜20% for stage 2 assembly (14 out of 27 PCR verified, of which 3 out of 8 verified by restriction digest) (Table 2). This confirms that the Metclo system can be used for hierarchical assembly of large DNA construct using a single type IIS restriction enzyme.

### Discussion

Here we present Metclo, a modified type IIS restriction enzyme based DNA assembly system for one-pot assembly of DNA using common type IIS restriction enzymes such as BsaI, BpiI or LguI, that generates assembled DNA releasable by the same restriction enzyme. This enables hierarchical assembly of large DNA constructs using a single type IIS restriction enzyme. The system uses methylase-overexpressing strains during the vector preparation step for in vivo methylation of overlapping methylation/restriction sites to block selective restriction sites in the vector. The assembly process is the same as the type IIS DNA assembly systems through the use of one-pot reaction, thus preserves the simplicity of the existing systems. The part preparation and transformation steps utilize normal strains similar to the standard Moclo system, allowing the use of routine cloning strains for DNA assembly, similar to the current type IIS assembly systems.

The Metclo system has a number of advantages over the existing type IIS restriction enzyme-based hierarchical DNA assembly systems. Firstly, the Metclo approach reduces the burden to remove internal restriction sites during component part construction. Compared with the standard Moclo system that uses two or more enzymes, the BsaI- and BpiI-based single enzyme Metclo system uses a 6 bp cutter that reduces the frequency of forbidden restriction sites from 1 per ˜1 kb to 1 per ˜2 kb for random DNA with 50% GC content, and the LguI-based Metclo system uses a 7 bp cutter that reduces the frequency further to 1 per ˜8 kb.

Metclo improves the flexibility of the hierarchical type IIS assembly system. Because assembled DNA fragments from different stages of assembly can be released by the same restriction enzyme, with appropriate design of adaptors and choice of antibiotic resistant vector, the system potentially allows design of several assembly schemes not achievable using the existing system. These include level jumping—the assembly of DNA using parts from different stages of assembly—and level insertion—the assembly of DNA fragments through an intermediate assembly stage. These schemes are useful for modification of large DNA constructs using existing parts, and for subassembly of complex parts such as multi-part open reading frames for added reusability.

Additional schemes could also be devised for linear addition of DNA parts during the assembly process. For example, DNA parts containing selection markers flanked by internal methylation-blocked type IIS restriction sites could be used during DNA assembly. The assembled plasmid could then be used as an assembly vector for insertion of additional DNA parts in place of the selection marker in the next stage assembly. This is particularly useful for construction of common assembly vectors containing functional modules.

Metclo also increases the exchangeability of parts from different assembly standards. Independent development of part library and assembly standards lead to assembly systems that use different type IIS restriction enzymes. For example, for plant biology, Moclo uses BsaI/BpiI (and Esp3I), Goldenbraid uses BsaI and BsmBI (and BtgZI); for E. coli, Ecoflex uses BsaI/BsmBI, CIDAR uses BsaI/BpiI. This place constraints on exchangeability of parts among different collections. The Metclo system that uses a single type IIS restriction enzyme is compatible with any system that uses that restriction enzyme. In particular, the BsaI-based system is compatible with all the existing systems that use BsaI. Given that BsaI has been used in most of the type IIS restriction enzyme-based hierarchical modular assembly systems to date (ACS Synth Biol. 2016 Oct. 21; 5(10):1059-1069 (PMID 27096716); ACS Synth Biol. 2016 Jan. 15; 5(1):99-103. doi: 10.1021/acssynbio.5b00124 (PMID 26479688; ACS Synth Biol. 2015 Sep. 18; 4(9):975-86. doi: 10.1021/sb500366v (PMID 25871405); and PLOS One. 2011 Feb. 18; 6(2):e16765. doi: 10.1371/journal.pone.0016765 (PMID 21364738), the BsaI-based Metclo system has potential to be used as a standard for modular assembly that is compatible with most of the existing part libraries.

There are DNA assembly systems that use DNA methylation to block internal type IIS restriction sites during the assembly process. For example, the TNT assembly system (Sci Rep. 2016 Jan. 13; 6:19278. doi: 10.1038/srep19278 (PMID 26758940)) uses M.TaqI to methylate overlapping Earl recognition site, which results in an assembly system using 7 bp cutter LguI (GCTCTTCN\hat{ }NNN) and 6 bp cutter Earl (CTCTTCN\hat{ }NNN) both giving 3 bp sticky ends, thus requiring removal of a single 6 bp sequence from DNA parts (CTCTTC). However, because the M.TaqI recognition sequence (TCGA) that overlaps with the Earl restriction site intrudes the adhesive end sequence (CTCTTCG\hat{ }ANN), the system places limitations on the design of adaptors, thus could only assembly fragments three at a time, and could not be expanded to an LguI-only assembly system. As another example, Greengate used methylated linker DNA as parts during the DNA assembly process to introduce new BsaI sites for subsequent rounds of assembly. The resulting system however could only be used for linear addition of DNA parts, thus limiting the use of the system to construction of DNA constructs with a small number of transcription units. Compared to these systems, the Metclo system, with the use of in vivo methylation of overlapping methylation/restriction site outside the adaptor sequence, preserves the advantage of the hierarchical multiplicative assembly scheme of the Moclo system, and maintains the freedom of choice of adaptor sequences for DNA assembly. In practice, similar result could be achieved by adding methylated linker DNA as the first and last parts during the assembly process. However, this would increase the number of parts in the assembly by two, which reduces success rate of assembly compared to the in vivo vector methylation approach in the Metclo system.

In summary, the Metclo system, through the use of in vivo methylation to block specific type IIS restriction sites within the assembly vector, enables hierarchical assembly of large DNA fragments using a single type IIS restriction enzyme. The system simplifies the existing type IIS restriction enzyme-based assembly method and increases the flexibility of design of assembly schemes. The BsaI-based Metclo system in particular is compatible with all the existing part libraries that uses BsaI, and could be used as a standard type IIS-based assembly system.

TABLE 1
Proof of principle assembly of DNA fragment using BsaI, BpiI or Lgui-based Metclo systems
Table 1. Proof of principle assembly of DNA fragment using BsaI, BpiI or Lgui-based Metclo systems

| Plasmid | Vector | Inserts | Methylase | Enzmye | Size | Success rate |
|---|---|---|---|---|---|---|
| pMXP_test | pMXK2_A4E | pMXP_FragA pMXP_FragB pMXP_FragC pMXP_FragD | M.Osp807II | BsaI | 908 bp | 8/8 |
| pMYP_test | pMYK2_A5E | pMYP_FragA pMYP_FragB pMYP_FragC pMYP_FragD | M2.NmeMC58II | BpiI | 1152 bp | 8/8 |
| pMZP_test | pMZK2_P6T | pMZP_FragA pMZP_FragB pMZP_FragC pMZP_FragD | M.Xmnl | LguI | 1149 bp | 8/8 |

TABLE 2
Hierarchical assembly of a 218 kb DNA fragment using BsaI-based Metclo system

| | | | | Success |
|---|---|---|---|---|
| Plasmid | Vector | Inserts | Size | rate |
| pMXBG_3A1 | pMXBG_A7I | pMOLK_2A1 pMOLK_2B1 pMOLK_2C1 pMOLK_2D1 pMOLK_2E1 | 54 kb | 3/4 |
| | | pMOLK_2F1 pMOLK_2G1 | | |
| pMXBG_3I1 | pMXBG_I7G | pMOLK_2I1 pMOLK_2A2 pMOLK_2B2 pMOLK_2C2 pMOLK_2D2 | 55 kb | 2/4 |
| | | pMOBK_2E2 pMOLK_2F2 | | |
| pMXBG_3G1 | pMXBG_G7F | pMOLK_2G2 pMOLK_2I2 pMOLK_2A3 pMOLK_2B3 pMOLK_2C3 | 54 kb | 2/4 |
| | | pMOBK_2D3 pMOLK_2E3 | | |
| pMXBG_3F1 | pMXBG_F7E | pMOLK_2F3 pMOLK_2G3 pMOLK_2I3 pMOLK_2A4 pMOLK_2B4 | 54 kb | 2/4 |
| | | pMOLK_2C4 pMOLK_2D4 | | |
| pMXBK_4A1 | pMXBK_A7E | pMXBG_3A1 pMXBG_3I1 pMXBG_3G1 pMXBG_3F1 | 218 kb | 3/8* |

*14/27 PCR positive, of which 3/8 verified by restriction digest

### Example of an Insertional Composite Element

In another scenario, longer DNA sequences could be included between the methylation-protectable restriction site and the opposing non-protectable restriction site in the assembly vector, such that following DNA assembly, these sequences will be added into the 5′ or 3′ end of the assembled DNA plasmid. This design is convenient for adding common elements to the assembled plasmid by using specialized vectors.

An example is shown in FIG. 3 for assembly of a transcription unit for expression of a protein with three different domains, starting from insert plasmids containing the three individual domains D1, D2 and D3 using BsaI-based Metclo. Figure A shows the Metclo assembly using a special Metclo vector containing a promoter between the methylation-protectable restriction site and the non-protectable restriction site at 5′ of the LacZ (discard sequence), and a polyA element at 3′ (“pre-embedded vector”. Metclo using this special pre-embedded vector allows the assembly of the three domains into a transcription unit in a one-pot reaction from three insert plasmids, and the assembled transcription unit consisting a promoter, a coding region (D1 D2 and D3), and a polyA signal could be released from the assembled plasmid for next round of Metclo assembly. This is in contrast with the standard Metclo using “maintained composite element”, whereas the transcription unit has to be assembled from 5 different insert plasmids (Figure B). In the figures XXXX and YYYY represents the adaptor sequence at 5′ and 3′end of the assembled DNA fragment (transcription unit) that could be released for next round of DNA assembly. For DNA assembly using “pre-embedded vectors”, these two adaptor sequences can be the same as internal adaptor sequences because they are not involved in the one pot three fragment assembly. For DNA assembly using standard Metclo vector though, XXXX and YYYY adaptor sequences must be different from the internal adaptor sequences (AATG, ACTA, ATCT and GCTT). In addition, the method using pre-embedded vectors (Figure A) has advantage that less number of DNA parts need to be assembled in a single reaction, which increases the rate of success. The disadvantage is that the specialized vector is more difficult to prepare.

### EXAMPLE 2—SCARLESS DNA ASSEMBLY (SCARLESS METCLO)

A unique application of Metclo is a system for scarless hierarchical assembly of large DNA construct using a limited set of universal cloning vectors. Type IIS based DNA assembly using universal vectors relies on a specific arrangement of head-to-head type IIS restriction sites in the assembly vector that changes the adhesive end of the cloned fragment. Scarless assembly is achieved by using these universal vectors to generate assembled DNA fragments with adhesive ends (scars) defined by the DNA fragment for subsequent round of assembly, which buried the assembly scar into the assembled sequence. The use of the Metclo system allows maintenance of the same universal adhesive ends for the outer fragments during different stages of hierarchical assembly. A combination of these three features results in a universal scarless assembly system.

Here we describe the principles of scarless Metclo assembly system using the BsaI restriction enzyme. The system relies on a set of three types of universal cloning vectors that change the adhesive ends of the cloned fragments (FIG. 4A). The three vector types are named VL, VM and VR. The vectors are designed to clone DNA fragments with adhesive end CTCC at 5′ end and CGAG at 3′ end could be cloned into the above vectors. The cloned fragment, once released by BsaI from the vector, will have different adhesive ends depending on the vector used. Vector VL is designed to keep the 5′ adhesive end, and change the 3′ adhesive end to the four bases inside the 3′ CGAG sequence; Vector VR to keep the 3′ adhesive end, and change the 5′ adhesive end to the four bases inside the 5′ CTCC sequence; Vector VM to change both ends to the four bases inside. In this way a set of universal vectors could be used to generate DNA fragments with adhesive ends defined by the insert sequence.

The detail of adhesive end switching process is described in FIG. 4B using vector VL as an example. When the vector is prepared in methylase-expressing competent cells, methylation of adenine (bold) due to overlapping methylase recognition sequence results in blocking of the outside BsaI sites (boxed in dotted line), where the inside BsaI sites (boxed in solid line) are available to remove the LacZ insert. Note that the left arm of vector VL is similar to standard Metclo, that both the outside and inside BsaI sites give rise to the same adhesive end CTCC. The right arm of VL however is different that the adhesive end CGAG generated by the inside BsaI site overlaps with the outside BsaI site. The donor DNA carries an insert that could be released by flanking BsaI sites with compatible universal adhesive ends CTCC and CGAG. The double strand insert carries 4 bp sequence XXXX on the 5′ end and YYYY on the 3′ end inside the universal adhesive end. Following Metclo assembly of the donor DNA into vector VL, the resulting plasmid carries the corresponding insert that could be released by the flanking BsaI sites (boxed in solid line) in the vector. The released fragment would carry a 5′ adhesive end CTCC identical to the one used for cloning, but the 3′ adhesive end would be YYYY as defined by the insert sequence, which may be different from the universal 3′ adhesive end CGAG. In this way, vector VL switches the right hand adhesive end of the cloned fragment from universal adhesive end CGAG to the sequence-defined adhesive end YYYY.

The conversion process using the three types of universal cloning vectors could be described using a simple symbolic representation system (FIG. 41C). Here we use letter u to represent the 5′ universal adhesive end CTCC, and letter v to represent the 3′ universal adhesive end CGAG. x and y represents the 4 bp sequence at the 5′ and 3′end of the insert fragment inside the universal adhesive end. Insert (u,v) represents a plasmid carrying insert fragments flanked by BsaI sites that generate adhesive ends u and v. Assembly reaction with different vectors results in different adhesive ends being converted.

The same set of universal vectors could be used to assemble multiple fragments with compatible adhesive ends, as long as the 5′ end of the first fragment and the 3′ end of the last fragment have universal adhesive ends u and v (FIG. 4D). The assembled fragment carries different adhesive ends depending on the type of assembly vector used. Assembly of fragments into vector VL keeps the 5′ universal adhesive end, and converts the 3′ end into the internal 4 bp. Assembly into vector VM converts both ends, and assembly into vector VR converts the 5′ end and keeps the 3′ universal adhesive end. Note the fragment assembled into vector VL resembles fragment A that it carries only 5′ universal adhesive end u; the fragment assembled in vector VM resembles fragment B; and the fragment assembled in vector VR resembles fragment C. Therefore the process is self-similar, such that the assembled fragments could be used for next round DNA assembly using the same principle depending on the position of the fragment in the assembly order. This enables hierarchical scarless assembly of large DNA constructs using universal cloning vectors.

The whole process of hierarchical scarless assembly of DNA is described in FIG. 2, using DNA assembly from 9 fragments in 2 stages as an example. Starting with a DNA sequence free of internal BsaI sites, with the 4 bp at 5′ end named x, and 3′end named y, the aim is to assemble the DNA from 9 fragments into a single fragment that once released from the assembled plasmid carries adhesive ends x and y (FIG. 5A). The DNA sequence is first broken up into 9 fragments named A-I with 4 bp overlapping sequences, with the overlapping sequences named a-h (FIG. 5B). The positions of 4 bp overlaps could be chosen freely, as long as they are different to universal adhesive ends u and v, and to each other within each subassembly reaction. For the current set up, the following pairs of 4 bp overlaps should be different within each pair: (a,b), (d,e), (g,h) and (c,f). Appropriate adaptor sequences were added to the ends of sequences of fragments A-I, and double DNA synthesized by gene synthesis or PCR, so that following restriction digest with BsaI, the double stranded DNA could be trimmed into fragments A-I carrying universal adhesive ends u and v (FIG. 5C). These double stranded synthetic DNA fragments could then be cloned into appropriate types of universal cloning vectors to change the adhesive end of the fragment depending on the position of the fragment in the next stage assembly (FIG. 5D). Fragments A, D and G, which will locate at the 5′ end position in the next round of assembly, are cloned into vector type VL; B, E, H in the middle position in the next round are cloned into vector type VM; C, F, I at 3′ end position into vector type VR. Cloned fragments could be sequenced at this stage to check for errors in gene synthesis or PCR. These fragments could then be used for a new round of assembly, and again the type of assembly vector used depends on the position of the assembled fragment in the next round DNA assembly (FIG. 5E). Fragments A, B, C were assembled into vector VL, because the assembled fragment ABC will be used as the 5′ fragment in the next round assembly; fragments D, E, F into vector VM, and fragments G, H, I into vector VR. The final step is similar so that the three assembled fragments from step 2 is further assembled into vector VM, which generates the fully assembled fragment that carries adhesive ends x and y defined by the ends of the initial sequence (FIG. 5F).

A set of at least 6 universal cloning vectors representing 3 types (VL, VM, VR) and 2 antibiotic selection markers are required for hierarchical assembly. Ideally 12 vectors representing 2 different copy numbers are optimal in order to cope with different sizes of DNA fragments at different stages of assembly. Similar system could be developed for LguI based Metclo, but not BpiI, because the scarless system requires universal adaptor u and v to be different. For BsaI and SapI, the bases between the enzyme recognition sequence and adhesive end generated by the enzyme is flexible, therefore it is possible to design different adaptors u and v. For BpiI however, these bases are fixed because the two bases are both required to specify the methylase recognition sequence in BpiI based Metclo. In addition, the methylated base of BpiI based Metclo is located on the top strand of the restriction site. Head-to-head arrangement of BpiI sites for adhesive end switching inevitably results in the methylated base being carried by the LacZ selection marker, not the assembly vector backbone following BpiI digestion, which makes one pot Golden Gate assembly impossible.

### The Relationships Between Scarless Metclo and Metclo

Metclo was invented to solve the problem of using a single restriction enzyme for type IIS restriction enzyme-based hierarchical assembly of large DNA construct. Scarless Metclo is a further development of Metclo that utilizes key components of the Metclo system (methylase-expressing strains, overlapping methylation/restriction site) to achieve scarless hierarchical assembly of large DNA constructs using a set of universal assembly vectors.

Scarless Metclo is based on a combination of several different concepts of type IIS-based assembly system:
1. A special design of head-to-head type IIS restriction site for adhesive end switching, which has been used in the Moclo system for “level-1” universal cloning vector design. The Moclo “level-1” vector design uses a BpiI-BsaI head-to-head restriction site, which is different from the BsaI-only head-to-head design in the Metclo, but the concept of adhesive end switching is based on the Moclo “level-1” vector design.
2. The removal of the assembly scar by designing the scar sequence around the assembled DNA sequence. This hasn't been demonstrated in type IIS-based hierarchical assembly before.
3. The use of Metclo system to maintain the same adhesive end sequences for the outer blocks of DNA fragments for each stage of assembly.

The use of Metclo system is key to achieve multi-stage hierarchical assembly using the same universal adhesive end sequence. The first two concepts alone are sufficient for scarless single stage assembly of DNA using a universal assembly vector. The single stage scarless assembly system however could not be adapted easily for multi-stage hierarchical assembly using the traditional Moclo system, because the universal adaptor sequence of the adhesive end switching vector design for different enzymes are different (BsaI would be CTCN and NGAG, and BpiI ACNN and NNGT). This means that in order to achieve hierarchical scarless assembly using the two enzyme-based Moclo, very long repetitive adaptor sequences need to be added to the ends of the outer blocks depending on the number of stages of assembly, which will be “chewed back” following each stage of assembly (FIG. 6). Because Metclo system uses a single enzyme for different stages of assembly, the same universal adaptor sequence could be used for the outer blocks during different stages of assembly, which greatly simplifies the design of the assembly process.

### EXAMPLE 3—IDENTIFIED METHYLASE AND TYPE IIS RESTRICTION ENZYME COMBINATIONS

Table 3 contains a list of methylase/restriction enzyme combinations that may result in blocking of overlapping methylation/restriction site for all known type IIS restriction enzymes (78 enzymes representing 19 different specificity).

The list was generated using a custom Perl script based on a list of known type IIS restriction enzymes and a list of known type II DNA methylases, both from Rebase (PMID 25378308. (Nucleic Acids Res. 2015 43: D298-9).

The listed methylases are limited to methylases from type II restriction/modification systems with recognition sequences of at least 4 bp and for which the position of the methylated base is known. Methylases from the type IIG restriction/modification system that contain both methylase and restriction enzyme activity in the same protein are excluded. In addition to the restriction enzyme recognition sequence, further bases must be required to specify the methylase se activity towards the overlapping methylation/restriction site. Any methylase whose recognition sequence is identical to or enclosed by the restriction site are removed. In addition, the methylation sequence must not intrude into the adhesive end sequence generated by the type IIS restriction enzyme.

The DNA sequences are listed using the IUPAC Ambiguity Codes. M represents A or C; R represents A or G; W represents A or T; S represents C or G; Y represents C or T; K represents G or T; V represents A or C or G; H represents A or C or T; D represents A or G or T; B represents C or G or T; N represents A or T or C or G.

‘Restriction enzyme’ lists the name of type IIS restriction enzyme. Restriction enzymes that recognize the same sequence are grouped together.

‘Restriction site’ lists the recognition sequence of the restriction enzyme.

‘Methylase’ listed the name of the methylase that could block the overlapping methylation/restriction site. Methylases that recognize the same sequence and generate the same pattern of methylation are grouped together.

‘Methylation sequence’ lists the recognition sequence of the methylase.

‘Position of methylated base’ listed the position of the methylated base or opposite base in the methylase recognition sequence.

‘Type of methylation’ lists the types of methylation for the methylated bases in the order of the positions of methylated bases listed in the previous column. m6A represents N6-methyladenine; m5C represents C5-methylcytosine; m4C represents N4-methylcytosine; ‘Unknown’ represents unknown type of methylation.

‘Overlapping methylation/restriction site’ lists the sequence of the overlapping methylation/restriction site that may result in blocking of the action of listed restriction enzyme through methylation by the listed methylase. For some pairs of restriction enzyme/methylase combination, there are several different overlapping methylation/restriction sites to choose from. The bases bracketed in ‘[ ]’ represent the adhesive ends generated by the restriction enzyme.

TABLE 3

| | | | | Posi- | | |
|---|---|---|---|---|---|---|
| | | | | tion | | |
| | Re- | | | of | | |
| | stric- | | Methyla- | methyl- | Type of | |
| Restriction | tion | | tion | ated | methyl- | Overlapping methylation/ |
| enzyme | site | Methylase | sequence | base | ation | restriction site |
| Sth132I | CCCG | M.AciI | CCGC | 1, 3 | m5C | CCCGCNNN[NNNN] |
| Sth132I | CCCG | M.Ama55I, M.CnaPQ2III, M.Rmo1926II, | CGGCCG | 1, 6 | m5C | CCCGGCCG[NNNN] |
| | | M.SenC1765IV, M.SspG1I, M.XorPXII | | | | |
| Sth132I | CCCG | M.ApaI | GGGCCC | 3, 4 | m5C | GGGCCCGNNNN[NNNN] |
| Sth132I | CCCG | M.AquI | CYCGRG | 1, 6 | m5C | CCCGRGNN[NNNN] |
| Sth132I | CCCG | M.AspDUT2 II, M.BstYI, M.CagIII, | RGATCY | 2, 5 | m4C | RGATCCCGNNNN[NNNN] |
| | | M.Mpa1757V, M.Mru1279V, M.RsaIII, | | | | |
| | | M.XhoII | | | | |
| Sth132I | CCCG | M.AspWA102I, M.AvaVII, M.BceJII, M.CbeI, | GGCC | 2, 3 | m4C | GGCCCGNNNN[NNNN] |
| | | M.Ckr177II, M.CmaLM2IV, M.CpeAVII, | | | | |
| | | M.Csp323II, M.PhoI, M.Ssp6714III, M.SuaI | | | | |
| Sth132I | CCCG | M.AvaV, M.SliSI | GATC | 1, 4 | m4C | GATCCCGNNNN[NNNN], |
| | | | | | | CCCGATCN[NNNN] |
| Sth1321 | CCCG | M.BamHI | GGATCC | 2, 5 | m4C | GGATCCCGNNNN[NNNN] |
| Sth132I | CCCG | M.BanII, M.EcoT38I | GRGCYC | 3, 4 | m5C | GRGCCCGNNNN[NNNN] |
| Sth132I | CCCG | M.BanLII, M.CpeAII, M.SinI | GGWCC | 2, 4 | m5C | GGWCCCGNNNN[NNNN] |
| Sth132I | CCCG | M.BceSII | GGWCC | 1, 5 | m5C | GGWCCCCGNNNN[NNNN], |
| | | | | | | GGWCCCGNNNN[NNNN], |
| | | | | | | CCCGGWCC[NNNN] |
| Sth132I | CCCG | M.BepI, M.FpsJV, M.Gel16401III, M.Pdi8503I | CGCG | 1, 4 | m5C | CCCGCGNN[NNNN] |
| Sth132I | CCCG | M.Bgl2196III | CGTACG | 2, 5 | m4C | CCCGTACG[NNNN] |
| Sth132I | CCCG | M.BhaII, M.BspRI, M.BsuRI, M.CthVI, | GGCC | 2, 3 | m5C | GGCCCGNNNN[NNNN] |
| | | M.DthLII, M.HaeIII, M.MthTI, M.NgoAII, | | | | |
| | | M.NgoPII, M.Pod15391I | | | | |
| Sth132I | CCCG | M.BsoBI | CYCGRG | 1, 6 | m4C | CCCGRGNN[NNNN] |
| Sth132I | CCCG | M.Bsp14III, M.Bsp3003I, M.Bsp3004I, | GATC | 1, 4 | m5C | GATCCCGNNNN[NNNN], |
| | | M.CalB3III, M.CpeAIII, M.Esu35585I, | | | | CCCGATCN[NNNN] |
| | | M.FpsJIV, M.Lba2031I, M.Lbo2004I, | | | | |
| | | M.LlaKR2I, M.Lmo1042I, M.Lmo7956II, | | | | |
| | | M.Lsp10393II, M.MspI7I, M.Sau3AI, M.Sba3I, | | | | |
| | | M.Sin395I, M.Spn23FI, M.Sth368I | | | | |
| Sth132I | CCCG | M.BsuFI, M.CpeAIV, M.MspI | CCGG | 1, 4 | m5C | CCCGGNNN[NNNN] |
| Sth132I | CCCG | M.Cac8I | GCNNGC | 2, 5 | m5C | GCCCGCNNN[NNNN] |
| Sth132I | CCCG | M.Cdi630III | CCSSGG | 2, 5 | m4C | CCCCGGNNN[NNNN], CCCGGGNN[NNNN], |
| | | | | | | CCCGSGGN[NNNN] |
| Sth132I | CCCG | M.Cfr9I, M.SmaI, M.Sma36365II, M.Xca85III, | CCCGGG | 2, 5 | m4C | CCCGGGNN[NNNN] |
| | | M.XcyI, M.XmaI, M.XveII | | | | |
| Sth132I | CCCG | M.CocIII, M.Fsi4947II, M.TdeIII, | GGNCC | 1, 5 | m4C | GGNCCCCGNNNN[NNNN], |
| | | M.Tph12270I | | | | GGNCCCGNNNN[NNNN], |
| | | | | | | GGCCCGNNNN[NNNN], CCCGGNCC[NNNN] |
| Sth132I | CCCG | M.CviJI | RGCB | 2, 3 | m5C | CCCGCYNN[NNNN], VGCCCGNNNN[NNNN] |
| Sth132I | CCCG | M.Dac11109II, M.Hfo6591I, M.MhuII, | GTAC | 1, 4 | m4C | GTACCCGNNNN[NNNN], |
| | | M.MjaV, M.MlaZII, M.MspLW5I, M.RsaI | | | | CCCGTACN[NNNN] |
| Sth132I | CCCG | M.Djo10085II, M.Ssp6803II | GGCC | 2, 3 | Unknown | GGCCCGNNNN[NNNN] |
| Sth132I | CCCG | M.DsaV, M.Ecl18kI, M.FpsJVI, M.NlaX, | CCNGG | 2, 4 | m5C | CCCGGNNN[NNNN] |
| | | M.SenPI, M.SsoII | | | | |
| Sth132I | CCCG | M.Eco29kI, M.NgoAIII, M.SacII | CCGCGG | 2, 5 | m5C | CCCGCGGN[NNNN] |
| Sth132I | CCCG | M.EcoO109I | RGGNCCY | 3, 5 | m5C | RGGNCCCGNNNN[NNNN] |
| Sth132I | CCCG | M.EfaRFII, M.Mae7806II | CCGG | 2, 3 | m4C | CCCGGNNN[NNNN] |
| Sth132I | CCCG | M.FisAW1I, M.ThaI, M.Tph12270II | CGCG | 1, 4 | m4C | CCCGCGNN[NNNN] |
| Sth132I | CCCG | M.FnoB1II, M.Hal24586I, M.Hpy298V, | CCGG | 1, 4 | m4C | CCCGGNNN[NNNN] |
| | | M.Hpy299IX, M.Hpy30V, M.Hpy32XI, | | | | |
| | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| Sth132I | CCCG | M.FpsJI, M.HpaII, M.Nme18II, M.NmeAI | CCGG | 2, 3 | m5C | CCCGGNNN[NNNN] |
| Sth1321 | CCCG | M.HgiCI | GGYRCC | 2, 5 | m5C | GGYRCCCGNNNN[NNNN] |
| Sth132I | CCCG | M.Hpy66I, M.Hpy991 | CGWCG | 2, 4 | m4C | CCCGWCGN[NNNN] |
| Sth132I | CCCG | M.Hpy99IV, M.HpyFORFX | CCNNGG | 1, 6 | m4C | CCCGGGNN[NNNN], CCCGNGGN[NNNN] |
| Sth132I | CCCG | M.Mae7806III, M.PspPI, M.Sau96I | GGNCC | 2, 4 | m5C | GGNCCCGNNNN[NNNN], |
| | | | | | | GGCCCGNNNN[NNNN] |
| Sth132I | CCCG | M.MjaII | GGNCC | 1, 5 | m5C | GGNCCCCGNNNN[NNNN], |
| | | | | | | GGNCCCGNNNN[NNNN], |
| | | | | | | GGCCCGNNNN[NNNN], CCCGGNCC[NNNN] |
| Sth132I | CCCG | M.MpeII | RGCY | 2, 3 | m5C | RGCCCGNNNN[NNNN] |
| Sth132I | CCCG | M.NgoAXV, M.NgoMV | GGNNCC | 2, 5 | m5C | GGNNCCCGNNNN[NNNN], |
| | | | | | | GGNCCCGNNNN[NNNN] |
| Sth132I | CCCG | M.NheI | GCTAGC | 1, 6 | m4C | GCTAGCCCGNNNN[NNNN] |
| Sth132I | CCCG | M.NmeDI | VCCGGY | 2, 5 | m5C | CCCGGYNN[NNNN] |
| Sth132I | CCCG | M.Pfa23572II | CGATCG | 1, 6 | m5C | CCCGATCG[NNNN] |
| Sth132I | CCCG | M.Psp737I | CCCCCD | 4 | m4C | CCCCCGNNNN[NNNN] |
| Sth132I | CCCG | M.RpaV | GCCCGCC | 4 | m4C | GCCCGCCNN[NNNN] |
| Sth132I | CCCG | M.SuaII | RGATCY | 2, 5 | m5C | RGATCCCGNNNN[NNNN] |
| Sth132I | CCCG | M1.Bag18758II, M2.Bag18758II | CCCGTG | 2, 4 | Unknown | CCCGTGNN[NNNN] |
| Sth132I | CCCG | M1.BcnI, M2.BcnI, M.NciI | CCSGG | 2, 4 | m4C | CCCGGNNN[NNNN] |
| Sth1321 | CCCG | M1.BspACI | CCGC | 1 | m5C | CCCGCNNN[NNNN] |
| Sth132I | CCCG | M1.CspNS6II | TCCGCC | 5 | m5C | TCCGCCCGNNNN[NNNN] |
| Alw26I, BcoDI, | GTCTC | M.AatII, M.Lsp6406III | GACGTC | 2, 5 | m6A | GACGTCTCN[NNNN] |
| BsmAI, BsoMAI, | | | | | | |
| BstMAI | | | | | | |
| Alw26I, BcoDI, | GTCTC | M.AbaBs12I, M.Awo1030I, M.Cdu23823I, | CCWGG | 1, 5 | m5C | CCWGGTCTCN[NNNN] |
| BsmAI, BsoMAI, | | M.Csa8155II, M.Cun9529I, M.EasL1Dcm, | | | | |
| BstMAI | | M.Eba57Dcm, M.Ecl34399Dcm, | | | | |
| | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| Alw26I, BcoDI, | GTCTC | M.AbrSp7I, M.Apa101655II, M.Ath21654I, | GANTC | 2, 4 | m6A | GAGTCTCN[NNNN] |
| BsmAI, BsoMAI, | | M.BdiNK6I, M.Bel76I, M.Bsp460I, | | | | |
| BstMAI | | M.BspJKG1II, M.BspLA1II, M.BspRAC04II, | | | | |
| | | M.CagII, M.CcrMI, M.CcrNAI, M.CnaPQ2I, | | | | |
| | | M.Csp16704III, M.CspNS6I, M.CviBI, | | | | |
| | | M.CviQVI, M.Eli8509I, M.Fnu419I, M.Fsp71I, | | | | |
| | | M.HbaI, M.HhaII, M.HinfI, M.HinHia1I, | | | | |
| | | M.Hpy298IV, M.Hpy299IV, M.Hpy32IV, | | | | |
| | | M.Hpy57IV, M.Hpy66IV, M.Hpy99IX, | | | | |
| | | M.HpyAIV, M.HpyFIV, M.HpyUM032IV, | | | | |
| | | M.HpyUM037IV, M.Mbo45III, M.Msp10I, | | | | |
| | | M.Msp73BI, M.MspAL11I, M.MspLW5II, | | | | |
| | | M.NhaXII, M.NpeUS61I, M.OspHL35I, | | | | |
| | | M.Pin17395II, M.Pla5III, M.PspLM6I, | | | | |
| | | M.RbaNRL2I, M.Rgi145II, M.Rmo1926I, | | | | |
| | | M.RpaIII, M.RsaII, M.RshIII, M.Rsp8I, | | | | |
| | | M.SmeC3I, M.SmoLV, M.Sna24252I, | | | | |
| | | M.SscL1I, M.SspM41II, M.SstE37II, M.ThaIII, | | | | |
| | | M.Xsp91I, M.ZmoCP4I | | | | |
| Alw26I, BcoDI, | GTCTC | M.AciI | GCGG | 2, 4 | m5C | GCGGTCTCN[NNNN] |
| BsmAI, BsoMAI, | | | | | | |
| BstMAI | | | | | | |
| Alw26I, BcoDI, | GTCTC | M.AflIII | ACRYGT | 2, 5 | m4C | ACRYGTCTCN[NNNN] |
| BsmAI, BsoMAI, | | | | | | |
| BstMAI | | | | | | |
| Alw26I, BcoDI, | GTCTC | M.AhdI | GACNNNN | 2, 10 | m6A | GACNNNNNGTCTCN[NNNN] |
| BsmAI, BsoMAI, | | | NGTC | | | |
| BstMAI | | | | | | |
| Alw26I, BcoDI, | GTCTC | M.Ama55I, M.CnaPQ2III, M.Rmo1926II, | CGGCCG | 1, 6 | m5C | CGGCCGTCTCN[NNNN] |
| BsmAI, BsoMAI, | | M.SenC1765IV, M.SspG1I, M.XorPXII | | | | |
| BstMAI | | | | | | |
| Alw26I, BcoDI, | GTCTC | M.AquI | CYCGRG | 1, 6 | m5C | CYCGRGTCTCN[NNNN] |
| BsmAI, BsoMAI, | | | | | | |
| BstMAI | | | | | | |
| Alw26I, BcoDI, | GTCTC | M.AvaIX, M.Cfr10I, M.Nme18VI, M.VchAI, | RCCGGY | 2, 5 | m5C | RCCGGTCTCN[NNNN] |
| BsmAI, BsoMAI, | | M.Vch0395I | | | | |
| BstMAI | | | | | | |
| Alw26I, BcoDI, | GTCTC | M.BcoSlacII, M.MspLW5III, M.Nbr128I, | CTCGAG | 1, 6 | m4C | CTCGAGTCTCN[NNNN] |
| BsmAI, BsoMAI, | | M.Psy18884II, M.Tam77409II, M.TbrGE1II, | | | | |
| BstMAI | | M.TpaRLII | | | | |
| Alw26I, BcoDI, | GTCTC | M.BepI, M.FpsJV, M.Gel16401III, M.Pdi8503I | CGCG | 1, 4 | m5C | CGCGTCTCN[NNNN] |
| BsmAI, BsoMAI, | | | | | | |
| BstMAI | | | | | | |
| Alw26I, BcoDI, | GTCTC | M.BsoBI | CYCGRG | 1, 6 | m4C | CYCGRGTCTCN[NNNN] |
| BsmAI, BsoMAI, | | | | | | |
| BstMAI | | | | | | |
| Alw26I, BcoDI, | GTCTC | M.BsuFI, M.CpeAIV, M.MspI | CCGG | 1, 4 | m5C | CCGGTCTCN[NNNN] |
| BsmAI, BsoMAI, | | | | | | |
| BstMAI | | | | | | |
| Alw26I, BcoDI, | GTCTC | M.Cab13497I, M.Dac11109III, M.Dco13527I, | CTAG | 1, 4 | m4C | CTAGTCTCN[NNNN] |
| BsmAI, BsoMAI, | | M.Djo10085I, M.Hbo11551I, M.Hka19301III, | | | | |
| BstMAI | | M.Hme33500I, M.HsaR1I, M.Hsi33800I, | | | | |
| | | M.HspKM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| Alw26I, BcoDI, | GTCTC | M.CagVIII | ACGCGT | 2, 5 | m4C | ACGCGTCTCN[NNNN] |
| BsmAI, BsoMAI, | | | | | | |
| BstMAI | | | | | | |
| Alw26I, BcoDI, | GTCTC | M.CfrBI, M.Sen1735IV, M.SenAnaIV | CCWWGG | 1, 6 | m4C | CCWWGGTCTCN[NNNN] |
| BsmAI, BsoMAI, | | | | | | |
| BstMAI | | | | | | |
| Alw26I, BcoDI, | GTCTC | M.Cmu51329I, M.EcoJA03PDcm, | CCWGG | 1, 5 | Unknown | CCWGGTCTCN[NNNN] |
| BsmAI, BsoMAI, | | M.RorR1Dcm, M.Sen8391 Dcm, M.ZalSM2II | | | | |
| BstMAI | | | | | | |
| Alw26I, BcoDI, | GTCTC | M.DdeI | CTNAG | 1, 5 | m5C | CTNAGTCTCN[NNNN] |
| BsmAI, BsoMAI, | | | | | | |
| BstMAI | | | | | | |
| Alw26I, BcoDI, | GTCTC | M.Esp3I | CGTCTC | 4, 3 | m5C, | |
| BsmAI, BsoMAI, | | | | | m6A | |
| BstMAI | | | | | | |
| Alw26I, BcoDI, | GTCTC | M.FatI | CATG | 1, 4 | m5C | CATGTCTCN[NNNN] |
| BsmAI, BsoMAI, | | | | | | |
| BstMAI | | | | | | |
| Alw26I, BcoDI, | GTCTC | M.FisAW1I, M.ThaI, M.Tph12270II | CGCG | 1, 4 | m4C | CGCGTCTCN[NNNN] |
| BsmAI, BsoMAI, | | | | | | |
| BstMAI | | | | | | |
| Alw26I, BcoDI, | GTCTC | M.FnoB1II, M.Hal24586I, M.Hpy298V, | CCGG | 1, 4 | m4C | CCGGTCTCN[NNNN] |
| BsmAI, BsoMAI, | | M.Hpy299IX, M.Hpy30V, M.Hpy32XI, | | | | |
| BstMAI | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| Alw26I, BcoDI, | GTCTC | M.GsuRed1IV, M.Osp807II, M.Tth111I | GACNNNG | 2, 8 | m6A | GACNNNGTCTCN[NNNN] |
| BsmAI, BsoMAI, | | | TC | | | |
| BstMAI | | | | | | |
| Alw26I, BcoDI, | GTCTC | M.Hpy298II, M.Hpy299II, M.Hpy66II, | ACNGT | 2, 4 | m4C | ACNGTCTCN[NNNN] |
| BsmAI, BsoMAI, | | M.HpyUM032II, M.HpyUM037II | | | | |
| BstMAI | | | | | | |
| Alw26I, BcoDI, | GTCTC | M.Hpy30X, M.Hpy57VIII, M.HpyFXI, M.MhuI | CTNAG | 1, 5 | m4C | CTNAGTCTCN[NNNN] |
| BsmAI, BsoMAI, | | | | | | |
| BstMAI | | | | | | |
| Alw26I, BcoDI, | GTCTC | M.Hpy99IV, M.HpyFORFX | CCNNGG | 1, 6 | m4C | CCNNGGTCTCN[NNNN] |
| BsmAI, BsoMAI, | | | | | | |
| BstMAI | | | | | | |
| Alw26I, BcoDI, | GTCTC | M.Hpy99XI | ACGT | 2, 3 | m5C | ACGTCTCN[NNNN] |
| BsmAI, BsoMAI, | | | | | | |
| BstMAI | | | | | | |
| Alw26I, BcoDI, | GTCTC | M.HpyD27I | GAGG | 2, 4 | m6A, | GAGGTCTCN[NNNN] |
| BsmAI, BsoMAI, | | | | | m5C | |
| BstMAI | | | | | | |
| Alw26I, BcoDI, | GTCTC | M.Lph145I, M.Lph93I | TCWCC | 1 | m6A | GTCTCC[NNNN] |
| BsmAI, BsoMAI, | | | | | | |
| BstMAI | | | | | | |
| Alw26I, BcoDI, | GTCTC | M.MlyI, M.Pen113I | GASTC | 2, 4 | m6A | GAGTCTCN[NNNN] |
| BsmAI, BsoMAI, | | | | | | |
| BstMAI | | | | | | |
| Alw26I, BcoDI, | GTCTC | M.Msp1II | CCAGG | 5 | m4C | CCAGGTCTCN[NNNN] |
| BsmAI, BsoMAI, | | | | | | |
| BstMAI | | | | | | |
| Alw26I, BcoDI, | GTCTC | M.Msp42II | CCWGG | 1, 5 | m4C | CCWGGTCTCN[NNNN] |
| BsmAI, BsoMAI, | | | | | | |
| BstMAI | | | | | | |
| Alw26I, BcoDI, | GTCTC | M.NmeDI | VCCGGY | 2, 5 | m5C | VCCGGTCTCN[NNNN] |
| BsmAI, BsoMAI, | | | | | | |
| BstMAI | | | | | | |
| Alw26I, BcoDI, | GTCTC | M.NmeMC58III | TCTGG | 5 | m5C | TCTGGTCTCN[NNNN] |
| BsmAI, BsoMAI, | | | | | | |
| BstMAI | | | | | | |
| Alw26I, BcoDI, | GTCTC | M.Nsp209III, M.Nsp51109II | CTCGAG | 1, 6 | m5C | CTCGAGTCTCN[NNNN] |
| BsmAI, BsoMAI, | | | | | | |
| BstMAI | | | | | | |
| Alw26I, BcoDI, | GTCTC | M.NspI, M.NspHI | RCATGY | 2, 5 | m5C | RCATGTCTCN[NNNN] |
| BsmAI, BsoMAI, | | | | | | |
| BstMAI | | | | | | |
| Alw26I, BcoDI, | GTCTC | M.Pam7686I | CCATGG | 1, 6 | m5C | CCATGGTCTCN[NNNN] |
| BsmAI, BsoMAI, | | | | | | |
| BstMAI | | | | | | |
| Alw26I, BcoDI, | GTCTC | M.Pfa23572II | CGATCG | 1, 6 | m5C | CGATCGTCTCN[NNNN] |
| BsmAI, BsoMAI, | | | | | | |
| BstMAI | | | | | | |
| Alw26I, BcoDI, | GTCTC | M.Pla5I | CTAG | 1, 4 | Unknown | CTAGTCTCN[NNNN] |
| BsmAI, BsoMAI, | | | | | | |
| BstMAI | | | | | | |
| Alw26I, BcoDI, | GTCTC | M.Sam53668II | CCATGG | 1, 6 | Unknown | CCATGGTCTCN[NNNN] |
| BsmAI, BsoMAI, | | | | | | |
| BstMAI | | | | | | |
| Alw26I, BcoDI, | GTCTC | M.Sen1781III | CCWWGG | 1, 6 | Unknown | CCWWGGTCTCN[NNNN] |
| BsmAI, BsoMAI, | | | | | | |
| BstMAI | | | | | | |
| Alw26I, BcoDI, | GTCTC | M.SonIII | GCANNNN | 3, 9 | m6A | |
| BsmAI, BsoMAI, | | | GTC | | | |
| BstMAI | | | | | | |
| Alw26I, BcoDI, | GTCTC | M.TdeII | GAAGAG | 5, 6 | m6A, | GAAGAGTCTCN[NNNN] |
| BsmAI, BsoMAI, | | | | | m4C | |
| BstMAI | | | | | | |
| Alw26I, BcoDI, | GTCTC | M.TspRI | CASTG | 1, 5 | m5C | CASTGTCTCN[NNNN] |
| BsmAI, BsoMAI, | | | | | | |
| BstMAI | | | | | | |
| Alw26I, BcoDI, | GTCTC | M1.AgoG2I, M2.AgoG2I | CCAGT | 1, 4 | m4C | ACTGGTCTCN[NNNN], |
| BsmAI, BsoMAI, | | | | | | CCAGTCTCN[NNNN] |
| BstMAI | | | | | | |
| Alw26I, BcoDI, | GTCTC | M1.BceSIII | GCCGT | 4 | m4C | GCCGTCTCN[NNNN] |
| BsmAI, BsoMAI, | | | | | | |
| BstMAI | | | | | | |
| Alw26I, BcoDI, | GTCTC | M1.BspACI | GCGG | 4 | m5C | GCGGTCTCN[NNNN] |
| BsmAI, BsoMAI, | | | | | | |
| BstMAI | | | | | | |
| Alw26I, BcoDI, | GTCTC | M1.Dsh12II, M2.Dsh12II | GCCGTC | 3, 4 | m4C | GCCGTCTCN[NNNN] |
| BsmAI, BsoMAI, | | | | | | |
| BstMAI | | | | | | |
| Alw26I, BcoDI, | GTCTC | M1.Eco31I, M1.EcoMI | GGTCTC | 3 | m6A | GGTCTCN[NNNN] |
| BsmAI, BsoMAI, | | | | | | |
| BstMAI | | | | | | |
| Alw26I, BcoDI, | GTCTC | M1.Hpy32II | TCYYC | 1 | m6A | GTCTCC[NNNN] |
| BsmAI, BsoMAI, | | | | | | |
| BstMAI | | | | | | |
| Alw26I, BcoDI, | GTCTC | M1.LplAG30I | GAAGG | 5 | m5C | GAAGGTCTCN[NNNN] |
| BsmAI, BsoMAI, | | | | | | |
| BstMAI | | | | | | |
| Alw26I, BcoDI, | GTCTC | M2.BceSIII, M1.Pru9I, M2.Pru9I | GCCGT | 2, 4 | m4C | GCCGTCTCN[NNNN] |
| BsmAI, BsoMAI, | | | | | | |
| BstMAI | | | | | | |
| Alw26I, BcoDI, | GTCTC | M2.BfiI | CCCAGT | 5 | m4C | CCCAGTCTCN[NNNN] |
| BsmAI, BsoMAI, | | | | | | |
| BstMAI | | | | | | |
| Alw26I, BcoDI, | GTCTC | M2.BfuAI | GCAGGT | 5 | m5C | GCAGGTCTCN[NNNN] |
| BsmAI, BsoMAI, | | | | | | |
| BstMAI | | | | | | |
| Alw26I, BcoDI, | GTCTC | M2.Eco31I | GGTCTC | 4 | m5C | GGTCTCN[NNNN] |
| BsmAI, BsoMAI, | | | | | | |
| BstMAI | | | | | | |
| Alw26I, BcoDI, | GTCTC | M2.HgaI, M1.LlaJI | GCGTC | 3 | m5C | GCGTCTCN[NNNN] |
| BsmAI, BsoMAI, | | | | | | |
| BstMAI | | | | | | |
| Alw26I, BcoDI, | GTCTC | M2.HpyAVI, M1.MnlI | GAGG | 4 | m5C | GAGGTCTCN[NNNN] |
| BsmAI, BsoMAI, | | | | | | |
| BstMAI | | | | | | |
| Alw26I, BcoDI, | GTCTC | M2.Nme579IV, M2.NmeMC58II | GCGTC | 4 | m6A | GCGTCTCN[NNNN] |
| BsmAI, BsoMAI, | | | | | | |
| BstMAI | | | | | | |
| AlwXI, BbvI, | GCAG | M.AbaBs12I, M.Awo1030I, M.Cdu23823I, | CCWGG | 1, 5 | m5C | CCWGGCAGCNNNNNNNN[NNNN], |
| BseKI, BseXI, | C | M.Csa8155II, M.Cun9529I, M.EasL1Dcm, | | | | GCAGCCWGGNNNN[NNNN] |
| Bsp423I, Bst12I, | | M.Eba57Dcm, M.Ecl34399Dcm, | | | | |
| Bst71I, BstV1I, | | M.Eco12761Dcm, M.Eco3609Dcm, | | | | |
| GeoICI, Lsp1109I | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| AlwXI, BbvI, | GCAG | M.Acc65VI, M.SenAZIII | CTKMAG | 2, 5 | m6A | CTGCAGCNNNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.AciI | CCGC | 1, 3 | m5C | GCGGCAGCNNNNNNNN[NNNN], |
| BseKI, BseXI, | C | | | | | GCAGCGGNNNNNN[NNNN], |
| Bsp423I, Bst12I, | | | | | | CCGCAGCNNNNNNNN[NNNN], |
| Bst71I, BstV1I, | | | | | | GCAGCCGCNNNNN[NNNN] |
| GeoICI, Lsp1109I | | | | | | |
| AlwXI, BbvI, | GCAG | M.Ade56712I, M.Asp588III, M.AveCB51II, | CTGCAG | 2, 5 | m6A | CTGCAGCNNNNNNNN[NNNN] |
| BseKI, BseXI, | C | M.BbrUIII, M.Blo30698I, M.BsuBI, | | | | |
| Bsp423I, Bst12I, | | M.CpaYL7III, M.Dba7044I, M.Dfa45958II, | | | | |
| Bst71I, BstV1I, | | M.Eco12581I, M.Eco2012I, M.EcoGIII, | | | | |
| GeoICI, Lsp1109I | | M.EcoG089I, M.Eho16691II, M.Hal5920II, | | | | |
| | | M.Lsp6406II, M.MspNR41II, M.Pae490I, | | | | |
| | | M.Pox23570I, M.PstI, M.RpaII, M.Sca3403II, | | | | |
| | | M.Tsp61I, M.XmnII, M.YenWI, M.YinY228I | | | | |
| AlwXI, BbvI, | GCAG | M.AluBI | AGCT | 1, 4 | m6A | GCAGCTNNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.AluI, M.CspCY2I | AGCT | 2, 3 | m5C | GCAGCTNNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.Ama55I, M.CnaPQ2III, M.Rmo1926II, | CGGCCG | 1, 6 | m5C | CGGCCGCAGCNNNNNNNN[NNNN], |
| BseKI, BseXI, | C | M.SenC1765IV, M.SspG1I, M.XorPXII | | | | GCAGCGGCCGNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.AplI | CTGCAG | 3, 4 | m5C | CTGCAGCNNNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.AquI | CYCGRG | 1, 6 | m5C | CYCGRGCAGCNNNNNNNN[NNNN], |
| BseKI, BseXI, | C | | | | | GCAGCYCGRGNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.AsiSI | GCGATCGC | 2, 7 | m5C | GCGATCGCAGCNNNNNNNN[NNNN], |
| BseKI, BseXI, | C | | | | | GCAGCGATCGCNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.AvaIX, M.Cfr10I, M.Nme18VI, M.VchAI, | RCCGGY | 2, 5 | m5C | RCCGGCAGCNNNNNNNN[NNNN], |
| BseKI, BseXI, | C | M.Vch0395I | | | | GCAGCCGGYNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.BcoSlacII, M.MspLW5III, M.Nbr128I, | CTCGAG | 1, 6 | m4C | CTCGAGCAGCNNNNNNNN[NNNN], |
| BseKI, BseXI, | C | M.Psy18884II, M.Tam77409II, M.TbrGE1II, | | | | GCAGCTCGAGNNN[NNNN] |
| Bsp423I, Bst12I, | | M.TpaRLII | | | | |
| Bst71I, BstV1I, | | | | | | |
| GeoICI, Lsp1109I | | | | | | |
| AlwXI, BbvI, | GCAG | M.BepI, M.FpsJV, M.Gel16401III, M.Pdi8503I | CGCG | 1, 4 | m5C | CGCGCAGCNNNNNNNN[NNNN], |
| BseKI, BseXI, | C | | | | | GCAGCGCGNNNNN[NNNN] |
| Bsp423I, Bst12I, | | | | | | |
| Bst71I, BstV1I, | | | | | | |
| GeoICI, Lsp1109I | | | | | | |
| AlwXI, BbvI | GCAG | M.BglI, M.CagI, M.DdeVCDI, M.GsuRed1I | GCCNNNN | 2, 10 | m4C | GCCNNNNNGGCAGCNNNNNNNN[NNNN] |
| BseKI, BseXI, | C | | NGGC | | | |
| Bsp423I, Bst12I, | | | | | | |
| Bst71I BstV1I, | | | | | | |
| GeoICI, Lsp1109I | | | | | | |
| AlwXI, BbvI, | GCAG | M.Ble402III, M.Psp10HI | SAGCTS | 3, 4 | m4C | GCAGCTSNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.BsoBI | CYCGRG | 1, 6 | m4C | CYCGRGCAGCNNNNNNNN[NNNN], |
| BseKI, BseXI, | C | | | | | GCAGCYCGRGNNN[NNNN] |
| Bsp423I, Bst12I, | | | | | | |
| Bst71I, BstV1I, | | | | | | |
| GeoICI, Lsp11091 | | | | | | |
| AlwXI, BbvI, | GCAG | M.BsuFI, M.CpeAIV, M.MspI | CCGG | 1, 4 | m5C | CCGGCAGCNNNNNNNN[NNNN], |
| BseKI, BseXI, | C | | | | | GCAGCCGGNNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.Cab13497I, M.Dac11109III, M.Dco13527I, | CTAG | 1, 4 | m4C | CTAGCAGCNNNNNNNN[NNNN], |
| BseKI, BseXI, | C | M.Djo10085I, M.Hbo11551I, M.Hka19301III, | | | | GCAGCTAGNNNNN[NNNN] |
| Bsp423I, Bst12I, | | M.Hme33500I, M.HsaR1I, M.Hsi33800I, | | | | |
| Bst71I, BstV1I, | | M.HspKM1WII, M.HspNII, M.HtuI, M.HvoDSI, | | | | |
| GeoICI, Lsp1109I | | M.Lba2029II, M.Mbo45II, M.MjaI, M.MthZI, | | | | |
| | | M.Nas12278I, M.Rfl3010I, M.SsmMI | | | | |
| AlwXI, BbvI, | GCAG | M.Cac8I | GCNNGC | 2, 5 | m5C | GCNNGCAGCNNNNNNNN[NNNN], |
| BseKI, BseXI, | C | | | | | GCAGCNNGCNNNN[NNNN] |
| Bsp423I, Bst12I, | | | | | | |
| Bst71I, BstV1I, | | | | | | |
| GeoICI, Lsp1109I | | | | | | |
| AlwXI, BbvI | GCAG | M.CcrNAV | YGCCGGCR | 3, 6 | m5C | YGCCGGCAGCNNNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI | GCAG | M.CfrBI, M.Sen1735IV, M.SenAnaIV | CCWWGG | 1, 6 | m4C | CCWWGGCAGCNNNNNNNN[NNNN], |
| BseKI, BseXI, | C | | | | | GCAGCCWWGGNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.Cmu51329I, M.EcoJA03PDcm, | CCWGG | 1, 5 | Unknown | CCWGGCAGCNNNNNNNN[NNNN], |
| BseKI, BseXI, | C | M.RorR1Dcm, M.Sen8391Dcm, M.ZalSM2II | | | | GCAGCCWGGNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.CsiFSMII, M.MfoCT6IV, M.NgoAIV, | GCCGGC | 2, 5 | m5C | GCCGGCAGCNNNNNNNN[NNNN], |
| BseKI, BseXI, | C | M.NgoMIV, M1.Sgr13350II | | | | GCAGCCGGCNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.CviJI | RGCB | 2, 3 | m5C | GCAGCBNNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.CviRI, M.Hpy298VIII, M.Hpy299VIII, | TGCA | 1, 4 | m6A | TGCAGCNNNNNNNN[NNNN] |
| BseKI, BseXI, | C | M.Hpy30VI, M.Hpy32V, M.Hpy57II, | | | | |
| Bsp423I, Bst12I, | | M.Hpy66VIII, M.HpyFVI, M.HpyUM032VIII, | | | | |
| Bst71I, BstV1I, | | M.HpyUM037VIII | | | | |
| GeoICI, Lsp1109I | | | | | | |
| AlwXI, BbvI, | GCAG | M.Ddel | CTNAG | 1, 5 | m5C | CTNAGCAGCNNNNNNNN[NNNN], |
| BseKI, BseXI, | C | | | | | GCAGCTNAGNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.DfeI, M.Fnu419II, M.Pen113II | CTNNAG | 2, 5 | m6A | CTGCAGCNNNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.FatI | CATG | 1, 4 | m5C | CATGCAGCNNNNNNNN[NNNN], |
| BseKI, BseXI, | C | | | | | GCAGCATGNNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.FisAW1I, M.ThaI, M.Tph12270II | CGCG | 1, 4 | m4C | CGCGCAGCNNNNNNNN[NNNN], |
| BseKI, BseXI, | C | | | | | GCAGCGCGNNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.FnoB1II, M.Hal24586I, M.Hpy298V, | CCGG | 1, 4 | m4C | CCGGCAGCNNNNNNNN[NNNN], |
| BseKI, BseXI, | C | M.Hpy299IX, M.Hpy30V, M.Hpy32XI, | | | | GCAGCCGGNNNNN[NNNN] |
| Bsp423I, Bst12I, | | M.Hpy57V, M.Hpy66IX, M.Hpy99VIII, | | | | |
| Bst71I, BstV1I, | | M.HpyFV, M.HpyUM032IX, M.HpyUM037IX, | | | | |
| GeoICI, Lsp1109I | | M.MjaVI, M.PspJDRI, M.TmeBII | | | | |
| AlwXI, BbvI, | GCAG | M.Fsi4947I, M.Mru1279IV, M.NsoJS138I, | CAGCTG | 3, 4 | m4C | GCAGCTGNNNNNN[NNNN] |
| BseKI, BseXI, | C | M1.Psp320WII, M.PvuII, M.SenA46IV, | | | | |
| Bsp423I, Bst12I, | | M.SptAI | | | | |
| Bst71I, BstV1I, | | | | | | |
| GeoICI, Lsp1109I | | | | | | |
| AlwXI, BbvI, | GCAG | M.HhaI, M.HinP1I, M.Hpy99III, M.HpyAVIII, | GCGC | 2, 3 | m5C | GCGCAGCNNNNNNNN[NNNN], |
| BseKI, BseXI, | C | M.HpyUM032XV | | | | GCAGCGCNNNNNN[NNNN] |
| Bsp423I, Bst12I, | | | | | | |
| Bst71I, BstV1I, | | | | | | |
| GeoICI, Lsp11091 | | | | | | |
| AlwXI, BbvI, | GCAG | M.Hme33500II | HGCWGCK | 3 | m4C | MGCWGCAGCNNNNNNNN[NNNN], |
| BseKI, BseXI, | C | | | | | MGCAGCDNNNNNNN[NNNN], |
| Bsp423I, Bst12I, | | | | | | HGCAGCKNNNNNNN[NNNN], |
| Bst71I, BstV1I, | | | | | | GCAGCWGCKNNNN[NNNN] |
| GeoICI, Lsp11091 | | | | | | |
| AlwXI, BbvI, | GCAG | M.Hpy30X, M.Hpy57VIII, M.HpyFXI, M.MhuI | CTNAG | 1, 5 | m4C | CTNAGCAGCNNNNNNNN[NNNN], |
| BseKI, BseXI, | C | | | | | GCAGCTNAGNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.Hpy57X | CTRYAG | 2, 5 | m6A | CTGCAGCNNNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.Hpy99IV, M.HpyFORFX | CCNNGG | 1, 6 | m4C | CCNNGGCAGCNNNNNNNN[NNNN], |
| BseKI, BseXI, | C | | | | | GCAGCCNNGGNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.HpyD27I | GAGG | 2, 4 | m6A, | GCAGCCTCNNNNN[NNNN], |
| BseKI, BseXI, | C | | | | m5C | GAGGCAGCNNNNNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.Mma5219II, M.TarMY3I | AGCT | 2, 3 | m4C | GCAGCTNNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.MpeII | RGCY | 2, 3 | m5C | GCAGCYNNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.Msp1II | CCAGG | 5 | m4C | GCAGCCTGGNNNN[NNNN], |
| BseKI, BseXI, | C | | | | | CCAGGCAGCNNNNNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.Msp42II | CCWGG | 1, 5 | m4C | CCWGGCAGCNNNNNNNN[NNNN], |
| BseKI, BseXI, | C | | | | | GCAGCCWGGNNNN[NNNN] |
| Bsp4231 Bst12I, | | | | | | |
| Bst71I, BstV1I, | | | | | | |
| GeoICI, Lsp1109I | | | | | | |
| AlwXI, BbvI, | GCAG | M.NgoAI | RGCGCY | 3, 4 | m5C | GCAGCGCYNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.NheI | GCTAGC | 1, 6 | m4C | GCTAGCAGCNNNNNNNN[NNNN], |
| BseKI, BseXI, | C | | | | | GCAGCTAGCNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.Nme18V | GCGCGC | 2, 5 | m5C | GCGCGCAGCNNNNNNNN[NNNN], |
| BseKI, BseXI, | C | | | | | GCAGCGCGCNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.NmeDI | VCCGGY | 2, 5 | m5C | VCCGGCAGCNNNNNNNN[NNNN], |
| BseKI, BseXI, | C | | | | | GCAGCCGGBNNNN[NNNN] |
| Bsp423I, Bst12I, | | | | | | |
| Bst71I, BstV1I | | | | | | |
| GeoICI, Lsp1109I | | | | | | |
| AlwXI, BbvI, | GCAG | M.NmeMC58III | CCAGA | 1 | m5C | TCTGGCAGCNNNNNNNN[NNNN], |
| BseKI, BseXI, | C | | | | | GCAGCCAGANNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.Nsp209III, M.Nsp51109II | CTCGAG | 1, 6 | m5C | CTCGAGCAGCNNNNNNNN[NNNN], |
| BseKI, BseXI, | C | | | | | GCAGCTCGAGNNN[NNNN] |
| Bsp423I, Bst12I, | | | | | | |
| Bst71I, BstV1I, | | | | | | |
| GeoICI, Lsp11091 | | | | | | |
| AlwXI, BbvI, | GCAG | M.NspI, M.NspHI | RCATGY | 2, 5 | m5C | RCATGCAGCNNNNNNNN[NNNN], |
| BseKI, BseXI, | C | | | | | GCAGCATGYNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.Pae10332II, M1.Pae50071I, | CAGCTC | 3, 4 | m4C | GCAGCTCNNNNNN[NNNN] |
| BseKI, BseXI, | C | M2.Pae50071I | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.Pam7686I | CCATGG | 1, 6 | m5C | CCATGGCAGCNNNNNNNN[NNNN], |
| BseKI, BseXI, | C | | | | | GCAGCCATGGNNN[NNNN] |
| Bsp423I, Bst12I, | | | | | | |
| Bst71I, BstV1I, | | | | | | |
| GeoICI, Lsp11091 | | | | | | |
| AlwXI, BbvI, | GCAG | M.Pfa23572II | CGATCG | 1, 6 | m5C | CGATCGCAGCNNNNNNNN[NNNN], |
| BseKI, BseXI, | C | | | | | GCAGCGATCGNNN[NNNN] |
| Bsp423I, Bst12I, | | | | | | |
| Bst71I, BstV1I, | | | | | | |
| GeoICI, Lsp11091 | | | | | | |
| AlwXI, BbvI, | GCAG | M.Pla5I | CTAG | 1, 4 | Unknown | CTAGCAGCNNNNNNNN[NNNN], |
| BseKI, BseXI, | C | | | | | GCAGCTAGNNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.Sam53668II | CCATGG | 1, 6 | Unknown | CCATGGCAGCNNNNNNNN[NNNN], |
| BseKI, BseXI, | C | | | | | GCAGCCATGGNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.SbaUII | CAGCTG | 2, 5 | m6A | GCAGCTGNNNNNN[NNNN] |
| BseKI, BseXI, | C | | | | | |
| Bsp423I, Bst12I, | | | | | | |
| Bst71I, BstV1I, | | | | | | |
| GeoICI, Lsp1109I | | | | | | |
| AlwXI, BbvI, | GCAG | M.Sen1781III | CCWWGG | 1, 6 | Unknown | CCWWGGCAGCNNNNNNNN[NNNN], |
| BseKI, BseXI, | C | | | | | GCAGCCWWGGNNN[NNNN] |
| Bsp423I Bst12I, | | | | | | |
| Bst71I, BstV1I, | | | | | | |
| GeoICI, Lsp1109I | | | | | | |
| AlwXI, BbvI, | GCAG | M.SonIII | GCANNNN | 3, 9 | m6A | GCAGCNNGTCNNN[NNNN] |
| BseKI, BseXI, | C | | GTC | | | |
| Bsp423I, Bst12I, | | | | | | |
| Bst71I, BstV1I, | | | | | | |
| GeoICI, Lsp11091 | | | | | | |
| AlwXI, BbvI, | GCAG | M.TdeII | CTCTTC | 1, 2 | m4C, | GAAGAGCAGCNNNNNNNN[NNNN], |
| BseKI, BseXI, | C | | | | m6A | GCAGCTCTTCNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.TspRI | CASTG | 1, 5 | m5C | CASTGCAGCNNNNNNNN[NNNN], |
| BseKI, BseXI, | C | | | | | GCAGCASTGNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M.Yen2516I | GCGCGC | 2, 5 | Unknown | GCGCGCAGCNNNNNNNN[NNNN], |
| BseKI, BseXI, | C | | | | | GCAGCGCGCNNNN[NNNN] |
| Bsp423I, Bst12I, | | | | | | |
| Bst71I, BstV1I, | | | | | | |
| GeoICI, Lsp11091 | | | | | | |
| AlwXI, BbvI, | GCAG | M1.AgoG2I, M2.AgoG2I | CCAGT | 1, 4 | m4C | ACTGGCAGCNNNNNNNN[NNNN], |
| BseKI, BseXI, | C | | | | | GCAGCCAGTNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M1.BspACI | CCGC | 1 | m5C | GCGGCAGCNNNNNNNN[NNNN], |
| BseKI, BseXI, | C | | | | | GCAGCCGCNNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M1.HgaI, M2.LlaJI | GCGTC | 2 | m5C | GACGCAGCNNNNNNNN[NNNN], |
| BseKI, BseXI, | C | | | | | GCAGCGTCNNNNN[NNNN] |
| Bsp423I, Bst12I, | | | | | | |
| Bst71I, BstV1I, | | | | | | |
| GeoICI, Lsp1109I | | | | | | |
| AlwXI BbvI | GCAG | M1.LplAG30I | GAAGG | 5 | m5C | GCAGCCTTCNNNN[NNNN], |
| BseKI, BseXI, | C | | | | | GAAGGCAGCNNNNNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M1.Sgr13350III, M2.Sgr13350III | GCTCTTC | 2, 3 | m4C, | GAAGAGCAGCNNNNNNNN[NNNN], |
| BseKI, BseXI, | C | | | | m6A | GCAGCTCTTCNNN[NNNN] |
| Bsp423I, Bst12I, | | | | | | |
| Bst71I, BstV1I, | | | | | | |
| GeoICI, Lsp1109I | | | | | | |
| AlwXI, BbvI | GCAG | M2.BceSIII, M1.Pru9I, M2.Pru9I | ACGGC | 2, 4 | m4C | GCAGCCGTNNNNN[NNNN], |
| BseKI, BseXI, | C | | | | | ACGGCAGCNNNNNNNN[NNNN] |
| Bsp423I, Bst12I, | | | | | | |
| Bst71I, BstV1I, | | | | | | |
| GeoICI, Lsp1109I | | | | | | |
| AlwXI, BbvI, | GCAG | M2.HpyAVI, M1.MnlI | GAGG | 4 | m5C | GCAGCCTCNNNNN[NNNN], |
| BseKI, BseXI, | C | | | | | GAGGCAGCNNNNNNNN[NNNN] |
| Bsp423I, Bst12I, | | | | | | |
| Bst71I, BstV1I, | | | | | | |
| GeoICI, Lsp1109I | | | | | | |
| BmsI, BspST5I, | GCAT | M.AbaBs12I, M.Awo1030I, M.Cdu23823I, | CCWGG | 1, 5 | m5C | CCWGGCATCNNNNN[NNNN], |
| LweI, PhaI, SfaNI | C | M.Csa8155II, M.Cun9529I, M.EasL1Dcm, | | | | GCATCCWGGN[NNNN] |
| | | M.Eba57Dcm, M.Ec134399Dcm, | | | | |
| | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| BmsI, BspST5I, | GCAT | M.AciI | CCGC | 1, 3 | m5C | GCGGCATCNNNNN[NNNN], |
| LweI, PhaI, SfaNI | C | | | | | CCGCATCNNNNN[NNNN], |
| | | | | | | GCATCCGCNN[NNNN] |
| BmsI, BspST5I, | GCAT | M.Ama55I, M.CnaPQ2III, M.Rmo1926II, | CGGCCG | 1, 6 | m5C | CGGCCGCATCNNNNN[NNNN], |
| LweI, PhaI, SfaNI | C | M.SenC1765IV, M.SspG1I, M.XorPXII | | | | GCATCGGCCG[NNNN] |
| BmsI, BspST5I, | GCAT | M.AquI | CYCGRG | 1, 6 | m5C | CYCGRGCATCNNNNN[NNNN], |
| LweI, PhaI, SfaNI | C | | | | | GCATCYCGRG[NNNN] |
| BmsI, BspST5I, | GCAT | M.AsiSI | GCGATCGC | 2, 7 | m5C | GCGATCGCATCNNNNN[NNNN] |
| LweI, PhaI, SfaNI | C | | | | | |
| BmsI, BspST5I, | GCAT | M.AvaIX, M.Cfr10I, M.Nme18VI, M.VchAI, | RCCGGY | 2, 5 | m5C | RCCGGCATCNNNNN[NNNN] |
| LweI, PhaI, SfaNI | C | M.Vch0395I | | | | |
| BmsI, BspST5I, | GCAT | M.BamWS8I, M.CthV, M.EfaRFI, M.Saf8902II, | GCWGC | 2, 4 | m5C | GCWGCATCNNNNN[NNNN] |
| LweI, PhaI, SfaNI | C | M.TacII, M.TcoKWC4III | | | | |
| BmsI, BspST5I, | GCAT | M.BbvI, M.BceSIV | GCAGC | 2, 4 | m5C | GCTGCATCNNNNN[NNNN], |
| LweI, PhaI, SfaNI | C | | | | | GCAGCATCNNNNN[NNNN] |
| BmsI, BspST5I, | GCAT | M.BcoSlacII, M.MspLW5III, M.Nbr128I, | CTCGAG | 1, 6 | m4C | CTCGAGCATCNNNNN[NNNN], |
| LweI, PhaI, SfaNI | C | M.Psy18884II, M.Tam77409II, M.TbrGE1II, | | | | GCATCTCGAG[NNNN] |
| | | M.TpaRLII | | | | |
| BmsI, BspST5I, | GCAT | M.BepI, M.FpsJV, M.Gel16401III, M.Pdi8503I | CGCG | 1, 4 | m5C | CGCGCATCNNNNN[NNNN], |
| LweI, PhaI, SfaNI | C | | | | | GCATCGCGNN[NNNN] |
| BmsI, BspST5I, | GCAT | M.BfaSIII, M.Ccl10114I, M.CfrB38I, | ATGCAT | 2, 5 | m6A | ATGCATCNNNNN[NNNN] |
| LweI, PhaI, SfaNI | C | M.CfrH1I, M.EcoGVI, M.EcoKII, | | | | |
| | | M.Fnu10562II, M.SbaUIV, M.Sbo12419III, | | | | |
| | | M.Sbo268II, M.Sen10384II, M.Sen10708II, | | | | |
| | | M.Sen1080II, M.Sen1175III, M.Sen13IV, | | | | |
| | | M.Sen13311IV, M.Sen1387II, M.Sen1427III, | | | | |
| | | M.Sen14882III, M.Sen15791III, M.Sen158III, | | | | |
| | | M.Sen16I, M.Sen1655III, M.Sen1676III, | | | | |
| | | M.Sen1677III, M.Sen1728III, M.Sen1735III, | | | | |
| | | M.Sen1736IV, M.Sen1764III, M.Sen1766III, | | | | |
| | | M.Sen1781IV, M.Sen1783III, M.Sen18569II, | | | | |
| | | M.Sen1878III, M.Sen1880III, M.Sen1896III, | | | | |
| | | M.Sen1898III, M.Sen1899III, M.Sen1903III, | | | | |
| | | M.Sen1906III, M.Sen1908III, M.Sen1910III, | | | | |
| | | M.Sen1921III, M.Sen1927II, M.Sen195II, | | | | |
| | | M.Sen2050II, M.Sen2064III, M.Sen2069III, | | | | |
| | | M.Sen22462I, M.Sen255II, M.Sen318III, | | | | |
| | | M.Sen3387III, M.Sen3890III, M.Sen483III, | | | | |
| | | M.Sen624III, M.Sen641III, M.Sen7378II, | | | | |
| | | M.Sen8391III, M.Sen8692III, M.SenA46III, | | | | |
| | | M.SenAbaII, M.SenAboII, M.SenAnaIII, | | | | |
| | | M.SenC1736III, M.SenC1765III, | | | | |
| | | M.SenC1808IV, M.SenC1810III, M.SenJIII, | | | | |
| | | M.SenL1II, M.SenL15III, M.SenN1660II, | | | | |
| | | M.SenSARA26II, M.SenSPBIII, M.SenTFIII | | | | |
| BmsI, BspST5I, | GCAT | M.BglI, M.CagI, M.DdeVCDI, M.GsuRed1I | GCCNNNN | 2, 10 | m4C | GCCNNNNNGGCATCNNNNN[NNNN] |
| LweI, PhaI, SfaNI | C | | NGGC | | | |
| BmsI, BspST5I, | GCAT | M.BseCI, M.ClaI, M.MfoCT6III, M.Nfa11134I, | ATCGAT | 2, 5 | m6A | GCATCGATNN[NNNN] |
| LweI, PhaI, SfaNI | C | M.Nsp209I, M.Nsp51109I, M.Pfu17724II, | | | | |
| | | M.SonI | | | | |
| BmsI, BspST5I, | GCAT | M.BsoBI | CYCGRG | 1, 6 | m4C | CYCGRGCATCNNNNN[NNNN], |
| LweI, PhaI, SfaNI | C | | | | | GCATCYCGRG[NNNN] |
| BmsI, BspST5I, | GCAT | M.BsuFI, M.CpeAIV, M.MspI | CCGG | 1, 4 | m5C | CCGGCATCNNNNN[NNNN], |
| LweI, PhaI, SfaNI | C | | | | | GCATCCGGNN[NNNN] |
| BmsI, BspST5I, | GCAT | M.Cab13497I, M.Dac11109III, M.Dco13527I, | CTAG | 1, 4 | m4C | CTAGCATCNNNNN[NNNN], |
| LweI, PhaI, SfaNI | C | M.Djo10085I, M.Hbo11551I, M.Hka19301III, | | | | GCATCTAGNN[NNNN] |
| | | M.Hme33500I, M.HsaR1I, M.Hsi33800I, | | | | |
| | | M.Hsp KM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| BmsI, BspST5I, | GCAT | M.Cac8I | GCNNGC | 2, 5 | m5C | GCNNGCATCNNNNN[NNNN] |
| LweI, PhaI, SfaNI | C | | | | | |
| BmsI, BspST5I, | GCAT | M.CagVI | TCCGGA | 1, 6 | m6A | GCATCCGGAN[NNNN] |
| LweI, PhaI, SfaNI | C | | | | | |
| BmsI, BspST5I, | GCAT | M.CcrNAV | YGCCGGCR | 3, 6 | m5C | YGCCGGCATCNNNNN[NNNN] |
| LweI, PhaI, SfaNI | C | | | | | |
| BmsI, BspST5I, | GCAT | M.Cdi630IV, M.Ckr177III, M.CmaLM2II, | GCNGC | 2, 4 | m5C | GCNGCATCNNNNN[NNNN] |
| LweI, PhaI, SfaNI | C | M.CocII, M.Fnu4HI, M.Fsp4HI | | | | |
| BmsI, BspST5I, | GCAT | M.CfrBI, M.Sen1735IV, M.SenAnaIV | CCWWGG | 1, 6 | m4C | CCWWGGCATCNNNNN[NNNN], |
| LweI, PhaI, SfaNI | C | | | | | GCATCCWWGG[NNNN] |
| BmsI, BspST5I, | GCAT | M.Cmu51329I, M.EcoJA03PDcm, | CCWGG | 1, 5 | Unknown | CCWGGCATCNNNNN[NNNN], |
| LweI, PhaI, SfaNI | C | M.RorR1Dcm, M.Sen8391 Dcm, M.ZalSM2II | | | | GCATCCWGGN[NNNN] |
| BmsI, BspST5I, | GCAT | M.CsiFSMII, M.MfoCT6IV, M.NgoAIV, | GCCGGC | 2, 5 | m5C | GCCGGCATCNNNNN[NNNN] |
| LweI, PhaI, SfaNI | C | M.NgoMIV, M1.Sgr13350II | | | | |
| BmsI, BspST5I, | GCAT | M.CviQIII, M.CviSIII, M.Hpy299XI, | TCGA | 1, 4 | m6A | GCATCGANNN[NNNN] |
| LweI, PhaI, SfaNI | C | M.Hpy30II, M.Hpy32X, M.Hpy99VII, | | | | |
| | | M.HpyAX, M.HpyFII, M.HpyUM032XVII, | | | | |
| | | M.Tam77409III, M.TaqI, M.TpaRLI, | | | | |
| | | M.Tth HB8I | | | | |
| BmsI, BspST5I, | GCAT | M.CviRI, M.Hpy298VIII, M.Hpy299VIII, | TGCA | 1, 4 | m6A | TGCATCNNNNN[NNNN] |
| LweI, PhaI, SfaNI | C | M.Hpy30VI, M.Hpy32V, M.Hpy57II, | | | | |
| | | M.Hpy66VIII, M.HpyFVI, M.HpyUM032VIII, | | | | |
| | | M.HpyUM037VIII | | | | |
| BmsI, BspST5I, | GCAT | M.Ddel | CTNAG | 1, 5 | m5C | CTNAGCATCNNNNN[NNNN], |
| LweI, PhaI, SfaNI | C | | | | | GCATCTNAGN[NNNN] |
| BmsI, BspST5I, | GCAT | M.FatI | CATG | 1, 4 | m5C | CATGCATCNNNNN[NNNN], |
| LweI, PhaI, SfaNI | C | | | | | GCATCATGNN[NNNN] |
| BmsI, BspST5I, | GCAT | M.FisAW1I, M.ThaI, M.Tph12270II | CGCG | 1, 4 | m4C | CGCGCATCNNNNN[NNNN], |
| LweI, PhaI, SfaNI | C | | | | | GCATCGCGNN[NNNN] |
| BmsI, BspST5I, | GCAT | M.FnoB1II, M.Hal24586I, M.Hpy298V, | CCGG | 1, 4 | m4C | CCGGCATCNNNNN[NNNN], |
| LweI, PhaI, SfaNI | C | M.Hpy299IX, M.Hpy30V, M.Hpy32XI, | | | | GCATCCGGNN[NNNN] |
| | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| BmsI, BspST5I, | GCAT | M.HhaI, M.HinP1I, M.Hpy99III, M.HpyAVIII, | GCGC | 2, 3 | m5C | GCGCATCNNNNN[NNNN] |
| LweI, PhaI, SfaNI | C | M.HpyUM032XV | | | | |
| BmsI, BspST5I, | GCAT | M.Hka19301IV | TCGCGA | 2, 5 | m4C | GCATCGCGAN[NNNN] |
| LweI, PhaI, SfaNI | C | | | | | |
| BmsI, BspST5I, | GCAT | M.Hme33500II | MGCWGCD | 5 | m4C | MGCWGCATCNNNNN[NNNN] |
| LweI, PhaI, SfaNI | C | | | | | |
| BmsI, BspST5I, | GCAT | M.Hpy188I, M.Hpy298IX, M.Hpy299V, | TCNGA | 1, 5 | m6A | GCATCNGANN[NNNN] |
| LweI, PhaI, SfaNI | C | M.Hpy66V, M.HpyUM032V | | | | |
| BmsI, BspST5I, | GCAT | M.Hpy30VIII, M.Hpy32VIII, M.Hpy57III, | TCNNGA | 1, 6 | m6A | GCATCNNGAN[NNNN] |
| LweI, PhaI, SfaNI | C | M.Hpy66VI, M.Hpy99XVIII, M.HpyFVIII, | | | | |
| | | M.HpyPVIII | | | | |
| BmsI, BspST5I, | GCAT | M.Hpy30X, M.Hpy57VIII, M.HpyFXI, M.MhuI | CTNAG | 1, 5 | m4C | CTNAGCATCNNNNN[NNNN], |
| LweI, PhaI, SfaNI | C | | | | | GCATCTNAGN[NNNN] |
| BmsI, BspST5I, | GCAT | M.Hpy99IV, M.HpyFORFX | CCNNGG | 1, 6 | m4C | CCNNGGCATCNNNNN[NNNN], |
| LweI, PhaI, SfaNI | C | | | | | GCATCCNNGG[NNNN] |
| BmsI, BspST5I, | GCAT | M.HpyD27I | GAGG | 2, 4 | m6A, | GCATCCTCNN[NNNN], |
| LweI, PhaI, SfaNI | C | | | | m5C | GAGGCATCNNNNN[NNNN] |
| BmsI, BspST5I, | GCAT | M.Lph145I, M.Lph93I | TCWCC | 1 | m6A | GCATCWCCNN[NNNN] |
| LweI, PhaI, SfaNI | C | | | | | |
| BmsI, BspST5I, | GCAT | M.Msp1II | CCAGG | 5 | m4C | GCATCCTGGN[NNNN], |
| LweI, PhaI, SfaNI | C | | | | | CCAGGCATCNNNNN[NNNN] |
| BmsI, BspST5I, | GCAT | M.Msp42II | CCWGG | 1, 5 | m4C | CCWGGCATCNNNNN[NNNN], |
| LweI, PhaI, SfaNI | C | | | | | GCATCCWGGN[NNNN] |
| BmsI, BspST5I, | GCAT | M.NheI | GCTAGC | 1, 6 | m4C | GCTAGCATCNNNNN[NNNN] |
| LweI, PhaI, SfaNI | C | | | | | |
| BmsI, BspST5I, | GCAT | M.Nme18III | TCTGG | 2, 4 | m5C | GCATCTGGNN[NNNN] |
| LweI, PhaI, SfaNI | C | | | | | |
| BmsI, BspST5I, | GCAT | M.Nme18V | GCGCGC | 2, 5 | m5C | GCGCGCATCNNNNN[NNNN] |
| LweI, PhaI, SfaNI | C | | | | | |
| BmsI, BspST5I, | GCAT | M.NmeDI | VCCGGY | 2, 5 | m5C | VCCGGCATCNNNNN[NNNN] |
| LweI, PhaI, SfaNI | C | | | | | |
| BmsI, BspST5I, | GCAT | M.NmeMC58III | CCAGA | 1 | m5C | TCTGGCATCNNNNN[NNNN], |
| LweI, PhaI, SfaNI | C | | | | | GCATCCAGAN[NNNN] |
| BmsI, BspST5I, | GCAT | M.Nsp209III, M.Nsp51109II | CTCGAG | 1, 6 | m5C | CTCGAGCATCNNNNN[NNNN], |
| LweI, PhaI, SfaNI | C | | | | | GCATCTCGAG[NNNN] |
| BmsI, BspST5I, | GCAT | M.NspI, M.NspHI | RCATGY | 2, 5 | m5C | RCATGCATCNNNNN[NNNN] |
| LweI, PhaI, SfaNI | C | | | | | |
| BmsI, BspST5I, | GCAT | M.Pam7686I | CCATGG | 1, 6 | m5C | CCATGGCATCNNNNN[NNNN], |
| LweI, PhaI, SfaNI | C | | | | | GCATCCATGG[NNNN] |
| BmsI, BspST5I, | GCAT | M.Pfa23572II | CGATCG | 1, 6 | m5C | CGATCGCATCNNNNN[NNNN], |
| LweI, PhaI, SfaNI | C | | | | | GCATCGATCG[NNNN] |
| BmsI, BspST5I, | GCAT | M.Pla5I | CTAG | 1, 4 | Unknown | CTAGCATCNNNNN[NNNN], |
| LweI, PhaI, SfaNI | C | | | | | GCATCTAGNN[NNNN] |
| BmsI, BspST5I, | GCAT | M.Sam53668II | CCATGG | 1, 6 | Unknown | CCATGGCATCNNNNN[NNNN], |
| LweI, PhaI, SfaNI | C | | | | | GCATCCATGG[NNNN] |
| BmsI, BspST5I, | GCAT | M.Sen1781III | CCWWGG | 1, 6 | Unknown | CCWWGGCATCNNNNN[NNNN], |
| LweI, PhaI, SfaNI | C | | | | | GCATCCWWGG[NNNN] |
| BmsI, BspST5I, | GCAT | M.SmoLVI, M.Spn23FII, M.Spn556I, | TCTAGA | 1, 6 | m6A | GCATCTAGAN[NNNN] |
| LweI, PhaI, SfaNI | C | M.Spn7465I, M.SpnRI, M.XbaI | | | | |
| BmsI, BspST5I, | GCAT | M.SonIII | GCANNNN | 3, 9 | m6A | GCATCNNGTC[NNNN] |
| LweI, PhaI, SfaNI | C | | GTC | | | |
| BmsI, BspST5I, | GCAT | M.TdeII | CTCTTC | 1, 2 | m4C, | GAAGAGCATCNNNNN[NNNN], |
| LweI, PhaI, SfaNI | C | | | | m6A | GCATCTCTTC[NNNN] |
| BmsI, BspST5I, | GCAT | M.TspRI | CASTG | 1, 5 | m5C | CASTGCATCNNNNN[NNNN], |
| LweI, PhaI, SfaNI | C | | | | | GCATCASTGN[NNNN] |
| BmsI, BspST5I, | GCAT | M.Yen2516I | GCGCGC | 2, 5 | Unknown | GCGCGCATCNNNNN[NNNN] |
| LweI, PhaI, SfaNI | C | | | | | |
| BmsI, BspST5I, | GCAT | M1.AgoG2I, M2.AgoG2I | CCAGT | 1, 4 | m4C | ACTGGCATCNNNNN[NNNN], |
| LweI, PhaI, SfaNI | C | | | | | GCATCCAGTN[NNNN] |
| BmsI, BspST5I, | GCAT | M1.BspACI | CCGC | 1 | m5C | GCGGCATCNNNNN[NNNN], |
| LweI, PhaI, SfaNI | C | | | | | GCATCCGCNN[NNNN] |
| BmsI, BspST5I, | GCAT | M1.BstF5I, M3.BstF5I, M2.Csp12AI | CATCC | 3 | m6A | GCATCCNNNN[NNNN] |
| LweI, PhaI, SfaNI | C | | | | | |
| BmsI, BspST5I, | GCAT | M1.HgaI, M2.LlaJI | GACGC | 4 | m5C | GACGCATCNNNNN[NNNN] |
| LweI, PhaI, SfaNI | C | | | | | |
| BmsI, BspST5I, | GCAT | M1.HphI | TCACC | 2 | m5C | GCATCACCNN[NNNN] |
| LweI, PhaI, SfaNI | C | | | | | |
| BmsI, BspST5I, | GCAT | M1.Hpy32II | TCYYC | 1 | m6A | GCATCYYCNN[NNNN] |
| LweI, PhaI, SfaNI | C | | | | | |
| BmsI, BspST5I, | GCAT | M1.HpyAII, M1.MboII, M1.NcuI | TCTTC | 1 | m6A | GCATCTTCNN[NNNN] |
| LweI, PhaI, SfaNI | C | | | | | |
| BmsI, BspST5I, | GCAT | M1.LplAG30I | GAAGG | 5 | m5C | GCATCCTTCN[NNNN], |
| LweI, PhaI, SfaNI | C | | | | | GAAGGCATCNNNNN[NNNN] |
| BmsI, BspST5I, | GCAT | M1.Sgr13350III, M2.Sgr13350III | GAAGAGC | 5, 6 | m6A, | GAAGAGCATCNNNNN[NNNN] |
| LweI, PhaI, SfaNI | C | | | | m4C | |
| BmsI, BspST5I, | GCAT | M2.BceSIII, M1.Pru9I, M2.Pru91 | ACGGC | 2, 4 | m4C | ACGGCATCNNNNN[NNNN] |
| LweI, PhaI, SfaNI | C | | | | | |
| BmsI, BspST5I, | GCAT | M2.BstF5I | CATCC | 2 | m6A | GCATCCNNNN[NNNN] |
| LweI, PhaI, SfaNI | C | | | | | |
| BmsI, BspST5I, | GCAT | M2.ClaSBI, M1.Fli6794IV, M2.HphI, | TCACC | 1 | m6A | GCATCACCNN[NNNN] |
| LweI, PhaI, SfaNI | C | M2.Ngo3502II, M1.Ngo6140II, M1.NgoAXVI, | | | | |
| | | M1.NgoBVIII, M1.NgoFA19I, M2.NgoMVIII, | | | | |
| | | M2.PspHUN102I, M1.Ssp10II | | | | |
| BmsI, BspST5I, | GCAT | M2.Hpy32II, M2.HpyAII, M2.MboII, M2.NcuI | TCTTC | 2 | m4C | GCATCTTCNN[NNNN] |
| LweI, PhaI, SfaNI | C | | | | | |
| BmsI, BspST5I, | GCAT | M2.HpyAVI, M1.MnlI | GAGG | 4 | m5C | GCATCCTCNN[NNNN], |
| LweI, PhaI, SfaNI | C | | | | | GAGGCATCNNNNN[NNNN] |
| BmsI, BspST5I, | GCAT | M4.BstF5I, M1.Cin755I, M2.Cin755I, | CATCC | 2, 3 | m6A | GCATCCNNNN[NNNN] |
| LweI, PhaI, SfaNI | C | M1.CmeSR3I, M2.CmeSR3I, M1.Csp12AI, | | | | |
| | | M1.Csp323I, M2.Csp323I, M1.Csp410I, | | | | |
| | | M2.Csp410I, M.FokI, M1.Msp315I, | | | | |
| | | M2.Msp315I, M.Sau13435I, M.StsI, | | | | |
| | | M1.Tsu2489IV, M2.Tsu2489IV, | | | | |
| | | M3.Tsu2489IV | | | | |
| BpuSI, BslFI, | GGGA | M.AatII, M.Lsp6406III | GACGTC | 2, 5 | m6A | GGGACGTCNNNNNNN[NNNN] |
| BsmFI | C | | | | | |
| BspLU11III, | | | | | | |
| BstOZ616I, FaqI | | | | | | |
| BpuSI, BslFI, | GGGA | M.AbaBs12I, M.Awo1030I, M.Cdu23823I, | CCWGG | 1, 5 | m5C | CCWGGGGACNNNNNNNNNN[NNNN], |
| BsmFI | C | M.Csa8155II, M.Cun9529I, M.EasL1Dcm, | | | | CCWGGGACNNNNNNNNNN[NNNN], |
| BspLU11III, | | M.Eba57Dcm, M.Ec134399Dcm, | | | | GGGACCWGGNNNNNN[NNNN] |
| BstOZ616I, FaqI | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| BpuSI, BslFI, | GGGA | M.AbrSp7I, M.Apa101655II, M.Ath21654I, | GANTC | 2, 4 | m6A | GGGACTCNNNNNNNN[NNNN] |
| BsmFI, | C | M.BdiNK6I, M.Bel76I, M.Bsp460I, | | | | |
| BspLU11III, | | M.BspJKG1II, M.BspLA1II, M.BspRAC04II, | | | | |
| BstOZ616I, FaqI | | M.CagII, M.CcrMI, M.CcrNAI, M.CnaPQ2I, | | | | |
| | | M.Csp16704III, M.CspNS6I, M.CviBI, | | | | |
| | | M.CviQVI, M.Eli8509I, M.Fnu419I, M.Fsp71I, | | | | |
| | | M.HbaI, M.HhaII, M.HinfI, M.HinHia1I, | | | | |
| | | M.Hpy298IV, M.Hpy299IV, M.Hpy32IV, | | | | |
| | | M.Hpy57IV, M.Hpy66IV, M.Hpy99IX, | | | | |
| | | M.HpyAIV, M.Hpy FIV, M.HpyUM032IV, | | | | |
| | | M.HpyUM037IV, M.Mbo45III, M.Msp10I, | | | | |
| | | M.Msp73BI, M.MspAL11I, M.MspLW5II, | | | | |
| | | M.NhaXII, M.NpeUS61I, M.OspHL35I, | | | | |
| | | M.Pin17395II, M.Pla5III, M.PspLM6I, | | | | |
| | | M.RbaNRL2I, M.Rgi145II, M.Rmo1926I, | | | | |
| | | M.RpaIII, M.RsaII, M.RshIII, M.Rsp8I, | | | | |
| | | M.SmeC3I, M.SmoLV, M.Sna24252I, | | | | |
| | | M.SscL1I, M.SspM41II, M.SstE37II, M.ThaIII, | | | | |
| | | M.Xsp91I, M.ZmoCP4I | | | | |
| BpuSI, BsIFI, | GGGA | M.AciI | CCGC | 1, 3 | m5C | GCGGGGACNNNNNNNNNN[NNNN], |
| BsmFI, | C | | | | | GCGGGACNNNNNNNNNN[NNNN], |
| BspLU11III, | | | | | | GGGACCGCNNNNNNN[NNNN] |
| BstOZ616I, FaqI | | | | | | |
| BpuSI, BsIFI, | GGGA | M.AflIII | ACRYGT | 2, 5 | m4C | GGGACRYGTNNNNNN[NNNN] |
| BsmFI, | C | | | | | |
| BspLU11III, | | | | | | |
| BstOZ616I, FaqI | | | | | | |
| BpuSI, BslFI, | GGGA | M.AhdI | GACNNNN | 2, 10 | m6A | GGGACNNNNNGTCNN[NNNN] |
| BsmFI, | C | | NGTC | | | |
| BspLU11III, | | | | | | |
| BstOZ616I, FaqI | | | | | | |
| BpuSI, Bs1FI, | GGGA | M.Ama55I, M.CnaPQ2III, M.Rmo1926II, | CGGCCG | 1, 6 | m5C | CGGCCGGGACNNNNNNNNNN[NNNN], |
| BsmFI, | C | M.SenC1765IV, M.SspG1I, M.XorPXII | | | | GGGACGGCCGNNNNN[NNNN] |
| BspLU11III, | | | | | | |
| BstOZ616I, FaqI | | | | | | |
| BpuSI, BslFI, | GGGA | M.AquI | CYCGRG | 1, 6 | m5C | CYCGRGGGACNNNNNNNNNN[NNNN], |
| BsmFI, | C | | | | | CYCGGGGACNNNNNNNNNN[NNNN], |
| BspLU11III, | | | | | | CYCGGGACNNNNNNNNNN[NNNN], |
| BstOZ616I, FaqI | | | | | | GGGACYCGRGNNNNN[NNNN] |
| BpuSI, BslFI, | GGGA | M.AspU41II, M.BisIII, M.BsuW23II, | CCWGG | 2, 4 | m5C | CCWGGGACNNNNNNNNNN[NNNN] |
| BsmFI, | C | M.CnaPQ2II, M.EcoO6Dcm, M.Eco12581Dcm, | | | | |
| BspLU11III, | | M.Eco1655Dcm, M.Eco29787Dcm, | | | | |
| BstOZ616I, FaqI | | M.Eco3024Dcm, M.Eco3317Dcm, | | | | |
| | | M.Eco3325Dcm, M.Eco3740Dcm, | | | | |
| | | M.Eco3911Dcm, M.Eco4211Dcm, | | | | |
| | | M.Eco4465Dcm, M.Eco600Dcm, | | | | |
| | | M.Eco644Dcm, M.Eco86Dcm, | | | | |
| | | M.Eco9001Dcm, M.Eco9387Dcm, | | | | |
| | | M.EcoE1140Dcm, M.EcoE455Dcm, | | | | |
| | | M.EcoGDcm, M.EcoNU14Dcm, M.EcoRII, | | | | |
| | | M.Kpn223III, M.Kpn35657II, M.Kpn36Dcm, | | | | |
| | | M.Kpn555I, M.Kpn62629II, M.KpnAATV, | | | | |
| | | M.Kqu603Dcm, M.Psp345I, M.Sen158Dcm, | | | | |
| | | M.Sen641Dcm, M.SenA46Dcm, | | | | |
| | | M.SenAboDcm, M.TcoKWC4II | | | | |
| BpuSI, BslFI, | GGGA | M.AvaIX, M.Cfr10I, M.Nme18VI, M.VchAI, | RCCGGY | 2, 5 | m5C | GGGACCGGYNNNNNN[NNNN] |
| BsmFI, | C | M.Vch0395I | | | | |
| BspLU11III, | | | | | | |
| BstOZ616I, FaqI | | | | | | |
| BpuSI, BsIFI, | GGGA | M.BanLII, M.CpeAII, M.SinI | GGWCC | 2, 4 | m5C | GGGACCNNNNNNNNN[NNNN] |
| BsmFI, | C | | | | | |
| BspLU11III, | | | | | | |
| BstOZ616I, FaqI | | | | | | |
| BpuSI, BsIFI, | GGGA | M.BceSII | GGWCC | 1, 5 | m5C | GGGACCNNNNNNNNN[NNNN] |
| BsmFI, | C | | | | | |
| BspLU11III, | | | | | | |
| BstOZ616I, FaqI | | | | | | |
| BpuSI, BsIFI, | GGGA | M.BcoSlacII, M.MspLW5III, M.Nbr128I, | CTCGAG | 1, 6 | m4C | CTCGAGGGACNNNNNNNNNN[NNNN], |
| BsmFI, | C | M.Psy18884II, M.Tam77409II, M.TbrGE1II, | | | | GGGACTCGAGNNNNN[NNNN] |
| BspLU11III, | | M.TpaRLII | | | | |
| BstOZ616I, FaqI | | | | | | |
| BpuSI, BslFI, | GGGA | M.BepI, M.FpsJV, M.Gel16401III, M.Pdi8503I | CGCG | 1, 4 | m5C | CGCGGGACNNNNNNNNNN[NNNN], |
| BsmFI, | C | | | | | GGGACGCGNNNNNNN[NNNN] |
| BspLU11III, | | | | | | |
| BstOZ616I, FaqI | | | | | | |
| BpuSI, BsIFI, | GGGA | M.Bhy27164IV, M.BstNI, M.EcoDHB4Dcm, | CCWGG | 2, 4 | m4C | CCWGGGACNNNNNNNNNN[NNNN] |
| BsmFI, | C | M.EcoK54Dcm, M.Gth3570I, M.Hla49239I, | | | | |
| BspLU11III, | | M.MvaI, M.Pdi8503II, M.Sso30807Dcm, | | | | |
| BstOZ616I, FaqI | | M.YpeI, M.Ype41I, M.YpeShI | | | | |
| BpuSI, BsIFI, | GGGA | M.BsoBI | CYCGRG | 1, 6 | m4C | CYCGRGGGACNNNNNNNNNN[NNNN], |
| BsmFI, | C | | | | | CYCGGGGACNNNNNNNNNN[NNNN], |
| BspLU11III, | | | | | | CYCGGGACNNNNNNNNNN[NNNN], |
| BstOZ616I, FaqI | | | | | | GGGACYCGRGNNNNN[NNNN] |
| BpuSI, BslFI, | GGGA | M.BsuFI, M.CpeAIV, M.MspI | CCGG | 1, 4 | m5C | CCGGGGACNNNNNNNNNN[NNNN], |
| BsmFI, | C | | | | | CCGGGACNNNNNNNNNN[NNNN], |
| BspLU11III, | | | | | | GGGACCGGNNNNNNN[NNNN] |
| BstOZ616I, FaqI | | | | | | |
| BpuSI, BslFI, | GGGA | M.Cab13497I, M.Dac11109III, M.Dco13527I, | CTAG | 1, 4 | m4C | CTAGGGACNNNNNNNNNN[NNNN], |
| BsmFI, | C | M.Djo10085I, M.Hbo11551I, M.Hka19301III, | | | | GGGACTAGNNNNNNN[NNNN] |
| BspLU11III, | | M.Hme33500I, M.HsaR1I, M.Hsi33800I, | | | | |
| BstOZ616I, FaqI | | M.HspKM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| BpuSI, BsIFI, | GGGA | M.CagVIII | ACGCGT | 2, 5 | m4C | GGGACGCGTNNNNNN[NNNN] |
| BsmFI, | C | | | | | |
| BspLU11III, | | | | | | |
| BstOZ616I, FaqI | | | | | | |
| BpuSI, BsIFI, | GGGA | M.Cdi630III | CCSSGG | 2, 5 | m4C | CCSSGGGACNNNNNNNNNN[NNNN], |
| BsmFI, | C | | | | | CCSGGGACNNNNNNNNNN[NNNN] |
| BspLU11III, | | | | | | |
| BstOZ616I, FaqI | | | | | | |
| BpuSI, BsIFI, | GGGA | M.Cfr9I, M.SmaI, M.Sma36365II, M.Xca85III, | CCCGGG | 2, 5 | m4C | CCCGGGGACNNNNNNNNNN[NNNN], |
| BsmFI, | C | M.XcyI, M.XmaI, M.XveII | | | | CCCGGGACNNNNNNNNNN[NNNN] |
| BspLU11III, | | | | | | |
| BstOZ616I, FaqI | | | | | | |
| BpuSI, BslFI, | GGGA | M.CfrBI, M.Sen1735IV, M.SenAnaIV | CCWWGG | 1, 6 | m4C | CCWWGGGGACNNNNNNNNNN[NNNN], |
| BsmFI, | C | | | | | CCWWGGGACNNNNNNNNNN[NNNN], |
| BspLU11III, | | | | | | GGGACCWWGGNNNNN[NNNN] |
| BstOZ616I, FaqI | | | | | | |
| BpuSI, BslFI, | GGGA | M.Cmu51329I, M.EcoJA03PDcm, | CCWGG | 1, 5 | Unknown | CCWGGGGACNNNNNNNNNN[NNNN], |
| BsmFI, | C | M.RorR1Dcm, M.Sen8391 Dcm, M.ZalSM2II | | | | CCWGGGACNNNNNNNNNN[NNNN], |
| BspLU11III, | | | | | | GGGACCWGGNNNNNN[NNNN] |
| BstOZ616I, FaqI | | | | | | |
| BpuSI, BslFI, | GGGA | M.CocIII, M.Fsi4947II, M.TdeIII, | GGNCC | 1, 5 | m4C | GGGACCNNNNNNNNN[NNNN] |
| BsmFI, | C | M.Tph12270I | | | | |
| BspLU11III, | | | | | | |
| BstOZ616I, FaqI | | | | | | |
| BpuSI, BsIFI, | GGGA | M.Cpa18696III, M.Ype19I, M.YpeDodI, | CCWGG | 2, 4 | Unknown | CCWGGGACNNNNNNNNNN[NNNN] |
| BsmFI, | C | M.YpeEDI, M.YpeJ9I, M.YpePGI | | | | |
| BspLU11III, | | | | | | |
| BstOZ616I, FaqI | | | | | | |
| BpuSI, BsIFI, | GGGA | M.DdeI | CTNAG | 1, 5 | m5C | CTNAGGGACNNNNNNNNNN[NNNN], |
| BsmFI, | C | | | | | GGGACTNAGNNNNNN[NNNN] |
| BspLU11III, | | | | | | |
| BstOZ616I, FaqI | | | | | | |
| BpuSI, BsIFI, | GGGA | M.DsaV, M.Ecl18kI, M.FpsJVI, M.NlaX, | CCNGG | 2, 4 | m5C | CCNGGGACNNNNNNNNNN[NNNN], |
| BsmFI, | C | M.SenPI, M.SsoII | | | | CCGGGACNNNNNNNNNN[NNNN] |
| BspLU11III, | | | | | | |
| BstOZ616I, FaqI | | | | | | |
| BpuSI, BslFI, | GGGA | M.Eco29kI, M.NgoAIII, M.SacII | CCGCGG | 2, 5 | m5C | CCGCGGGACNNNNNNNNNN[NNNN] |
| BsmFI, | C | | | | | |
| BspLU11III, | | | | | | |
| BstOZ616I, FaqI | | | | | | |
| BpuSI, BsIFI, | GGGA | M.EcoNI | CCTNNNN | 2, 10 | m4C | CCTNNNNNAGGGACNNNNNNNNNN[NNNN] |
| BsmFI, | C | | NAGG | | | |
| BspLU11III, | | | | | | |
| BstOZ616I, FaqI | | | | | | |
| BpuSI, BsIFI, | GGGA | M.EcoO109I | RGGNCCY | 3, 5 | m5C | GGGACCYNNNNNNNN[NNNN] |
| BsmFI, | C | | | | | |
| BspLU11III, | | | | | | |
| BstOZ616I, FaqI | | | | | | |
| BpuSI, BslFI, | GGGA | M.EfaRFII, M.Mae7806II | CCGG | 2, 3 | m4C | CCGGGACNNNNNNNNNN[NNNN] |
| BsmFI, | C | | | | | |
| BspLU11III, | | | | | | |
| BstOZ616I, FaqI | | | | | | |
| BpuSI, BslFI, | GGGA | M.FatI | CATG | 1, 4 | m5C | CATGGGACNNNNNNNNNN[NNNN], |
| BsmFI, | C | | | | | GGGACATGNNNNNNN[NNNN] |
| BspLU11III, | | | | | | |
| BstOZ616I, FaqI | | | | | | |
| BpuSI, BsIFI, | GGGA | M.FisAW1I, M.ThaI, M.Tph12270II | CGCG | 1, 4 | m4C | CGCGGGACNNNNNNNNNN[NNNN], |
| BsmFI, | C | | | | | GGGACGCGNNNNNNN[NNNN] |
| BspLU11III, | | | | | | |
| BstOZ616I, FaqI | | | | | | |
| BpuSI, BslFI, | GGGA | M.FnoB1II, M.Hal24586I, M.Hpy298V, | CCGG | 1, 4 | m4C | CCGGGGACNNNNNNNNNN[NNNN], |
| BsmFI, | C | M.Hpy299IX, M.Hpy30V, M.Hpy32XI, | | | | CCGGGACNNNNNNNNNN[NNNN], |
| BspLU11III, | | M.Hpy57V, M.Hpy66IX, M.Hpy99VIII, | | | | GGGACCGGNNNNNNN[NNNN] |
| BstOZ616I, FaqI | | M.HpyFV, M.HpyUM032IX, M.HpyUM037IX, | | | | |
| | | M.MjaVI, M.PspJDRI, M.TmeBII | | | | |
| BpuSI, BsIFI, | GGGA | M.FpsJI, M.HpaII, M.Nme18II, M.NmeAI | CCGG | 2, 3 | m5C | CCGGGACNNNNNNNNNN[NNNN] |
| BsmFI, | C | | | | | |
| BspLU11III, | | | | | | |
| BstOZ616I, FaqI | | | | | | |
| BpuSI, BslFI, | GGGA | M.Gel16401IV | CTGG | 3 | m5C | CTGGGACNNNNNNNNNN[NNNN] |
| BsmFI, | C | | | | | |
| BspLU11III, | | | | | | |
| BstOZ616I, FaqI | | | | | | |
| BpuSI, BsIFI, | GGGA | M.GsuRed1IV, M.Osp807II, M.Tth111I | GACNNNG | 2, 8 | m6A | GGGACNNNGTCNNNN[NNNN] |
| BsmFI, | C | | TC | | | |
| BspLU11III, | | | | | | |
| BstOZ616I, FaqI | | | | | | |
| BpuSI, BslFI, | GGGA | M.Hpy298II, M.Hpy299II, M.Hpy66II, | ACNGT | 2, 4 | m4C | GGGACNGTNNNNNNN[NNNN] |
| BsmFI, | C | M.HpyUM032II, M.HpyUM037II | | | | |
| BspLU11III, | | | | | | |
| BstOZ616I, FaqI | | | | | | |
| BpuSI, BslFI, | GGGA | M.Hpy30VIII, M.Hpy32VIII, M.Hpy57III, | TCNNGA | 1, 6 | m6A | TCGGGACNNNNNNNNNN[NNNN] |
| BsmFI, | C | M.Hpy66VI, M.Hpy99XVIII, M.HpyFVIII, | | | | |
| BspLU11III, | | M.HpyPVIII | | | | |
| BstOZ616I, FaqI | | | | | | |
| BpuSI, BslFI, | GGGA | M.Hpy30X, M.Hpy57VIII, M.HpyFXI, M.MhuI | CTNAG | 1, 5 | m4C | CTNAGGGACNNNNNNNNNN[NNNN], |
| BsmFI, | C | | | | | GGGACTNAGNNNNNN[NNNN] |
| BspLU11III, | | | | | | |
| BstOZ616I, FaqI | | | | | | |
| BpuSI, BsIFI, | GGGA | M.Hpy99IV, M.HpyFORFX | CCNNGG | 1, 6 | m4C | CCNNGGGGACNNNNNNNNNN[NNNN], |
| BsmFI, | C | | | | | CCNNGGGACNNNNNNNNNN[NNNN], |
| BspLU11III, | | | | | | CCNGGGACNNNNNNNNNN[NNNN], |
| BstOZ616I, FaqI | | | | | | GGGACCNNGGNNNNN[NNNN] |
| BpuSI, BsIFI, | GGGA | M.Hpy99XI | ACGT | 2, 3 | m5C | GGGACGTNNNNNNNN[NNNN] |
| BsmFI, | C | | | | | |
| BspLU11III, | | | | | | |
| BstOZ616I, FaqI | | | | | | |
| BpuSI, BsIFI, | GGGA | M.HpyAV, M.Nme18IV | GAAGG | 3, 4 | m6A, | GAAGGGACNNNNNNNNNN[NNNN] |
| BsmFI, | C | | | | m5C | |
| BspLU11III, | | | | | | |
| BstOZ616I, FaqI | | | | | | |
| BpuSI, BsIFI, | GGGA | M.Hpy D27I | GAGG | 2, 4 | m6A, | GGGACCTCNNNNNNN[NNNN], |
| BsmFI, | C | | | | m5C | GAGGGGACNNNNNNNNNN[NNNN], |
| BspLU11III, | | | | | | GAGGGACNNNNNNNNNN[NNNN] |
| BstOZ616I, FaqI | | | | | | |
| BpuSI, BsIFI, | GGGA | M.Mae7806III, M.PspPI, M.Sau96I | GGNCC | 2, 4 | m5C | GGGACCNNNNNNNNN[NNNN] |
| BsmFI | C | | | | | |
| BspLU11III, | | | | | | |
| BstOZ616I, FaqI | | | | | | |
| BpuSI, BslFI, | GGGA | M.MjaII | GGNCC | 1, 5 | m5C | GGGACCNNNNNNNNN[NNNN] |
| BsmFI, | C | | | | | |
| BspLU11III, | | | | | | |
| BstOZ616I, FaqI | | | | | | |
| BpuSI, BsIFI, | GGGA | M.MlyI, M.Pen113I | GASTC | 2, 4 | m6A | GGGACTCNNNNNNNN[NNNN] |
| BsmFI, | C | | | | | |
| BspLU11III, | | | | | | |
| BstOZ616I, FaqI | | | | | | |
| BpuSI, BslFI, | GGGA | M.Msp1II | CCAGG | 5 | m4C | GGGACCTGGNNNNNN[NNNN], |
| BsmFI, | C | | | | | CCAGGGGACNNNNNNNNNN[NNNN], |
| BspLU11III, | | | | | | CCAGGGACNNNNNNNNNN[NNNN] |
| BstOZ616I, FaqI | | | | | | |
| BpuSI, BslFI, | GGGA | M.Msp42II | CCWGG | 1, 5 | m4C | CCWGGGGACNNNNNNNNNN[NNNN], |
| BsmFI, | C | | | | | CCWGGGACNNNNNNNNNN[NNNN], |
| BspLU11III, | | | | | | GGGACCWGGNNNNNN[NNNN] |
| BstOZ616I, FaqI | | | | | | |
| BpuSI, BsIFI, | GGGA | M.NgoAXV, M.NgoMV | GGNNCC | 2, 5 | m5C | GGGACCNNNNNNNNN[NNNN] |
| BsmFI, | C | | | | | |
| BspLU11III, | | | | | | |
| BstOZ616I, FaqI | | | | | | |
| BpuSI, BsIFI, | GGGA | M.Nme18III | TCTGG | 2, 4 | m5C | TCTGGGACNNNNNNNNNN[NNNN] |
| BsmFI, | C | | | | | |
| BspLU11III, | | | | | | |
| BstOZ616I, FaqI | | | | | | |
| BpuSI, BslFI, | GGGA | M.NmeDI | VCCGGY | 2, 5 | m5C | RCCGGGGACNNNNNNNNNN[NNNN], |
| BsmFI, | C | | | | | GGGACCGGBNNNNNN[NNNN], |
| BspLU11III, | | | | | | RCCGGGACNNNNNNNNNN[NNNN] |
| BstOZ616I, FaqI | | | | | | |
| BpuSI, BslFI, | GGGA | M.NmeMC58III | CCAGA | 1 | m5C | TCTGGGGACNNNNNNNNNN[NNNN], |
| BsmFI, | C | | | | | TCTGGGACNNNNNNNNNN[NNNN], |
| BspLU11III, | | | | | | GGGACCAGANNNNNN[NNNN] |
| BstOZ616I, FaqI | | | | | | |
| BpuSI, BsIFI, | GGGA | M.Nsp209III, M.Nsp51109II | CTCGAG | 1, 6 | m5C | CTCGAGGGACNNNNNNNNNN[NNNN], |
| BsmFI, | C | | | | | GGGACTCGAGNNNNN[NNNN] |
| BspLU11III, | | | | | | |
| BstOZ616I, FaqI | | | | | | |
| BpuSI, BslFI, | GGGA | M.NspI, M.NspHI | RCATGY | 2, 5 | m5C | GGGACATGYNNNNNN[NNNN] |
| BsmFI, | C | | | | | |
| BspLU11III, | | | | | | |
| BstOZ616I, FaqI | | | | | | |
| BpuSI, Bs1FI, | GGGA | M.Pam7686I | CCATGG | 1, 6 | m5C | CCATGGGGACNNNNNNNNNN[NNNN], |
| BsmFI, | C | | | | | CCATGGGACNNNNNNNNNN[NNNN], |
| BspLU11III, | | | | | | GGGACCATGGNNNNN[NNNN] |
| BstOZ616I, FaqI | | | | | | |
| BpuSI, BslFI, | GGGA | M.Pfa23572II | CGATCG | 1, 6 | m5C | CGATCGGGACNNNNNNNNNN[NNNN], |
| BsmFI, | C | | | | | GGGACGATCGNNNNN[NNNN] |
| BspLU11III, | | | | | | |
| BstOZ616I, FaqI | | | | | | |
| BpuSI, BslFI, | GGGA | M.Pla5I | CTAG | 1, 4 | Unknown | CTAGGGACNNNNNNNNNN[NNNN], |
| BsmFI, | C | | | | | GGGACTAGNNNNNNN[NNNN] |
| BspLU11III, | | | | | | |
| BstOZ616I, FaqI | | | | | | |
| BpuSI, BslFI, | GGGA | M.Sam53668II | CCATGG | 1, 6 | Unknown | CCATGGGGACNNNNNNNNNN[NNNN], |
| BsmFI, | C | | | | | CCATGGGACNNNNNNNNNN[NNNN], |
| BspLU11III, | | | | | | GGGACCATGGNNNNN[NNNN] |
| BstOZ616I, FaqI | | | | | | |
| BpuSI, BsIFI, | GGGA | M.Sen1781III | CCWWGG | 1, 6 | Unknown | CCWWGGGGACNNNNNNNNNN[NNNN], |
| BsmFI, | C | | | | | CCWWGGGACNNNNNNNNNN[NNNN], |
| BspLU11III, | | | | | | GGGACCWWGGNNNNN[NNNN] |
| BstOZ616I, FaqI | | | | | | |
| BpuSI, BsIFI, | GGGA | M.SonIII | GACNNNN | 2, 8 | m6A | GGGACNNNNTGCNNN[NNNN] |
| BsmFI, | C | | TGC | | | |
| BspLU11III, | | | | | | |
| BstOZ616I, FaqI | | | | | | |
| BpuSI, BsIFI, | GGGA | M.TdeII | CTCTTC | 1, 2 | m4C | GAAGAGGGACNNNNNNNNNN[NNNN], |
| BsmFI, | C | | | | m6A | GGGACTCTTCNNNNN[NNNN] |
| BspLU11III, | | | | | | |
| BstOZ616I, FaqI | | | | | | |
| BpuSI, BsIFI, | GGGA | M.TspRI | CASTG | 1, 5 | m5C | CASTGGGACNNNNNNNNNN[NNNN], |
| BsmFI, | C | | | | | GGGACASTGNNNNNN[NNNN] |
| BspLU11III, | | | | | | |
| BstOZ616I, FaqI | | | | | | |
| BpuSI, BslFI, | GGGA | M1.AgoG2I, M2.AgoG2I | CCAGT | 1, 4 | m4C | ACTGGGGACNNNNNNNNNN[NNNN], |
| BsmFI, | C | | | | | ACTGGGACNNNNNNNNNN[NNNN], |
| BspLU11III, | | | | | | GGGACTGGNNNNNNN[NNNN], |
| BstOZ616I, FaqI | | | | | | GGGACCAGTNNNNNN[NNNN] |
| BpuSI, BsIFI, | GGGA | M1.Bag18758II, M2.Bag18758II | CACGGG | 3, 5 | Unknown | CACGGGGACNNNNNNNNNN[NNNN], |
| BsmFI, | C | | | | | CACGGGACNNNNNNNNNN[NNNN] |
| BspLU11III, | | | | | | |
| BstOZ616I, FaqI | | | | | | |
| BpuSI, BsIFI, | GGGA | M1.BceSIII | ACGGC | 2 | m4C | GGGACGGCNNNNNNN[NNNN] |
| BsmFI, | C | | | | | |
| BspLU11III, | | | | | | |
| BstOZ616I, FaqI | | | | | | |
| BpuSI, BsIFI, | GGGA | M1.BcnI, M2.BcnI, M.NciI | CCSGG | 2, 4 | m4C | CCSGGGACNNNNNNNNNN[NNNN], |
| BsmFI, | C | | | | | CCGGGACNNNNNNNNNN[NNNN] |
| BspLU11III, | | | | | | |
| BstOZ616I, FaqI | | | | | | |
| BpuSI, BsIFI, | GGGA | M1.BfiI | ACTGGG | 5 | m4C | ACTGGGGACNNNNNNNNNN[NNNN], |
| BsmFI | C | | | | | ACTGGGACNNNNNNNNNN[NNNN] |
| BspLU11III, | | | | | | |
| BstOZ616I, FaqI | | | | | | |
| BpuSI, BsIFI, | GGGA | M1.BspACI | CCGC | 1 | m5C | GCGGGGACNNNNNNNNNN[NNNN], |
| BsmFI, | C | | | | | GCGGGACNNNNNNNNNN[NNNN], |
| BspLU11III, | | | | | | GGGACCGCNNNNNNN[NNNN] |
| BstOZ616I, FaqI | | | | | | |
| BpuSI, | GGGA | M1.Dsh12II, M2.Dsh12II | GACGGC | 3, 4 | m4C | GGGACGGCNNNNNNN[NNNN] |
| BsIFI, | C | | | | | |
| BsmFI, | | | | | | |
| BspLU11III, | | | | | | |
| BstOZ616I, FaqI | | | | | | |
| BpuSI, BsIFI, | GGGA | M1.Hpy32II | GRRGA | 5 | m6A | GGGGACNNNNNNNNNN[NNNN] |
| BsmFI, | C | | | | | |
| BspLU11III, | | | | | | |
| BstOZ616I, FaqI | | | | | | |
| BpuSI, BslFI, | GGGA | M1.LplAG30I | GAAGG | 5 | m5C | GGGACCTTCNNNNNN[NNNN], |
| BsmFI, | C | | | | | GAAGGGGACNNNNNNNNNN[NNNN], |
| BspLU11III, | | | | | | GAAGGGACNNNNNNNNNN[NNNN] |
| BstOZ616I, FaqI | | | | | | |
| BpuSI, BslFI, | GGGA | M2.BceSIII, M1.Pru9I, M2.Pru9I | ACGGC | 2, 4 | m4C | GGGACGGCNNNNNNN[NNNN] |
| BsmFI, | C | | | | | |
| BspLU11III, | | | | | | |
| BstOZ616I, FaqI | | | | | | |
| BpuSI, BsIFI, | GGGA | M2.BfiI | ACTGGG | 2 | m4C | GGGACTGGGNNNNNN[NNNN] |
| BsmFI, | C | | | | | |
| BspLU11III, | | | | | | |
| BstOZ616I, FaqI | | | | | | |
| BpuSI, BslFI, | GGGA | M2.BfuAI | ACCTGC | 2 | m5C | GGGACCTGCNNNNNN[NNNN] |
| BsmFI, | C | | | | | |
| BspLU11III, | | | | | | |
| BstOZ616I, FaqI | | | | | | |
| BpuSI, BslFI, | GGGA | M2.HgaI, M1.LlaJI | GACGC | 3 | m5C | GGGACGCNNNNNNNN[NNNN] |
| BsmFI, | C | | | | | |
| BspLU11III, | | | | | | |
| BstOZ616I, FaqI | | | | | | |
| BpuSI, BslFI, | GGGA | M2.HpyAVI, M1.MnlI | GAGG | 4 | m5C | GGGACCTCNNNNNNN[NNNN], |
| BsmFI, | C | | | | | GAGGGGACNNNNNNNNNN[NNNN], |
| BspLU11III, | | | | | | GAGGGACNNNNNNNNNN[NNNN] |
| BstOZ616I, FaqI | | | | | | |
| BpuSI, BslFI, | GGGA | M2.Nme579IV, M2.NmeMC58II | GACGC | 2 | m6A | GGGACGCNNNNNNNN[NNNN] |
| BsmFI, | C | | | | | |
| BspLU11III, | | | | | | |
| BstOZ616I, FaqI | | | | | | |
| CseI, HgaI | GACG | M.AbaBs12I, M.Awo1030I, M.Cdu23823I, | CCWGG | 1, 5 | m5C | CCWGGACGCNNNNN[NNNNN], |
| | C | M.Csa8155II, M.Cun9529I, M.EasL1Dcm, | | | | GACGCCWGGN[NNNNN] |
| | | M.Eba57Dcm, M.Ec134399Dcm, | | | | |
| | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| CseI, HgaI | GACG | M.AccI, M.FpsFPG3II, M.SdeAIV | GTMKAC | 2, 5 | m6A | GTMGACGCNNNNN[NNNNN] |
| | C | | | | | |
| CseI, HgaI | GACG | M.AciI | CCGC | 1, 3 | m5C | GCGGACGCNNNNN[NNNNN], |
| | C | | | | | GACGCGGNNN[NNNNN], |
| | | | | | | GACGCCGCNN[NNNNN] |
| CseI, HgaI | GACG | M.AflIII | ACRYGT | 2, 5 | m4C | GACGCGTNNN[NNNNN] |
| | C | | | | | |
| CseI, HgaI | GACG | M.Alw26I | GAGAC | 4, 3 | m6A, | GAGACGCNNNNN[NNNNN] |
| | C | | | | m5C | |
| CseI, HgaI | GACG | M.Ama55I, M.CnaPQ2III, M.Rmo1926II, | CGGCCG | 1, 6 | m5C | CGGCCGACGCNNNNN[NNNNN], |
| | C | M.SenC1765IV, M.SspG1I, M.XorPXII | | | | GACGCGGCCG[NNNNN] |
| CseI, HgaI | GACG | M.AorT14III, M.GsuRed1III, M.SalI | GTCGAC | 2, 5 | m6A | GTCGACGCNNNNN[NNNNN] |
| | C | | | | | |
| CseI, HgaI | GACG | M.AquI | CYCGRG | 1, 6 | m5C | CYCGRGACGCNNNNN[NNNNN], |
| | C | | | | | GACGCYCGRG[NNNNN] |
| CseI, HgaI | GACG | M.AvaIX, M.Cfr10I, M.Nme18VI, M.VchAI, | RCCGGY | 2, 5 | m5C | GACGCCGGYN[NNNNN] |
| | C | M.Vch0395I | | | | |
| CseI, HgaI | GACG | M.BamWS8I, M.CthV, M.EfaRFI, M.Saf8902II, | GCWGC | 2, 4 | m5C | GACGCWGCNN[NNNNN] |
| | C | M.TacII, M.TcoKWC4III | | | | |
| CseI, HgaI | GACG | M.BbvI, M.BceSIV | GCAGC | 2, 4 | m5C | GACGCTGCNN[NNNNN], |
| | C | | | | | GACGCAGCNN[NNNNN] |
| CseI, HgaI | GACG | M.BcoSlacII, M.MspLW5III, M.Nbr128I, | CTCGAG | 1, 6 | m4C | CTCGAGACGCNNNNN[NNNNN], |
| | C | M.Psy18884II, M.Tam77409II, M.TbrGE1II, | | | | GACGCTCGAG[NNNNN] |
| | | M.TpaRLII | | | | |
| CseI, HgaI | GACG | M.BepI, M.FpsJV, M.Gel16401III, M.Pdi8503I | CGCG | 1, 4 | m5C | GACGCGNNNN[NNNNN], |
| | C | | | | | CGCGACGCNNNNN[NNNNN] |
| CseI, HgaI | GACG | M.BsoBI | CYCGRG | 1, 6 | m4C | CYCGRGACGCNNNNN[NNNNN], |
| | C | | | | | GACGCYCGRG[NNNNN] |
| CseI, HgaI | GACG | M.BsuFI, M.CpeAIV, M.MspI | CCGG | 1, 4 | m5C | CCGGACGCNNNNN[NNNNN], |
| | C | | | | | GACGCCGGNN[NNNNN] |
| CseI, HgaI | GACG | M.Cab13497I, M.Dac11109III, M.Dco13527I, | CTAG | 1, 4 | m4C | CTAGACGCNNNNN[NNNNN], |
| | C | M.Djo10085I, M.Hbo11551I, M.Hka19301III, | | | | GACGCTAGNN[NNNNN] |
| | | M.Hme33500I, M.HsaR1I, M.Hsi33800I, | | | | |
| | | M.HspKM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| CseI, HgaI | GACG | M.Cac8I | GCNNGC | 2, 5 | m5C | GACGCNNGCN[NNNNN] |
| | C | | | | | |
| CseI, HgaI | GACG | M.CagVI | TCCGGA | 1, 6 | m6A | TCCGGACGCNNNNN[NNNNN] |
| | C | | | | | |
| CseI, HgaI | GACG | M.CagVIII | ACGCGT | 2, 5 | m4C | GACGCGTNNN[NNNNN] |
| | C | | | | | |
| CseI, HgaI | GACG | M.CcrNAV | YGCCGGCR | 3, 6 | m5C | GACGCCGGCR[NNNNN] |
| | C | | | | | |
| CseI, HgaI | GACG | M.Cdi630IV, M.Ckr177III, M.CmaLM2II, | GCNGC | 2, 4 | m5C | GACGCNGCNN[NNNNN] |
| | C | M.CocII, M.Fnu4HI, M.Fsp 4HI | | | | |
| CseI, HgaI | GACG | M.CfrBI, M.Sen1735IV, M.SenAnaIV | CCWWGG | 1, 6 | m4C | CCWWGGACGCNNNNN[NNNNN], |
| | C | | | | | GACGCCWWGG[NNNNN] |
| CseI, HgaI | GACG | M.Cmu51329I, M.EcoJA03PDcm, | CCWGG | 1, 5 | Unknown | CCWGGACGCNNNNN[NNNNN], |
| | C | M.RorR1Dcm, M.Sen8391Dcm, M.ZalSM2II | | | | GACGCCWGGN[NNNNN] |
| CseI, HgaI | GACG | M.CocI, M.Fli6794II, M.HincII, M.HindII, | GTYRAC | 2, 5 | m6A | GTYGACGCNNNNN[NNNNN] |
| | C | M.HinHia1II | | | | |
| CseI, HgaI | GACG | M.CsiFSMII, M.MfoCT6IV, M.NgoAIV, | GCCGGC | 2, 5 | m5C | GACGCCGGCN[NNNNN] |
| | C | M.NgoMIV, M1.Sgr13350II | | | | |
| CseI, HgaI | GACG | M.CviJI | VGCY | 2, 3 | m5C | GACGCYNNNN[NNNNN] |
| | C | | | | | |
| CseI, HgaI | GACG | M.CviQIII, M.CviSIII, M.Hpy299XI, | TCGA | 1, 4 | m6A | TCGACGCNNNNN[NNNNN] |
| | C | M.Hpy30II, M.Hpy32X, M.Hpy99VII, | | | | |
| | | M.HpyAX, M.HpyFII, M.HpyUM032XVII, | | | | |
| | | M.Tam77409III, M.TaqI, M.TpaRLI, | | | | |
| | | M.TthHB8I | | | | |
| CseI, HgaI | GACG | M.DdeI | CTNAG | 1, 5 | m5C | CTNAGACGCNNNNN[NNNNN], |
| | C | | | | | GACGCTNAGN[NNNNN] |
| CseI, HgaI | GACG | M.Esp3I | GAGACG | 4, 3 | m6A, | GAGACGCNNNNN[NNNNN] |
| | C | | | | m5C | |
| CseI, HgaI | GACG | M.FatI | CATG | 1, 4 | m5C | CATGACGCNNNNN[NNNNN], |
| | C | | | | | GACGCATGNN[NNNNN] |
| CseI, HgaI | GACG | M.FisAW1I, M.ThaI, M.Tph12270II | CGCG | 1, 4 | m4C | GACGCGNNNN[NNNNN], |
| | C | | | | | CGCGACGCNNNNN[NNNNN] |
| CseI, HgaI | GACG | M.FnoB1II, M.Hal24586I, M.Hpy298V, | CCGG | 1, 4 | m4C | CCGGACGCNNNNN[NNNNN], |
| | C | M.Hpy299IX, M.Hpy30V, M.Hpy32XI, | | | | GACGCCGGNN[NNNNN] |
| | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| CseI, HgaI | GACG | M.GsuRed1IV, M.Osp807II, M.Tth111I | GACNNNG | 2, 8 | m6A | GACGCNGTCN[NNNNN] |
| | C | | TC | | | |
| CseI, HgaI | GACG | M.HhaI, M.HinP1I, M.Hpy99III, M.HpyAVIII, | GCGC | 2, 3 | m5C | GACGCGCNNN[NNNNN] |
| | C | M.HpyUM032XV | | | | |
| CseI, HgaI | GACG | M.Hka19301IV | TCGCGA | 2, 5 | m4C | TCGCGACGCNNNNN[NNNNN] |
| | C | | | | | |
| CseI, HgaI | GACG | M.Hme33500II | HGCWGCK | 3 | m4C | GACGCWGCKN[NNNNN] |
| | C | | | | | |
| CseI, HgaI | GACG | M.Hpy188I, M.Hpy298IX, M.Hpy299V, | TCNGA | 1, 5 | m6A | TCNGACGCNNNNN[NNNNN] |
| | C | M.Hpy66V, M.HpyUM032V | | | | |
| CseI, HgaI | GACG | M.Hpy30IX, M.Hpy32IX, M.Hpy57IX, | GTNNAC | 2, 5 | m6A | GTNGACGCNNNNN[NNNNN] |
| | C | M.Hpy66XIII, M.Hpy8I, M.HpyFIX, | | | | |
| | | M.HpyUM032XI, M.HpyUM037V, M.MjaIV, | | | | |
| | | M.MspRAC08II | | | | |
| CseI, HgaI | GACG | M.Hpy30VIII, M.Hpy32VIII, M.Hpy57III, | TCNNGA | 1, 6 | m6A | TCNNGACGCNNNNN[NNNNN] |
| | C | M.Hpy66VI, M.Hpy99XVIII, M.HpyFVIII, | | | | |
| | | M.HpyPVIII | | | | |
| CseI, HgaI | GACG | M.Hpy30X, M.Hpy57VIII, M.HpyFXI, M.MhuI | CTNAG | 1, 5 | m4C | CTNAGACGCNNNNN[NNNNN], |
| | C | | | | | GACGCTNAGN[NNNNN] |
| CseI, HgaI | GACG | M.Hpy66I, M.Hpy99I | CGWCG | 2, 4 | m4C | CGACGCNNNNN[NNNNN] |
| | C | | | | | |
| CseI, HgaI | GACG | M.Hpy99II, M.Tsp45I, M.Tsu2489II | GTSAC | 2, 4 | m6A | GTGACGCNNNNN[NNNNN] |
| | C | | | | | |
| CseI, HgaI | GACG | M.Hpy99IV, M.HpyFORFX | CCNNGG | 1, 6 | m4C | CCNNGGACGCNNNNN[NNNNN], |
| | C | | | | | GACGCCNNGG[NNNNN] |
| CseI, HgaI | GACG | M.HpyD27I | GAGG | 2, 4 | m6A, | GACGCCTCNN[NNNNN], |
| | C | | | | m5C | GAGGACGCNNNNN[NNNNN] |
| CseI, HgaI | GACG | M.Lph145I, M.Lph93I | GGWGA | 5 | m6A | GGWGACGCNNNNN[NNNNN] |
| | C | | | | | |
| CseI, HgaI | GACG | M.Msp1II | CCAGG | 5 | m4C | GACGCCTGGN[NNNNN], |
| | C | | | | | CCAGGACGCNNNNN[NNNNN] |
| CseI, HgaI | GACG | M.Msp42II | CCWGG | 1, 5 | m4C | CCWGGACGCNNNNN[NNNNN], |
| | C | | | | | GACGCCWGGN[NNNNN] |
| CseI, HgaI | GACG | M.NheI | GCTAGC | 1, 6 | m4C | GACGCTAGCN[NNNNN] |
| | C | | | | | |
| CseI, HgaI | GACG | M.Nme18III | CCAGA | 2, 4 | m5C | CCAGACGCNNNNN[NNNNN] |
| | C | | | | | |
| CseI, HgaI | GACG | M.Nme18V | GCGCGC | 2, 5 | m5C | GACGCGCGCN[NNNNN] |
| | C | | | | | |
| CseI, HgaI | GACG | M.NmeDI | VCCGGY | 2, 5 | m5C | GACGCCGGBN[NNNNN] |
| | C | | | | | |
| CseI, HgaI | GACG | M.NmeMC58III | CCAGA | 1 | m5C | TCTGGACGCNNNNN[NNNNN], |
| | C | | | | | GACGCCAGAN[NNNNN] |
| CseI, HgaI | GACG | M.Nsp209III, M.Nsp51109II | CTCGAG | 1, 6 | m5C | CTCGAGACGCNNNNN[NNNNN], |
| | C | | | | | GACGCTCGAG[NNNNN] |
| CseI, HgaI | GACG | M.NspI, M.NspHI | RCATGY | 2, 5 | m5C | GACGCATGYN[NNNNN] |
| | C | | | | | |
| CseI, HgaI | GACG | M.Pam7686I | CCATGG | 1, 6 | m5C | CCATGGACGCNNNNN[NNNNN], |
| | C | | | | | GACGCCATGG[NNNNN] |
| CseI, HgaI | GACG | M.Pfa23572II | CGATCG | 1, 6 | m5C | CGATCGACGCNNNNN[NNNNN], |
| | C | | | | | GACGCGATCG[NNNNN] |
| CseI, HgaI | GACG | M.Pla5I | CTAG | 1, 4 | Unknown | CTAGACGCNNNNN[NNNNN], |
| | C | | | | | GACGCTAGNN[NNNNN] |
| CseI, HgaI | GACG | M.Sam53668II | CCATGG | 1, 6 | Unknown | CCATGGACGCNNNNN[NNNNN], |
| | C | | | | | GACGCCATGG[NNNNN] |
| CseI, HgaI | GACG | M.Sen1781III | CCWWGG | 1, 6 | Unknown | CCWWGGACGCNNNNN[NNNNN], |
| | C | | | | | GACGCCWWGG[NNNNN] |
| CseI, HgaI | GACG | M.SmoLVI, M.Spn23FII, M.Spn556I, | TCTAGA | 1, 6 | m6A | TCTAGACGCNNNNN[NNNNN] |
| | C | M.Spn7465I, M.SpnRI, M.XbaI | | | | |
| CseI, HgaI | GACG | M.SonIII | GACNNNN | 2, 8 | m6A | GACGCNNTGC[NNNNN] |
| | C | | TGC | | | |
| CseI, HgaI | GACG | M.TdeII | CTCTTC | 1, 2 | m4C, | GAAGAGACGCNNNNN[NNNNN], |
| | C | | | | m6A | GACGCTCTTC[NNNNN] |
| CseI, HgaI | GACG | M.TspRI | CASTG | 1, 5 | m5C | CASTGACGCNNNNN[NNNNN], |
| | C | | | | | GACGCASTGN[NNNNN] |
| CseI, HgaI | GACG | M.Yen2516I | GCGCGC | 2, 5 | Unknown | GACGCGCGCN[NNNNN] |
| | C | | | | | |
| CseI, HgaI | GACG | M1.AgoG2I, M2.AgoG2I | CCAGT | 1, 4 | m4C | ACTGGACGCNNNNN[NNNNN], |
| | C | | | | | GACGCCAGTN[NNNNN] |
| CseI, HgaI | GACG | M1.BspACI | CCGC | 1 | m5C | GCGGACGCNNNNN[NNNNN], |
| | C | | | | | GACGCCGCNN[NNNNN] |
| CseI, HgaI | GACG | M1.HphI | GGTGA | 4 | m5C | GGTGACGCNNNNN[NNNNN] |
| | C | | | | | |
| CseI, HgaI | GACG | M1.Hpy32II | GRRGA | 5 | m6A | GRRGACGCNNNNN[NNNNN] |
| | C | | | | | |
| CseI, HgaI | GACG | M1.HpyAII, M1.MboII, M1.NcuI | GAAGA | 5 | m6A | GAAGACGCNNNNN[NNNNN] |
| | C | | | | | |
| CseI, HgaI | GACG | M1.LplAG30I | GAAGG | 5 | m5C | GACGCCTTCN[NNNNN], |
| | C | | | | | GAAGGACGCNNNNN[NNNNN] |
| CseI, HgaI | GACG | M1.Sgr13350III, M2.Sgr13350III | GCTCTTC | 2, 3 | m4C, | GACGCTCTTC[NNNNN] |
| | C | | | | m6A | |
| CseI, HgaI | GACG | M2.BceSIII, M1.Pru9I, M2.Pru9I | GCCGT | 2, 4 | m4C | GACGCCGTNN[NNNNN] |
| | C | | | | | |
| CseI, HgaI | GACG | M2.ClaSBI, M1.Fli6794IV, M2.HphI, | GGTGA | 5 | m6A | GGTGACGCNNNNN[NNNNN] |
| | C | M2.Ngo3502II, M1.Ngo6140II, M1.NgoAXVI, | | | | |
| | | M1.NgoBVIII, M1.NgoFA19I, M2.NgoMVIII, | | | | |
| | | M2.PspHUN102I, M1.Ssp10II | | | | |
| CseI, HgaI | GACG | M2.Hpy32II, M2.HpyAII, M2.MboII, M2.NcuI | GAAGA | 4 | m4C | GAAGACGCNNNNN[NNNNN] |
| | C | | | | | |
| CseI, HgaI | GACG | M2.HpyAVI, M1.MnlI | GAGG | 4 | m5C | GACGCCTCNN[NNNNN], |
| | C | | | | | GAGGACGCNNNNN[NNNNN] |
| CseI, HgaI | GACG | M2.RspA76I | GGGAC | 4 | m6A | GGGACGCNNNNN[NNNNN] |
| | C | | | | | |
| FokI | GGAT | M.AbaBs12I, M.Awo1030I, M.Cdu23823I, | CCWGG | 1, 5 | m5C | CCWGGGATGNNNNNNNNN[NNNN], |
| | G | M.Csa8155II, M.Cun9529I, M.EasL1Dcm, | | | | CCWGGATGNNNNNNNNN[NNNN] |
| | | M.Eba57Dcm, M.Ec134399Dcm, | | | | |
| | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| FokI | GGAT | M.AciI | GCGG | 2, 4 | m5C | GCGGGATGNNNNNNNNN[NNNN], |
| | G | | | | | GCGGATGNNNNNNNNN[NNNN] |
| FokI | GGAT | M.Ama55I, M.CnaPQ2III, M.Rmo1926II, | CGGCCG | 1, 6 | m5C | CGGCCGGATGNNNNNNNNN[NNNN] |
| | G | M.SenC1765IV, M.SspG1I, M.XorPXII | | | | |
| FokI | GGAT | M.AquI | CYCGRG | 1, 6 | m5C | CYCGRGGATGNNNNNNNNN[NNNN], |
| | G | | | | | CYCGGGATGNNNNNNNNN[NNNN] |
| FokI | GGAT | M.AspU41II, M.BisIII, M.BsuW23II, | CCWGG | 2, 4 | m5C | CCWGGATGNNNNNNNNN[NNNN] |
| | G | M.CnaPQ2II, M.EcoO6Dcm, M.Eco12581Dcm, | | | | |
| | | M.Eco1655Dcm, M.Eco29787Dcm, | | | | |
| | | M.Eco3024Dcm, M.Eco3317Dcm, | | | | |
| | | M.Eco3325Dcm, M.Eco3740Dcm, | | | | |
| | | M.Eco3911Dcm, M.Eco4211Dcm, | | | | |
| | | M.Eco4465Dcm, M.Eco600Dcm, | | | | |
| | | M.Eco644Dcm, M.Eco86Dcm, | | | | |
| | | M.Eco9001Dcm, M.Eco9387Dcm, | | | | |
| | | M.EcoE1140Dcm, M.EcoE455Dcm, | | | | |
| | | M.EcoGDcm, M.EcoNU14Dcm, M.EcoRII, | | | | |
| | | M.Kpn223III, M.Kpn35657II, M.Kpn36Dcm, | | | | |
| | | M.Kpn555I, M.Kpn62629II, M.KpnAATV, | | | | |
| | | M.Kqu603Dcm, M.Psp345I, M.Sen158Dcm, | | | | |
| | | M.Sen641Dcm, M.SenA46Dcm, | | | | |
| | | M.SenAboDcm, M.TcoKWC4II | | | | |
| FokI | GGAT | M.AvaV, M.SliSI | GATC | 1, 4 | m4C | GGATGATCNNNNNN[NNNN] |
| | G | | | | | |
| FokI | GGAT | M.BceSII | GGWCC | 1, 5 | m5C | GGATGGWCCNNNNN[NNNN] |
| | G | | | | | |
| FokI | GGAT | M.BcoSlacII, M.MspLW5III, M.Nbr128I, | CTCGAG | 1, 6 | m4C | CTCGAGGATGNNNNNNNNN[NNNN] |
| | G | M.Psy18884II, M.Tam77409II, M.TbrGE1II, | | | | |
| | | M.TpaRLII | | | | |
| FokI | GGAT | M.BepI, M.FpsJV, M.Gel16401III, M.Pdi8503I | CGCG | 1, 4 | m5C | CGCGGATGNNNNNNNNN[NNNN] |
| | G | | | | | |
| FokI | GGAT | M.BfaSIII, M.Ccl10114I, M.CfrB38I, | ATGCAT | 2, 5 | m6A | GGATGCATNNNNNN[NNNN] |
| | G | M.CfrH1I, M.EcoGVI, M.EcoKII, | | | | |
| | | M.Fnu10562II, M.SbaUIV, M.Sbo12419III, | | | | |
| | | M.Sbo268II, M.Sen10384II, M.Sen10708II, | | | | |
| | | M.Sen1080II, M.Sen1175III, M.Sen13IV, | | | | |
| | | M.Sen13311IV, M.Sen1387II, M.Sen1427III, | | | | |
| | | M.Sen14882III, M.Sen15791III, M.Sen158III, | | | | |
| | | M.Sen16I, M.Sen1655III, M.Sen1676III, | | | | |
| | | M.Sen1677III, M.Sen1728III, M.Sen1735III, | | | | |
| | | M.Sen1736IV, M.Sen1764III, M.Sen1766III, | | | | |
| | | M.Sen1781IV, M.Sen1783III, M.Sen18569II, | | | | |
| | | M.Sen1878III, M.Sen1880III, M.Sen1896III, | | | | |
| | | M.Sen1898III, M.Sen1899III, M.Sen1903III, | | | | |
| | | M.Sen1906III, M.Sen1908III, M.Sen1910III, | | | | |
| | | M.Sen1921III, M.Sen1927II, M.Sen195II, | | | | |
| | | M.Sen2050II, M.Sen2064III, M.Sen2069III, | | | | |
| | | M.Sen22462I, M.Sen255II, M.Sen318III, | | | | |
| | | M.Sen3387III, M.Sen3890III, M.Sen483III, | | | | |
| | | M.Sen624III, M.Sen641III, M.Sen7378II, | | | | |
| | | M.Sen8391III, M.Sen8692III, M.SenA46III, | | | | |
| | | M.SenAbaII, M.SenAboII, M.SenAnaIII, | | | | |
| | | M.SenC1736III, M.SenC1765III, | | | | |
| | | M.SenC1808IV, M.SenC1810III, M.SenJIII, | | | | |
| | | M.SenL1II, M.SenL15III, M.SenN1660II, | | | | |
| | | M.SenSARA26II, M.SenSPBIII, M.SenTFIII | | | | |
| FokI | GGAT | M.Bhy27164IV, M.BstNI, M.EcoDHB4Dcm, | CCWGG | 2, 4 | m4C | CCWGGATGNNNNNNNNN[NNNN] |
| | G | M.EcoK54Dcm, M.Gth3570I, M.Hla49239I, | | | | |
| | | M.MvaI, M.Pdi8503II, M.Sso30807Dcm, | | | | |
| | | M.YpeI, M.Ype41I, M.YpeShI | | | | |
| FokI | GGAT | M.BsoBI | CYCGRG | 1, 6 | m4C | CYCGRGGATGNNNNNNNNN[NNNN], |
| | G | | | | | CYCGGGATGNNNNNNNNN[NNNN] |
| FokI | GGAT | M.Bsp14III, M.Bsp3003I, M.Bsp3004I, | GATC | 1, 4 | m5C | GGATGATCNNNNNN[NNNN] |
| | G | M.CalB3III, M.CpeAIII, M.Esu35585I, | | | | |
| | | M.FpsJIV, M.Lba2031I, M.Lbo2004I, | | | | |
| | | M.LlaKR2I, M.Lmo1042I, M.Lmo7956II, | | | | |
| | | M.Lsp10393II, M.MspI7I, M.Sau3AI, M.Sba3I, | | | | |
| | | M.Sin395I, M.Spn23FI, M.Sth368I | | | | |
| FokI | GGAT | M.BstXI | CCANNNN | 3, 10 | m6A | CCANNNGGATGGNNNNNNNN[NNNN] |
| | G | | NNTGG | | | |
| FokI | GGAT | M.BsuFI, M.CpeAIV, M.MspI | CCGG | 1, 4 | m5C | CCGGGATGNNNNNNNNN[NNNN], |
| | G | | | | | CCGGATGNNNNNNNNN[NNNN] |
| FokI | GGAT | M.Cab13497I, M.Dac11109III, M.Dco13527I, | CTAG | 1, 4 | m4C | CTAGGATGNNNNNNNNN[NNNN] |
| | G | M.Djo10085I, M.Hbo11551I, M.Hka19301III, | | | | |
| | | M.Hme33500I, M.HsaR1I, M.Hsi33800I, | | | | |
| | | M.Hsp KM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| FokI | GGAT | M.CagVI | TCCGGA | 1, 6 | m6A | TCCGGATGNNNNNNNNN[NNNN] |
| | G | | | | | |
| FokI | GGAT | M.Cdi630III | CCSSGG | 2, 5 | m4C | CCSSGGATGNNNNNNNNN[NNNN] |
| | G | | | | | |
| FokI | GGAT | M.Cfr9I, M.SmaI, M.Sma36365II, M.Xca85III, | CCCGGG | 2, 5 | m4C | CCCGGGATGNNNNNNNNN[NNNN] |
| | G | M.XcyI, M.XmaI, M.XveII | | | | |
| FokI | GGAT | M.CfrBI, M.Sen1735IV, M.SenAnaIV | CCWWGG | 1, 6 | m4C | CCWWGGGATGNNNNNNNNN[NNNN], |
| | G | | | | | CCWWGGATGNNNNNNNNN[NNNN] |
| FokI | GGAT | M.Cmu51329I, M.EcoJA03PDcm, | CCWGG | 1, 5 | Unknown | CCWGGGATGNNNNNNNNN[NNNN], |
| | G | M.RorR1Dcm, M.Sen8391Dcm, M.ZalSM2II | | | | CCWGGATGNNNNNNNNN[NNNN] |
| FokI | GGAT | M.CocIII, M.Fsi4947II, M.TdeIII, | GGNCC | 1, 5 | m4C | GGATGGNCCNNNNN[NNNN] |
| | G | M.Tph12270I | | | | |
| FokI | GGAT | M.Cpa18696III, M.Ype19I, M.YpeDodI, | CCWGG | 2, 4 | Unknown | CCWGGATGNNNNNNNNN[NNNN] |
| | G | M.YpeEDI, M.YpeJ9I, M.YpePGI | | | | |
| FokI | GGAT | M.CviRI, M.Hpy298VIII, M.Hpy299VIII, | TGCA | 1, 4 | m6A | GGATGCANNNNNNN[NNNN] |
| | G | M.Hpy30VI, M.Hpy32V, M.Hpy57II, | | | | |
| | | M.Hpy66VIII, M.HpyFVI, M.HpyUM032VIII, | | | | |
| | | M.HpyUM037VIII | | | | |
| FokI | GGAT | M.Dac11109II, M.Hfo6591I, M.MhuII, | GTAC | 1, 4 | m4C | GGATGTACNNNNNN[NNNN] |
| | G | M.MjaV, M.MlaZII, M.MspLW5I, M.RsaI | | | | |
| FokI | GGAT | M.DdeI | CTNAG | 1, 5 | m5C | CTNAGGATGNNNNNNNNN[NNNN] |
| | G | | | | | |
| FokI | GGAT | M.DsaV, M.Ecl18kI, M.FpsJVI, M.NlaX, | CCNGG | 2, 4 | m5C | CCNGGATGNNNNNNNNN[NNNN] |
| | G | M.SenPI, M.SsoII | | | | |
| FokI | GGAT | M.Eco29kI, M.NgoAIII, M.SacII | CCGCGG | 2, 5 | m5C | CCGCGGATGNNNNNNNNN[NNNN] |
| | G | | | | | |
| FokI | GGAT | M.EcoNI | CCTNNNN | 2, 10 | m4C | CCTNNNNNAGGATGNNNNNNNNN[NNNN] |
| | G | | NAGG | | | |
| FokI | GGAT | M.EfaRFII, M.Mae7806II | CCGG | 2, 3 | m4C | CCGGATGNNNNNNNNN[NNNN] |
| | G | | | | | |
| FokI | GGAT | M.Esp1396I | CCANNNN | 3, 9 | m6A | CCANNGGATGGNNNNNNNN[NNNN] |
| | G | | NTGG | | | |
| FokI | GGAT | M.FatI | CATG | 1, 4 | m5C | CATGGATGNNNNNNNNN[NNNN] |
| | G | | | | | |
| FokI | GGAT | M.FisAW1I, M.ThaI, M.Tph12270II | CGCG | 1, 4 | m4C | CGCGGATGNNNNNNNNN[NNNN] |
| | G | | | | | |
| FokI | GGAT | M.FnoB1II, M.Hal24586I, M.Hpy298V, | CCGG | 1, 4 | m4C | CCGGGATGNNNNNNNNN[NNNN], |
| | G | M.Hpy299IX, M.Hpy30V, M.Hpy32XI, | | | | CCGGATGNNNNNNNNN[NNNN] |
| | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| FokI | GGAT | M.FpsJI, M.HpaII, M.Nme18II, M.NmeAI | CCGG | 2, 3 | m5C | CCGGATGNNNNNNNNN[NNNN] |
| | G | | | | | |
| FokI | GGAT | M.Gel16401IV | CTGG | 3 | m5C | CTGGATGNNNNNNNNN[NNNN] |
| | G | | | | | |
| FokI | GGAT | M.Hpy188I, M.Hpy298IX, M.Hpy299V, | TCNGA | 1, 5 | m6A | TCGGATGNNNNNNNNN[NNNN] |
| | G | M.Hpy66V, M.HpyUM032V | | | | |
| FokI | GGAT | M.Hpy30VIII, M.Hpy32VIII, M.Hpy57III, | TCNNGA | 1, 6 | m6A | TCNGGATGNNNNNNNNN[NNNN] |
| | G | M.Hpy66VI, M.Hpy99XVIII, M.HpyFVIII, | | | | |
| | | M.HpyPVIII | | | | |
| FokI | GGAT | M.Hpy30X, M.Hpy57VIII, M.HpyFXI, M.MhuI | CTNAG | 1, 5 | m4C | CTNAGGATGNNNNNNNNN[NNNN] |
| | G | | | | | |
| FokI | GGAT | M.Hpy99IV, M.HpyFORFX | CCNNGG | 1, 6 | m4C | CCNNGGGATGNNNNNNNNN[NNNN], |
| | G | | | | | CCNNGGATGNNNNNNNNN[NNNN] |
| FokI | GGAT | M.HpyAV, M.Nme18IV | GAAGG | 3, 4 | m6A, | GAAGGATGNNNNNNNNN[NNNN] |
| | G | | | | m5C | |
| FokI | GGAT | M.HpyD27I | GAGG | 2, 4 | m6A, | GAGGGATGNNNNNNNNN[NNNN], |
| | G | | | | m5C | GAGGATGNNNNNNNNN[NNNN] |
| FokI | GGAT | M.MjaII | GGNCC | 1, 5 | m5C | GGATGGNCCNNNNN[NNNN] |
| | G | | | | | |
| FokI | GGAT | M.Msp1II | CCAGG | 5 | m4C | CCAGGGATGNNNNNNNNN[NNNN], |
| | G | | | | | CCAGGATGNNNNNNNNN[NNNN] |
| FokI | GGAT | M.Msp42II | CCWGG | 1, 5 | m4C | CCWGGGATGNNNNNNNNN[NNNN], |
| | G | | | | | CCWGGATGNNNNNNNNN[NNNN] |
| FokI | GGAT | M.NheI | GCTAGC | 1, 6 | m4C | GGATGCTAGCNNNN[NNNN] |
| | G | | | | | |
| FokI | GGAT | M.Nme18III | TCTGG | 2, 4 | m5C | TCTGGATGNNNNNNNNN[NNNN] |
| | G | | | | | |
| FokI | GGAT | M.NmeDI | RCCGGB | 2, 5 | m5C | RCCGGGATGNNNNNNNNN[NNNN] |
| | G | | | | | |
| FokI | GGAT | M.NmeMC58III | TCTGG | 5 | m5C | TCTGGGATGNNNNNNNNN[NNNN], |
| | G | | | | | TCTGGATGNNNNNNNNN[NNNN] |
| FokI | GGAT | M.Nsp209III, M.Nsp51109II | CTCGAG | 1, 6 | m5C | CTCGAGGATGNNNNNNNNN[NNNN] |
| | G | | | | | |
| FokI | GGAT | M.Pam7686I | CCATGG | 1, 6 | m5C | CCATGGGATGNNNNNNNNN[NNNN], |
| | G | | | | | CCATGGATGNNNNNNNNN[NNNN] |
| FokI | GGAT | M.Pfa23572II | CGATCG | 1, 6 | m5C | CGATCGGATGNNNNNNNNN[NNNN] |
| | G | | | | | |
| FokI | GGAT | M.Pla5I | CTAG | 1, 4 | Unknown | CTAGGATGNNNNNNNNN[NNNN] |
| | G | | | | | |
| FokI | GGAT | M.Sam53668II | CCATGG | 1, 6 | Unknown | CCATGGGATGNNNNNNNNN[NNNN], |
| | G | | | | | CCATGGATGNNNNNNNNN[NNNN] |
| FokI | GGAT | M.Sen1781III | CCWWGG | 1, 6 | Unknown | CCWWGGGATGNNNNNNNNN[NNNN], |
| | G | | | | | CCWWGGATGNNNNNNNNN[NNNN] |
| FokI | GGAT | M.SonIII | GACNNNN | 2, 8 | m6A | GACNGGATGCNNNNNNNN[NNNN] |
| | G | | TGC | | | |
| FokI | GGAT | M.TdeII | GAAGAG | 5, 6 | m6A, | GAAGAGGATGNNNNNNNNN[NNNN] |
| | G | | | | m4C | |
| FokI | GGAT | M.TspRI | CASTG | 1, 5 | m5C | CASTGGATGNNNNNNNNN[NNNN] |
| | G | | | | | |
| FokI | GGAT | M.XcmI | CCANNNN | 3, 13 | m6A | CCANNNNNNGGATGGNNNNNNNN[NNNN] |
| | G | | NNNNNTG | | | |
| | | | G | | | |
| FokI | GGAT | M1.AgoG2I, M2.AgoG2I | ACTGG | 2, 5 | m4C | ACTGGGATGNNNNNNNNN[NNNN], |
| | G | | | | | ACTGGATGNNNNNNNNN[NNNN] |
| FokI | GGAT | M1.Bag18758II, M2.Bag18758II | CACGGG | 3, 5 | Unknown | CACGGGATGNNNNNNNNN[NNNN] |
| | G | | | | | |
| FokI | GGAT | M1.BccI, M1.Hpy30XI, M2.Hpy66X, | GATGG | 2 | m6A | GGATGGNNNNNNNN[NNNN] |
| | G | M1.Jsp2502I, M2.MmyCVI | | | | |
| FokI | GGAT | M1.BcnI, M2.BcnI, M.NciI | CCSGG | 2, 4 | m4C | CCSGGATGNNNNNNNNN[NNNN] |
| | G | | | | | |
| FokI | GGAT | M1.BfiI | ACTGGG | 5 | m4C | ACTGGGATGNNNNNNNNN[NNNN] |
| | G | | | | | |
| FokI | GGAT | M1.BhaI, M1.Bst19I, M.Fnu10562III, | GATGC | 3 | m6A | GGATGCNNNNNNNN[NNNN] |
| | G | M1.Mbo45IV, M2.Mbo45IV | | | | |
| FokI | GGAT | M1.BspACI | GCGG | 4 | m5C | GCGGGATGNNNNNNNNN[NNNN], |
| | G | | | | | GCGGATGNNNNNNNNN[NNNN] |
| FokI | GGAT | M1.Csp16704IV, M2.Csp16704IV, | GATGC | 2, 3 | m6A | GGATGCNNNNNNNN[NNNN] |
| | G | M.Lsp10393I, M.Mha183II, M.Mha185II, | | | | |
| | | M.Mha807II, M.Mva1312II | | | | |
| FokI | GGAT | M1.Hpy299X, M2.Hpy32XIII, M1.HpyFXIII, | GATGG | 2, 3 | m6A | GGATGGNNNNNNNN[NNNN] |
| | G | M2.HpyFXIII, M2.HpyUM037VI, M1.McaCI, | | | | |
| | | M2.McaCI, M1.PspHUN102II, | | | | |
| | | M2.PspHUN102II | | | | |
| FokI | GGAT | M1.Hpy32II | GRRGA | 5 | m6A | GRGGATGNNNNNNNNN[NNNN] |
| | G | | | | | |
| FokI | GGAT | M1.LplAG30I | GAAGG | 5 | m5C | GAAGGGATGNNNNNNNNN[NNNN], |
| | G | | | | | GAAGGATGNNNNNNNNN[NNNN] |
| FokI | GGAT | M2.BccI, M2.Hpy299X, M1.Hpy66X, | GATGG | 3 | m6A | GGATGGNNNNNNNN[NNNN] |
| | G | M1.HpyC1I, M2.HpyC1I, M1.HpyUM032X, | | | | |
| | | M2.Jsp2502I, M1.MmyCVI | | | | |
| FokI | GGAT | M2.BhaI | GATGC | 2 | m6A | GGATGCNNNNNNNN[NNNN] |
| | G | | | | | |
| FokI | GGAT | M2.HpyAVI, M1.MnlI | GAGG | 4 | m5C | GAGGGATGNNNNNNNNN[NNNN], |
| | G | | | | | GAGGATGNNNNNNNNN[NNNN] |
| StsI | GGAT | M.AbaBs12I, M.Awo1030I, M.Cdu23823I, | CCWGG | 1, 5 | m5C | CCWGGGATGNNNNNNNNNN[NNNN], |
| | G | M.Csa8155II, M.Cun9529I, M.EasL1Dcm, | | | | CCWGGATGNNNNNNNNNN[NNNN] |
| | | M.Eba57Dcm, M.Ec134399Dcm, | | | | |
| | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| StsI | GGAT | M.AciI | GCGG | 2, 4 | m5C | GCGGGATGNNNNNNNNNN[NNNN], |
| | G | | | | | GCGGATGNNNNNNNNNN[NNNN] |
| StsI | GGAT | M.Ama55I, M.CnaPQ2III, M.Rmo1926II, | CGGCCG | 1, 6 | m5C | CGGCCGGATGNNNNNNNNNN[NNNN] |
| | G | M.SenC1765IV, M.SspG1I, M.XorPXII | | | | |
| StsI | GGAT | M.AquI | CYCGRG | 1, 6 | m5C | CYCGRGGATGNNNNNNNNNN[NNNN], |
| | G | | | | | CYCGGGATGNNNNNNNNNN[NNNN] |
| StsI | GGAT | M.AspU41II, M.BisIII, M.BsuW23II, | CCWGG | 2, 4 | m5C | CCWGGATGNNNNNNNNNN[NNNN] |
| | G | M.CnaPQ2II, M.EcoO6Dcm, M.Eco12581Dcm, | | | | |
| | | M.Eco1655Dcm, M.Eco29787Dcm, | | | | |
| | | M.Eco3024Dcm, M.Eco3317Dcm, | | | | |
| | | M.Eco3325Dcm, M.Eco3740Dcm, | | | | |
| | | M.Eco3911Dcm, M.Eco4211Dcm, | | | | |
| | | M.Eco4465Dcm, M.Eco600Dcm, | | | | |
| | | M.Eco644Dcm, M.Eco86Dcm, | | | | |
| | | M.Eco9001Dcm, M.Eco9387Dcm, | | | | |
| | | M.EcoE1140Dcm, M.EcoE455Dcm, | | | | |
| | | M.EcoGDcm, M.EcoNU14Dcm, M.EcoRII, | | | | |
| | | M.Kpn223III, M.Kpn35657II, M.Kpn36Dcm, | | | | |
| | | M.Kpn555I, M.Kpn62629II, M.KpnAATV, | | | | |
| | | M.Kqu603Dcm, M.Psp345I, M.Sen158Dcm, | | | | |
| | | M.Sen641Dcm, M.SenA46Dcm, | | | | |
| | | M.SenAboDcm, M.TcoKWC4II | | | | |
| StsI | GGAT | M.AvaV, M.SliSI | GATC | 1, 4 | m4C | GGATGATCNNNNNNN[NNNN] |
| | G | | | | | |
| StsI | GGAT | M.BceSII | GGWCC | 1, 5 | m5C | GGATGGWCCNNNNNN[NNNN] |
| | G | | | | | |
| StsI | GGAT | M.BcoSlacII, M.MspLW5III, M.Nbr128I, | CTCGAG | 1, 6 | m4C | CTCGAGGATGNNNNNNNNNN[NNNN] |
| | G | M.Psy18884II, M.Tam77409II, M.TbrGE1II, | | | | |
| | | M.TpaRLII | | | | |
| StsI | GGAT | M.BepI, M.FpsJV, M.Gel16401III, M.Pdi8503I | CGCG | 1, 4 | m5C | CGCGGATGNNNNNNNNNN[NNNN] |
| | G | | | | | |
| StsI | GGAT | M.BfaSIII, M.Ccl10114I, M.CfrB38I, | ATGCAT | 2, 5 | m6A | GGATGCATNNNNNNN[NNNN] |
| | G | M.CfrH1I, M.EcoGVI, M.EcoKII, | | | | |
| | | M.Fnu10562II, M.SbaUIV, M.Sbo12419III, | | | | |
| | | M.Sbo268II, M.Sen10384II, M.Sen10708II, | | | | |
| | | M.Sen1080II, M.Sen1175III, M.Sen13IV, | | | | |
| | | M.Sen13311IV, M.Sen1387II, M.Sen1427III, | | | | |
| | | M.Sen14882III, M.Sen15791III, M.Sen158III, | | | | |
| | | M.Sen16I, M.Sen1655III, M.Sen1676III, | | | | |
| | | M.Sen1677III, M.Sen1728III, M.Sen1735III, | | | | |
| | | M.Sen1736IV, M.Sen1764III, M.Sen1766III, | | | | |
| | | M.Sen1781IV, M.Sen1783III, M.Sen18569II, | | | | |
| | | M.Sen1878III, M.Sen1880III, M.Sen1896III, | | | | |
| | | M.Sen1898III, M.Sen1899III, M.Sen1903III, | | | | |
| | | M.Sen1906III, M.Sen1908III, M.Sen1910III, | | | | |
| | | M.Sen1921III, M.Sen1927II, M.Sen195II, | | | | |
| | | M.Sen2050II, M.Sen2064III, M.Sen2069III, | | | | |
| | | M.Sen22462I, M.Sen255II, M.Sen318III, | | | | |
| | | M.Sen3387III, M.Sen3890III, M.Sen483III, | | | | |
| | | M.Sen624III, M.Sen641III, M.Sen7378II, | | | | |
| | | M.Sen8391III, M.Sen8692III, M.SenA46III, | | | | |
| | | M.SenAbaII, M.SenAboII, M.SenAnaIII, | | | | |
| | | M.SenC1736III, M.SenC1765III, | | | | |
| | | M.SenC1808IV, M.SenC1810III, M.SenJIII, | | | | |
| | | M.SenL1II, M.SenL15III, M.SenN1660II, | | | | |
| | | M.SenSARA26II, M.SenSPBIII, M.SenTFIII | | | | |
| StsI | GGAT | M.Bhy27164IV, M.BstNI, M.EcoDHB4Dcm, | CCWGG | 2, 4 | m4C | CCWGGATGNNNNNNNNNN[NNNN] |
| | G | M.EcoK54Dcm, M.Gth3570I, M.Hla49239I, | | | | |
| | | M.MvaI, M.Pdi8503II, M.Sso30807Dcm, | | | | |
| | | M.YpeI, M.Ype41I, M.YpeShI | | | | |
| StsI | GGAT | M.BsoBI | CYCGRG | 1, 6 | m4C | CYCGRGGATGNNNNNNNNNN[NNNN], |
| | G | | | | | CYCGGGATGNNNNNNNNNN[NNNN] |
| StsI | GGAT | M.Bsp14III, M.Bsp3003I, M.Bsp3004I, | GATC | 1, 4 | m5C | GGATGATCNNNNNNN[NNNN] |
| | G | M.CalB3III, M.CpeAIII, M.Esu35585I, | | | | |
| | | M.FpsJIV, M.Lba2031I, M.Lbo2004I, | | | | |
| | | M.LlaKR2I, M.Lmo1042I, M.Lmo7956II, | | | | |
| | | M.Lsp10393II, M.MspI7I, M.Sau3AI, M.Sba3I, | | | | |
| | | M.Sin395I, M.Spn23FI, M.Sth368I | | | | |
| StsI | GGAT | M.BstXI | CCANNNN | 3, 10 | m6A | CCANNNGGATGGNNNNNNNNN[NNNN] |
| | G | | NNTGG | | | |
| StsI | GGAT | M.BsuFI, M.CpeAIV, M.MspI | CCGG | 1, 4 | m5C | CCGGGATGNNNNNNNNNN[NNNN], |
| | G | | | | | CCGGATGNNNNNNNNNN[NNNN] |
| StsI | GGAT | M.Cab13497I, M.Dac11109III, M.Dco13527I, | CTAG | 1, 4 | m4C | CTAGGATGNNNNNNNNNN[NNNN] |
| | G | M.Djo10085I, M.Hbo11551I, M.Hka19301III, | | | | |
| | | M.Hme33500I, M.HsaR1I, M.Hsi33800I, | | | | |
| | | M.HspKM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| StsI | GGAT | M.CagVI | TCCGGA | 1, 6 | m6A | TCCGGATGNNNNNNNNNN[NNNN] |
| | G | | | | | |
| StsI | GGAT | M.Cdi630III | CCSSGG | 2, 5 | m4C | CCSSGGATGNNNNNNNNNN[NNNN] |
| | G | | | | | |
| StsI | GGAT | M.Cfr9I, M.SmaI, M.Sma36365II, M.Xca85III, | CCCGGG | 2, 5 | m4C | CCCGGGATGNNNNNNNNNN[NNNN] |
| | G | M.XcyI, M.XmaI, M.XveII | | | | |
| StsI | GGAT | M.CfrBI, M.Sen1735IV, M.SenAnaIV | CCWWGG | 1, 6 | m4C | CCWWGGGATGNNNNNNNNNN[NNNN], |
| | G | | | | | CCWWGGATGNNNNNNNNNN[NNNN] |
| StsI | GGAT | M.Cmu51329I, M.EcoJA03PDcm, | CCWGG | 1, 5 | Unknown | CCWGGGATGNNNNNNNNNN[NNNN], |
| | G | M.RorR1Dcm, M.Sen8391Dcm, M.ZalSM2II | | | | CCWGGATGNNNNNNNNNN[NNNN] |
| StsI | GGAT | M.CocIII, M.Fsi4947II, M.TdeIII, | GGNCC | 1, 5 | m4C | GGATGGNCCNNNNNN[NNNN] |
| | G | M.Tph12270I | | | | |
| StsI | GGAT | M.Cpa18696III, M.Ype19I, M.YpeDodI, | CCWGG | 2, 4 | Unknown | CCWGGATGNNNNNNNNNN[NNNN] |
| | G | M.YpeEDI, M.YpeJ9I, M.YpePGI | | | | |
| StsI | GGAT | M.CviRI, M.Hpy298VIII, M.Hpy299VIII, | TGCA | 1, 4 | m6A | GGATGCANNNNNNNN[NNNN] |
| | G | M.Hpy30VI, M.Hpy32V, M.Hpy57II, | | | | |
| | | M.Hpy66VIII, M.HpyFVI, M.HpyUM032VIII, | | | | |
| | | M.HpyUM037VIII | | | | |
| StsI | GGAT | M.Dac11109II, M.Hfo6591I, M.MhuII, | GTAC | 1, 4 | m4C | GGATGTACNNNNNNN[NNNN] |
| | G | M.MjaV, M.MlaZII, M.MspLW5I, M.RsaI | | | | |
| StsI | GGAT | M.DdeI | CTNAG | 1, 5 | m5C | CTNAGGATGNNNNNNNNNN[NNNN] |
| | G | | | | | |
| StsI | GGAT | M.DsaV, M.Ecl18kI, M.FpsJVI, M.NlaX, | CCNGG | 2, 4 | m5C | CCNGGATGNNNNNNNNNN[NNNN] |
| | G | M.SenPI, M.SsoII | | | | |
| StsI | GGAT | M.Eco29kI, M.NgoAIII, M.SacII | CCGCGG | 2, 5 | m5C | CCGCGGATGNNNNNNNNNN[NNNN] |
| | G | | | | | |
| StsI | GGAT | M.EcoNI | CCTNNNN | 2, 10 | m4C | CCTNNNNNAGGATGNNNNNNNNNN[NNNN] |
| | G | | NAGG | | | |
| StsI | GGAT | M.EfaRFII, M.Mae7806II | CCGG | 2, 3 | m4C | CCGGATGNNNNNNNNNN[NNNN] |
| | G | | | | | |
| StsI | GGAT | M.Esp1396I | CCANNNN | 3, 9 | m6A | CCANNGGATGGNNNNNNNNN[NNNN] |
| | G | | NTGG | | | |
| StsI | GGAT | M.FatI | CATG | 1, 4 | m5C | CATGGATGNNNNNNNNNN[NNNN] |
| | G | | | | | |
| StsI | GGAT | M.FisAW1I, M.ThaI, M.Tph12270II | CGCG | 1, 4 | m4C | CGCGGATGNNNNNNNNNN[NNNN] |
| | G | | | | | |
| StsI | GGAT | M.FnoB1II, M.Hal24586I, M.Hpy298V, | CCGG | 1, 4 | m4C | CCGGGATGNNNNNNNNNN[NNNN], |
| | G | M.Hpy299IX, M.Hpy30V, M.Hpy32XI, | | | | CCGGATGNNNNNNNNNN[NNNN] |
| | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| StsI | GGAT | M.FpsJI, M.HpaII, M.Nme18II, M.NmeAI | CCGG | 2, 3 | m5C | CCGGATGNNNNNNNNNN[NNNN] |
| | G | | | | | |
| StsI | GGAT | M.Gel16401IV | CTGG | 3 | m5C | CTGGATGNNNNNNNNNN[NNNN] |
| | G | | | | | |
| StsI | GGAT | M.Hpy188I, M.Hpy298IX, M.Hpy299V, | TCNGA | 1, 5 | m6A | TCGGATGNNNNNNNNNN[NNNN] |
| | G | M.Hpy66V, M.HpyUM032V | | | | |
| StsI | GGAT | M.Hpy30VIII, M.Hpy32VIII, M.Hpy57III, | TCNNGA | 1, 6 | m6A | TCNGGATGNNNNNNNNNN[NNNN] |
| | G | M.Hpy66VI, M.Hpy99XVIII, M.HpyFVIII, | | | | |
| | | M.HpyPVIII | | | | |
| StsI | GGAT | M.Hpy30X, M.Hpy57VIII, M.HpyFXI, M.MhuI | CTNAG | 1, 5 | m4C | CTNAGGATGNNNNNNNNNN[NNNN] |
| | G | | | | | |
| StsI | GGAT | M.Hpy99IV, M.HpyFORFX | CCNNGG | 1, 6 | m4C | CCNNGGGATGNNNNNNNNNN[NNNN], |
| | G | | | | | CCNNGGATGNNNNNNNNNN[NNNN] |
| StsI | GGAT | M.HpyAV, M.Nme18IV | GAAGG | 3, 4 | m6A, | GAAGGATGNNNNNNNNNN[NNNN] |
| | G | | | | m5C | |
| StsI | GGAT | M.HpyD27I | GAGG | 2, 4 | m6A, | GAGGGATGNNNNNNNNNN[NNNN], |
| | G | | | | m5C | GAGGATGNNNNNNNNNN[NNNN] |
| StsI | GGAT | M.MjaII | GGNCC | 1, 5 | m5C | GGATGGNCCNNNNNN[NNNN] |
| | G | | | | | |
| StsI | GGAT | M.Msp1II | CCAGG | 5 | m4C | CCAGGGATGNNNNNNNNNN[NNNN], |
| | G | | | | | CCAGGATGNNNNNNNNNN[NNNN] |
| StsI | GGAT | M.Msp42II | CCWGG | 1, 5 | m4C | CCWGGGATGNNNNNNNNNN[NNNN], |
| | G | | | | | CCWGGATGNNNNNNNNNN[NNNN] |
| StsI | GGAT | M.NheI | GCTAGC | 1, 6 | m4C | GGATGCTAGCNNNNN[NNNN] |
| | G | | | | | |
| Stsl | GGAT | M.Nme18III | TCTGG | 2, 4 | m5C | TCTGGATGNNNNNNNNNN[NNNN] |
| | G | | | | | |
| StsI | GGAT | M.NmeDI | RCCGGB | 2, 5 | m5C | RCCGGGATGNNNNNNNNNN[NNNN] |
| | G | | | | | |
| StsI | GGAT | M.NmeMC58III | TCTGG | 5 | m5C | TCTGGGATGNNNNNNNNNN[NNNN], |
| | G | | | | | TCTGGATGNNNNNNNNNN[NNNN] |
| StsI | GGAT | M.Nsp209III, M.Nsp51109II | CTCGAG | 1, 6 | m5C | CTCGAGGATGNNNNNNNNNN[NNNN] |
| | G | | | | | |
| StsI | GGAT | M.Pam7686I | CCATGG | 1, 6 | m5C | CCATGGGATGNNNNNNNNNN[NNNN], |
| | G | | | | | CCATGGATGNNNNNNNNNN[NNNN] |
| StsI | GGAT | M.Pfa23572II | CGATCG | 1, 6 | m5C | CGATCGGATGNNNNNNNNNN[NNNN] |
| | G | | | | | |
| StsI | GGAT | M.Pla5I | CTAG | 1, 4 | Unknown | CTAGGATGNNNNNNNNNN[NNNN] |
| | G | | | | | |
| StsI | GGAT | M.Sam53668II | CCATGG | 1, 6 | Unknown | CCATGGGATGNNNNNNNNNN[NNNN], |
| | G | | | | | CCATGGATGNNNNNNNNNN[NNNN] |
| StsI | GGAT | M.Sen1781III | CCWWGG | 1, 6 | Unknown | CCWWGGGATGNNNNNNNNNN[NNNN], |
| | G | | | | | CCWWGGATGNNNNNNNNNN[NNNN] |
| StsI | GGAT | M.SonIII | GACNNNN | 2, 8 | m6A | GACNGGATGCNNNNNNNNN[NNNN] |
| | G | | TGC | | | |
| StsI | GGAT | M.TdeII | GAAGAG | 5, 6 | m6A, | GAAGAGGATGNNNNNNNNNN[NNNN] |
| | G | | | | m4C | |
| StsI | GGAT | M.TspRI | CASTG | 1, 5 | m5C | CASTGGATGNNNNNNNNNN[NNNN] |
| | G | | | | | |
| StsI | GGAT | M.XcmI | CCANNNN | 3, 13 | m6A | CCANNNNNNGGATGGNNNNNNNNN[NNNN] |
| | G | | NNNNNTG | | | |
| | | | G | | | |
| StsI | GGAT | M1.AgoG2I, M2.AgoG2I | ACTGG | 2, 5 | m4C | ACTGGGATGNNNNNNNNNN[NNNN], |
| | G | | | | | ACTGGATGNNNNNNNNNN[NNNN] |
| StsI | GGAT | M1.Bag18758II, M2.Bag18758II | CACGGG | 3, 5 | Unknown | CACGGGATGNNNNNNNNNN[NNNN] |
| | G | | | | | |
| StsI | GGAT | M1.BccI, M1.Hpy30XI, M2.Hpy66X, | GATGG | 2 | m6A | GGATGGNNNNNNNNN[NNNN] |
| | G | M1.Jsp2502I, M2.MmyCVI | | | | |
| StsI | GGAT | M1.BcnI, M2.BcnI, M.NciI | CCSGG | 2, 4 | m4C | CCSGGATGNNNNNNNNNN[NNNN] |
| | G | | | | | |
| StsI | GGAT | M1.BfiI | ACTGGG | 5 | m4C | ACTGGGATGNNNNNNNNNN[NNNN] |
| | G | | | | | |
| StsI | GGAT | M1.BhaI, M1.Bst19I, M.Fnu10562III, | GATGC | 3 | m6A | GGATGCNNNNNNNNN[NNNN] |
| | G | M1.Mbo45IV, M2.Mbo45IV | | | | |
| StsI | GGAT | M1.BspACI | GCGG | 4 | m5C | GCGGGATGNNNNNNNNNN[NNNN], |
| | G | | | | | GCGGATGNNNNNNNNNN[NNNN] |
| StsI | GGAT | M1.Csp16704IV, M2.Csp16704IV, | GATGC | 2, 3 | m6A | GGATGCNNNNNNNNN[NNNN] |
| | G | M.Lsp10393I, M.Mha183II, M.Mha185II, | | | | |
| | | M.Mha807II, M.Mva1312II | | | | |
| StsI | GGAT | M1.Hpy299X, M2.Hpy32XIII, M1.HpyFXIII, | GATGG | 2, 3 | m6A | GGATGGNNNNNNNNN[NNNN] |
| | G | M2.HpyFXIII, M2.HpyUM037VI, M1.McaCI, | | | | |
| | | M2.McaCI, M1.PspHUN102II, | | | | |
| | | M2.PspHUN102II | | | | |
| StsI | GGAT | M1.Hpy32II | GRRGA | 5 | m6A | GRGGATGNNNNNNNNNN[NNNN] |
| | G | | | | | |
| StsI | GGAT | M1.LplAG30I | GAAGG | 5 | m5C | GAAGGGATGNNNNNNNNNN[NNNN], |
| | G | | | | | GAAGGATGNNNNNNNNNN[NNNN] |
| StsI | GGAT | M2.BccI, M2.Hpy299X, M1.Hpy66X, | GATGG | 3 | m6A | GGATGGNNNNNNNNN[NNNN] |
| | G | M1.HpyC1I, M2.HpyC1I, M1.HpyUM032X, | | | | |
| | | M2.Jsp2502I, M1.MmyCVI | | | | |
| StsI | GGAT | M2.BhaI | GATGC | 2 | m6A | GGATGCNNNNNNNNN[NNNN] |
| | G | | | | | |
| StsI | GGAT | M2.HpyAVI, M1.MnlI | GAGG | 4 | m5C | GAGGGATGNNNNNNNNNN[NNNN], |
| | G | | | | | GAGGATGNNNNNNNNNN[NNNN] |
| Acc36I, BfuAI, | ACCT | M.AauTCI, M.AchA6II, M.Asp146I, | TTAA | 1, 4 | m6A | TTAACCTGCNNNN[NNNN] |
| BspMI, BveI | GC | M.Asp24188I, M.Asp31YII, M.AspU41I, | | | | |
| | | M.BspJKG1III, M.EsaDix4I, M.EsaDix5I, | | | | |
| | | M.Llu27648II, M.Mch9957II, M.Mru1279VI, | | | | |
| | | M.MseI, M.Msi9946I, M.Sam53668III, | | | | |
| | | M.Sth20745I, M.Tam 77409I | | | | |
| Acc36I, BfuAI, | ACCT | M.AbaBs12I, M.Awo1030I, M.Cdu23823I, | CCWGG | 1, 5 | m5C | ACCTGCCWGG[NNNN] |
| BspMI, BveI | GC | M.Csa8155II, M.Cun9529I, M.EasL1Dcm, | | | | |
| | | M.Eba57Dcm, M.Ecl34399Dcm, | | | | |
| | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| Acc36I, BfuAI, | ACCT | M.Acc65I, M.Eco86I, M.KpnI, M.Lal216III, | GGTACC | 3, 4 | m6A | GGTACCTGCNNNN[NNNN] |
| BspMI, BveI | GC | M.Sen3387IV, M.Sen8391IV | | | | |
| Acc36I, BfuAI, | ACCT | M.Acc65VI, M.SenAZIII | CTKMAG | 2, 5 | m6A | ACCTGCAGNN[NNNN] |
| BspMI, BveI | GC | | | | | |
| Acc36I, BfuAI, | ACCT | M.AccI, M.FpsFPG3II, M.SdeAIV | GTMKAC | 2, 5 | m6A | GTMKACCTGCNNNN[NNNN] |
| BspMI, BveI | GC | | | | | |
| Acc36I, BfuAI, | ACCT | M.AciI | CCGC | 1, 3 | m5C | ACCTGCGGNN[NNNN], |
| BspMI, BveI | GC | | | | | ACCTGCCGCN[NNNN] |
| Acc36I, BfuAI, | ACCT | M.Ade56712I, M.Asp588III, M.AveCB51II, | CTGCAG | 2, 5 | m6A | ACCTGCAGNN[NNNN] |
| BspMI, BveI | GC | M.BbrUIII, M.Blo30698I, M.BsuBI, | | | | |
| | | M.CpaYL7III, M.Dba7044I, M.Dfa45958II, | | | | |
| | | M.Eco12581I, M.Eco2012I, M.EcoGIII, | | | | |
| | | M.EcoG089I, M.Eho16691II, M.Hal5920II, | | | | |
| | | M.Lsp6406II, M.MspNR41II, M.Pae490I, | | | | |
| | | M.Pox23570I, M.PstI, M.RpaII, M.Sca3403II, | | | | |
| | | M.Tsp61I, M.XmnII, M.YenWI, M.YinY228I | | | | |
| Acc36I, BfuAI, | ACCT | M.AgsI | TTSAA | 1, 5 | m6A | TTSAACCTGCNNNN[NNNN] |
| BspMI, BveI | GC | | | | | |
| Acc36I, BfuAI, | ACCT | M.AhdI | GACNNNN | 2, 10 | m6A | GACCTGCNGTC[NNNN] |
| BspMI, BveI | GC | | NGTC | | | |
| Acc36I, BfuAI, | ACCT | M.Alw26I | GAGAC | 4, 3 | m6A, | GAGACCTGCNNNN[NNNN] |
| BspMI, BveI | GC | | | | m5C | |
| Acc36I, BfuAI, | ACCT | M.AorT14III, M.GsuRed1III, M.SalI | GTCGAC | 2, 5 | m6A | GTCGACCTGCNNNN[NNNN] |
| BspMI, BveI | GC | | | | | |
| Acc36I, BfuAI, | ACCT | M.AplI | CTGCAG | 3, 4 | m5C | ACCTGCAGNN[NNNN] |
| BspMI, BveI | GC | | | | | |
| Acc36I, BfuAI, | ACCT | M.AvaIX, M.Cfr10I, M.Nme18VI, M.VchAI, | RCCGGY | 2, 5 | m5C | ACCTGCCGGY [NNNN] |
| BspMI, BveI | GC | M.Vch0395I | | | | |
| Acc36I, BfuAI, | ACCT | M.BamWS8I, M.CthV, M.EfaRFI, M.Saf8902II, | GCWGC | 2, 4 | m5C | ACCTGCWGCN[NNNN] |
| BspMI, BveI | GC | M.TacII, M.TcoKWC4III | | | | |
| Acc36I, BfuAI, | ACCT | M.BanLII, M.CpeAII, M.SinI | GGWCC | 2, 4 | m5C | GGACCTGCNNNN[NNNN] |
| BspMI, BveI | GC | | | | | |
| Acc36I, BfuAI, | ACCT | M.BbvI, M.BceSIV | GCAGC | 2, 4 | m5C | ACCTGCTGCN[NNNN], |
| BspMI, BveI | GC | | | | | ACCTGCAGCN[NNNN] |
| Acc36I, BfuAI, | ACCT | M.Bce16656II, M.Bce22E1II, M.Bce25416II, | GTWWAC | 2, 5 | m6A | GTWWACCTGCNNNN[NNNN] |
| BspMI, BveI | GC | M.Bce842II, M.Bce895II, M.BceJIV, | | | | |
| | | M.BceK56II, M.Bgl2196I, M.BmaBMKII, | | | | |
| | | M.BmaBMZII, M.Bps10134II, M.Bps1651II, | | | | |
| | | M.Bps7894II, M.Bps840III, M.BpsR668II, | | | | |
| | | M.BspRB39I, M.Bth1643II, M.Cba4G11II, | | | | |
| | | M.MspNR41I, M.Oba14I, M.Pap16535I, | | | | |
| | | M.Pfa23572I, M.Pox23570II, M.PpnI, | | | | |
| | | M.PpnRB38I, M.Pth25325I | | | | |
| Acc36I, BfuAI, | ACCT | M.BceSII | GGWCC | 1, 5 | m5C | GGACCTGCNNNN[NNNN] |
| BspMI, BveI | GC | | | | | |
| Acc36I, BfuAI, | ACCT | M.BepI, M.FpsJV, M.Gel16401III, M.Pdi8503I | CGCG | 1, 4 | m5C | ACCTGCGCGN[NNNN] |
| BspMI, BveI | GC | | | | | |
| Acc36I, BfuAI, | ACCT | M.Bhy27164V, M.DraI, M.Lsp6406V, | TTTAAA | 1, 6 | m6A | TTTAAACCTGCNNNN[NNNN] |
| BspMI, BveI | GC | M.Tsu2489V | | | | |
| Acc36I, BfuAI, | ACCT | M.BspRAC04I, M.EcaI, M.Eco3936IV | GGTNACC | 3, 5 | m6A | GGTNACCTGCNNNN[NNNN] |
| BspMI, BveI | GC | | | | | |
| Acc36I, BfuAI, | ACCT | M.BstXI | CCANNNN | 3, 10 | m6A | CCACCTGCNTGG[NNNN] |
| BspMI, BveI | GC | | NNTGG | | | |
| Acc36I, BfuAI, | ACCT | M.BsuFI, M.CpeAIV, M.MspI | CCGG | 1, 4 | m5C | ACCTGCCGGN[NNNN] |
| BspMI, BveI | GC | | | | | |
| Acc36I, BfuAI, | ACCT | M.Cab13497I, M.Dac11109III, M.Dco13527I, | CTAG | 1, 4 | m4C | ACCTGCTAGN[NNNN] |
| BspMI, BveI | GC | M.Djo10085I, M.Hbo11551I, M.Hka19301III, | | | | |
| | | M.Hme33500I, M.HsaR1I, M.Hsi33800I, | | | | |
| | | M.HspKM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| Acc36I, BfuAI, | ACCT | M.Cac8I | GCNNGC | 2, 5 | m5C | ACCTGCNNGC[NNNN] |
| BspMI, BveI | GC | | | | | |
| Acc36I, BfuAI, | ACCT | M.CagV, M.Lsp6406I, M.Rsp7740I, | TTCGAA | 1, 6 | m6A | TTCGAACCTGCNNNN[NNNN] |
| BspMI, BveI | GC | M.RspPBTS2II, M.VbaLP2AI | | | | |
| Acc36I, BfuAI, | ACCT | M.CagVI | TCCGGA | 1, 6 | m6A | TCCGGACCTGCNNNN[NNNN] |
| BspMI, BveI | GC | | | | | |
| Acc36I, BfuAI, | ACCT | M.Cdi630IV, M.Ckr177III, M.CmaLM2II, | GCNGC | 2, 4 | m5C | ACCTGCNGCN[NNNN] |
| BspMI, BveI | GC | M.CocII, M.Fnu4HI, M.Fsp4HI | | | | |
| Acc36I, BfuAI, | ACCT | M.Cdi630V, M.Cdi81I, M.CdiG46II, | CAAAAA | 6 | m6A | CAAAAACCTGCNNNN[NNNN] |
| BspMI, BveI | GC | M.CmaLM2I, M.CpaYL7II, M.Pat19335II, | | | | |
| | | M.PbaVA2I, M.Pdi9689I | | | | |
| Acc36I, BfuAI, | ACCT | M.Cmu51329I, M.EcoJA03PDcm, | CCWGG | 1, 5 | Unknown | ACCTGCCWGG[NNNN] |
| BspMI, BveI | GC | M.RorR1Dcm, M.Sen8391Dcm, M.ZalSM2II | | | | |
| Acc36I, BfuAI, | ACCT | M.CocI, M.Fli6794II, M.HincII, M.HindII, | GTYRAC | 2, 5 | m6A | GTYRACCTGCNNNN[NNNN] |
| BspMI, BveI | GC | M.HinHia1II | | | | |
| Acc36I, BfuAI, | ACCT | M.CocIII, M.Fsi4947II, M.TdeIII, | GGNCC | 1, 5 | m4C | GGACCTGCNNNN[NNNN] |
| BspMI, BveI | GC | M.Tph12270I | | | | |
| Acc36I, BfuAI, | ACCT | M.CpeAVI, M.TcoKWC4I | GTATAC | 2, 5 | m6A | GTATACCTGCNNNN[NNNN] |
| BspMI, BveI | GC | | | | | |
| Acc36I, BfuAI, | ACCT | M.CsiFSMII, M.MfoCT6IV, M.NgoAIV, | GCCGGC | 2, 5 | m5C | ACCTGCCGGC[NNNN] |
| BspMI, BveI | GC | M.NgoMIV, M1.Sgr13350II | | | | |
| Acc36I, BfuAI, | ACCT | M.CviQI, M.Hpy32XII, M.Hpy57XI, | GTAC | 2, 3 | m6A | GTACCTGCNNNN[NNNN] |
| BspMI, BveI | GC | M.Hpy66XII, M.Hpy99XII, M.HpyFXII, | | | | |
| | | M.HpyPIV, M.HpyUM037XI, M.PabI | | | | |
| Acc36I, BfuAI, | ACCT | M.CviQIII, M.CviSIII, M.Hpy299XI, | TCGA | 1, 4 | m6A | TCGACCTGCNNNN[NNNN] |
| BspMI, BveI | GC | M.Hpy30II, M.Hpy32X, M.Hpy99VII, | | | | |
| | | M.HpyAX, M.HpyFII, M.HpyUM032XVII, | | | | |
| | | M.Tam77409III, M.TaqI, M.TpaRLI, | | | | |
| | | M.Tth HB8I | | | | |
| Acc36I, BfuAI, | ACCT | M.CviRI, M.Hpy298VIII, M.Hpy299VIII, | TGCA | 1, 4 | m6A | TGCACCTGCNNNN[NNNN], |
| BspMI, BveI | GC | M.Hpy30VI, M.Hpy32V, M.Hpy57II, | | | | ACCTGCANNN[NNNN] |
| | | M.Hpy66VIII, M.HpyFVI, M.HpyUM032VIII, | | | | |
| | | M.HpyUM037VIII | | | | |
| Acc36I, BfuAI, | ACCT | M.Dac11109II, M.Hfo6591I, M.MhuII, | GTAC | 1, 4 | m4C | GTACCTGCNNNN[NNNN] |
| BspMI, BveI | GC | M.MjaV, M.MlaZII, M.MspLW5I, M.RsaI | | | | |
| Acc36I, BfuAI, | ACCT | M.DdeI | CTNAG | 1, 5 | m5C | ACCTGCTNAG[NNNN] |
| BspMI, BveI | GC | | | | | |
| Acc36I, BfuAI, | ACCT | M.DfeI, M.Fnu419II, M.Pen113II | CTNNAG | 2, 5 | m6A | ACCTGCAGNN[NNNN] |
| BspMI, BveI | GC | | | | | |
| Acc36I, BfuAI, | ACCT | M.EcoO109I | RGGNCCY | 3, 5 | m5C | RGGACCTGCNNNN[NNNN] |
| BspMI, BveI | GC | | | | | |
| Acc36I, BfuAI, | ACCT | M.Esp1396I | CCANNNN | 3, 9 | m6A | CCACCTGCTGGN[NNNN] |
| BspMI, BveI | GC | | NTGG | | | |
| Acc36I, BfuAI, | ACCT | M.FatI | CATG | 1, 4 | m5C | ACCTGCATGN[NNNN] |
| BspMI, BveI | GC | | | | | |
| Acc36I, BfuAI, | ACCT | M.FisAW1I, M.ThaI, M.Tph12270II | CGCG | 1, 4 | m4C | ACCTGCGCGN[NNNN] |
| BspMI, BveI | GC | | | | | |
| Acc36I, BfuAI, | ACCT | M.FnoB1II, M.Hal24586I, M.Hpy298V, | CCGG | 1, 4 | m4C | ACCTGCCGGN[NNNN] |
| BspMI, BveI | GC | M.Hpy299IX, M.Hpy30V, M.Hpy32XI, | | | | |
| | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| Acc36I, BfuAI, | ACCT | M.HgiCI | GGYRCC | 2, 5 | m5C | GGYACCTGCNNNN[NNNN] |
| BspMI, BveI | GC | | | | | |
| Acc36I, BfuAI, | ACCT | M.HhaI, M.HinP1I, M.Hpy99III, M.HpyAVIII, | GCGC | 2, 3 | m5C | ACCTGCGCNN[NNNN] |
| BspMI, BveI | GC | M.HpyUM032XV | | | | |
| Acc36I, BfuAI, | ACCT | M.Hme33500II | HGCWGCK | 3 | m4C | ACCTGCWGCK[NNNN] |
| BspMI, BveI | GC | | | | | |
| Acc36I, BfuAI, | ACCT | M.HpaI, M.Mwh3033I, M.Rsp7I | GTTAAC | 2, 5 | m6A | GTTAACCTGCNNNN[NNNN] |
| BspMI, BveI | GC | | | | | |
| Acc36I, BfuAI, | ACCT | M.Hpy188I, M.Hpy298IX, M.Hpy299V, | TCNGA | 1, 5 | m6A | TCNGACCTGCNNNN[NNNN] |
| BspMI, BveI | GC | M.Hpy66V, M.HpyUM032V | | | | |
| Acc36I, BfuAI, | ACCT | M.Hpy30IX, M.Hpy32IX, M.Hpy57IX, | GTNNAC | 2, 5 | m6A | GTNNACCTGCNNNN[NNNN] |
| BspMI, BveI | GC | M.Hpy66XIII, M.Hpy8I, M.HpyFIX, | | | | |
| | | M.HpyUM032XI, M.HpyUM037V, M.MjaIV, | | | | |
| | | M.MspRAC08II | | | | |
| Acc36I, BfuAI, | ACCT | M.Hpy30VIII, M.Hpy32VIII, M.Hpy57III, | TCNNGA | 1, 6 | m6A | TCNNGACCTGCNNNN[NNNN] |
| BspMI, BveI | GC | M.Hpy66VI, M.Hpy99XVIII, M.HpyFVIII, | | | | |
| | | M.HpyPVIII | | | | |
| Acc36I, BfuAI, | ACCT | M.Hpy30X, M.Hpy57VIII, M.HpyFXI, M.MhuI | CTNAG | 1, 5 | m4C | ACCTGCTNAG[NNNN] |
| BspMI, BveI | GC | | | | | |
| Acc36I, BfuAI, | ACCT | M.Hpy57X | CTRYAG | 2, 5 | m6A | ACCTGCAGNN[NNNN] |
| BspMI, BveI | GC | | | | | |
| Acc36I, BfuAI, | ACCT | M.Hpy99II, M.Tsp45I, M.Tsu2489II | GTSAC | 2, 4 | m6A | GTSACCTGCNNNN[NNNN] |
| BspMI, BveI | GC | | | | | |
| Acc36I, BfuAI, | ACCT | M.HpyD27I | CCTC | 1, 3 | m5C, | ACCTGCCTCN[NNNN] |
| BspMI, BveI | GC | | | | m6A | |
| Acc36I, BfuAI, | ACCT | M.Lph145I, M.Lph93I | GGWGA | 5 | m6A | GGWGACCTGCNNNN[NNNN] |
| BspMI, BveI | GC | | | | | |
| Acc36I, BfuAI, | ACCT | M.Mae7806III, M.PspPI, M.Sau96I | GGNCC | 2, 4 | m5C | GGACCTGCNNNN[NNNN] |
| BspMI, BveI | GC | | | | | |
| Acc36I, BfuAI, | ACCT | M.MjaII | GGNCC | 1, 5 | m5C | GGACCTGCNNNN[NNNN] |
| BspMI, BveI | GC | | | | | |
| Acc36I, BfuAI, | ACCT | M.Msp1II | CCTGG | 1 | m4C | ACCTGCCTGG[NNNN] |
| BspMI, BveI | GC | | | | | |
| Acc36I, BfuAI, | ACCT | M.Msp42II | CCWGG | 1, 5 | m4C | ACCTGCCWGG[NNNN] |
| BspMI, BveI | GC | | | | | |
| Acc36I, BfuAI, | ACCT | M.NgoAXV, M.NgoMV | GGNNCC | 2, 5 | m5C | GGNACCTGCNNNN[NNNN] |
| BspMI, BveI | GC | | | | | |
| Acc36I, BfuAI, | ACCT | M.NheI | GCTAGC | 1, 6 | m4C | ACCTGCTAGC[NNNN] |
| BspMI, BveI | GC | | | | | |
| Acc36I, BfuAI, | ACCT | M.Nme18V | GCGCGC | 2, 5 | m5C | ACCTGCGCGC[NNNN] |
| BspMI, BveI | GC | | | | | |
| Acc36I, BfuAI, | ACCT | M.NmeDI | VCCGGY | 2, 5 | m5C | ACCTGCCGGB[NNNN] |
| BspMI, BveI | GC | | | | | |
| Acc36I, BfuAI, | ACCT | M.NmeMC58III | CCAGA | 1 | m5C | ACCTGCCAGA[NNNN] |
| BspMI, BveI | GC | | | | | |
| Acc36I, BfuAI, | ACCT | M.NspI, M.NspHI | RCATGY | 2, 5 | m5C | ACCTGCATGY [NNNN] |
| BspMI, BveI | GC | | | | | |
| Acc36I, BfuAI, | ACCT | M.Pla5I | CTAG | 1, 4 | Unknown | ACCTGCTAGN[NNNN] |
| BspMI, BveI | GC | | | | | |
| Acc36I, BfuAI, | ACCT | M.SmoLVI, M.Spn23FII, M.Spn556I, | TCTAGA | 1, 6 | m6A | TCTAGACCTGCNNNN[NNNN] |
| BspMI, BveI | GC | M.Spn7465I, M.SpnRI, M.XbaI | | | | |
| Acc36I, BfuAI, | ACCT | M.SonIII | GACNNNN | 2, 8 | m6A | GACCTGCTGCN[NNNN], |
| BspMI, BveI | GC | | TGC | | | GACNACCTGCNNNN[NNNN] |
| Acc36I, BfuAI, | ACCT | M.TspRI | CASTG | 1, 5 | m5C | ACCTGCASTG[NNNN] |
| BspMI, BveI | GC | | | | | |
| Acc36I, BfuAI, | ACCT | M.Yen2516I | GCGCGC | 2, 5 | Unknown | ACCTGCGCGC[NNNN] |
| BspMI, BveI | GC | | | | | |
| Acc36I, BfuAI, | ACCT | M1.AgoG2I, M2.AgoG2I | CCAGT | 1, 4 | m4C | ACCTGCCAGT[NNNN] |
| BspMI, BveI | GC | | | | | |
| Acc36I, BfuAI, | ACCT | M1.BspACI | CCGC | 1 | m5C | ACCTGCCGCN[NNNN] |
| BspMI, BveI | GC | | | | | |
| Acc36I, BfuAI, | ACCT | M1.Eco31I, M1.EcoMI | GAGACC | 4 | m6A | GAGACCTGCNNNN[NNNN] |
| BspMI, BveI | GC | | | | | |
| Acc36I, BfuAI, | ACCT | M1.HgaI, M2.LlaJI | GCGTC | 2 | m5C | ACCTGCGTCN[NNNN] |
| BspMI, BveI | GC | | | | | |
| Acc36I, BfuAI, | ACCT | M1.Hpy32II | GRRGA | 5 | m6A | GRRGACCTGCNNNN[NNNN] |
| BspMI, BveI | GC | | | | | |
| Acc36I, BfuAI, | ACCT | M1.HpyAII, M1.MboII, M1.NcuI | GAAGA | 5 | m6A | GAAGACCTGCNNNN[NNNN] |
| BspMI, BveI | GC | | | | | |
| Acc36I, BfuAI, | ACCT | M1.LplAG30I | CCTTC | 1 | m5C | ACCTGCCTTC[NNNN] |
| BspMI, BveI | GC | | | | | |
| Acc36I, BfuAI, | ACCT | M2.BceSIII, M1.Pru9I, M2.Pru9I | GCCGT | 2, 4 | m4C | ACCTGCCGTN[NNNN] |
| BspMI, BveI | GC | | | | | |
| Acc36I, BfuAI, | ACCT | M2.ClaSBI, M1.Fli6794IV, M2.HphI, | GGTGA | 5 | m6A | GGTGACCTGCNNNN[NNNN] |
| BspMI, BveI | GC | M2.Ngo3502II, M1.Ngo6140II, M1.NgoAXVI, | | | | |
| | | M1.NgoBVIII, M1.NgoFA19I, M2.NgoMVIII, | | | | |
| | | M2.PspHUN102I, M1.Ssp10II | | | | |
| Acc36I, BfuAI, | ACCT | M2.HpyAVI, M1.MnlI | CCTC | 1 | m5C | ACCTGCCTCN[NNNN] |
| BspMI, BveI | GC | | | | | |
| Acc36I, BfuAI, | ACCT | M2.RspA76I | GGGAC | 4 | m6A | GGGACCTGCNNNN[NNNN] |
| BspMI, BveI | GC | | | | | |
| AceIII | CAGC | M.AbaBs12I, M.Awo1030I, M.Cdu23823I, | CCWGG | 1, 5 | m5C | CAGCTCCWGGNNN[NNNN] |
| | TC | M.Csa8155II, M.Cun9529I, M.EasL1Dcm, | | | | |
| | | M.Eba57Dcm, M.Ec134399Dcm, | | | | |
| | | M.Eco12761Dcm M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| AceIII | CAGC | M.Acc65VI, M.SenAZIII | CTKMAG | 2, 5 | m6A | CTKCAGCTCNNNNNNN[NNNN] |
| | TC | | | | | |
| AceIII | CAGC | M.AciI | CCGC | 1, 3 | m5C | CAGCTCCGCNNNN[NNNN] |
| | TC | | | | | |
| AceIII | CAGC | M.Ade56712I, M.Asp588III, M.AveCB51II, | CTGCAG | 2, 5 | m6A | CTGCAGCTCNNNNNNN[NNNN] |
| | TC | M.BbrUIII, M.Blo30698I, M.BsuBI, | | | | |
| | | M.CpaYL7III, M.Dba7044I, M.Dfa45958II, | | | | |
| | | M.Eco12581I, M.Eco2012I, M.EcoGIII, | | | | |
| | | M.EcoG089I, M.Eho16691II, M.Hal5920II, | | | | |
| | | M.Lsp6406II, M.MspNR41II, M.Pae490I, | | | | |
| | | M.Pox23570I, M.PstI, M.RpaII, M.Sca3403II, | | | | |
| | | M.Tsp61I, M.XmnII, M.YenWI, M.YinY228I | | | | |
| AceIII | CAGC | M.Ama55I, M.CnaPQ2III, M.Rmo1926II, | CGGCCG | 1, 6 | m5C | CAGCTCGGCCGNN[NNNN] |
| | TC | M.SenC1765IV, M.SspG1I, M.XorPXII | | | | |
| AceIII | CAGC | M.AplI | CTGCAG | 3, 4 | m5C | CTGCAGCTCNNNNNNN[NNNN] |
| | TC | | | | | |
| AceIII | CAGC | M.AquI | CYCGRG | 1, 6 | m5C | CAGCTCGRGNNNN[NNNN], |
| | TC | | | | | CAGCTCYCGRGNN[NNNN] |
| AceIII | CAGC | M.Asp588II, M.BstVI, M.Dsp964I, M.DvuII, | CTCGAG | 2, 5 | m6A | CAGCTCGAGNNNN[NNNN] |
| | TC | M.GsuRed1II, M.Hka19301II, M.Mch9957IV, | | | | |
| | | M.MfoCT6II, M.Mhy14884I, M.Mru1279II, | | | | |
| | | M.MsmI, M.Msm8159I, M.Nal45188I, | | | | |
| | | M.Osp807I, M.Pae63I, M.PaeR7I, M.XhoI | | | | |
| AceIII | CAGC | M.AvaV, M.SliSI | GATC | 1, 4 | m4C | GATCAGCTCNNNNNNN[NNNN] |
| | TC | | | | | |
| AceIII | CAGC | M.BamWS8I, M.CthV, M.EfaRFI, M.Saf8902II, | GCWGC | 2, 4 | m5C | GCAGCTCNNNNNNN[NNNN] |
| | TC | M.TacII, M.TcoKWC4III | | | | |
| AceIII | CAGC | M.BbvI, M.BceSIV | GCAGC | 2, 4 | m5C | GCAGCTCNNNNNNN[NNNN] |
| | TC | | | | | |
| AceIII | CAGC | M.BceSII | GGWCC | 1, 5 | m5C | GGWCCAGCTCNNNNNNN[NNNN] |
| | TC | | | | | |
| AceIII | CAGC | M.BcoSlacII, M.MspLW5III, M.Nbr128I, | CTCGAG | 1, 6 | m4C | CAGCTCGAGNNNN[NNNN], |
| | TC | M.Psy18884II, M.Tam77409II, M.TbrGE1II, | | | | CAGCTCTCGAGNN[NNNN] |
| | | M.TpaRLII | | | | |
| AceIII | CAGC | M.BepI, M.FpsJV, M.Gel16401III, M.Pdi8503I | CGCG | 1, 4 | m5C | CAGCTCGCGNNNN[NNNN] |
| | TC | | | | | |
| AceIII | CAGC | M.BsoBI | CYCGRG | 1, 6 | m4C | CAGCTCGRGNNNN[NNNN], |
| | TC | | | | | CAGCTCYCGRGNN[NNNN] |
| AceIII | CAGC | M.Bsp14III, M.Bsp3003I, M.Bsp3004I, | GATC | 1, 4 | m5C | GATCAGCTCNNNNNNN[NNNN] |
| | TC | M.CalB3III, M.CpeAIII, M.Esu35585I, | | | | |
| | | M.FpsJIV, M.Lba2031I, M.Lbo2004I, | | | | |
| | | M.LlaKR2I, M.Lmo1042I, M.Lmo7956II, | | | | |
| | | M.Lsp10393II, M.MspI7I, M.Sau3AI, M.Sba3I, | | | | |
| | | M.Sin395I, M.Spn23FI, M.Sth368I | | | | |
| AceIII | CAGC | M.BstXI | CCANNNN | 3, 10 | m6A | CCAGCTCNNTGGNN[NNNN] |
| | TC | | NNTGG | | | |
| AceIII | CAGC | M.BsuFI, M.CpeAIV, M.MspI | CCGG | 1, 4 | m5C | CAGCTCCGGNNNN[NNNN] |
| | TC | | | | | |
| AceIII | CAGC | M.Cab13497I, M.Dac11109III, M.Dco13527I, | CTAG | 1, 4 | m4C | CAGCTCTAGNNNN[NNNN] |
| | TC | M.Djo10085I, M.Hbo11551I, M.Hka19301III, | | | | |
| | | M.Hme33500I, M.HsaR1I, M.Hsi33800I, | | | | |
| | | M.HspKM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| AceIII | CAGC | M.Cac8I | GCNNGC | 2, 5 | m5C | GCCAGCTCNNNNNNN[NNNN], |
| | TC | | | | | CAGCTCGCNNNNN[NNNN] |
| AceIII | CAGC | M.CagVI | TCCGGA | 1, 6 | m6A | CAGCTCCGGANNN[NNNN] |
| | TC | | | | | |
| AceIII | CAGC | M.Cdi630IV, M.Ckr177III, M.CmaLM2II, | GCNGC | 2, 4 | m5C | GCAGCTCNNNNNNN[NNNN] |
| | TC | M.CocII, M.Fnu4HI, M.Fsp4HI | | | | |
| AceIII | CAGC | M.CfrBI, M.Sen1735IV, M.SenAnaIV | CCWWGG | 1, 6 | m4C | CAGCTCCWWGGNN[NNNN] |
| | TC | | | | | |
| AceIII | CAGC | M.Cmu51329I, M.EcoJA03PDcm, | CCWGG | 1, 5 | Unknown | CAGCTCCWGGNNN[NNNN] |
| | TC | M.RorR1Dcm, M.Sen8391Dcm, M.ZalSM2II | | | | |
| AceIII | CAGC | M.CocIII, M.Fsi4947II, M.TdeIII, | GGNCC | 1, 5 | m4C | GGNCCAGCTCNNNNNNN[NNNN] |
| | TC | M.Tph12270I | | | | |
| AceIII | CAGC | M.Cph17I, M.Cph41I | CTYCAG | 5 | m6A | CTYCAGCTCNNNNNNN[NNNN] |
| | TC | | | | | |
| AceIII | CAGC | M.CviQIII, M.CviSIII, M.Hpy299XI, | TCGA | 1, 4 | m6A | CAGCTCGANNNNN[NNNN] |
| | TC | M.Hpy30II, M.Hpy32X, M.Hpy99VII, | | | | |
| | | M.HpyAX, M.HpyFII, M.HpyUM032XVII, | | | | |
| | | M.Tam77409III, M.TaqI, M.TpaRLI, | | | | |
| | | M.TthHB8I | | | | |
| AceIII | CAGC | M.CviRI, M.Hpy298VIII, M.Hpy299VIII, | TGCA | 1, 4 | m6A | TGCAGCTCNNNNNNN[NNNN] |
| | TC | M.Hpy30VI, M.Hpy32V, M.Hpy57II, | | | | |
| | | M.Hpy66VIII, M.HpyFVI, M.HpyUM032VIII, | | | | |
| | | M.HpyUM037VIII | | | | |
| AceIII | CAGC | M.Dac11109II, M.Hfo6591I, M.MhuII, | GTAC | 1, 4 | m4C | GTACAGCTCNNNNNNN[NNNN] |
| | TC | M.MjaV, M.MlaZII, M.MspLW5I, M.RsaI | | | | |
| AceIII | CAGC | M.DdeI | CTNAG | 1, 5 | m5C | CTCAGCTCNNNNNNN[NNNN], |
| | TC | | | | | CAGCTCAGNNNNN[NNNN], |
| | | | | | | CAGCTCTNAGNNN[NNNN] |
| AceIII | CAGC | M.DfeI, M.Fnu419II, M.Pen113II | CTNNAG | 2, 5 | m6A | CTNCAGCTCNNNNNNN[NNNN], |
| | TC | | | | | CAGCTCNAGNNNN[NNNN] |
| AceIII | CAGC | M.Esp1396I | CCANNNN | 3, 9 | m6A | CCAGCTCNTGGNNN[NNNN] |
| | TC | | NTGG | | | |
| AceIII | CAGC | M.FatI | CATG | 1, 4 | m5C | CAGCTCATGNNNN[NNNN] |
| | TC | | | | | |
| AceIII | CAGC | M.FisAW1I, M.ThaI, M.Tph12270II | CGCG | 1, 4 | m4C | CAGCTCGCGNNNN[NNNN] |
| | TC | | | | | |
| AceIII | CAGC | M.FnoB1II, M.Hal24586I, M.Hpy298V, | CCGG | 1, 4 | m4C | CAGCTCCGGNNNN[NNNN] |
| | TC | M.Hpy299IX, M.Hpy30V, M.Hpy32XI, | | | | |
| | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| AceIII | CAGC | M.Gel16401IV | CCAG | 2 | m5C | CCAGCTCNNNNNNN[NNNN] |
| | TC | | | | | |
| AceIII | CAGC | M.Hka19301IV | TCGCGA | 2, 5 | m4C | CAGCTCGCGANNN[NNNN] |
| | TC | | | | | |
| AceIII | CAGC | M.Hme33500II | HGCWGCK | 3 | m4C | HGCAGCTCNNNNNNN[NNNN] |
| | TC | | | | | |
| AceIII | CAGC | M.Hpy188I, M.Hpy298IX, M.Hpy299V, | TCNGA | 1, 5 | m6A | CAGCTCNGANNNN[NNNN] |
| | TC | M.Hpy66V, M.HpyUM032V | | | | |
| AceIII | CAGC | M.Hpy30VIII, M.Hpy32VIII, M.Hpy57III, | TCNNGA | 1, 6 | m6A | CAGCTCNNGANNN[NNNN] |
| | TC | M.Hpy66VI, M.Hpy99XVIII, M.HpyFVIII, | | | | |
| | | M.HpyPVIII | | | | |
| AceIII | CAGC | M.Hpy30X, M.Hpy57VIII, M.HpyFXI, M.MhuI | CTNAG | 1, 5 | m4C | CTCAGCTCNNNNNNN[NNNN], |
| | TC | | | | | CAGCTCAGNNNNN[NNNN], |
| | | | | | | CAGCTCTNAGNNN[NNNN] |
| AceIII | CAGC | M.Hpy57X | CTRYAG | 2, 5 | m6A | CTRCAGCTCNNNNNNN[NNNN] |
| | TC | | | | | |
| AceIII | CAGC | M.Hpy99IV, M.HpyFORFX | CCNNGG | 1, 6 | m4C | CAGCTCCNNGGNN[NNNN] |
| | TC | | | | | |
| AceIII | CAGC | M.HpyD27I | CCTC | 1, 3 | m5C, | CAGCTCCTCNNNN[NNNN] |
| | TC | | | | m6A | |
| AceIII | CAGC | M.Kpn555II, M.Mha183III | CTTCAG | 2, 5 | m6A | CTTCAGCTCNNNNNNN[NNNN] |
| | TC | | | | | |
| AceIII | CAGC | M.Lph145I, M.Lph93I | TCWCC | 1 | m6A | CAGCTCWCCNNNN[NNNN] |
| | TC | | | | | |
| AceIII | CAGC | M.Lph145II, M.Lph93II | CYAG | 3 | m6A | CCAGCTCNNNNNNN[NNNN] |
| | TC | | | | | |
| AceIII | CAGC | M.Lph15I | CCAG | 3 | m6A | CCAGCTCNNNNNNN[NNNN] |
| | TC | | | | | |
| AceIII | CAGC | M.Maf25III, M.Mbo26III, M.Mbo30III, | CTCCAG | 2, 5 | m6A | CTCCAGCTCNNNNNNN[NNNN], |
| | TC | M.MboBCG21I, M.MkaDI, M.Mtu22103III, | | | | CAGCTCCAGNNNN[NNNN] |
| | | M.Mtu22115II, M.Mtu26105II, M.Mtu28I, | | | | |
| | | M.Mtu37004II, M.MtuF1I, M.MtuHIII, | | | | |
| | | M.MtuJKIII | | | | |
| AceIII | CAGC | M.MjaII | GGNCC | 1, 5 | m5C | GGNCCAGCTCNNNNNNN[NNNN] |
| | TC | | | | | |
| AceIII | CAGC | M.Msp1II | CCTGG | 1 | m4C | CAGCTCCTGGNNN[NNNN] |
| | TC | | | | | |
| AceIII | CAGC | M.Msp42II | CCWGG | 1, 5 | m4C | CAGCTCCWGGNNN[NNNN] |
| | TC | | | | | |
| AceIII | CAGC | M.NheI | GCTAGC | 1, 6 | m4C | GCTAGCAGCTCNNNNNNN[NNNN] |
| | TC | | | | | |
| AceIII | CAGC | M.Nme18III | TCTGG | 2, 4 | m5C | CAGCTCTGGNNNN[NNNN] |
| | TC | | | | | |
| AceIII | CAGC | M.NmeMC58III | CCAGA | 1 | m5C | CAGCTCCAGANNN[NNNN] |
| | TC | | | | | |
| AceIII | CAGC | M.Nsp209III, M.Nsp51109II | CTCGAG | 1, 6 | m5C | CAGCTCGAGNNNN[NNNN], |
| | TC | | | | | CAGCTCTCGAGNN[NNNN] |
| AceIII | CAGC | M.Pam7686I | CCATGG | 1, 6 | m5C | CAGCTCCATGGNN[NNNN] |
| | TC | | | | | |
| AceIII | CAGC | M.Pfa23572II | CGATCG | 1, 6 | m5C | CAGCTCGATCGNN[NNNN] |
| | TC | | | | | |
| AceIII | CAGC | M.Pla5I | CTAG | 1, 4 | Unknown | CAGCTCTAGNNNN[NNNN] |
| | TC | | | | | |
| AceIII | CAGC | M.RpaIV | CTCGAG | 3, 4 | m5C | CAGCTCGAGNNNN[NNNN] |
| | TC | | | | | |
| AceIII | CAGC | M.Sam53668II | CCATGG | 1, 6 | Unknown | CAGCTCCATGGNN[NNNN] |
| | TC | | | | | |
| AceIII | CAGC | M.Sen1781III | CCWWGG | 1, 6 | Unknown | CAGCTCCWWGGNN[NNNN] |
| | TC | | | | | |
| AceIII | CAGC | M.SmoLVI, M.Spn23FII, M.Spn556I, | TCTAGA | 1, 6 | m6A | CAGCTCTAGANNN[NNNN] |
| | TC | M.Spn7465I, M.SpnRI, M.XbaI | | | | |
| AceIII | CAGC | M.SonIII | GCANNNN | 3, 9 | m6A | GCAGCTCGTCNNNN[NNNN] |
| | TC | | GTC | | | |
| AceIII | CAGC | M.TdeII | CTCTTC | 1, 2 | m4C, | CAGCTCTTCNNNN[NNNN], |
| | TC | | | | m6A | CAGCTCTCTTCNN[NNNN] |
| AceIII | CAGC | M.TspRI | CASTG | 1, 5 | m5C | CAGCTCASTGNNN[NNNN] |
| | TC | | | | | |
| AceIII | CAGC | M1.AgoG2I, M2.AgoG2I | CCAGT | 1, 4 | m4C | CAGCTCCAGTNNN[NNNN] |
| | TC | | | | | |
| AceIII | CAGC | M1.BspACI | CCGC | 1 | m5C | CAGCTCCGCNNNN[NNNN] |
| | TC | | | | | |
| AceIII | CAGC | M1.HphI | TCACC | 2 | m5C | CAGCTCACCNNNN[NNNN] |
| | TC | | | | | |
| AceIII | CAGC | M1.Hpy32II | TCYYC | 1 | m6A | CAGCTCYYCNNNN[NNNN] |
| | TC | | | | | |
| AceIII | CAGC | M1.HpyAII, M1.MboII, M1.NcuI | TCTTC | 1 | m6A | CAGCTCTTCNNNN[NNNN] |
| | TC | | | | | |
| AceIII | CAGC | M1.LplAG30I | CCTTC | 1 | m5C | CAGCTCCTTCNNN[NNNN] |
| | TC | | | | | |
| AceIII | CAGC | M1.Sgr13350III, M2.Sgr13350III | GCTCTTC | 2, 3 | m4C, | CAGCTCTTCNNNN[NNNN] |
| | TC | | | | m6A | |
| AceIII | CAGC | M2.ClaSBI, M1.Fli6794IV, M2.HphI, | TCACC | 1 | m6A | CAGCTCACCNNNN[NNNN] |
| | TC | M2.Ngo3502II, M1.Ngo6140II, M1.NgoAXVI, | | | | |
| | | M1.NgoBVIII, M1.NgoFA19I, M2.NgoMVIII, | | | | |
| | | M2.PspHUN102I, M1.Ssp10II | | | | |
| AceIII | CAGC | M2.Hpy32II, M2.HpyAII, M2.MboII, M2.NcuI | TCTTC | 2 | m4C | CAGCTCTTCNNNN[NNNN] |
| | TC | | | | | |
| AceIII | CAGC | M2.HpyAVI, M1.MnlI | CCTC | 1 | m5C | CAGCTCCTCNNNN[NNNN] |
| | TC | | | | | |
| Bbr7I | GAAG | M.AatII, M.Lsp6406III | GACGTC | 2, 5 | m6A | GAAGACGTCNNNN[NNNN] |
| | AC | | | | | |
| Bbr7I | GAAG | M.AbaBs12I, M.Awo1030I, M.Cdu23823I, | CCWGG | 1, 5 | m5C | CCWGGAAGACNNNNNNN[NNNN], |
| | AC | M.Csa8155II, M.Cun9529I, M.EasL1Dcm, | | | | GAAGACCWGGNNN[NNNN] |
| | | M.Eba57Dcm, M.Ecl34399Dcm, | | | | |
| | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| Bbr7I | GAAG | M.AbrSp7I, M.Apa101655II, M.Ath21654I, | GANTC | 2, 4 | m6A | GAAGACTCNNNNN[NNNN] |
| | AC | M.BdiNK6I, M.Bel76I, M.Bsp460I, | | | | |
| | | M.BspJKG1II, M.BspLA1II, M.BspRAC04II, | | | | |
| | | M.CagII, M.CcrMI, M.CcrNAI, M.CnaPQ2I, | | | | |
| | | M.Csp16704III, M.CspNS6I, M.CviBI, | | | | |
| | | M.CviQVI, M.Eli8509I, M.Fnu419I, M.Fsp71I, | | | | |
| | | M.HbaI, M.HhaII, M.HinfI, M.HinHia1I, | | | | |
| | | M.Hpy298IV, M.Hpy299IV, M.Hpy 32IV, | | | | |
| | | M.Hpy57IV, M.Hpy66IV, M.Hpy99IX, | | | | |
| | | M.HpyAIV, M.HpyFIV, M.HpyUM032IV, | | | | |
| | | M.HpyUM037IV, M.Mbo45III, M.Msp10I, | | | | |
| | | M.Msp73BI, M.MspAL11I, M.MspLW5II, | | | | |
| | | M.NhaXII, M.NpeUS61I, M.OspHL35I, | | | | |
| | | M.Pin17395II, M.Pla5III, M.PspLM6I, | | | | |
| | | M.RbaNRL2I, M.Rgi145II, M.Rmo1926I, | | | | |
| | | M.RpaIII, M.RsaII, M.RshIII, M.Rsp8I, | | | | |
| | | M.SmeC3I, M.SmoLV, M.Sna24252I, | | | | |
| | | M.SscL1I, M.SspM41II, M.SstE37II, M.ThaIII, | | | | |
| | | M.Xsp91I, M.ZmoCP4I | | | | |
| Bbr7I | GAAG | M.Acc65VI, M.SenAZIII | CTKMAG | 2, 5 | m6A | CTGAAGACNNNNNNN[NNNN] |
| | AC | | | | | |
| Bbr7I | GAAG | M.AciI | CCGC | 1, 3 | m5C | GCGGAAGACNNNNNNN[NNNN], |
| | AC | | | | | GAAGACCGCNNNN[NNNN] |
| Bbr7I | GAAG | M.AflIII | ACRYGT | 2, 5 | m4C | GAAGACRYGTNNN[NNNN] |
| | AC | | | | | |
| Bbr7I | GAAG | M.AgsI | TTSAA | 1, 5 | m6A | TTGAAGACNNNNNNN[NNNN] |
| | AC | | | | | |
| Bbr7I | GAAG | M.Ama55I, M.CnaPQ2III, M.Rmo1926II, | CGGCCG | 1, 6 | m5C | CGGCCGAAGACNNNNNNN[NNNN], |
| | AC | M.SenC1765IV, M.SspG1I, M.XorPXII | | | | GAAGACGGCCGNN[NNNN] |
| Bbr7I | GAAG | M.AquI | CYCGRG | 1, 6 | m5C | CYCGRGAAGACNNNNNNN[NNNN], |
| | AC | | | | | GAAGACYCGRGNN[NNNN] |
| Bbr7I | GAAG | M.AvaIX, M.Cfr10I, M.Nme18VI, M.VchAI, | RCCGGY | 2, 5 | m5C | GAAGACCGGYNNN[NNNN] |
| | AC | M.Vch0395I | | | | |
| Bbr7I | GAAG | M.BcoSlacII, M.MspLW5III, M.Nbr128I, | CTCGAG | 1, 6 | m4C | CTCGAGAAGACNNNNNNN[NNNN], |
| | AC | M.Psy18884II, M.Tam77409II, M.TbrGE1II, | | | | GAAGACTCGAGNN[NNNN] |
| | | M.TpaRLII | | | | |
| Bbr7I | GAAG | M.BepI, M.FpsJV, M.Gel16401III, M.Pdi8503I | CGCG | 1, 4 | m5C | CGCGAAGACNNNNNNN[NNNN], |
| | AC | | | | | GAAGACGCGNNNN[NNNN] |
| Bbr7I | GAAG | M.BsoBI | CYCGRG | 1, 6 | m4C | CYCGRGAAGACNNNNNNN[NNNN], |
| | AC | | | | | GAAGACYCGRGNN[NNNN] |
| Bbr7I | GAAG | M.BsuFI, M.CpeAIV, M.MspI | CCGG | 1, 4 | m5C | CCGGAAGACNNNNNNN[NNNN], |
| | AC | | | | | GAAGACCGGNNNN[NNNN] |
| Bbr7I | GAAG | M.Cab13497I, M.Dac11109III, M.Dco13527I, | CTAG | 1, 4 | m4C | CTAGAAGACNNNNNNN[NNNN], |
| | AC | M.Djo10085I, M.Hbo11551I, M.Hka19301III, | | | | GAAGACTAGNNNN[NNNN] |
| | | M.Hme33500I, M.HsaR1I, M.Hsi33800I, | | | | |
| | | M.HspKM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| Bbr7I | GAAG | M.CagV, M.Lsp6406I, M.Rsp7740I, | TTCGAA | 1, 6 | m6A | TTCGAAGACNNNNNNN[NNNN] |
| | AC | M.RspPBTS2II, M.VbaLP2AI | | | | |
| Bbr7I | GAAG | M.CagVI | TCCGGA | 1, 6 | m6A | TCCGGAAGACNNNNNNN[NNNN] |
| | AC | | | | | |
| Bbr7I | GAAG | M.CagVIII | ACGCGT | 2, 5 | m4C | GAAGACGCGTNNN[NNNN] |
| | AC | | | | | |
| Bbr7I | GAAG | M.CfrBI, M.Sen1735IV, M.SenAnaIV | CCWWGG | 1, 6 | m4C | CCWWGGAAGACNNNNNNN[NNNN], |
| | AC | | | | | GAAGACCWWGGNN[NNNN] |
| Bbr7I | GAAG | M.Cmu51329I, M.EcoJA03PDcm, | CCWGG | 1, 5 | Unknown | CCWGGAAGACNNNNNNN[NNNN], |
| | AC | M.RorR1Dcm, M.Sen8391Dcm, M.ZalSM2II | | | | GAAGACCWGGNNN[NNNN] |
| Bbr7I | GAAG | M.CviQIII, M.CviSIII, M.Hpy299XI, | TCGA | 1, 4 | m6A | TCGAAGACNNNNNNN[NNNN] |
| | AC | M.Hpy30II, M.Hpy32X, M.Hpy99VII, | | | | |
| | | M.HpyAX, M.HpyFII, M.HpyUM032XVII, | | | | |
| | | M.Tam77409III, M.TaqI, M.TpaRLI, | | | | |
| | | M.TthHB8I | | | | |
| Bbr7I | GAAG | M.DdeI | CTNAG | 1, 5 | m5C | CTNAGAAGACNNNNNNN[NNNN], |
| | AC | | | | | GAAGACTNAGNNN[NNNN] |
| Bbr7I | GAAG | M.DfeI, M.Fnu419II, M.Pen113II | CTNNAG | 2, 5 | m6A | CTGAAGACNNNNNNN[NNNN] |
| | AC | | | | | |
| Bbr7I | GAAG | M.FatI | CATG | 1, 4 | m5C | CATGAAGACNNNNNNN[NNNN], |
| | AC | | | | | GAAGACATGNNNN[NNNN] |
| Bbr7I | GAAG | M.FisAW1I, M.ThaI, M.Tph12270II | CGCG | 1, 4 | m4C | CGCGAAGACNNNNNNN[NNNN], |
| | AC | | | | | GAAGACGCGNNNN[NNNN] |
| Bbr71 | GAAG | M.FnoB1II, M.Hal24586I, M.Hpy298V, | CCGG | 1, 4 | m4C | CCGGAAGACNNNNNNN[NNNN], |
| | AC | M.Hpy299IX, M.Hpy30V, M.Hpy32XI, | | | | GAAGACCGGNNNN[NNNN] |
| | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| Bbr7I | GAAG | M.FpsJIII | CTGAAG | 5 | m6A | CTGAAGACNNNNNNN[NNNN] |
| | AC | | | | | |
| Bbr7I | GAAG | M.GsuRed1IV, M.Osp807II, M.Tth111I | GACNNNG | 2, 8 | m6A | GAAGACNNNGTCN[NNNN] |
| | AC | | TC | | | |
| Bbr7I | GAAG | M.Hka19301IV | TCGCGA | 2, 5 | m4C | TCGCGAAGACNNNNNNN[NNNN] |
| | AC | | | | | |
| Bbr7I | GAAG | M.Hpy188I, M.Hpy298IX, M.Hpy299V, | TCNGA | 1, 5 | m6A | TCNGAAGACNNNNNNN[NNNN] |
| | AC | M.Hpy66V, M.HpyUM032V | | | | |
| Bbr7I | GAAG | M.Hpy298II, M.Hpy299II, M.Hpy66II, | ACNGT | 2, 4 | m4C | GAAGACNGTNNNN[NNNN] |
| | AC | M.HpyUM032II, M.HpyUM037II | | | | |
| Bbr7I | GAAG | M.Hpy30VIII, M.Hpy32VIII, M.Hpy57III, | TCNNGA | 1, 6 | m6A | TCNNGAAGACNNNNNNN[NNNN] |
| | AC | M.Hpy66VI, M.Hpy99XVIII, M.HpyFVIII, | | | | |
| | | M.HpyPVIII | | | | |
| Bbr7I | GAAG | M.Hpy30X, M.Hpy57VIII, M.HpyFXI, M.MhuI | CTNAG | 1, 5 | m4C | CTNAGAAGACNNNNNNN[NNNN], |
| | AC | | | | | GAAGACTNAGNNN[NNNN] |
| Bbr7I | GAAG | M.Hpy99IV, M.HpyFORFX | CCNNGG | 1, 6 | m4C | CCNNGGAAGACNNNNNNN[NNNN], |
| | AC | | | | | GAAGACCNNGGNN[NNNN] |
| Bbr7I | GAAG | M.Hpy99XI | ACGT | 2, 3 | m5C | GAAGACGTNNNNN[NNNN] |
| | AC | | | | | |
| Bbr7I | GAAG | M.HpyD27I | GAGG | 2, 4 | m6A, | GAAGACCTCNNNN[NNNN], |
| | AC | | | | m5C | GAGGAAGACNNNNNNN[NNNN] |
| Bbr7I | GAAG | M.Kpn555II, M.Mha183III | CTGAAG | 2, 5 | m6A | CTGAAGACNNNNNNN[NNNN] |
| | AC | | | | | |
| Bbr7I | GAAG | M.Lph145I, M.Lph93I | GGWGA | 5 | m6A | GGWGAAGACNNNNNNN[NNNN] |
| | AC | | | | | |
| Bbr7I | GAAG | M.MlyI, M.Pen113I | GASTC | 2, 4 | m6A | GAAGACTCNNNNN[NNNN] |
| | AC | | | | | |
| Bbr7I | GAAG | M.Msp1II | CCAGG | 5 | m4C | GAAGACCTGGNNN[NNNN], |
| | AC | | | | | CCAGGAAGACNNNNNNN[NNNN] |
| Bbr7I | GAAG | M.Msp42II | CCWGG | 1, 5 | m4C | CCWGGAAGACNNNNNNN[NNNN], |
| | AC | | | | | GAAGACCWGGNNN[NNNN] |
| Bbr7I | GAAG | M.Nme18III | CCAGA | 2, 4 | m5C | CCAGAAGACNNNNNNN[NNNN] |
| | AC | | | | | |
| Bbr7I | GAAG | M.NmeDI | VCCGGY | 2, 5 | m5C | GAAGACCGGBNNN[NNNN] |
| | AC | | | | | |
| Bbr7I | GAAG | M.NmeMC58III | CCAGA | 1 | m5C | TCTGGAAGACNNNNNNN[NNNN], |
| | AC | | | | | GAAGACCAGANNN[NNNN] |
| Bbr7I | GAAG | M.Nsp209III, M.Nsp51109II | CTCGAG | 1, 6 | m5C | CTCGAGAAGACNNNNNNN[NNNN], |
| | AC | | | | | GAAGACTCGAGNN[NNNN] |
| Bbr7I | GAAG | M.NspI, M.NspHI | RCATGY | 2, 5 | m5C | GAAGACATGYNNN[NNNN] |
| | AC | | | | | |
| Bbr7I | GAAG | M.Pam7686I | CCATGG | 1, 6 | m5C | CCATGGAAGACNNNNNNN[NNNN], |
| | AC | | | | | GAAGACCATGGNN[NNNN] |
| Bbr7I | GAAG | M.Pfa23572II | CGATCG | 1, 6 | m5C | CGATCGAAGACNNNNNNN[NNNN], |
| | AC | | | | | GAAGACGATCGNN[NNNN] |
| Bbr7I | GAAG | M.Pla5I | CTAG | 1, 4 | Unknown | CTAGAAGACNNNNNNN[NNNN], |
| | AC | | | | | GAAGACTAGNNNN[NNNN] |
| Bbr7I | GAAG | M.Sam53668II | CCATGG | 1, 6 | Unknown | CCATGGAAGACNNNNNNN[NNNN], |
| | AC | | | | | GAAGACCATGGNN[NNNN] |
| Bbr7I | GAAG | M.Sen1781III | CCWWGG | 1, 6 | Unknown | CCWWGGAAGACNNNNNNN[NNNN], |
| | AC | | | | | GAAGACCWWGGNN[NNNN] |
| Bbr7I | GAAG | M.SmoLVI, M.Spn23FII, M.Spn556I, | TCTAGA | 1, 6 | m6A | TCTAGAAGACNNNNNNN[NNNN] |
| | AC | M.Spn7465I, M.SpnRI, M.XbaI | | | | |
| Bbr7I | GAAG | M.SonIII | GACNNNN | 2, 8 | m6A | GAAGACNNNNTGC[NNNN] |
| | AC | | TGC | | | |
| Bbr7I | GAAG | M.TdeII | CTCTTC | 1, 2 | m4C, | GAAGAGAAGACNNNNNNN[NNNN], |
| | AC | | | | m6A | GAAGACTCTTCNN[NNNN] |
| Bbr7I | GAAG | M.TspRI | CASTG | 1, 5 | m5C | CASTGAAGACNNNNNNN[NNNN], |
| | AC | | | | | GAAGACASTGNNN[NNNN] |
| Bbr7I | GAAG | M.XmnI | GAANNNN | 2, 9 | m6A | GAAGACNTTCNNN[NNNN] |
| | AC | | TTC | | | |
| Bbr7I | GAAG | M1.AgoG2I, M2.AgoG2I | CCAGT | 1, 4 | m4C | ACTGGAAGACNNNNNNN[NNNN], |
| | AC | | | | | GAAGACTGGNNNN[NNNN], |
| | | | | | | GAAGACCAGTNNN[NNNN] |
| Bbr7I | GAAG | M1.BceSIII | ACGGC | 2 | m4C | GAAGACGGCNNNN[NNNN] |
| | AC | | | | | |
| Bbr7I | GAAG | M1.BspACI | CCGC | 1 | m5C | GCGGAAGACNNNNNNN[NNNN], |
| | AC | | | | | GAAGACCGCNNNN[NNNN] |
| Bbr7I | GAAG | M1.Dsh12II, M2.Dsh12II | GACGGC | 3, 4 | m4C | GAAGACGGCNNNN[NNNN] |
| | AC | | | | | |
| Bbr7I | GAAG | M1.HphI | GGTGA | 4 | m5C | GGTGAAGACNNNNNNN[NNNN] |
| | AC | | | | | |
| Bbr7I | GAAG | M1.LplAG30I | GAAGG | 5 | m5C | GAAGACCTTCNNN[NNNN], |
| | AC | | | | | GAAGGAAGACNNNNNNN[NNNN] |
| Bbr7I | GAAG | M2.BceSIII, M1.Pru9I, M2.Pru9I | ACGGC | 2, 4 | m4C | GAAGACGGCNNNN[NNNN] |
| | AC | | | | | |
| Bbr7I | GAAG | M2.BfiI | ACTGGG | 2 | m4C | GAAGACTGGGNNN[NNNN] |
| | AC | | | | | |
| Bbr7I | GAAG | M2.BfuAI | ACCTGC | 2 | m5C | GAAGACCTGCNNN[NNNN] |
| | AC | | | | | |
| Bbr71 | GAAG | M2.ClaSBI, M1.Fli6794IV, M2.HphI, | GGTGA | 5 | m6A | GGTGAAGACNNNNNNN[NNNN] |
| | AC | M2.Ngo3502II, M1.Ngo6140II, M1.NgoAXVI, | | | | |
| | | M1.NgoBVIII, M1.NgoFA19I, M2.NgoMVIII, | | | | |
| | | M2.PspHUN102I, M1.Ssp10II | | | | |
| Bbr7I | GAAG | M2.HgaI, M1.LlaJI | GACGC | 3 | m5C | GAAGACGCNNNNN[NNNN] |
| | AC | | | | | |
| Bbr7I | GAAG | M2.HpyAVI, M1.MnlI | GAGG | 4 | m5C | GAAGACCTCNNNN[NNNN], |
| | AC | | | | | GAGGAAGACNNNNNNN[NNNN] |
| Bbr7I | GAAG | M2.Nme579IV, M2.NmeMC58II | GACGC | 2 | m6A | GAAGACGCNNNNN[NNNN] |
| | AC | | | | | |
| BbsI, BbvII, | GAAG | M.AbaBs12I, M.Awo1030I, M.Cdu23823I, | CCWGG | 1, 5 | m5C | CCWGGAAGACNN[NNNN] |
| Bbv16II, BpiI, | AC | M.Csa8155II, M.Cun9529I, M.EasL1Dcm, | | | | |
| BpuAI, Bsc91I, | | M.Eba57Dcm, M.Ecl34399Dcm, | | | | |
| BspBS31I, | | M.Eco12761Dcm, M.Eco3609Dcm, | | | | |
| BspIS4I, | | M.Eco4255Dcm, M.EcoVR50Dcm, | | | | |
| BspTS514I, | | M.EsaSP1II, M.Kpn34618Dcm, | | | | |
| BstBS32I, BstTS5I, | | M.Kpn38547Dcm, M.Kpn39I, M.Kpn43816I, | | | | |
| BstV2I | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| BbsI, BbvII, | GAAG | M.AbrSp7I, M.Apa101655II, M.Ath21654I, | GANTC | 2, 4 | m6A | GAAGACTC[NNNN] |
| Bbv16II, BpiI, | AC | M.BdiNK6I, M.Bel76I, M.Bsp460I, | | | | |
| BpuAI, Bsc91I, | | M.BspJKG1II, M.BspLA1II, M.BspRAC04II, | | | | |
| BspBS31I, | | M.CagII, M.CcrMI, M.CcrNAI, M.CnaPQ2I, | | | | |
| BspIS4I, | | M.Csp16704III, M.CspNS6I, M.CviBI, | | | | |
| BspTS514I, | | M.CviQVI, M.Eli8509I, M.Fnu419I, M.Fsp71I, | | | | |
| BstBS32I, BstTS5I, | | M.HbaI, M.HhaII, M.HinfI, M.HinHia1I, | | | | |
| BstV2I | | M.Hpy298IV, M.Hpy299IV, M.Hpy32IV, | | | | |
| | | M.Hpy57IV, M.Hpy66IV, M.Hpy99IX, | | | | |
| | | M.HpyAIV, M.HpyFIV, M.HpyUM032IV, | | | | |
| | | M.HpyUM037IV, M.Mbo45III, M.Msp10I, | | | | |
| | | M.Msp73BI, M.MspAL11I, M.MspLW5II, | | | | |
| | | M.NhaXII, M.NpeUS61I, M.OspHL35I, | | | | |
| | | M.Pin17395II, M.Pla5III, M.PspLM6I, | | | | |
| | | M.RbaNRL2I, M.Rgi145II, M.Rmo1926I, | | | | |
| | | M.RpaIII, M.RsaII, M.RshIII, M.Rsp8I, | | | | |
| | | M.SmeC3I, M.SmoLV, M.Sna24252I, | | | | |
| | | M.SscL1I, M.SspM41II, M.SstE37II, M.ThaIII, | | | | |
| | | M.Xsp91I, M.ZmoCP4I | | | | |
| BbsI, BbvII, | GAAG | M.Acc65VI, M.SenAZIII | CTKMAG | 2, 5 | m6A | CTGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.AciI | GCGG | 2, 4 | m5C | GCGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.AgsI | TTSAA | 1, 5 | m6A | TTGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Ama55I, M.CnaPQ2III, M.Rmo1926II, | CGGCCG | 1, 6 | m5C | CGGCCGAAGACNN[NNNN] |
| Bbv16II, BpiI, | AC | M.SenC1765IV, M.SspG1I, M.XorPXII | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.AquI | CYCGRG | 1, 6 | m5C | CYCGRGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.BcoSlacII, M.MspLW5III, M.Nbr128I, | CTCGAG | 1, 6 | m4C | CTCGAGAAGACNN[NNNN] |
| Bbv16II, BpiI, | AC | M.Psy18884II, M.Tam77409II, M.TbrGE1II, | | | | |
| BpuAI, Bsc91I, | | M.TpaRLII | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.BepI, M.FpsJV, M.Gel16401III, M.Pdi8503I | CGCG | 1, 4 | m5C | CGCGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.BsoBI | CYCGRG | 1, 6 | m4C | CYCGRGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.BsuFI, M.CpeAIV, M.MspI | CCGG | 1, 4 | m5C | CCGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Cab13497I, M.Dac11109III, M.Dco13527I, | CTAG | 1, 4 | m4C | CTAGAAGACNN[NNNN] |
| Bbv16II, BpiI, | AC | M.Djo10085I, M.Hbo11551I, M.Hka19301III, | | | | |
| BpuAI, Bsc91I, | | M.Hme33500I, M.HsaR1I, M.Hsi33800I, | | | | |
| BspBS31I, | | M.HspKM1WII, M.HspNII, M.HtuI, M.HvoDSI, | | | | |
| BspIS4I, | | M.Lba2029II, M.Mbo45II, M.MjaI, M.MthZI, | | | | |
| BspTS514I, | | M.Nas12278I, M.Rfl3010I, M.SsmMI | | | | |
| BstBS32I, BstTS5I, | | | | | | |
| BstV2I | | | | | | |
| BbsI, BbvII, | GAAG | M.CagV, M.Lsp6406I, M.Rsp7740I, | TTCGAA | 1, 6 | m6A | TTCGAAGACNN[NNNN] |
| Bbv16II, BpiI, | AC | M.RspPBTS2II, M.VbaLP2AI | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.CagVI | TCCGGA | 1, 6 | m6A | TCCGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.CfrBI, M.Sen1735IV, M.SenAnaIV | CCWWGG | 1, 6 | m4C | CCWWGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Cmu51329I, M.EcoJA03PDcm, | CCWGG | 1, 5 | Unknown | CCWGGAAGACNN[NNNN] |
| Bbv16II, BpiI, | AC | M.RorR1Dcm, M.Sen8391Dcm, M.ZalSM2II | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.CviQIII, M.CviSIII, M.Hpy299XI, | TCGA | 1, 4 | m6A | TCGAAGACNN[NNNN] |
| Bbv16II, BpiI, | AC | M.Hpy30II, M.Hpy32X, M.Hpy99VII, | | | | |
| BpuAI, Bsc91I, | | M.HpyAX, M.HpyFII, M.HpyUM032XVII, | | | | |
| BspBS31I, | | M.Tam77409III, M.TaqI, M.TpaRLI, | | | | |
| BspIS4I, | | M.TthHB8I | | | | |
| BspTS514I, | | | | | | |
| BstBS32I, BstTS5I, | | | | | | |
| BstV2I | | | | | | |
| BbsI, BbvII, | GAAG | M.DdeI | CTNAG | 1, 5 | m5C | CTNAGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.DfeI, M.Fnu419II, M.Pen113II | CTNNAG | 2, 5 | m6A | CTGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.FatI | CATG | 1, 4 | m5C | CATGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.FisAW1I, M.ThaI, M.Tph12270II | CGCG | 1, 4 | m4C | CGCGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.FnoB1II, M.Hal24586I, M.Hpy298V, | CCGG | 1, 4 | m4C | CCGGAAGACNN[NNNN] |
| Bbv16II, BpiI, | AC | M.Hpy299IX, M.Hpy30V, M.Hpy32XI, | | | | |
| BpuAI, Bsc91I, | | M.Hpy57V, M.Hpy66IX, M.Hpy99VIII, | | | | |
| BspBS31I, | | M.HpyFV, M.HpyUM032IX, M.HpyUM037IX, | | | | |
| BspIS4I, | | M.MjaVI, M.PspJDRI, M.TmeBII | | | | |
| BspTS514I, | | | | | | |
| BstBS32I, BstTS5I, | | | | | | |
| BstV2I | | | | | | |
| BbsI, BbvII, | GAAG | M.FpsJIII | CTGAAG | 5 | m6A | CTGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Hka19301IV | TCGCGA | 2, 5 | m4C | TCGCGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Hpy188I, M.Hpy298IX, M.Hpy299V, | TCNGA | 1, 5 | m6A | TCNGAAGACNN[NNNN] |
| Bbv16II, BpiI, | AC | M.Hpy66V, M.HpyUM032V | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Hpy30VIII, M.Hpy32VIII, M.Hpy57III, | TCNNGA | 1, 6 | m6A | TCNNGAAGACNN[NNNN] |
| Bbv16II, BpiI, | AC | M.Hpy66VI, M.Hpy99XVIII, M.HpyFVIII, | | | | |
| BpuAI, Bsc91I, | | M.HpyPVIII | | | | |
| BspBS31I, | | | | | | |
| BspIS4I, | | | | | | |
| BspTS514I, | | | | | | |
| BstBS32I, BstTS5I, | | | | | | |
| BstV21 | | | | | | |
| BbsI, BbvII, | GAAG | M.Hpy30X, M.Hpy57VIII, M.HpyFXI, M.MhuI | CTNAG | 1, 5 | m4C | CTNAGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Hpy99IV, M.HpyFORFX | CCNNGG | 1, 6 | m4C | CCNNGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Hpy99XI | ACGT | 2, 3 | m5C | GAAGACGT[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.HpyD27I | GAGG | 2, 4 | m6A, | GAGGAAGACNN[NNNN] |
| Bbv16II, BpiI, | AC | | | | m5C | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Kpn555II, M.Mha183III | CTGAAG | 2, 5 | m6A | CTGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Lph145I, M.Lph93I | GGWGA | 5 | m6A | GGWGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.MlyI, M.Pen113I | GASTC | 2, 4 | m6A | GAAGACTC[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Msp1II | CCAGG | 5 | m4C | CCAGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Msp42II | CCWGG | 1, 5 | m4C | CCWGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Nme18III | CCAGA | 2, 4 | m5C | CCAGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.NmeMC58III | TCTGG | 5 | m5C | TCTGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Nsp209III, M.Nsp51109II | CTCGAG | 1, 6 | m5C | CTCGAGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Pam7686I | CCATGG | 1, 6 | m5C | CCATGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Pfa23572II | CGATCG | 1, 6 | m5C | CGATCGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Pla5I | CTAG | 1, 4 | Unknown | CTAGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Sam53668II | CCATGG | 1, 6 | Unknown | CCATGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.Sen1781III | CCWWGG | 1, 6 | Unknown | CCWWGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.SmoLVI, M.Spn23FII, M.Spn556I, | TCTAGA | 1, 6 | m6A | TCTAGAAGACNN[NNNN] |
| Bbv16II, BpiI, | AC | M.Spn7465I, M.SpnRI, M.XbaI | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.TdeII | GAAGAG | 5, 6 | m6A, | GAAGAGAAGACNN[NNNN] |
| Bbv16II, BpiI, | AC | | | | m4C | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M.TspRI | CASTG | 1, 5 | m5C | CASTGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV21 | | | | | | |
| BbsI, BbvII, | GAAG | M1.AgoG2I, M2.AgoG2I | ACTGG | 2, 5 | m4C | ACTGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M1.BspACI | GCGG | 4 | m5C | GCGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M1.HphI | GGTGA | 4 | m5C | GGTGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M1.LplAG30I | GAAGG | 5 | m5C | GAAGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M2.ClaSBI, M1.Fli6794IV, M2.HphI, | GGTGA | 5 | m6A | GGTGAAGACNN[NNNN] |
| Bbv16II, BpiI, | AC | M2.Ngo3502II, M1.Ngo6140II, M1.NgoAXVI, | | | | |
| BpuAI, Bsc91I, | | M1.NgoBVIII, M1.NgoFA19I, M2.NgoMVIII, | | | | |
| BspBS31I, | | M2.PspHUN102I, M1.Ssp10II | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M2.HgaI, M1.LlaJI | GACGC | 3 | m5C | GAAGACGC[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| BbsI, BbvII, | GAAG | M2.HpyAVI, M1.MnlI | GAGG | 4 | m5C | GAGGAAGACNN[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV21 | | | | | | |
| BbsI, BbvII, | GAAG | M2.Nme579IV, M2.NmeMC58II | GACGC | 2 | m6A | GAAGACGC[NNNN] |
| Bbv16II, BpiI, | AC | | | | | |
| BpuAI, Bsc91I, | | | | | | |
| BspBS31I, | | | | | | |
| BspIS4I, | | | | | | |
| BspTS514I, | | | | | | |
| BstBS32I, BstTS5I, | | | | | | |
| BstV2I | | | | | | |
| Bco5I, Bco116I, | CTCTT | M.AbrSp7I, M.Apa101655II, M.Ath21654I, | GANTC | 2, 4 | m6A | GACTCTTCN[NNN] |
| BcoKI, BseZI, | C | M.BdiNK6I, M.Bel76I, M.Bsp460I, | | | | |
| Bst6I, Bsu6I, | | M.BspJKG1II, M.BspLA1II, M.BspRAC04II, | | | | |
| CatHI, Eam1104I, | | M.CagII, M.CcrMI, M.CcrNAI, M.CnaPQ2I, | | | | |
| EarI, Ksp632I | | M.Csp16704III, M.CspNS6I, M.CviBI, | | | | |
| | | M.CviQVI, M.Eli8509I, M.Fnu419I, M.Fsp71I, | | | | |
| | | M.HbaI, M.HhaII, M.HinfI, M.HinHia1I, | | | | |
| | | M.Hpy298IV, M.Hpy299IV, M.Hpy32IV, | | | | |
| | | M.Hpy57IV, M.Hpy66IV, M.Hpy99IX, | | | | |
| | | M.HpyAIV, M.HpyFIV, M.HpyUM032IV, | | | | |
| | | M.HpyUM037IV, M.Mbo45III, M.Msp10I, | | | | |
| | | M.Msp73BI, M.MspAL11I, M.MspLW5II, | | | | |
| | | M.NhaXII, M.NpeUS61I, M.OspHL35I, | | | | |
| | | M.Pin17395II, M.Pla5III, M.PspLM6I, | | | | |
| | | M.RbaNRL2I, M.Rgi145II, M.Rmo1926I, | | | | |
| | | M.RpaIII, M.RsaII, M.RshIII, M.Rsp8I, | | | | |
| | | M.SmeC3I, M.SmoLV, M.Sna24252I, | | | | |
| | | M.SscL1I, M.SspM41II, M.SstE37II, M.ThaIII, | | | | |
| | | M.Xsp91I, M.ZmoCP4I | | | | |
| Bco5I, Bco116I, | CTCTT | M.AluBI | AGCT | 1, 4 | m6A | AGCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.AluI, M.CspCY2I | AGCT | 2, 3 | m5C | AGCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.Alw26I | GTCTC | 3, 2 | m5C, | GTCTCTTCN[NNN] |
| BcoKI, BseZI, | C | | | | m6A | |
| Bst6I, Bsu6I, | | | | | | |
| CatHI, Eam1104I, | | | | | | |
| EarI, Ksp632I | | | | | | |
| Bco5I, Bco116I, | CTCTT | M.AspDUT2II, M.BstYI, M.CagIII, | RGATCY | 2, 5 | m4C | |
| BcoKI, BseZI, | C | M.Mpa1757V, M.Mru1279V, M.RsaIII, | | | | |
| Bst6I, Bsu6I, | | M.XhoII | | | | |
| CatHI, Eam1104I, | | | | | | |
| EarI, Ksp632I | | | | | | |
| Bco5I, Bco116I, | CTCTT | M.AspJHL3I, M2.Psp320WII | GAGCTC | 3, 4 | m4C | GAGCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.AvaV, M.SliSI | GATC | 1, 4 | m4C | GATCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.BanII, M.EcoT38I | GRGCYC | 3, 4 | m5C | GRGCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.BceSII | GGWCC | 1, 5 | m5C | GGWCCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.BglII | AGATCT | 2, 5 | m4C | AGATCTCTTCN [NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.Ble402III, M.Psp10HI | SAGCTS | 3, 4 | m4C | SAGCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.Bsp14III, M.Bsp3003I, M.Bsp3004I, | GATC | 1, 4 | m5C | GATCTCTTCN[NNN] |
| BcoKI, BseZI, | C | M.CalB3III, M.CpeAIII, M.Esu35585I, | | | | |
| Bst6I, Bsu6I, | | M.FpsJIV, M.Lba2031I, M.Lbo2004I, | | | | |
| CatHI, Eam1104I, | | M.LlaKR2I, M.Lmo1042I, M.Lmo7956II, | | | | |
| EarI, Ksp632I | | M.Lsp10393II, M.MspI7I, M.Sau3AI, M.Sba3I, | | | | |
| | | M.Sin395I, M.Spn23FI, M.Sth368I | | | | |
| Bco5I, Bco116I, | CTCTT | M.CocIII, M.Fsi4947II, M.TdeIII, | GGNCC | 1, 5 | m4C | GGNCCTCTTCN[NNN] |
| BcoKI, BseZI, | C | M.Tph12270I | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.CviJI | RGCB | 2, 3 | m5C | VGCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.Dac11109II, M.Hfo6591I, M.MhuII, | GTAC | 1, 4 | m4C | GTACTCTTCN[NNN] |
| BcoKI, BseZI, | C | M.MjaV, M.MlaZII, M.MspLW5I, M.RsaI | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.Esp3I | CGTCTC | 4, 3 | m5C, | CGTCTCTTCN[NNN] |
| BcoKI, BseZI, | C | | | | m6A | |
| Bst6I, Bsu6I, | | | | | | |
| CatHI, Eam1104I, | | | | | | |
| EarI, Ksp632I | | | | | | |
| Bco5I, Bco116I, | CTCTT | M.HpyD27I | CCTC | 1, 3 | m5C, | CCTCTTCN[NNN] |
| BcoKI, BseZI, | C | | | | m6A | |
| Bst6I, Bsu6I, | | | | | | |
| CatHI, Eam1104I, | | | | | | |
| EarI, Ksp6321 | | | | | | |
| Bco5I, Bco116I, | CTCTT | M.MjaII | GGNCC | 1, 5 | m5C | GGNCCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.MlyI, M.Pen113I | GASTC | 2, 4 | m6A | GACTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.Mma5219II, M.TarMY3I | AGCT | 2, 3 | m4C | AGCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.MpeII | RGCY | 2, 3 | m5C | RGCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.NheI | GCTAGC | 1, 6 | m4C | GCTAGCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.Nme579III, M.NmeSI, M.ScaI, M.Ssp6714II | AGTACT | 2, 5 | m4C | AGTACTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.Pae10332II, M1.Pae50071I, | CAGCTC | 3, 4 | m4C | CAGCTCTTCN[NNN] |
| BcoKI, BseZI, | C | M2.Pae50071I | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.SacI, M.Sgr13350I | GAGCTC | 3, 4 | m5C | GAGCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.SuaII | RGATCY | 2, 5 | m5C | RGATCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.TacIII | GGCCT | 4 | m4C | GGCCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.Tam77409IV | CCRCTC | 4 | m4C | CCRCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M.TspPM5I | AGGCCT | 2, 5 | m4C | AGGCCTCTTCN[NNN] |
| BcoKI, BseZI, | C | | | | | |
| Bst6I, Bsu6I, | | | | | | |
| CatHI, Eam1104I, | | | | | | |
| EarI, Ksp6321 | | | | | | |
| Bco5I, Bco116I, | CTCTT | M.XmnI | GAANNNN | 2, 9 | m6A | GAANCTCTTCN[NNN] |
| BcoKI, BseZI, | C | | TTC | | | |
| Bst6I, Bsu6I, | | | | | | |
| CatHI, Eam1104I, | | | | | | |
| EarI, Ksp632I | | | | | | |
| Bco5I, Bco116I, | CTCTT | M1.Sgr13350III, M2.Sgr13350III | GCTCTTC | 2, 3 | m4C, | GCTCTTCN[NNN] |
| BcoKI, BseZI, | C | | | | m6A | |
| Bst6I, Bsu6I, | | | | | | |
| CatHI, Eam1104I, | | | | | | |
| EarI, Ksp632I | | | | | | |
| Bco5I, Bco116I, | CTCTT | M2.BstSEI | GACTC | 4 | m6A | GACTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bco5I, Bco116I, | CTCTT | M2.Csu24377II, M2.Hpy298VII, | CCTC | 3 | m6A | CCTCTTCN[NNN] |
| BcoKI, BseZI, | C | M2.Hpy299VI, M2.Hpy30XII, M1.Hpy32VI, | | | | |
| Bst6I, Bsu6I, | | M1.Hpy57VI, M2.Hpy66XIV, M1.Hpy99V, | | | | |
| CatHI, Eam1104I, | | M1.HpyAVI, M1.HpyPVI, M2.HpyUM032VI, | | | | |
| EarI, Ksp632I | | M.Mdi27140I, M2.MnlI | | | | |
| Bco5I, Bco116I, | CTCTT | M2.Eco31I | GGTCTC | 4 | m5C | GGTCTCTTCN[NNN] |
| BcoKI, BseZI, | C | | | | | |
| Bst6I, Bsu6I, | | | | | | |
| CatHI, Eam1104I, | | | | | | |
| EarI, Ksp632I | | | | | | |
| Bli736I, BsaI, | GGTC | M.AbaBs12I, M.Awo1030I, M.Cdu23823I, | CCWGG | 1, 5 | m5C | CCWGGGTCTCN[NNNN], |
| Bso31I, BspTNI, | TC | M.Csa8155II, M.Cun9529I, M.EasL1Dcm, | | | | CCWGGTCTCN[NNNN] |
| Bsu537I, Eco31I, | | M.Eba57Dcm, M.Ecl34399Dcm, | | | | |
| EcoA4I, EcoO44I | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48II, | | | | |
| | | M.Pmi1I, M.SpaD2III, M.SplRp8II, | | | | |
| | | M.SptADcm, M.Tsu2489III | | | | |
| Bli736I, BsaI, | GGTC | M.AciI | GCGG | 2, 4 | m5C | GCGGGTCTCN[NNNN], |
| Bso31I, BspTNI, | TC | | | | | GCGGTCTCN[NNNN] |
| Bsu537I, Eco31I, | | | | | | |
| EcoA4I, EcoO44I | | | | | | |
| Bli736I, BsaI, | GGTC | M.AhdI | GACNNNN | 2, 10 | m6A | GACNNNNGGTCTCN[NNNN] |
| Bso31I, BspTNI, | TC | | NGTC | | | |
| Bsu537I, Eco31I, | | | | | | |
| EcoA4I, EcoO44I | | | | | | |
| Bli736I, BsaI, | GGTC | M.Ama55I, M.CnaPQ2III, M.Rmo1926II, | CGGCCG | 1, 6 | m5C | CGGCCGGTCTCN[NNNN] |
| Bso31I, BspTNI, | TC | M.SenC1765IV, M.SspG1I, M.XorPXII | | | | |
| Bsu537I, Eco31I, | | | | | | |
| EcoA4I, EcoO44I | | | | | | |
| Bli736I, BsaI, | GGTC | M.AquI | CYCGRG | 1, 6 | m5C | CYCGRGGTCTCN[NNNN], |
| Bso31I, BspTNI, | TC | | | | | CYCGGGTCTCN[NNNN] |
| Bsu537I, Eco31I, | | | | | | |
| EcoA4I, EcoO44I | | | | | | |
| Bli736I, BsaI, | GGTC | M.AspU41II, M.BisIII, M.BsuW23II, | CCWGG | 2, 4 | m5C | CCWGGTCTCN[NNNN] |
| Bso31I, BspTNI, | TC | M.CnaPQ2II, M.EcoO6Dcm, M.Eco12581Dcm, | | | | |
| Bsu537I, Eco31I, | | M.Eco1655Dcm, M.Eco29787Dcm, | | | | |
| EcoA4I, EcoO44I | | M.Eco3024Dcm, M.Eco3317Dcm, | | | | |
| | | M.Eco3325Dcm, M.Eco3740Dcm, | | | | |
| | | M.Eco3911Dcm, M.Eco4211Dcm, | | | | |
| | | M.Eco4465Dcm, M.Eco600Dcm, | | | | |
| | | M.Eco644Dcm, M.Eco86Dcm, | | | | |
| | | M.Eco9001Dcm, M.Eco9387Dcm, | | | | |
| | | M.EcoE1140Dcm, M.EcoE455Dcm, | | | | |
| | | M.EcoGDcm, M.EcoNU14Dcm, M.EcoRII, | | | | |
| | | M.Kpn223III, M.Kpn35657II, M.Kpn36Dcm, | | | | |
| | | M.Kpn555I, M.Kpn62629II, M.KpnAATV, | | | | |
| | | M.Kqu603Dcm, M.Psp345I, M.Sen158Dcm, | | | | |
| | | M.Sen641Dcm, M.SenA46Dcm, | | | | |
| | | M.SenAboDcm, M.TcoKWC4II | | | | |
| Bli736I, BsaI, | GGTC | M.AvaIX, M.Cfr10I, M.Nme18VI, M.VchAI, | RCCGGY | 2, 5 | m5C | RCCGGTCTCN[NNNN] |
| Bso31I, BspTNI, | TC | M.Vch0395I | | | | |
| Bsu537I, Eco31I, | | | | | | |
| EcoA4I, EcoO44I | | | | | | |
| Bli736I, BsaI, | GGTC | M.BcoSlacII, M.MspLW5III, M.Nbr128I, | CTCGAG | 1, 6 | m4C | CTCGAGGTCTCN[NNNN] |
| Bso31I, BspTNI, | TC | M.Psy18884II, M.Tam77409II, M.TbrGE1II, | | | | |
| Bsu537I, Eco31I, | | M.TpaRLII | | | | |
| EcoA4I, EcoO44I | | | | | | |
| Bli736I, BsaI | GGTC | M.BepI, M.FpsJV, M.Gel16401III, M.Pdi8503I | CGCG | 1, 4 | m5C | CGCGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.Bhy27164IV, M.BstNI, M.EcoDHB4Dcm, | CCWGG | 2, 4 | m4C | CCWGGTCTCN[NNNN] |
| Bso31I, BspTNI, | TC | M.EcoK54Dcm, M.Gth3570I, M.Hla49239I, | | | | |
| Bsu537I, Eco31I, | | M.MvaI, M.Pdi8503II, M.Sso30807Dcm, | | | | |
| EcoA4I, EcoO44I | | M.YpeI, M.Ype41I, M.YpeShI | | | | |
| Bli736I, BsaI, | GGTC | M.BsoBI | CYCGRG | 1, 6 | m4C | CYCGRGGTCTCN[NNNN], |
| Bso31I, BspTNI, | TC | | | | | CYCGGGTCTCN[NNNN] |
| Bsu537I, Eco31I, | | | | | | |
| EcoA4I, EcoO44I | | | | | | |
| Bli736I, BsaI, | GGTC | M.BsuFI, M.CpeAIV, M.MspI | CCGG | 1, 4 | m5C | CCGGGTCTCN[NNNN], CCGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.Cab13497I, M.Dac11109III, M.Dco13527I, | CTAG | 1, 4 | m4C | CTAGGTCTCN[NNNN] |
| Bso31I, BspTNI, | TC | M.Djo10085I, M.Hbo11551I, M.Hka19301III, | | | | |
| Bsu537I, Eco31I, | | M.Hme33500I, M.HsaR1I, M.Hsi33800I, | | | | |
| EcoA4I, EcoO44I | | M.HspKM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| Bli736I, BsaI, | GGTC | M.Cdi630III | CCSSGG | 2, 5 | m4C | CCSSGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.Cfr9I, M.SmaI, M.Sma36365II, M.Xca85III, | CCCGGG | 2, 5 | m4C | CCCGGGTCTCN[NNNN] |
| Bso31I, BspTNI, | TC | M.XcyI, M.XmaI, M.XveII | | | | |
| Bsu537I, Eco31I, | | | | | | |
| EcoA4I, EcoO44I | | | | | | |
| Bli736I, BsaI, | GGTC | M.CfrBI, M.Sen1735IV, M.SenAnaIV | CCWWGG | 1, 6 | m4C | CCWWGGGTCTCN[NNNN], |
| Bso31I, BspTNI, | TC | | | | | CCWWGGTCTCN[NNNN] |
| Bsu537I, Eco31I, | | | | | | |
| EcoA4I, EcoO44I | | | | | | |
| Bli736I, BsaI | GGTC | M.Cmu51329I, M.EcoJA03PDcm, | CCWGG | 1, 5 | Unknown | CCWGGGTCTCN[NNNN], |
| Bso31I, BspTNI, | TC | M.RorR1Dcm, M.Sen8391Dcm, M.ZalSM2II | | | | CCWGGTCTCN[NNNN] |
| Bsu537I, Eco31I, | | | | | | |
| EcoA4I, EcoO44I | | | | | | |
| Bli736I, BsaI, | GGTC | M.Cpa18696III, M.Ype19I, M.YpeDodI, | CCWGG | 2, 4 | Unknown | CCWGGTCTCN[NNNN] |
| Bso31I, BspTNI, | TC | M.YpeEDI, M.YpeJ9I, M.YpePGI | | | | |
| Bsu537I, Eco31I, | | | | | | |
| EcoA4I, EcoO44I | | | | | | |
| Bli736I, BsaI, | GGTC | M.DdeI | CTNAG | 1, 5 | m5C | CTNAGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.DsaV, M.Ecl18kI, M.FpsJVI, M.NlaX, | CCNGG | 2, 4 | m5C | CCNGGTCTCN[NNNN] |
| Bso31I, BspTNI, | TC | M.SenPI, M.SsoII | | | | |
| Bsu537I, Eco31I, | | | | | | |
| EcoA4I, EcoO44I | | | | | | |
| Bli736I, BsaI, | GGTC | M.Eco29kI, M.NgoAIII, M.SacII | CCGCGG | 2, 5 | m5C | CCGCGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.EcoNI | CCTNNNN | 2, 10 | m4C | CCTNNNNNAGGTCTCN[NNNN] |
| Bso31I, BspTNI, | TC | | NAGG | | | |
| Bsu537I, Eco31I, | | | | | | |
| EcoA4I, EcoO44I | | | | | | |
| Bli736I, BsaI, | GGTC | M.EfaRFII, M.Mae7806II | CCGG | 2, 3 | m4C | CCGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.FatI | CATG | 1, 4 | m5C | CATGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.FisAW1I, M.ThaI, M.Tph12270II | CGCG | 1, 4 | m4C | CGCGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.FnoB1II, M.Hal24586I, M.Hpy298V, | CCGG | 1, 4 | m4C | CCGGGTCTCN[NNNN], CCGGTCTCN[NNNN] |
| Bso31I, BspTNI, | TC | M.Hpy299IX, M.Hpy30V, M.Hpy32XI, | | | | |
| Bsu537I, Eco31I, | | M.Hpy57V, M.Hpy66IX, M.Hpy99VIII, | | | | |
| EcoA4I, EcoO441 | | M.HpyFV, M.HpyUM032IX, M.HpyUM037IX, | | | | |
| | | M.MjaVI, M.PspJDRI, M.TmeBII | | | | |
| Bli736I, BsaI, | GGTC | M.FpsJI, M.HpaII, M.Nme18II, M.NmeAI | CCGG | 2, 3 | m5C | CCGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.Gel16401IV | CTGG | 3 | m5C | CTGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.GsuRed1IV, M.Osp807II, M.Tth111I | GACNNNG | 2, 8 | m6A | GACNNGGTCTCN[NNNN] |
| Bso31I, BspTNI, | TC | | TC | | | |
| Bsu537I, Eco31I, | | | | | | |
| EcoA4I, EcoO44I | | | | | | |
| Bli736I, BsaI | GGTC | M.Hpy298II, M.Hpy299II, M.Hpy66II, | ACNGT | 2, 4 | m4C | ACGGTCTCN[NNNN] |
| Bso31I, BspTNI, | TC | M.HpyUM032II, M.HpyUM037II | | | | |
| Bsu537I, Eco31I, | | | | | | |
| EcoA4I, EcoO44I | | | | | | |
| Bli736I, BsaI, | GGTC | M.Hpy30X, M.Hpy57VIII, M.HpyFXI, M.MhuI | CTNAG | 1, 5 | m4C | CTNAGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.Hpy99IV, M.HpyFORFX | CCNNGG | 1, 6 | m4C | CCNNGGGTCTCN[NNNN], |
| Bso31I, BspTNI, | TC | | | | | CCNNGGTCTCN[NNNN] |
| Bsu537I, Eco31I, | | | | | | |
| EcoA4I, EcoO44I | | | | | | |
| Bli736I, BsaI, | GGTC | M.HpyAV, M.Nme18IV | GAAGG | 3, 4 | m6A, | GAAGGTCTCN[NNNN] |
| Bso31I, BspTNI, | TC | | | | m5C | |
| Bsu537I, Eco31I, | | | | | | |
| EcoA4I, EcoO44I | | | | | | |
| Bli736I, BsaI, | GGTC | M.HpyD27I | GAGG | 2, 4 | m6A, | GAGGGTCTCN[NNNN], |
| Bso31I, BspTNI, | TC | | | | m5C | GAGGTCTCN[NNNN] |
| Bsu537I, Eco31I, | | | | | | |
| EcoA4I, EcoO44I | | | | | | |
| Bli736I, BsaI, | GGTC | M.Lph145I, M.Lph93I | TCWCC | 1 | m6A | GGTCTCC[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.Msp1II | CCAGG | 5 | m4C | CCAGGGTCTCN[NNNN], |
| Bso31I, BspTNI, | TC | | | | | CCAGGTCTCN[NNNN] |
| Bsu537I, Eco31I, | | | | | | |
| EcoA4I, EcoO44I | | | | | | |
| Bli736I, BsaI, | GGTC | M.Msp42II | CCWGG | 1, 5 | m4C | CCWGGGTCTCN[NNNN], |
| Bso31I, BspTNI, | TC | | | | | CCWGGTCTCN[NNNN] |
| Bsu537I, Eco31I, | | | | | | |
| EcoA4I, EcoO44I | | | | | | |
| Bli736I, BsaI, | GGTC | M.Nme18III | TCTGG | 2, 4 | m5C | TCTGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.NmeDI | VCCGGY | 2, 5 | m5C | VCCGGTCTCN[NNNN], |
| Bso31I, BspTNI, | TC | | | | | RCCGGGTCTCN[NNNN] |
| Bsu537I, Eco31I, | | | | | | |
| EcoA4I, EcoO44I | | | | | | |
| Bli736I, BsaI, | GGTC | M.NmeMC58III | TCTGG | 5 | m5C | TCTGGGTCTCN[NNNN], |
| Bso31I, BspTNI, | TC | | | | | TCTGGTCTCN[NNNN] |
| Bsu537I, Eco31I, | | | | | | |
| EcoA4I, EcoO44I | | | | | | |
| Bli736I, BsaI, | GGTC | M.Nsp209III, M.Nsp51109II | CTCGAG | 1, 6 | m5C | CTCGAGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.Pam7686I | CCATGG | 1, 6 | m5C | CCATGGGTCTCN[NNNN], |
| Bso31I, BspTNI, | TC | | | | | CCATGGTCTCN[NNNN] |
| Bsu537I, Eco31I, | | | | | | |
| EcoA4I, EcoO44I | | | | | | |
| Bli736I, BsaI, | GGTC | M.Pfa23572II | CGATCG | 1, 6 | m5C | CGATCGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.Pla5I | CTAG | 1, 4 | Unknown | CTAGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M.Sam53668II | CCATGG | 1, 6 | Unknown | CCATGGGTCTCN[NNNN], |
| Bso31I, BspTNI, | TC | | | | | CCATGGTCTCN[NNNN] |
| Bsu537I, Eco31I, | | | | | | |
| EcoA4I, EcoO44I | | | | | | |
| Bli736I, BsaI, | GGTC | M.Sen1781III | CCWWGG | 1, 6 | Unknown | CCWWGGGTCTCN[NNNN], |
| Bso31I, BspTNI, | TC | | | | | CCWWGGTCTCN[NNNN] |
| Bsu537I, Eco31I, | | | | | | |
| EcoA4I, EcoO44I | | | | | | |
| Bli736I, BsaI, | GGTC | M.SonIII | GCANNNN | 3, 9 | m6A | GCANNNGGTCTCN[NNNN] |
| Bso31I, BspTNI, | TC | | GTC | | | |
| Bsu537I, Eco31I, | | | | | | |
| EcoA4I, EcoO44I | | | | | | |
| Bli736I, BsaI, | GGTC | M.TdeII | GAAGAG | 5, 6 | m6A, | GAAGAGGTCTCN[NNNN] |
| Bso31I, BspTNI, | TC | | | | m4C | |
| Bsu537I, Eco31I, | | | | | | |
| EcoA4I, EcoO44I | | | | | | |
| Bli736I, BsaI, | GGTC | M.TspRI | CASTG | 1, 5 | m5C | CASTGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M1.AgoG2I, M2.AgoG2I | ACTGG | 2, 5 | m4C | ACTGGGTCTCN[NNNN], |
| Bso31I, BspTNI, | TC | | | | | ACTGGTCTCN[NNNN] |
| Bsu537I, Eco31I, | | | | | | |
| EcoA4I, EcoO44I | | | | | | |
| Bli736I, BsaI, | GGTC | M1.Bag18758II, M2.Bag18758II | CACGGG | 3, 5 | Unknown | CACGGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M1.BcnI, M2.BcnI, M.NciI | CCSGG | 2, 4 | m4C | CCSGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M1.BfiI | ACTGGG | 5 | m4C | ACTGGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M1.BspACI | GCGG | 4 | m5C | GCGGGTCTCN[NNNN], |
| Bso31I, BspTNI, | TC | | | | | GCGGTCTCN[NNNN] |
| Bsu537I, Eco31I, | | | | | | |
| EcoA4I, EcoO44I | | | | | | |
| Bli736I, BsaI, | GGTC | M1.Hpy32II | TCYYC | 1 | m6A | GGTCTCC[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M1.LplAG30I | GAAGG | 5 | m5C | GAAGGGTCTCN[NNNN], |
| Bso31I, BspTNI, | TC | | | | | GAAGGTCTCN[NNNN] |
| Bsu537I, Eco31I, | | | | | | |
| EcoA4I, EcoO44I | | | | | | |
| Bli736I, BsaI, | GGTC | M2.BfuAI | GCAGGT | 5 5 | m5C | GCAGGTCTCN[NNNN] |
| Bso31I, BspTNI, | TC | | | | | |
| Bsu537I, Eco31I, | | | | | | |
| EcoA4I, EcoO44I | | | | | | |
| Bli736I, BsaI, | GGTC | M2.HpyAVI, M1.MnlI | GAGG | 4 | m5C | GAGGGTCTCN[NNNN], |
| Bso31I, BspTNI, | TC | | | | | GAGGTCTCN[NNNN] |
| Bsu537I, Eco31I, | | | | | | |
| EcoA4I, EcoO44I | | | | | | |
| BsmBI, BstGZ53I, | CGTCT | M.AatII, M.Lsp6406III | GACGTC | 2, 5 | m6A | GACGTCTCN[NNNN] |
| Esp3I | C | | | | | |
| BsmBI, BstGZ53I, | CGTCT | M.AflIII | ACRYGT | 2, 5 | m4C | ACRCGTCTCN[NNNN] |
| Esp3I | C | | | | | |
| BsmBI, BstGZ53I, | CGTCT | M.AhdI | GACNNNN | 2, 10 | m6A | GACNNNNCGTCTCN[NNNN] |
| Esp3I | C | | NGTC | | | |
| BsmBI, BstGZ53I, | CGTCT | M.Ama55I, M.CnaPQ2III, M.Rmo1926II, | CGGCCG | 1, 6 | m5C | CGGCCGTCTCN[NNNN] |
| Esp3I | C | M.SenC1765IV, M.SspG1I, M.XorPXII | | | | |
| BsmBI, BstGZ53I, | CGTCT | M.AvaV, M.SliSI | GATC | 1, 4 | m4C | GATCGTCTCN[NNNN] |
| Esp3I | C | | | | | |
| BsmBI, BstGZ53I, | CGTCT | M.BceSII | GGWCC | 1, 5 | m5C | GGWCCGTCTCN[NNNN] |
| Esp3I | C | | | | | |
| BsmBI, BstGZ53I, | CGTCT | M.BepI, M.FpsJV, M.Gel16401III, M.Pdi8503I | CGCG | 1, 4 | m5C | CGCGTCTCN[NNNN] |
| Esp3I | C | | | | | |
| BsmBI, BstGZ53I, | CGTCT | M.Bg12196III | CGTACG | 2, 5 | m4C | CGTACGTCTCN[NNNN] |
| Esp3I | C | | | | | |
| BsmBI, BstGZ53I, | CGTCT | M.Bsp14III, M.Bsp3003I, M.Bsp3004I, | GATC | 1, 4 | m5C | GATCGTCTCN[NNNN] |
| Esp3I | C | M.CalB3III, M.CpeAIII, M.Esu35585I, | | | | |
| | | M.FpsJIV, M.Lba2031I, M.Lbo2004I, | | | | |
| | | M.LlaKR2I, M.Lmo1042I, M.Lmo7956II, | | | | |
| | | M.Lsp10393II, M.MspI7I, M.Sau3AI, M.Sba3I, | | | | |
| | | M.Sin395I, M.Spn23FI, M.Sth368I | | | | |
| BsmBI, BstGZ53I, | CGTCT | M.CagVIII | ACGCGT | 2, 5 | m4C | ACGCGTCTCN[NNNN] |
| Esp3I | C | | | | | |
| BsmBI, BstGZ53I, | CGTCT | M.CocIII, M.Fsi4947II, M.TdeIII, | GGNCC | 1, 5 | m4C | GGNCCGTCTCN[NNNN] |
| Esp3I | C | M.Tph12270I | | | | |
| BsmBI, BstGZ53I, | CGTCT | M.CviJI | RGCB | 2, 3 | m5C | RGCGTCTCN[NNNN] |
| Esp3I | C | | | | | |
| BsmBI, BstGZ53I, | CGTCT | M.Dac11109II, M.Hfo6591I, M.MhuII, | GTAC | 1, 4 | m4C | GTACGTCTCN[NNNN] |
| Esp3I | C | M.MjaV, M.MlaZII, M.MspLW5I, M.RsaI | | | | |
| BsmBI, BstGZ53I, | CGTCT | M.FisAW1I, M.ThaI, M.Tph12270II | CGCG | 1, 4 | m4C | CGCGTCTCN[NNNN] |
| Esp3I | C | | | | | |
| BsmBI, BstGZ53I, | CGTCT | M.GsuRed1IV, M.Osp807II, M.Tth111I | GACNNNG | 2, 8 | m6A | GACNNCGTCTCN[NNNN] |
| Esp31 | C | | TC | | | |
| BsmBI, BstGZ53I, | CGTCT | M.Hpy298II, M.Hpy299II, M.Hpy66II, | ACNGT | 2, 4 | m4C | ACCGTCTCN[NNNN] |
| Esp3I | C | M.HpyUM032II, M.HpyUM037II | | | | |
| BsmBI, BstGZ53I, | CGTCT | M.Hpy66I, M.Hpy99I | CGWCG | 2, 4 | m4C | CGWCGTCTCN[NNNN] |
| Esp3I | C | | | | | |
| BsmBI, BstGZ53I, | CGTCT | M.Hpy99XI | ACGT | 2, 3 | m5C | ACGTCTCN[NNNN] |
| Esp3I | C | | | | | |
| BsmBI, BstGZ53I, | CGTCT | M.Lph145I, M.Lph93I | TCWCC | 1 | m6A | CGTCTCC[NNNN] |
| Esp3I | C | | | | | |
| BsmBI, BstGZ53I, | CGTCT | M.MjaII | GGNCC | 1, 5 | m5C | GGNCCGTCTCN[NNNN] |
| Esp3I | C | | | | | |
| BsmBI, BstGZ53I, | CGTCT | M.NheI | GCTAGC | 1, 6 | m4C | GCTAGCGTCTCN[NNNN] |
| Esp3I | C | | | | | |
| BsmBI, BstGZ53I, | CGTCT | M.Pfa23572II | CGATCG | 1, 6 | m5C | CGATCGTCTCN[NNNN] |
| Esp3I | C | | | | | |
| BsmBI, BstGZ53I, | CGTCT | M.SonIII | GCANNNN | 3, 9 | m6A | GCANNNCGTCTCN[NNNN] |
| Esp3I | C | | GTC | | | |
| BsmBI, BstGZ53I, | CGTCT | M1.BceSIII | GCCGT | 4 | m4C | GCCGTCTCN[NNNN] |
| Esp3I | C | | | | | |
| BsmBI, BstGZ53I, | CGTCT | M1.Dsh12II, M2.Dsh12II | GCCGTC | 3, 4 | m4C | GCCGTCTCN[NNNN] |
| Esp3I | C | | | | | |
| BsmBI, BstGZ53I, | CGTCT | M1.HgaI, M2.LlaJI | GCGTC | 2 | m5C | GCGTCTCN[NNNN] |
| Esp3I | C | | | | | |
| BsmBI, BstGZ53I, | CGTCT | M1.Hpy32II | TCYYC | 1 | m6A | CGTCTCC[NNNN] |
| Esp3I | C | | | | | |
| BsmBI, BstGZ53I, | CGTCT | M2.BceSIII, M1.Pru9I, M2.Pru91 | GCCGT | 2, 4 | m4C | GCCGTCTCN[NNNN] |
| Esp3I | C | | | | | |
| BsmBI, BstGZ53I, | CGTCT | M2.HgaI, M1.LlaJI | GCGTC | 3 | m5C | GCGTCTCN[NNNN] |
| Esp3I | C | | | | | |
| BsmBI, BstGZ53I, | CGTCT | M2.Nme579IV, M2.NmeMC58II | GCGTC | 4 | m6A | GCGTCTCN[NNNN] |
| Esp3I | C | | | | | |
| BtgZI | GCGA | M.AbaBs12I, M.Awo1030I, M.Cdu23823I, | CCWGG | 1, 5 | m5C | CCWGGCGATGNNNNNNNNNN[NNNN] |
| | TG | M.Csa8155II, M.Cun9529I, M.EasL1Dcm, | | | | |
| | | M.Eba57Dcm, M.Ec134399Dcm, | | | | |
| | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| BtgZI | GCGA | M.AciI | CCGC | 1, 3 | m5C | GCGGCGATGNNNNNNNNNN[NNNN], |
| | TG | | | | | CCGCGATGNNNNNNNNNN[NNNN] |
| BtgZI | GCGA | M.Ama55I, M.CnaPQ2III, M.Rmo1926II, | CGGCCG | 1, 6 | m5C | CGGCCGCGATGNNNNNNNNNN[NNNN] |
| | TG | M.SenC1765IV, M.SspG1I, M.XorPXII | | | | |
| BtgZI | GCGA | M.AquI | CYCGRG | 1, 6 | m5C | CYCGRGCGATGNNNNNNNNNN[NNNN] |
| | TG | | | | | |
| BtgZI | GCGA | M.AsiSI | GCGATCGC | 2, 7 | m5C | GCGATCGCGATGNNNNNNNNNN[NNNN] |
| | TG | | | | | |
| BtgZI | GCGA | M.AvaIX, M.Cfr10I, M.Nme18VI, M.VchAI, | RCCGGY | 2, 5 | m5C | RCCGGCGATGNNNNNNNNNN[NNNN] |
| | TG | M.Vch0395I | | | | |
| BtgZI | GCGA | M.AvaV, M.SliSI | GATC | 1, 4 | m4C | GCGATGATCNNNNNNN[NNNN] |
| | TG | | | | | |
| BtgZI | GCGA | M.BamWS8I, M.CthV, M.EfaRFI, M.Saf8902II, | GCWGC | 2, 4 | m5C | GCWGCGATGNNNNNNNNNN[NNNN] |
| | TG | M.TacII, M.TcoKWC4III | | | | |
| BtgZI | GCGA | M.BbvI, M.BceSIV | GCAGC | 2, 4 | m5C | GCTGCGATGNNNNNNNNNN[NNNN], |
| | TG | | | | | GCAGCGATGNNNNNNNNNN[NNNN] |
| BtgZI | GCGA | M.BceSII | GGWCC | 1, 5 | m5C | GCGATGGWCCNNNNNN[NNNN] |
| | TG | | | | | |
| BtgZI | GCGA | M.BcoSlacII, M.MspLW5III, M.Nbr128I, | CTCGAG | 1, 6 | m4C | CTCGAGCGATGNNNNNNNNNN[NNNN] |
| | TG | M.Psy18884II, M.Tam77409II, M.TbrGE1II, | | | | |
| | | M.TpaRLII | | | | |
| BtgZI | GCGA | M.BepI, M.FpsJV, M.Gel16401III, M.Pdi8503I | CGCG | 1, 4 | m5C | CGCGCGATGNNNNNNNNNN[NNNN], |
| | TG | | | | | CGCGATGNNNNNNNNNN[NNNN] |
| BtgZI | GCGA | M.BfaSIII, M.Ccl10114I, M.CfrB38I, | ATGCAT | 2, 5 | m6A | GCGATGCATNNNNNNN[NNNN] |
| | TG | M.CfrH1I, M.EcoGVI, M.EcoKII, | | | | |
| | | M.Fnu10562II, M.SbaUIV, M.Sbo12419III, | | | | |
| | | M.Sbo268II, M.Sen10384II, M.Sen10708II, | | | | |
| | | M.Sen1080II, M.Sen1175III, M.Sen13IV, | | | | |
| | | M.Sen13311IV, M.Sen1387II, M.Sen1427III, | | | | |
| | | M.Sen14882III, M.Sen15791III, M.Sen158III, | | | | |
| | | M.Sen16I, M.Sen1655III, M.Sen1676III, | | | | |
| | | M.Sen1677III, M.Sen1728III, M.Sen1735III, | | | | |
| | | M.Sen1736IV, M.Sen1764III, M.Sen1766III, | | | | |
| | | M.Sen1781IV, M.Sen1783III, M.Sen18569II, | | | | |
| | | M.Sen1878III, M.Sen1880III, M.Sen1896III, | | | | |
| | | M.Sen1898III, M.Sen1899III, M.Sen1903III, | | | | |
| | | M.Sen1906III, M.Sen1908III, M.Sen1910III, | | | | |
| | | M.Sen1921III, M.Sen1927II, M.Sen195II, | | | | |
| | | M.Sen2050II, M.Sen2064III, M.Sen2069III, | | | | |
| | | M.Sen22462I, M.Sen255II, M.Sen318III, | | | | |
| | | M.Sen3387III, M.Sen3890III, M.Sen483III, | | | | |
| | | M.Sen624III, M.Sen641III, M.Sen7378II, | | | | |
| | | M.Sen8391III, M.Sen8692III, M.SenA46III, | | | | |
| | | M.SenAbaII, M.SenAboII, M.SenAnaIII, | | | | |
| | | M.SenC1736III, M.SenC1765III, | | | | |
| | | M.SenC1808IV, M.SenC1810III, M.SenJIII, | | | | |
| | | M.SenL1II, M.SenL15III, M.SenN1660II, | | | | |
| | | M.SenSARA26II, M.SenSPBIII, M.SenTFIII | | | | |
| BtgZI | GCGA | M.BglI, M.CagI, M.DdeVCDI, M.GsuRed1I | GCCNNNN | 2, 10 | m4C | GCCNNNNNGGCGATGNNNNNNNNNN[NNNN] |
| | TG | | NGGC | | | |
| BtgZI | GCGA | M.BsoBI | CYCGRG | 1, 6 | m4C | CYCGRGCGATGNNNNNNNNNN[NNNN] |
| | TG | | | | | |
| BtgZI | GCGA | M.Bsp14III, M.Bsp3003I, M.Bsp3004I, | GATC | 1, 4 | m5C | GCGATGATCNNNNNNN[NNNN] |
| | TG | M.CalB3III, M.CpeAIII, M.Esu35585I, | | | | |
| | | M.FpsJIV, M.Lba2031I, M.Lbo2004I, | | | | |
| | | M.LlaKR2I, M.Lmo1042I, M.Lmo7956II, | | | | |
| | | M.Lsp10393II, M.MspI7I, M.Sau3AI, M.Sba3I, | | | | |
| | | M.Sin395I, M.Spn23FI, M.Sth368I | | | | |
| BtgZI | GCGA | M.BstXI | CCANNNN | 3, 10 | m6A | CCANNGCGATGGNNNNNNNNN[NNNN] |
| | TG | | NNTGG | | | |
| BtgZI | GCGA | M.BsuFI, M.CpeAIV, M.MspI | CCGG | 1, 4 | m5C | CCGGCGATGNNNNNNNNNN[NNNN] |
| | TG | | | | | |
| BtgZI | GCGA | M.Cab13497I, M.Dac11109III, M.Dco13527I, | CTAG | 1, 4 | m4C | CTAGCGATGNNNNNNNNNN[NNNN] |
| | TG | M.Djo10085I, M.Hbo11551I, M.Hka19301III, | | | | |
| | | M.Hme33500I, M.HsaR1I, M.Hsi33800I, | | | | |
| | | M.HspKM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| BtgZI | GCGA | M.Cac8I | GCNNGC | 2, 5 | m5C | GCNNGCGATGNNNNNNNNNN[NNNN] |
| | TG | | | | | |
| BtgZI | GCGA | M.CcrNAV | YGCCGGCR | 3, 6 | m5C | YGCCGGCGATGNNNNNNNNNN[NNNN] |
| | TG | | | | | |
| BtgZI | GCGA | M.Cdi630IV, M.Ckr177III, M.CmaLM2II, | GCNGC | 2, 4 | m5C | GCNGCGATGNNNNNNNNNN[NNNN] |
| | TG | M.CocII, M.Fnu4HI, M.Fsp4HI | | | | |
| BtgZI | GCGA | M.CfrBI, M.Sen1735IV, M.SenAnaIV | CCWWGG | 1, 6 | m4C | CCWWGGCGATGNNNNNNNNNN[NNNN] |
| | TG | | | | | |
| BtgZI | GCGA | M.Cmu51329I, M.EcoJA03PDcm, | CCWGG | 1, 5 | Unknown | CCWGGCGATGNNNNNNNNNN[NNNN] |
| | TG | M.RorR1Dcm, M.Sen8391Dcm, M.ZalSM2II | | | | |
| BtgZI | GCGA | M.CocIII, M.Fsi4947II, M.TdeIII, | GGNCC | 1, 5 | m4C | GCGATGGNCCNNNNNN[NNNN] |
| | TG | M.Tph12270I | | | | |
| BtgZI | GCGA | M.CsiFSMII, M.MfoCT6IV, M.NgoAIV, | GCCGGC | 2, 5 | m5C | GCCGGCGATGNNNNNNNNNN[NNNN] |
| | TG | M.NgoMIV, M1.Sgr13350II | | | | |
| BtgZI | GCGA | M.CviJI | RGCB | 2, 3 | m5C | RGCGATGNNNNNNNNNN[NNNN] |
| | TG | | | | | |
| BtgZI | GCGA | M.CviRI, M.Hpy298VIII, M.Hpy299VIII, | TGCA | 1, 4 | m6A | GCGATGCANNNNNNNN[NNNN] |
| | TG | M.Hpy30VI, M.Hpy32V, M.Hpy57II, | | | | |
| | | M.Hpy66VIII, M.HpyFVI, M.HpyUM032VIII, | | | | |
| | | M.HpyUM037VIII | | | | |
| BtgZI | GCGA | M.Dac11109II, M.Hfo6591I, M.MhuII, | GTAC | 1, 4 | m4C | GCGATGTACNNNNNNN[NNNN] |
| | TG | M.MjaV, M.MlaZII, M.MspLW5I, M.RsaI | | | | |
| BtgZI | GCGA | M.DdeI | CTNAG | 1, 5 | m5C | CTNAGCGATGNNNNNNNNNN[NNNN] |
| | TG | | | | | |
| BtgZI | GCGA | M.Esp1396I | CCANNNN | 3, 9 | m6A | CCANGCGATGGNNNNNNNNN[NNNN] |
| | TG | | NTGG | | | |
| BtgZI | GCGA | M.FatI | CATG | 1, 4 | m5C | CATGCGATGNNNNNNNNNN[NNNN] |
| | TG | | | | | |
| BtgZI | GCGA | M.FisAW1I, M.ThaI, M.Tph12270II | CGCG | 1, 4 | m4C | CGCGCGATGNNNNNNNNNN[NNNN], |
| | TG | | | | | CGCGATGNNNNNNNNNN[NNNN] |
| BtgZI | GCGA | M.FnoB1II, M.Hal24586I, M.Hpy298V, | CCGG | 1, 4 | m4C | CCGGCGATGNNNNNNNNNN[NNNN] |
| | TG | M.Hpy299IX, M.Hpy30V, M.Hpy32XI, | | | | |
| | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| BtgZI | GCGA | M.HhaI, M.HinP1I, M.Hpy99III, M.HpyAVIII, | GCGC | 2, 3 | m5C | GCGCGATGNNNNNNNNNN[NNNN] |
| | TG | M.HpyUM032XV | | | | |
| BtgZI | GCGA | M.Hka19301IV | TCGCGA | 2, 5 | m4C | TCGCGATGNNNNNNNNNN[NNNN] |
| | TG | | | | | |
| BtgZI | GCGA | M.Hme33500II | MGCWGCD | 5 | m4C | MGCWGCGATGNNNNNNNNNN[NNNN] |
| | TG | | | | | |
| BtgZI | GCGA | M.Hpy30VIII, M.Hpy32VIII, M.Hpy57III, | TCNNGA | 1, 6 | m6A | TCGCGATGNNNNNNNNNN[NNNN] |
| | TG | M.Hpy66VI, M.Hpy99XVIII, M.HpyFVIII, | | | | |
| | | M.HpyPVIII | | | | |
| BtgZI | GCGA | M.Hpy30X, M.Hpy57VIII, M.HpyFXI, M.MhuI | CTNAG | 1, 5 | m4C | CTNAGCGATGNNNNNNNNNN[NNNN] |
| | TG | | | | | |
| BtgZI | GCGA | M.Hpy99IV, M.HpyFORFX | CCNNGG | 1, 6 | m4C | CCNNGGCGATGNNNNNNNNNN[NNNN] |
| | TG | | | | | |
| BtgZI | GCGA | M.HpyD27I | GAGG | 2, 4 | m6A, | GAGGCGATGNNNNNNNNNN[NNNN] |
| | TG | | | | m5C | |
| BtgZI | GCGA | M.MjaII | GGNCC | 1, 5 | m5C | GCGATGGNCCNNNNNN[NNNN] |
| | TG | | | | | |
| BtgZI | GCGA | M.Msp1II | CCAGG | 5 | m4C | CCAGGCGATGNNNNNNNNNN [NNNN] |
| | TG | | | | | |
| BtgZI | GCGA | M.Msp42II | CCWGG | 1, 5 | m4C | CCWGGCGATGNNNNNNNNNN[NNNN] |
| | TG | | | | | |
| BtgZI | GCGA | M.NheI | GCTAGC | 1, 6 | m4C | GCTAGCGATGNNNNNNNNNN[NNNN], |
| | TG | | | | | GCGATGCTAGCNNNNN[NNNN] |
| BtgZI | GCGA | M.Nme18V | GCGCGC | 2, 5 | m5C | GCGCGCGATGNNNNNNNNNN[NNNN] |
| | TG | | | | | |
| BtgZI | GCGA | M.NmeDI | VCCGGY | 2, 5 | m5C | VCCGGCGATGNNNNNNNNNN[NNNN] |
| | TG | | | | | |
| BtgZI | GCGA | M.NmeMC58III | TCTGG | 5 | m5C | TCTGGCGATGNNNNNNNNNN[NNNN] |
| | TG | | | | | |
| BtgZI | GCGA | M.Nsp209III, M.Nsp51109II | CTCGAG | 1, 6 | m5C | CTCGAGCGATGNNNNNNNNNN[NNNN] |
| | TG | | | | | |
| BtgZI | GCGA | M.NspI, M.NspHI | RCATGY | 2, 5 | m5C | RCATGCGATGNNNNNNNNNN[NNNN] |
| | TG | | | | | |
| BtgZI | GCGA | M.Pam7686I | CCATGG | 1, 6 | m5C | CCATGGCGATGNNNNNNNNNN[NNNN] |
| | TG | | | | | |
| BtgZI | GCGA | M.Pfa23572II | CGATCG | 1, 6 | m5C | CGATCGCGATGNNNNNNNNNN[NNNN] |
| | TG | | | | | |
| BtgZI | GCGA | M.Pla5I | CTAG | 1, 4 | Unknown | CTAGCGATGNNNNNNNNNN[NNNN] |
| | TG | | | | | |
| BtgZI | GCGA | M.Sam53668II | CCATGG | 1, 6 | Unknown | CCATGGCGATGNNNNNNNNNN[NNNN] |
| | TG | | | | | |
| BtgZI | GCGA | M.Sen1781III | CCWWGG | 1, 6 | Unknown | CCWWGGCGATGNNNNNNNNNN[NNNN] |
| | TG | | | | | |
| BtgZI | GCGA | M.SonIII | GACNNNN | 2, 8 | m6A | GACGCGATGCNNNNNNNNN[NNNN] |
| | TG | | TGC | | | |
| BtgZI | GCGA | M.TdeII | GAAGAG | 5, 6 | m6A, | GAAGAGCGATGNNNNNNNNNN[NNNN] |
| | TG | | | | m4C | |
| BtgZI | GCGA | M.TspRI | CASTG | 1, 5 | m5C | CASTGCGATGNNNNNNNNNN[NNNN] |
| | TG | | | | | |
| BtgZI | GCGA | M.XcmI | CCANNNN | 3, 13 | m6A | CCANNNNNGCGATGGNNNNNNNNN[NNNN] |
| | TG | | NNNNNTG | | | |
| | | | G | | | |
| BtgZI | GCGA | M.Yen2516I | GCGCGC | 2, 5 | Unkno | GCGCGCGATGNNNNNNNNNN[NNNN] |
| | TG | | | | wn | |
| BtgZI | GCGA | M1.AgoG2I, M2.AgoG2I | ACTGG | 2, 5 | m4C | ACTGGCGATGNNNNNNNNNN[NNNN] |
| | TG | | | | | |
| BtgZI | GCGA | M1.BccI, M1.Hpy30XI, M2.Hpy66X, | GATGG | 2 | m6A | GCGATGGNNNNNNNNN[NNNN] |
| | TG | M1.Jsp2502I, M2.MmyCVI | | | | |
| BtgZI | GCGA | M1.BhaI, M1.Bst19I, M.Fnu10562III, | GATGC | 3 | m6A | GCGATGCNNNNNNNNN[NNNN] |
| | TG | M1.Mbo45IV, M2.Mbo45IV | | | | |
| BtgZI | GCGA | M1.BspACI | GCGG | 4 | m5C | GCGGCGATGNNNNNNNNNN[NNNN] |
| | TG | | | | | |
| BtgZI | GCGA | M1.Csp16704IV, M2.Csp16704IV, | GATGC | 2, 3 | m6A | GCGATGCNNNNNNNNN[NNNN] |
| | TG | M.Lsp10393I, M.Mha183II, M.Mha185II, | | | | |
| | | M.Mha807II, M.Mva1312II | | | | |
| BtgZI | GCGA | M1.HgaI, M2.LlaJI | GACGC | 4 | m5C | GACGCGATGNNNNNNNNNN[NNNN] |
| | TG | | | | | |
| BtgZI | GCGA | M1.Hpy299X, M2.Hpy32XIII, M1.HpyFXIII, | GATGG | 2, 3 | m6A | GCGATGGNNNNNNNNN[NNNN] |
| | TG | M2.HpyFXIII, M2.HpyUM037VI, M1.McaCI, | | | | |
| | | M2.McaCI, M1.PspHUN102II, | | | | |
| | | M2.PspHUN102II | | | | |
| BtgZI | GCGA | M1.LplAG30I | GAAGG | 5 | m5C | GAAGGCGATGNNNNNNNNNN[NNNN] |
| | TG | | | | | |
| BtgZI | GCGA | M1.Sgr13350III, M2.Sgr13350III | GAAGAGC | 5, 6 | m6A, | GAAGAGCGATGNNNNNNNNNN[NNNN] |
| | TG | | | | m4C | |
| BtgZI | GCGA | M2.BccI, M2.Hpy299X, M1.Hpy66X, | GATGG | 3 | m6A | GCGATGGNNNNNNNNN[NNNN] |
| | TG | M1.HpyC1I, M2.HpyC1I, M1.HpyUM032X, | | | | |
| | | M2.Jsp2502I, M1.MmyCVI | | | | |
| BtgZI | GCGA | M2.BceSIII, M1.Pru9I, M2.Pru9I | ACGGC | 2, 4 | m4C | ACGGCGATGNNNNNNNNNN[NNNN] |
| | TG | | | | | |
| BtgZI | GCGA | M2.BhaI | GATGC | 2 | m6A | GCGATGCNNNNNNNNN[NNNN] |
| | TG | | | | | |
| BtgZI | GCGA | M2.HpyAVI, M1.MnlI | GAGG | 4 | m5C | GAGGCGATGNNNNNNNNNN[NNNN] |
| | TG | | | | | |
| RleAI | CCCAC | M.AflIII | ACRYGT | 2, 5 | m4C | CCCACAYGTNNNNNN[NNN] |
| | A | | | | | |
| RleAI | CCCAC | M.AluBI | AGCT | 1, 4 | m6A | CCCACAGCTNNNNNN[NNN] |
| | A | | | | | |
| RleAI | CCCAC | M.ApaI | GGGCCC | 3, 4 | m5C | GGGCCCACANNNNNNNNN[NNN] |
| | A | | | | | |
| RleAI | CCCAC | M.AspDUT2II, M.BstYI, M.CagIII, | RGATCY | 2, 5 | m4C | RGATCCCACANNNNNNNNN[NNN] |
| | A | M.Mpa1757V, M.Mru1279V, M.RsaIII, | | | | |
| | | M.XhoII | | | | |
| RleAI | CCCAC | M.AspWA102I, M.AvaVII, M.BceJII, M.CbeI, | GGCC | 2, 3 | m4C | GGCCCACANNNNNNNNN[NNN] |
| | A | M.Ckr177II, M.CmaLM2IV, M.CpeAVII, | | | | |
| | | M.Csp323II, M.PhoI, M.Ssp6714III, M.SuaI | | | | |
| RleAI | CCCAC | M.Asu14238I, M.Bhy27164III, M.CagIX, | CATG | 2, 3 | m6A | CCCACATGNNNNNNN[NNN] |
| | A | M.Cje11351II, M.CviAII, M.CviQVII, | | | | |
| | | M.Fli6794III, M.Gli15749I, M.HauIII, M.HpyI, | | | | |
| | | M.Hpy166IV, M.Hpy188II, M.Hpy298I, | | | | |
| | | M.Hpy299I, M.Hpy30I, M.Hpy32I, M.Hpy57I, | | | | |
| | | M.Hpy66XI, M.Hpy99X, M.HpyAI, M.HpyFI, | | | | |
| | | M.HpyPI, M.HpyUM032I, M.HpyUM037I, | | | | |
| | | M.Lsp48II, M.LspLP1II, M.NlaIII, M.NsaI, | | | | |
| | | M.Sba3II, M.SdeAIII, M.SspT436I, M.ThaIV, | | | | |
| | | M.TvoI | | | | |
| RleAI | CCCAC | M.AvaV, M.SliSI | GATC | 1, 4 | m4C | GATCCCACANNNNNNNNN[NNN] |
| | A | | | | | |
| RleAI | CCCAC | M.BamHI | GGATCC | 2, 5 | m4C | GGATCCCACANNNNNNNNN[NNN] |
| | A | | | | | |
| RleAI | CCCAC | M.BanII, M.EcoT38I | GRGCYC | 3, 4 | m5C | GRGCCCACANNNNNNNNN[NNN] |
| | A | | | | | |
| RleAI | CCCAC | M.BanLII, M.CpeAII, M.SinI | GGWCC | 2, 4 | m5C | GGWCCCACANNNNNNNNN[NNN] |
| | A | | | | | |
| RleAI | CCCAC | M.BceSII | GGWCC | 1, 5 | m5C | GGWCCCCACANNNNNNNNN[NNN], |
| | A | | | | | GGWCCCACANNNNNNNNN[NNN] |
| RleAI | CCCAC | M.BglI, M.CagI, M.DdeVCDI, M.GsuRed1I | GCCNNNN | 2, 10 | m4C | GCCCACANGGCNNNNN[NNN] |
| | A | | NGGC | | | |
| RleAI | CCCAC | M.BhaII, M.BspRI, M.BsuRI, M.CthVI, | GGCC | 2, 3 | m5C | GGCCCACANNNNNNNNN[NNN] |
| | A | M.DthLII, M.HaeIII, M.MthTI, M.NgoAII, | | | | |
| | | M.NgoPII, M.Pod15391I | | | | |
| RleAI | CCCAC | M.Bsp14III, M.Bsp3003I, M.Bsp3004I, | GATC | 1, 4 | m5C | GATCCCACANNNNNNNNN[NNN] |
| | A | M.CalB3III, M.CpeAIII, M.Esu35585I, | | | | |
| | | M.FpsJIV, M.Lba2031I, M.Lbo2004I, | | | | |
| | | M.LlaKR2I, M.Lmo1042I, M.Lmo7956II, | | | | |
| | | M.Lsp10393II, M.MspI7I, M.Sau3AI, M.Sba3I, | | | | |
| | | M.Sin395I, M.Spn23FI, M.Sth368I | | | | |
| RleAI | CCCAC | M.BstXI | CCANNNN | 3, 10 | m6A | CCCACANNNNTGGNN[NNN] |
| | A | | NNTGG | | | |
| RleAI | CCCAC | M.CocIII, M.Fsi4947II, M.TdeIII, | GGNCC | 1, 5 | m4C | GGNCCCCACANNNNNNNNN[NNN], |
| | A | M.Tph12270I | | | | GGNCCCACANNNNNNNNN[NNN], |
| | | | | | | GGCCCACANNNNNNNNN[NNN] |
| RleAI | CCCAC | M.CviJI | RGCB | 2, 3 | m5C | VGCCCACANNNNNNNNN[NNN] |
| | A | | | | | |
| RleAI | CCCAC | M.Dac11109II, M.Hfo6591I, M.MhuII, | GTAC | 1, 4 | m4C | GTACCCACANNNNNNNNN[NNN] |
| | A | M.MjaV, M.MlaZII, M.MspLW5I, M.RsaI | | | | |
| RleAI | CCCAC | M.Djo10085II, M.Ssp6803II | GGCC | 2, 3 | Unknown | GGCCCACANNNNNNNNN[NNN] |
| | A | | | | | |
| RleAI | CCCAC | M.EcoO109I | RGGNCCY | 3, 5 | m5C | RGGNCCCACANNNNNNNNN[NNN] |
| | A | | | | | |
| RleAI | CCCAC | M.EcoVIII, M.HindIII, M.Lad60222I, M.LlaCI | AAGCTT | 1, 6 | m6A | CCCACAAGCTTNNNN[NNN] |
| | A | | | | | |
| RleAI | CCCAC | M.Esp1396I | CCANNNN | 3, 9 | m6A | CCCACANNNTGGNNN[NNN] |
| | A | | NTGG | | | |
| RleAI | CCCAC | M.FatI | CATG | 1, 4 | m5C | CCCACATGNNNNNNN[NNN] |
| | A | | | | | |
| RleAI | CCCAC | M.HgiCI | GGYRCC | 2, 5 | m5C | GGYRCCCACANNNNNNNNN[NNN] |
| | A | | | | | |
| RleAI | CCCAC | M.Hpy298II, M.Hpy299II, M.Hpy66II, | ACNGT | 2, 4 | m4C | CCCACAGTNNNNNNN[NNN] |
| | A | M.HpyUM032II, M.HpyUM037II | | | | |
| RleAI | CCCAC | M.HtuII, M.Nas12278II | CATTC | 2 | m6A | CCCACATTCNNNNNN[NNN] |
| | A | | | | | |
| RleAI | CCCAC | M.Mae7806III, M.PspPI, M.Sau96I | GGNCC | 2, 4 | m5C | GGNCCCACANNNNNNNNN[NNN], |
| | A | | | | | GGCCCACANNNNNNNNN[NNN] |
| RleAI | CCCAC | M.MjaII | GGNCC | 1, 5 | m5C | GGNCCCCACANNNNNNNNN[NNN], |
| | A | | | | | GGNCCCACANNNNNNNNN[NNN], |
| | | | | | | GGCCCACANNNNNNNNN[NNN] |
| RleAI | CCCAC | M.MpeII | RGCY | 2, 3 | m5C | RGCCCACANNNNNNNNN[NNN] |
| | A | | | | | |
| RleAI | CCCAC | M.NgoAXV, M.NgoMV | GGNNCC | 2, 5 | m5C | GGNNCCCACANNNNNNNNN[NNN], |
| | A | | | | | GGNCCCACANNNNNNNNN[NNN] |
| RleAI | CCCAC | M.NheI | GCTAGC | 1, 6 | m4C | GCTAGCCCACANNNNNNNNN[NNN] |
| | A | | | | | |
| RleAI | CCCAC | M.NspI, M.NspHI | RCATGY | 2, 5 | m5C | CCCACATGYNNNNNN[NNN] |
| | A | | | | | |
| RleAI | CCCAC | M.Psp737I | CCCCCD | 4 | m4C | CCCCCACANNNNNNNNN[NNN] |
| | A | | | | | |
| RleAI | CCCAC | M.SbaUII | CAGCTG | 2, 5 | m6A | CCCACAGCTGNNNNN[NNN] |
| | A | | | | | |
| RleAI | CCCAC | M.SuaII | RGATCY | 2, 5 | m5C | RGATCCCACANNNNNNNNN[NNN] |
| | A | | | | | |
| RleAI | CCCAC | M.TspRI | CASTG | 1, 5 | m5C | CCCACASTGNNNNNN[NNN] |
| | A | | | | | |
| RleAI | CCCAC | M1.CspNS6II | TCCGCC | 5 | m5C | TCCGCCCACANNNNNNNNN[NNN] |
| | A | | | | | |
| RleAI | CCCAC | M2.BstF5I | CATCC | 2 | m6A | CCCACATCCNNNNNN[NNN] |
| | A | | | | | |
| RleAI | CCCAC | M4.BstF5I, M1.Cin755I, M2.Cin755I, | CATCC | 2, 3 | m6A | CCCACATCCNNNNNN[NNN] |
| | A | M1.CmeSR3I, M2.CmeSR3I, M1.Csp12AI, | | | | |
| | | M1.Csp323I, M2.Csp323I, M1.Csp410I, | | | | |
| | | M2.Csp410I, M.FokI, M1.Msp315I, | | | | |
| | | M2.Msp315I, M.Sau13435I, M.StsI, | | | | |
| | | M1.Tsu2489IV, M2.Tsu2489IV, | | | | |
| | | M3.Tsu2489IV | | | | |
| AarI | CACCT | M.AbaBs12I, M.Awo1030I, M.Cdu23823I, | CCWGG | 1, 5 | m5C | CACCTGCCWGG[NNNN] |
| | GC | M.Csa8155II, M.Cun9529I, M.EasL1Dcm, | | | | |
| | | M.Eba57Dcm, M.Ecl34399Dcm, | | | | |
| | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| AarI | CACCT | M.Acc65VI, M.SenAZIII | CTKMAG | 2, 5 | m6A | CACCTGCAGNN[NNNN] |
| | GC | | | | | |
| AarI | CACCT | M.AciI | CCGC | 1, 3 | m5C | CACCTGCGGNN[NNNN], |
| | GC | | | | | CACCTGCCGCN[NNNN] |
| AarI | CACCT | M.Ade56712I, M.Asp588III, M.AveCB51II, | CTGCAG | 2, 5 | m6A | CACCTGCAGNN[NNNN] |
| | GC | M.BbrUIII, M.Blo30698I, M.BsuBI, | | | | |
| | | M.CpaYL7III, M.Dba7044I, M.Dfa45958II, | | | | |
| | | M.Eco12581I, M.Eco2012I, M.EcoGIII, | | | | |
| | | M.EcoG089I, M.Eho16691II, M.Hal5920II, | | | | |
| | | M.Lsp6406II, M.MspNR41II, M.Pae490I, | | | | |
| | | M.Pox23570I, M.PstI, M.RpaII, M.Sca3403II, | | | | |
| | | M.Tsp61I, M.XmnII, M.YenWI, M.YinY228I | | | | |
| AarI | CACCT | M.AplI | CTGCAG | 3, 4 | m5C | CACCTGCAGNN[NNNN] |
| | GC | | | | | |
| AarI | CACCT | M.AvaIX, M.Cfr10I, M.Nme18VI, M.VchAI, | RCCGGY | 2, 5 | m5C | CACCTGCCGGY[NNNN] |
| | GC | M.Vch0395I | | | | |
| AarI | CACCT | M.AvaV, M.SliSI | GATC | 1, 4 | m4C | GATCACCTGCNNNN[NNNN] |
| | GC | | | | | |
| AarI | CACCT | M.BamWS8I, M.CthV, M.EfaRFI, M.Saf8902II, | GCWGC | 2, 4 | m5C | CACCTGCWGCN[NNNN] |
| | GC | M.TacII, M.TcoKWC4III | | | | |
| AarI | CACCT | M.BbvI, M.BceSIV | GCAGC | 2, 4 | m5C | CACCTGCTGCN[NNNN], |
| | GC | | | | | CACCTGCAGCN[NNNN] |
| AarI | CACCT | M.BceSII | GGWCC | 1, 5 | m5C | GGWCCACCTGCNNNN[NNNN] |
| | GC | | | | | |
| AarI | CACCT | M.BepI, M.FpsJV, M.Gel16401III, M.Pdi8503I | CGCG | 1, 4 | m5C | CACCTGCGCGN[NNNN] |
| | GC | | | | | |
| AarI | CACCT | M.Bsp14III, M.Bsp3003I, M.Bsp3004I, | GATC | 1, 4 | m5C | GATCACCTGCNNNN[NNNN] |
| | GC | M.CalB3III, M.CpeAIII, M.Esu35585I, | | | | |
| | | M.FpsJIV, M.Lba2031I, M.Lbo2004I, | | | | |
| | | M.LlaKR2I, M.Lmo1042I, M.Lmo7956II, | | | | |
| | | M.Lsp10393II, M.MspI7I, M.Sau3AI, M.Sba3I, | | | | |
| | | M.Sin395I, M.Spn23FI, M.Sth368I | | | | |
| AarI | CACCT | M.BspRAC04I, M.EcaI, M.Eco3936IV | GGTNACC | 3, 5 | m6A | GGTCACCTGCNNNN[NNNN] |
| | GC | | | | | |
| AarI | CACCT | M.BstXI | CCANNNN | 3, 10 | m6A | CCACCTGCNTGG[NNNN] |
| | GC | | NNTGG | | | |
| AarI | CACCT | M.BsuFI, M.CpeAIV, M.MspI | CCGG | 1, 4 | m5C | CACCTGCCGGN[NNNN] |
| | GC | | | | | |
| AarI | CACCT | M.Cab13497I, M.Dac11109III, M.Dco13527I, | CTAG | 1, 4 | m4C | CACCTGCTAGN[NNNN] |
| | GC | M.Djo10085I, M.Hbo11551I, M.Hka19301III, | | | | |
| | | M.Hme33500I, M.HsaR1I, M.Hsi33800I, | | | | |
| | | M.Hsp KM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| AarI | CACCT | M.Cac8I | GCNNGC | 2, 5 | m5C | CACCTGCNNGC[NNNN] |
| | GC | | | | | |
| AarI | CACCT | M.Cdi630IV, M.Ckr177III, M.CmaLM2II, | GCNGC | 2, 4 | m5C | CACCTGCNGCN[NNNN] |
| | GC | M.CocII, M.Fnu4HI, M.Fsp4HI | | | | |
| AarI | CACCT | M.Cmu51329I, M.EcoJA03PDcm, | CCWGG | 1, 5 | Unkno | CACCTGCCWGG[NNNN] |
| | GC | M.RorR1Dcm, M.Sen8391Dcm, M.ZalSM2II | | | wn | |
| AarI | CACCT | M.CocIII, M.Fsi4947II, M.TdeIII, | GGNCC | 1, 5 | m4C | GGNCCACCTGCNNNN[NNNN] |
| | GC | M.Tph12270I | | | | |
| AarI | CACCT | M.CsiFSMII, M.MfoCT6IV, M.NgoAIV, | GCCGGC | 2, 5 | m5C | CACCTGCCGGC[NNNN] |
| | GC | M.NgoMIV, M1.Sgr13350II | | | | |
| AarI | CACCT | M.CviRI, M.Hpy298VIII, M.Hpy299VIII, | TGCA | 1, 4 | m6A | TGCACCTGCNNNN[NNNN], |
| | GC | M.Hpy30VI, M.Hpy32V, M.Hpy57II, | | | | CACCTGCANNN[NNNN] |
| | | M.Hpy66VIII, M.HpyFVI, M.HpyUM032VIII, | | | | |
| | | M.HpyUM037VIII | | | | |
| AarI | CACCT | M.Dac11109II, M.Hfo6591I, M.MhuII, | GTAC | 1, 4 | m4C | GTACACCTGCNNNN[NNNN] |
| | GC | M.MjaV, M.MlaZII, M.MspLW5I, M.RsaI | | | | |
| AarI | CACCT | M.DdeI | CTNAG | 1, 5 | m5C | CACCTGCTNAG[NNNN] |
| | GC | | | | | |
| AarI | CACCT | M.DfeI, M.Fnu419II, M.Pen113II | CTNNAG | 2, 5 | m6A | CACCTGCAGNN[NNNN] |
| | GC | | | | | |
| AarI | CACCT | M.Esp1396I | CCANNNN | 3, 9 | m6A | CCACCTGCTGGN[NNNN] |
| | GC | | NTGG | | | |
| AarI | CACCT | M.FatI | CATG | 1, 4 | m5C | CACCTGCATGN[NNNN] |
| | GC | | | | | |
| AarI | CACCT | M.FisAW1I, M.ThaI, M.Tph12270II | CGCG | 1, 4 | m4C | CACCTGCGCGN[NNNN] |
| | GC | | | | | |
| AarI | CACCT | M.FnoB1II, M.Hal24586I, M.Hpy298V, | CCGG | 1, 4 | m4C | CACCTGCCGGN[NNNN] |
| | GC | M.Hpy299IX, M.Hpy30V, M.Hpy32XI, | | | | |
| | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| AarI | CACCT | M.HgiCI | GGYRCC | 2, 5 | m5C | GGCACCTGCNNNN[NNNN] |
| | GC | | | | | |
| AarI | CACCT | M.HhaI, M.HinP1I, M.Hpy99III, M.HpyAVIII, | GCGC | 2, 3 | m5C | CACCTGCGCNN[NNNN] |
| | GC | M.HpyUM032XV | | | | |
| AarI | CACCT | M.Hme33500II | HGCWGCK | 3 | m4C | CACCTGCWGCK[NNNN] |
| | GC | | | | | |
| AarI | CACCT | M.Hpy30IX, M.Hpy32IX, M.Hpy57IX, | GTNNAC | 2, 5 | m6A | GTNCACCTGCNNNN[NNNN] |
| | GC | M.Hpy66XIII, M.Hpy8I, M.HpyFIX, | | | | |
| | | M.HpyUM032XI, M.HpyUM037V, M.MjaIV, | | | | |
| | | M.MspRAC08II | | | | |
| AarI | CACCT | M.Hpy30X, M.Hpy57VIII, M.HpyFXI, M.MhuI | CTNAG | 1, 5 | m4C | CACCTGCTNAG[NNNN] |
| | GC | | | | | |
| AarI | CACCT | M.Hpy57X | CTRYAG | 2, 5 | m6A | CACCTGCAGNN[NNNN] |
| | GC | | | | | |
| AarI | CACCT | M.Hpy99II, M.Tsp45I, M.Tsu2489II | GTSAC | 2, 4 | m6A | GTCACCTGCNNNN[NNNN] |
| | GC | | | | | |
| AarI | CACCT | M.HpyD27I | CCTC | 1, 3 | m5C, | CACCTGCCTCN[NNNN] |
| | GC | | | | m6A | |
| AarI | CACCT | M.MjaII | GGNCC | 1, 5 | m5C | GGNCCACCTGCNNNN[NNNN] |
| | GC | | | | | |
| AarI | CACCT | M.Msp1II | CCTGG | 1 | m4C | CACCTGCCTGG[NNNN] |
| | GC | | | | | |
| AarI | CACCT | M.Msp42II | CCWGG | 1, 5 | m4C | CACCTGCCWGG[NNNN] |
| | GC | | | | | |
| AarI | CACCT | M.NgoAXV, M.NgoMV | GGNNCC | 2, 5 | m5C | GGCACCTGCNNNN[NNNN] |
| | GC | | | | | |
| AarI | CACCT | M.NheI | GCTAGC | 1, 6 | m4C | GCTAGCACCTGCNNNN[NNNN], |
| | GC | | | | | CACCTGCTAGC[NNNN] |
| AarI | CACCT | M.Nme18V | GCGCGC | 2, 5 | m5C | CACCTGCGCGC[NNNN] |
| | GC | | | | | |
| AarI | CACCT | M.NmeDI | VCCGGY | 2, 5 | m5C | CACCTGCCGGB[NNNN] |
| | GC | | | | | |
| AarI | CACCT | M.NmeMC58III | CCAGA | 1 | m5C | CACCTGCCAGA[NNNN] |
| | GC | | | | | |
| AarI | CACCT | M.NspI, M.NspHI | RCATGY | 2, 5 | m5C | CACCTGCATGY [NNNN] |
| | GC | | | | | |
| AarI | CACCT | M.Pla5I | CTAG | 1, 4 | Unknown | CACCTGCTAGN[NNNN] |
| | GC | | | | | |
| AarI | CACCT | M.SonIII | GACNNNN | 2, 8 | m6A | GACCACCTGCNNNN[NNNN] |
| | GC | | TGC | | | |
| AarI | CACCT | M.TspRI | CASTG | 1, 5 | m5C | CACCTGCASTG[NNNN] |
| | GC | | | | | |
| AarI | CACCT | M.Yen2516I | GCGCGC | 2, 5 | Unknown | CACCTGCGCGC[NNNN] |
| | GC | | | | | |
| AarI | CACCT | M1.AgoG2I, M2.AgoG2I | CCAGT | 1, 4 | m4C | CACCTGCCAGT[NNNN] |
| | GC | | | | | |
| AarI | CACCT | M1.BspACI | CCGC | 1 | m5C | CACCTGCCGCN[NNNN] |
| | GC | | | | | |
| AarI | CACCT | M1.HgaI, M2.LlaJI | GCGTC | 2 | m5C | CACCTGCGTCN[NNNN] |
| | GC | | | | | |
| AarI | CACCT | M1.HphI | TCACC | 2 | m5C | TCACCTGCNNNN[NNNN] |
| | GC | | | | | |
| AarI | CACCT | M1.LplAG30I | CCTTC | 1 | m5C | CACCTGCCTTC[NNNN] |
| | GC | | | | | |
| AarI | CACCT | M2.BceSIII, M1.Pru9I, M2.Pru9I | GCCGT | 2, 4 | m4C | CACCTGCCGTN[NNNN] |
| | GC | | | | | |
| AarI | CACCT | M2.HpyAVI, M1.MnlI | CCTC | 1 | m5C | CACCTGCCTCN[NNNN] |
| | GC | | | | | |
| BspQI, LguI, PciSI, | GCTCT | M.AbaBs12I, M.Awo1030I, M.Cdu23823I, | CCWGG | 1, 5 | m5C | CCWGGCTCTTCN[NNN] |
| SapI, VpaK32I | TC | M.Csa8155II, M.Cun9529I, M.EasL1Dcm, | | | | |
| | | M.Eba57Dcm, M.Ec134399Dcm, | | | | |
| | | M.Eco12761Dcm, M.Eco3609Dcm, | | | | |
| | | M.Eco4255Dcm, M.EcoVR50Dcm, | | | | |
| | | M.EsaSP1II, M.Kpn34618Dcm, | | | | |
| | | M.Kpn38547Dcm, M.Kpn39I, M.Kpn43816I, | | | | |
| | | M.Kpn677ORFDcm, M.Kpn928Dcm, | | | | |
| | | M.KpnNIH1Dcm, M.KpnNIH10Dcm, | | | | |
| | | M.KpnNIH30Dcm, M.Kva120Dcm, M.Lsp48I, | | | | |
| | | M.Pmi1I, M.SpaD2III, M.SplRp8II, | | | | |
| | | M.SptADcm, M.Tsu2489III | | | | |
| BspQI, LguI, PciSI, | GCTCT | M.AciI | CCGC | 1, 3 | m5C | GCGGCTCTTCN[NNN], CCGCTCTTCN[NNN] |
| SapI, VpaK32I | TC | | | | | |
| BspQI, LguI, PciSI, | GCTCT | M.AluBI | AGCT | 1, 4 | m6A | AGCTCTTCN[NNN] |
| SapI, VpaK32I | TC | | | | | |
| BspQI, LguI, PciSI, | GCTCT | M.AluI, M.CspCY2I | AGCT | 2, 3 | m5C | AGCTCTTCN[NNN] |
| SapI, VpaK32I | TC | | | | | |
| BspQI, LguI, PciSI, | GCTCT | M.Ama55I, M.CnaPQ2III, M.Rmo1926II, | CGGCCG | 1, 6 | m5C | CGGCCGCTCTTCN[NNN] |
| SapI, VpaK32I | TC | M.SenC1765IV, M.SspG1I, M.XorPXII | | | | |
| BspQI, LguI, PciSI, | GCTCT | M.AquI | CYCGRG | 1, 6 | m5C | CYCGRGCTCTTCN[NNN] |
| SapI, VpaK32I | TC | | | | | |
| BspQI, LguI, PciSI, | GCTCT | M.AsiSI | GCGATCGC | 2, 7 | m5C | GCGATCGCTCTTCN[NNN] |
| SapI, VpaK32I | TC | | | | | |
| BspQI, LguI, PciSI, | GCTCT | M.AspJHL3I, M2.Psp32OWII | GAGCTC | 3, 4 | m4C | GAGCTCTTCN[NNN] |
| SapI, VpaK32I | TC | | | | | |
| BspQI, LguI, PciSI, | GCTCT | M.AvaIX, M.Cfr10I, M.Nme18VI, M.VchAI, | RCCGGY | 2, 5 | m5C | RCCGGCTCTTCN[NNN] |
| SapI, VpaK32I | TC | M.Vch0395I | | | | |
| BspQI, LguI, PciSI, | GCTCT | M.BamWS8I, M.CthV, M.EfaRFI, M.Saf8902II, | GCWGC | 2, 4 | m5C | GCWGCTCTTCN[NNN] |
| SapI, VpaK32I | TC | M.TacII, M.TcoKWC4III | | | | |
| BspQI, LguI, PciSI, | GCTCT | M.BanII, M.EcoT38I | GRGCYC | 3, 4 | m5C | GRGCTCTTCN[NNN] |
| SapI, VpaK32I | TC | | | | | |
| BspQI, LguI, PciSI, | GCTCT | M.BbvI, M.BceSIV | GCAGC | 2, 4 | m5C | GCTGCTCTTCN[NNN], |
| SapI, VpaK32I | TC | | | | | GCAGCTCTTCN[NNN] |
| BspQI, LguI, PciSI, | GCTCT | M.BcoSlacII, M.MspLW5III, M.Nbr128I, | CTCGAG | 1, 6 | m4C | CTCGAGCTCTTCN[NNN] |
| SapI, VpaK32I | TC | M.Psy18884II, M.Tam77409II, M.TbrGE1II, | | | | |
| | | M.TpaRLII | | | | |
| BspQI, LguI, PciSI, | GCTCT | M.BepI, M.FpsJV, M.Gel16401III, M.Pdi8503I | CGCG | 1, 4 | m5C | CGCGCTCTTCN[NNN] |
| SapI, VpaK32I | TC | | | | | |
| BspQI, LguI, PciSI, | GCTCT | M.BglI, M.CagI, M.DdeVCDI, M.GsuRed1I | GCCNNNN | 2, 10 | m4C | GCCNNNNNGGCTCTTCN[NNN] |
| SapI, VpaK32I | TC | | NGGC | | | |
| BspQI, LguI, PciSI, | GCTCT | M.Ble402III, M.Psp10HI | SAGCTS | 3, 4 | m4C | SAGCTCTTCN[NNN] |
| SapI, VpaK32I | TC | | | | | |
| BspQI, LguI, PciSI, | GCTCT | M.BsoBI | CYCGRG | 1, 6 | m4C | CYCGRGCTCTTCN[NNN] |
| SapI, VpaK32I | TC | | | | | |
| BspQI, LguI, PciSI, | GCTCT | M.BsuFI, M.CpeAIV, M.MspI | CCGG | 1, 4 | m5C | CCGGCTCTTCN[NNN] |
| SapI, VpaK32I | TC | | | | | |
| BspQI, LguI, PciSI, | GCTCT | M.Cab13497I, M.Dac11109III, M.Dco13527I, | CTAG | 1, 4 | m4C | CTAGCTCTTCN[NNN] |
| SapI, VpaK32I | TC | M.Djo10085I, M.Hbo11551I, M.Hka19301III, | | | | |
| | | M.Hme33500I, M.HsaR1I, M.Hsi33800I, | | | | |
| | | M.HspKM1WII, M.HspNII, M.HtuI, M.HvoDSI, | | | | |
| | | M.Lba2029II, M.Mbo45II, M.MjaI, M.MthZI, | | | | |
| | | M.Nas12278I, M.Rfl3010I, M.SsmMI | | | | |
| BspQI, LguI, PciSI, | GCTCT | M.Cac8I | GCNNGC | 2, 5 | m5C | GCNNGCTCTTCN[NNN] |
| SapI, VpaK32I | TC | | | | | |
| BspQI, LguI, PciSI, | GCTCT | M.Cdi630IV, M.Ckr177III, M.CmaLM2II, | GCNGC | 2, 4 | m5C | GCNGCTCTTCN[NNN] |
| SapI, VpaK32I | TC | M.CocII, M.Fnu4HI, M.Fsp4HI | | | | |
| BspQI, LguI, PciSI, | GCTCT | M.CfrBI, M.Sen1735IV, M.SenAnaIV | CCWWGG | 1, 6 | m4C | CCWWGGCTCTTCN[NNN] |
| SapI, VpaK32I | TC | | | | | |
| BspQI, LguI, PciSI, | GCTCT | M.Cmu51329I, M.EcoJA03PDcm, | CCWGG | 1, 5 | Unknown | CCWGGCTCTTCN[NNN] |
| SapI, VpaK32I | TC | M.RorR1Dcm, M.Sen8391Dcm, M.ZalSM2II | | | | |
| BspQI, LguI, PciSI, | GCTCT | M.CsiFSMII, M.MfoCT6IV, M.NgoAIV, | GCCGGC | 2, 5 | m5C | GCCGGCTCTTCN[NNN] |
| SapI, VpaK32I | TC | M.NgoMIV, M1.Sgr13350II | | | | |
| BspQI, LguI, PciSI, | GCTCT | M.CviJI | RGCB | 2, 3 | m5C | VGCTCTTCN[NNN] |
| SapI, VpaK32I | TC | | | | | |
| BspQI, LguI, PciSI, | GCTCT | M.DdeI | CTNAG | 1, 5 | m5C | CTNAGCTCTTCN[NNN] |
| SapI, VpaK32I | TC | | | | | |
| BspQI, LguI, PciSI, | GCTCT | M.FatI | CATG | 1, 4 | m5C | CATGCTCTTCN[NNN] |
| SapI, VpaK32I | TC | | | | | |
| BspQI, LguI, PciSI, | GCTCT | M.FisAW1I, M.ThaI, M.Tph12270II | CGCG | 1, 4 | m4C | CGCGCTCTTCN[NNN] |
| SapI, VpaK32I | TC | | | | | |
| BspQI, LguI, PciSI, | GCTCT | M.FnoB1II, M.Hal24586I, M.Hpy298V, | CCGG | 1, 4 | m4C | CCGGCTCTTCN[NNN] |
| SapI, VpaK32I | TC | M.Hpy299IX, M.Hpy30V, M.Hpy32XI, | | | | |
| | | M.Hpy57V, M.Hpy66IX, M.Hpy99VIII, | | | | |
| | | M.HpyFV, M.HpyUM032IX, M.HpyUM037IX, | | | | |
| | | M.MjaVI, M.PspJDRI, M.TmeBII | | | | |
| BspQI, LguI, PciSI, | GCTCT | M.HhaI, M.HinP1I, M.Hpy9911I, M.HpyAVIII, | GCGC | 2, 3 | m5C | GCGCTCTTCN[NNN] |
| SapI, VpaK32I | TC | M.HpyUM032XV | | | | |
| BspQI, LguI, PciSI, | GCTCT | M.Hme33500II | MGCWGCD | 5 | m4C | MGCWGCTCTTCN[NNN] |
| SapI, VpaK32I | TC | | | | | |
| BspQI, LguI, PciSI, | GCTCT | M.Hpy30X, M.Hpy57VIII, M.HpyFXI, M.MhuI | CTNAG | 1, 5 | m4C | CTNAGCTCTTCN[NNN] |
| SapI, VpaK32I | TC | | | | | |
| BspQI, LguI, PciSI, | GCTCT | M.Hpy99IV, M.HpyFORFX | CCNNGG | 1, 6 | m4C | CCNNGGCTCTTCN[NNN] |
| SapI, VpaK32I | TC | | | | | |
| BspQI, LguI, PciSI, | GCTCT | M.HpyD27I | GAGG | 2, 4 | m6A, | GAGGCTCTTCN[NNN] |
| SapI, VpaK32I | TC | | | | m5C | |
| BspQI, LguI, PciSI, | GCTCT | M.Mma5219II, M.TarMY3I | AGCT | 2, 3 | m4C | AGCTCTTCN[NNN] |
| SapI, VpaK32I | TC | | | | | |
| BspQI, LguI, PciSI, | GCTCT | M.MpeII | RGCY | 2, 3 | m5C | RGCTCTTCN[NNN] |
| SapI, VpaK32I | TC | | | | | |
| BspQI, LguI, PciSI, | GCTCT | M.Msp1II | CCAGG | 5 | m4C | CCAGGCTCTTCN[NNN] |
| SapI, VpaK32I | TC | | | | | |
| BspQI, LguI, PciSI, | GCTCT | M.Msp42II | CCWGG | 1, 5 | m4C | CCWGGCTCTTCN[NNN] |
| SapI, VpaK32I | TC | | | | | |
| BspQI, LguI, PciSI, | GCTCT | M.NgoAI | RGCGCY | 3, 4 | m5C | RGCGCTCTTCN[NNN] |
| SapI, VpaK32I | TC | | | | | |
| BspQI, LguI, PciSI, | GCTCT | M.NheI | GCTAGC | 1, 6 | m4C | GCTAGCTCTTCN[NNN] |
| SapI, VpaK32I | TC | | | | | |
| BspQI, LguI, PciSI, | GCTCT | M.Nme18V | GCGCGC | 2, 5 | m5C | GCGCGCTCTTCN[NNN] |
| SapI, VpaK32I | TC | | | | | |
| BspQI, LguI, PciSI, | GCTCT | M.NmeDI | VCCGGY | 2, 5 | m5C | VCCGGCTCTTCN[NNN] |
| SapI, VpaK32I | TC | | | | | |
| BspQI, LguI, PciSI, | GCTCT | M.NmeMC58III | TCTGG | 5 | m5C | TCTGGCTCTTCN[NNN] |
| SapI, VpaK32I | TC | | | | | |
| BspQI, LguI, PciSI, | GCTCT | M.Nsp209III, M.Nsp51109II | CTCGAG | 1, 6 | m5C | CTCGAGCTCTTCN[NNN] |
| SapI, VpaK32I | TC | | | | | |
| BspQI, LguI, PciSI, | GCTCT | M.NspI, M.NspHI | RCATGY | 2, 5 | m5C | RCATGCTCTTCN[NNN] |
| SapI, VpaK32I | TC | | | | | |
| BspQI, LguI, PciSI, | GCTCT | M.Pae10332II, M1.Pae50071I, | CAGCTC | 3, 4 | m4C | CAGCTCTTCN[NNN] |
| SapI, VpaK32I | TC | M2.Pae50071I | | | | |
| BspQI, LguI, PciSI, | GCTCT | M.Pam7686I | CCATGG | 1, 6 | m5C | CCATGGCTCTTCN[NNN] |
| SapI, VpaK32I | TC | | | | | |
| BspQI, LguI, PciSI, | GCTCT | M.Pfa23572II | CGATCG | 1, 6 | m5C | CGATCGCTCTTCN[NNN] |
| SapI, VpaK32I | TC | | | | | |
| BspQI, LguI, PciSI, | GCTCT | M.Pla5I | CTAG | 1, 4 | Unknown | CTAGCTCTTCN[NNN] |
| SapI, VpaK32I | TC | | | | | |
| BspQI, LguI, PciSI, | GCTCT | M.SacI, M.Sgr13350I | GAGCTC | 3, 4 | m5C | GAGCTCTTCN[NNN] |
| SapI, VpaK32I | TC | | | | | |
| BspQI, LguI, PciSI, | GCTCT | M.Sam53668II | CCATGG | 1, 6 | Unknown | CCATGGCTCTTCN[NNN] |
| SapI, VpaK32I | TC | | | | | |
| BspQI, LguI, PciSI, | GCTCT | M.Sen1781III | CCWWGG | 1, 6 | Unknown | CCWWGGCTCTTCN[NNN] |
| SapI, VpaK32I | TC | | | | | |
| BspQI, LguI, PciSI, | GCTCT | M.Tam77409IV | CCRCTC | 4 | m4C | CCGCTCTTCN[NNN] |
| SapI, VpaK32I | TC | | | | | |
| BspQI, LguI, PciSI, | GCTCT | M.TspRI | CASTG | 1, 5 | m5C | CASTGCTCTTCN[NNN] |
| SapI, VpaK32I | TC | | | | | |
| BspQI, LguI, PciSI, | GCTCT | M.XmnI | GAANNNN | 2, 9 | m6A | GAAGCTCTTCN[NNN] |
| SapI, VpaK32I | TC | | TTC | | | |
| BspQI, LguI, PciSI, | GCTCT | M.Yen2516I | GCGCGC | 2, 5 | Unknown | GCGCGCTCTTCN[NNN] |
| SapI, VpaK32I | TC | | | | | |
| BspQI, LguI, PciSI, | GCTCT | M1.AgoG2I, M2.AgoG2I | ACTGG | 2, 5 | m4C | ACTGGCTCTTCN[NNN] |
| SapI, VpaK32I | TC | | | | | |
| BspQI, LguI, PciSI, | GCTCT | M1.BspACI | GCGG | 4 | m5C | GCGGCTCTTCN[NNN] |
| SapI, VpaK32I | TC | | | | | |
| BspQI, LguI, PciSI, | GCTCT | M1.HgaI, M2.LlaJI | GACGC | 4 | m5C | GACGCTCTTCN[NNN] |
| SapI, VpaK32I | TC | | | | | |
| BspQI, LguI, PciSI, | GCTCT | M1.LplAG30I | GAAGG | 5 | m5C | GAAGGCTCTTCN[NNN] |
| SapI, VpaK32I | TC | | | | | |
| BspQI, LguI, PciSI, | GCTCT | M2.BceSIII, M1.Pru9I, M2.Pru9I | ACGGC | 2, 4 | m4C | ACGGCTCTTCN[NNN] |
| SapI, VpaK32I | TC | | | | | |
| BspQI, LguI, PciSI, | GCTCT | M2.HpyAVI, M1.MnlI | GAGG | 4 | m5C | GAGGCTCTTCN[NNN] |
| SapI, VpaK32I | TC | | | | | |

### Use of Other Types of Methyltransferases

Despite using type II DNA methlyase enzymes in the examples, type I restriction-modification enzymes may also be considered. Type I restriction-modification enzymes are multi-subunit enzymes that have both methyltransferase and restriction endonuclease activity. They are usually consists of three types of subunits encoded by three different genes: methylation subunit, restriction subunit and DNA sequence-recognition subunit (specificity subunit). Both the methylation subunit and specificity subunit are required for methyltransferase activity.

For example, M.EcoCI5I is known to recognize GACNNNNNNRTAAY (methylated base/opposite base in bold, reverse complementary RTTAYNNNNNNGTC). A methylation-protectable restriction site according to the invention could be provided using this sequence to protect an overlapping BsaI site: RTTAYNNNNNGGTCTC (methylated base/opposite base in bold, BsaI recognition sequence underlined, M.EcoCI5I recognition sequence in italic). An E. coli strain expressing both the methylation subunit M.EcoCI5I and the specificity subunit S.EcoCI5I may be able to methylate the corresponding bases in the cut control element, which would block the methylation-protected BsaI site.

Table 4 provides some further example restriction enzyme and type I restriction-modification enzyme combinations, or recognition sequences thereof, that may be provided in accordance with the invention. The skilled person will understand that these are examples and not an exhaustive list of potential combinations that would be encompassed by the claims.

TABLE 4

| | | Type I | | Posi- | | |
|---|---|---|---|---|---|---|
| | | restric- | | tion | | |
| | | tion- | | of | | Overlapping |
| Restric- | Restric- | modifi- | | methyl- | Type of | methylation/ |
| tion | tion | cation | Methylation | ated | methyl- | restriction |
| enzyme | site | enzyme | sequence | base | ation | site |
| AlwXI, | GCAG | M.AspU | GGCANNNN | 4, 11 | m6A | GGCAGCNNNNTGAN |
| BbvI, | C | 41III | NNTGA | | | [NNNN] |
| BseKI, | | | | | | |
| BseXI, | | | | | | |
| Bsp42 | | | | | | |
| 3I, | | | | | | |
| Bst12I, | | | | | | |
| Bst71I, | | | | | | |
| BstV1I, | | | | | | |
| GeolCI, | | | | | | |
| Lsp11 | | | | | | |
| 09I | | | | | | |
| AlwXI, | GCAG | M.Sth3 | GCANNNNN | 3, 11 | m6A | GCAGCNNNNNTCG |
| BbvI, | C | 01I | NNTCG | | | [NNNN] |
| BseKI, | | | | | | |
| BseXI, | | | | | | |
| Bsp42 | | | | | | |
| 3I, | | | | | | |
| Bst12I, | | | | | | |
| Bst71I, | | | | | | |
| BstV1I, | | | | | | |
| GeolCI, | | | | | | |
| Lsp11 | | | | | | |
| 09I | | | | | | |
| StsI | GGAT | M.Bme | GAGNNNNN | 2, 11 | m6A | GAGNNNNGGATGGNNNNN |
| | G | D34I | NNTGG | | | NNNN[NNNN] |
| BpmI, | CTGGA | M.Cba1 | GAGNNNNN | 2, 10 | m6A | CTGGAGNNNNNRTGTNNN |
| GsuI | G | 31 | RTGT | | | NN[NN] |
| BsmBI, | CGTCT | M.SmaF | CAYNNNNNN | 2, 10 | m6A | CAYNNCGTCTCA[NNNN] |
| BstGZ | C | 1I | TCA | | | |
| 53I, | | | | | | |
| Esp3I | | | | | | |
| RdeGB | ACCCA | M.Lal21 | CCAANNNNN | 4, 13 | m6A | GCACCCAGNNNTTGGNNN |
| II | G | 61 | NNNTGC | | | NNNNNNNN[NN], |
| | | | | | | CCAACCCAGNNNTGCNNNN |
| | | | | | | NNNNNNNN[NN] |

### Example Vector Sequences

The sequences of plasmids for scarless Metclo are listed below in fasta format. The underlined sequence is the “discard sequence” containing the LacZ selection fragment and a replication origin for high copy vector DNA preparation. The [symbol] is the “recessive/truncating composite element” that contains a “methylation-protectable element” and a “nonprotectable element” arranged head-to-head at appropriate distance for adapter switching. The rest of the sequence is the vector backbone containing a low copy replication origin (p15A based) and a selection marker (either kanamycin or spectinomycin).

For BsaI/M.Osp807II based scarless Metclo, the plasmids are:

$$pMXLK\_VL , pMXLK\_VM , pMXLK\_VR ( kanamycin ) \\ pMXLS\_VL , pMXLS\_VM , pMXLS\_VR ( spectinomycin )$$

For LguI/M.XmnI based scarless Metclo, the plasmids are:

$$pMZLK\_VL , pMZLK\_VM , pMZLK\_VR ( kanamycin ) \\ pMZLS\_VL , pMZLS\_VM , pMZLS\_VR ( spectinomycin )$$

The sequences of plasmids for standard 4-fragment assembly of 1 kb DNA and 2-stage assembly of ˜200 kb DNA from 28 fragments are listed in fasta format as well. The underlined sequence for 1 kb assembly vectors contain LacZ selection marker. The underlined sequence for 200 kb assembly vectors contain LacZ selection marker and a replication origin for high copy vector DNA preparation. The rest of the sequences are vector backbones.

For 1 kb assembly, the plasmids are:

$$pMXP\_A4E , pMYP\_A5E , pMZP\_P6T$$

For 200 kb assembly, the plasmids are:

$$pMXBG\_A7I , pMXBG\_I7G , pMXBG\_G7F , pMXBG\_F7E , \\ pMXBK\_A7E$$

Such vector sequences are applicable for the vectors of the examples of the invention, with the specific truncating, maintained or insertional composite elements exchanged as appropriate. A skilled person familiar with molecular cloning can re-construct the vectors herein by PCR from the commercially available Moclo kit.

>pMXLK_VL
(SEQ ID NO: 28)

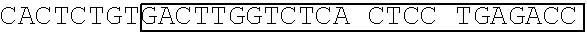

GTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGAC

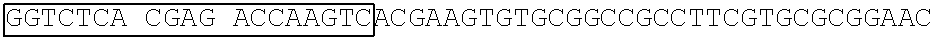

CCCTATTTGTTTATTTTTCTAAATACATTCAAATATGTATCCGCTCATGA
GACAATAACCCTGATAAATGCTTCAATAATATTGAAAAAGGAAGAGTATG
GCTAAAATGAGAATATCACCGGAATTGAAAAAACTGATCGAAAAATACCG
CTGCGTAAAAGATACGGAAGGAATGTCTCCTGCTAAGGTATATAAGCTGG
TGGGAGAAAATGAAAACCTATATTTAAAAATGACGGACAGCCGGTATAAA
GGGACCACCTATGATGTGGAACGGGAAAAGGACATGATGCTATGGCTGGA
AGGAAAGCTGCCTGTTCCAAAGGTCCTGCACTTTGAACGGCATGATGGCT
GGAGCAATCTGCTCATGAGTGAGGCCGATGGCGTCCTTTGCTCGGAAGAG
TATGAAGATGAACAAAGCCCTGAAAAGATTATCGAGCTGTATGCGGAGTG
CATCAGGCTCTTTCACTCCATCGACATATCGGATTGTCCCTATACGAATA
GCTTAGACAGCCGCTTAGCCGAATTGGATTACTTACTGAATAACGATCTG
GCCGATGTGGATTGCGAAAACTGGGAAGAGGACACTCCATTTAAAGATCC
GCGCGAGCTGTATGATTTTTTAAAGACGGAAAAGCCCGAAGAGGAACTTG
TCTTTTCCCACGGCGACCTGGGAGACAGCAACATCTTTGTGAAAGATGGC
AAAGTAAGTGGCTTTATTGATCTTGGGAGAAGCGGCAGGGCGGACAAGTG
GTATGACATTGCCTTCTGCGTCCGGTCGCTCAGGGAGGATATCGGGGAAG
AACAGTATGTCGAGCTATTTTTTGACTTACTGGGGATCAAGCCTGATTGG
GAGAAAATAAAATATTATATTTTACTGGATGAATTGTTTTAGGAGCCTGT
TTAATAAGATGATCTTCTTGAGATCGTTTTGGTCTGCGCGTAATCTCTTG
CTCTGAAAACGAAAAAACCGCCTTGCAGGGCGGTTTTTCGAAGGTTCTCT
GAGCTACCAACTCTTTGAACCGAGGTAACTGGCTTGGAGGAGCGCAGTCA
CCAAAACTTGTCCTTTCAGTTTAGCCTTAACCGGCGCATGACTTCAAGAC
TAACTCCTCTAAATCAATTACCAGTGGCTGCTGCCAGTGGTGCTTTTGCA
TGTCTTTCCGGGTTGGACTCAAGACGATAGTTACCGGATAAGGCGCAGCG
GTCGGACTGAACGGGGGGTTCGTGCATACAGTCCAGCTTGGAGCGAACTG
CCTACCCGGAACTGAGTGTCAGGCGTGGAATGAGACAAACGCGGCCATAA
CAGCGGAATGACACCGGTAAACCGAAAGGCAGGAACAGGAGAGCGCACGA
GGGAGCCGCCAGGGGGAAACGCCTGGTATCTTTATAGTCCTGTCGGGTTT
CGCCACCACTGATTTGAGCGTCAGATTTCGTGATGCTTGTCAGGGGGGCG
GAGCCTATGGAAAAACGGCTTTGCCGCGGCCCTCTCACTTCCCTGTTAAG
TATCTTCCTGGCATCTTCCAGGAAATCTCCGCCCCGTTCGTAAGCCATTT
CCGCTCGCCGCAGTCGAACGACCGAGCGTAGCGAGTCAGTGAGCGAGGAA
GCGGAATATATCCTGTATCACATATTCTGCTGACGCACCGGTGCAGCCTT
TTTTCTCCTGCCACATGAAGCACTTCACTGACACCCTCATCAGTGCCAAC
ATAGTAAGCCAGTATACACTCCGCTAGCGCGCAAGCGGCCGCA
>pMXLK_VM
(SEQ ID NO: 29)

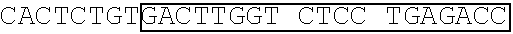

GTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGAC

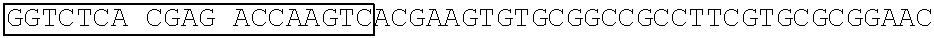

CCCTATTTGTTTATTTTTCTAAATACATTCAAATATGTATCCGCTCATGA
GACAATAACCCTGATAAATGCTTCAATAATATTGAAAAAGGAAGAGTATG
GCTAAAATGAGAATATCACCGGAATTGAAAAAACTGATCGAAAAATACCG
CTGCGTAAAAGATACGGAAGGAATGTCTCCTGCTAAGGTATATAAGCTGG
TGGGAGAAAATGAAAACCTATATTTAAAAATGACGGACAGCCGGTATAAA
GGGACCACCTATGATGTGGAACGGGAAAAGGACATGATGCTATGGCTGGA
AGGAAAGCTGCCTGTTCCAAAGGTCCTGCACTTTGAACGGCATGATGGCT
GGAGCAATCTGCTCATGAGTGAGGCCGATGGCGTCCTTTGCTCGGAAGAG
TATGAAGATGAACAAAGCCCTGAAAAGATTATCGAGCTGTATGCGGAGTG
CATCAGGCTCTTTCACTCCATCGACATATCGGATTGTCCCTATACGAATA
GCTTAGACAGCCGCTTAGCCGAATTGGATTACTTACTGAATAACGATCTG
GCCGATGTGGATTGCGAAAACTGGGAAGAGGACACTCCATTTAAAGATCC
GCGCGAGCTGTATGATTTTTTAAAGACGGAAAAGCCCGAAGAGGAACTTG
TCTTTTCCCACGGCGACCTGGGAGACAGCAACATCTTTGTGAAAGATGGC
AAAGTAAGTGGCTTTATTGATCTTGGGAGAAGCGGCAGGGCGGACAAGTG
GTATGACATTGCCTTCTGCGTCCGGTCGCTCAGGGAGGATATCGGGGAAG
AACAGTATGTCGAGCTATTTTTTGACTTACTGGGGATCAAGCCTGATTGG
GAGAAAATAAAATATTATATTTTACTGGATGAATTGTTTTAGGAGCCTGT
TTAATAAGATGATCTTCTTGAGATCGTTTTGGTCTGCGCGTAATCTCTTG
CTCTGAAAACGAAAAAACCGCCTTGCAGGGCGGTTTTTCGAAGGTTCTCT
GAGCTACCAACTCTTTGAACCGAGGTAACTGGCTTGGAGGAGCGCAGTCA
CCAAAACTTGTCCTTTCAGTTTAGCCTTAACCGGCGCATGACTTCAAGAC
TAACTCCTCTAAATCAATTACCAGTGGCTGCTGCCAGTGGTGCTTTTGCA
TGTCTTTCCGGGTTGGACTCAAGACGATAGTTACCGGATAAGGCGCAGCG
GTCGGACTGAACGGGGGGTTCGTGCATACAGTCCAGCTTGGAGCGAACTG
CCTACCCGGAACTGAGTGTCAGGCGTGGAATGAGACAAACGCGGCCATAA
CAGCGGAATGACACCGGTAAACCGAAAGGCAGGAACAGGAGAGCGCACGA
GGGAGCCGCCAGGGGGAAACGCCTGGTATCTTTATAGTCCTGTCGGGTTT
CGCCACCACTGATTTGAGCGTCAGATTTCGTGATGCTTGTCAGGGGGGCG
GAGCCTATGGAAAAACGGCTTTGCCGCGGCCCTCTCACTTCCCTGTTAAG
TATCTTCCTGGCATCTTCCAGGAAATCTCCGCCCCGTTCGTAAGCCATTT
CCGCTCGCCGCAGTCGAACGACCGAGCGTAGCGAGTCAGTGAGCGAGGAA
GCGGAATATATCCTGTATCACATATTCTGCTGACGCACCGGTGCAGCCTT
TTTTCTCCTGCCACATGAAGCACTTCACTGACACCCTCATCAGTGCCAAC
ATAGTAAGCCAGTATACACTCCGCTAGCGCGCAAGCGGCCGCA
>pMXLK_VR
(SEQ ID NO: 30)

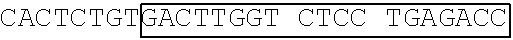

GTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGAC

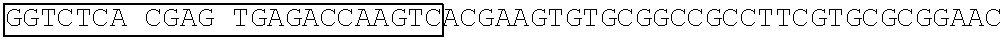

CCCTATTTGTTTATTTTTCTAAATACATTCAAATATGTATCCGCTCATGA
GACAATAACCCTGATAAATGCTTCAATAATATTGAAAAAGGAAGAGTATG
GCTAAAATGAGAATATCACCGGAATTGAAAAAACTGATCGAAAAATACCG
CTGCGTAAAAGATACGGAAGGAATGTCTCCTGCTAAGGTATATAAGCTGG
TGGGAGAAAATGAAAACCTATATTTAAAAATGACGGACAGCCGGTATAAA
GGGACCACCTATGATGTGGAACGGGAAAAGGACATGATGCTATGGCTGGA
AGGAAAGCTGCCTGTTCCAAAGGTCCTGCACTTTGAACGGCATGATGGCT
GGAGCAATCTGCTCATGAGTGAGGCCGATGGCGTCCTTTGCTCGGAAGAG
TATGAAGATGAACAAAGCCCTGAAAAGATTATCGAGCTGTATGCGGAGTG
CATCAGGCTCTTTCACTCCATCGACATATCGGATTGTCCCTATACGAATA
GCTTAGACAGCCGCTTAGCCGAATTGGATTACTTACTGAATAACGATCTG
GCCGATGTGGATTGCGAAAACTGGGAAGAGGACACTCCATTTAAAGATCC
GCGCGAGCTGTATGATTTTTTAAAGACGGAAAAGCCCGAAGAGGAACTTG
TCTTTTCCCACGGCGACCTGGGAGACAGCAACATCTTTGTGAAAGATGGC
AAAGTAAGTGGCTTTATTGATCTTGGGAGAAGCGGCAGGGCGGACAAGTG
GTATGACATTGCCTTCTGCGTCCGGTCGCTCAGGGAGGATATCGGGGAAG
AACAGTATGTCGAGCTATTTTTTGACTTACTGGGGATCAAGCCTGATTGG
GAGAAAATAAAATATTATATTTTACTGGATGAATTGTTTTAGGAGCCTGT
TTAATAAGATGATCTTCTTGAGATCGTTTTGGTCTGCGCGTAATCTCTTG
CTCTGAAAACGAAAAAACCGCCTTGCAGGGCGGTTTTTCGAAGGTTCTCT
GAGCTACCAACTCTTTGAACCGAGGTAACTGGCTTGGAGGAGCGCAGTCA
CCAAAACTTGTCCTTTCAGTTTAGCCTTAACCGGCGCATGACTTCAAGAC
TAACTCCTCTAAATCAATTACCAGTGGCTGCTGCCAGTGGTGCTTTTGCA
TGTCTTTCCGGGTTGGACTCAAGACGATAGTTACCGGATAAGGCGCAGCG
GTCGGACTGAACGGGGGGTTCGTGCATACAGTCCAGCTTGGAGCGAACTG
CCTACCCGGAACTGAGTGTCAGGCGTGGAATGAGACAAACGCGGCCATAA
CAGCGGAATGACACCGGTAAACCGAAAGGCAGGAACAGGAGAGCGCACGA
GGGAGCCGCCAGGGGGAAACGCCTGGTATCTTTATAGTCCTGTCGGGTTT
CGCCACCACTGATTTGAGCGTCAGATTTCGTGATGCTTGTCAGGGGGGCG
GAGCCTATGGAAAAACGGCTTTGCCGCGGCCCTCTCACTTCCCTGTTAAG
TATCTTCCTGGCATCTTCCAGGAAATCTCCGCCCCGTTCGTAAGCCATTT
CCGCTCGCCGCAGTCGAACGACCGAGCGTAGCGAGTCAGTGAGCGAGGAA
GCGGAATATATCCTGTATCACATATTCTGCTGACGCACCGGTGCAGCCTT
TTTTCTCCTGCCACATGAAGCACTTCACTGACACCCTCATCAGTGCCAAC
ATAGTAAGCCAGTATACACTCCGCTAGCGCGCAAGCGGCCGCA
>pMZLK_VL
(SEQ ID NO: 31)

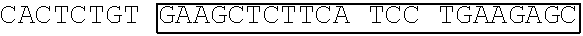

GGTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGACC

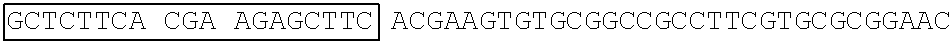

CCCTATTTGTTTATTTTTCTAAATACATTCAAATATGTATCCGCTCATGA
GACAATAACCCTGATAAATGCTTCAATAATATTGAAAAAGGAAGAGTATG
GCTAAAATGAGAATATCACCGGAATTGAAAAAACTGATCGAAAAATACCG
CTGCGTAAAAGATACGGAAGGAATGTCTCCTGCTAAGGTATATAAGCTGG
TGGGAGAAAATGAAAACCTATATTTAAAAATGACGGACAGCCGGTATAAA
GGGACCACCTATGATGTGGAACGGGAAAAGGACATGATGCTATGGCTGGA
AGGAAAGCTGCCTGTTCCAAAGGTCCTGCACTTTGAACGGCATGATGGCT
GGAGCAATCTGCTCATGAGTGAGGCCGATGGCGTCCTTTGCTCGGAAGAG
TATGAAGATGAACAAAGCCCTGAAAAGATTATCGAGCTGTATGCGGAGTG
CATCAGGCTCTTTCACTCCATCGACATATCGGATTGTCCCTATACGAATA
GCTTAGACAGCCGCTTAGCCGAATTGGATTACTTACTGAATAACGATCTG
GCCGATGTGGATTGCGAAAACTGGGAAGAGGACACTCCATTTAAAGATCC
GCGCGAGCTGTATGATTTTTTAAAGACGGAAAAGCCCGAAGAGGAACTTG
TCTTTTCCCACGGCGACCTGGGAGACAGCAACATCTTTGTGAAAGATGGC
AAAGTAAGTGGCTTTATTGATCTTGGGAGAAGCGGCAGGGCGGACAAGTG
GTATGACATTGCCTTCTGCGTCCGGTCGCTCAGGGAGGATATCGGGGAAG
AACAGTATGTCGAGCTATTTTTTGACTTACTGGGGATCAAGCCTGATTGG
GAGAAAATAAAATATTATATTTTACTGGATGAATTGTTTTAGGAGCCTGT
TTAATAAGATGATCTTCTTGAGATCGTTTTGGTCTGCGCGTAATCTCTTG
CTCTGAAAACGAAAAAACCGCCTTGCAGGGCGGTTTTTCGAAGGTTCTCT
GAGCTACCAACTCTTTGAACCGAGGTAACTGGCTTGGAGGAGCGCAGTCA
CCAAAACTTGTCCTTTCAGTTTAGCCTTAACCGGCGCATGACTTCAAGAC
TAACTCCTCTAAATCAATTACCAGTGGCTGCTGCCAGTGGTGCTTTTGCA
TGTCTTTCCGGGTTGGACTCAAGACGATAGTTACCGGATAAGGCGCAGCG
GTCGGACTGAACGGGGGGTTCGTGCATACAGTCCAGCTTGGAGCGAACTG
CCTACCCGGAACTGAGTGTCAGGCGTGGAATGAGACAAACGCGGCCATAA
CAGCGGAATGACACCGGTAAACCGAAAGGCAGGAACAGGAGAGCGCACGA
GGGAGCCGCCAGGGGGAAACGCCTGGTATCTTTATAGTCCTGTCGGGTTT
CGCCACCACTGATTTGAGCGTCAGATTTCGTGATGCTTGTCAGGGGGGCG
GAGCCTATGGAAAAACGGCTTTGCCGCGGCCCTCTCACTTCCCTGTTAAG
TATCTTCCTGGCATCTTCCAGGAAATCTCCGCCCCGTTCGTAAGCCATTT
CCGCTCGCCGCAGTCGAACGACCGAGCGTAGCGAGTCAGTGAGCGAGGAA
GCGGAATATATCCTGTATCACATATTCTGCTGACGCACCGGTGCAGCCTT
TTTTCTCCTGCCACATGAAGCACTTCACTGACACCCTCATCAGTGCCAAC
ATAGTAAGCCAGTATACACTCCGCTAGCGCGCAAGCGGCCGCA
>pMZLK_VM
(SEQ ID NO: 32)

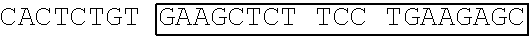

GGTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGACC

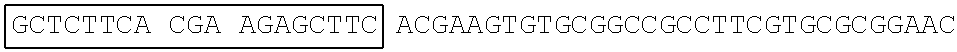

CCCTATTTGTTTATTTTTCTAAATACATTCAAATATGTATCCGCTCATGA
GACAATAACCCTGATAAATGCTTCAATAATATTGAAAAAGGAAGAGTATG
GCTAAAATGAGAATATCACCGGAATTGAAAAAACTGATCGAAAAATACCG
CTGCGTAAAAGATACGGAAGGAATGTCTCCTGCTAAGGTATATAAGCTGG
TGGGAGAAAATGAAAACCTATATTTAAAAATGACGGACAGCCGGTATAAA
GGGACCACCTATGATGTGGAACGGGAAAAGGACATGATGCTATGGCTGGA
AGGAAAGCTGCCTGTTCCAAAGGTCCTGCACTTTGAACGGCATGATGGCT
GGAGCAATCTGCTCATGAGTGAGGCCGATGGCGTCCTTTGCTCGGAAGAG
TATGAAGATGAACAAAGCCCTGAAAAGATTATCGAGCTGTATGCGGAGTG
CATCAGGCTCTTTCACTCCATCGACATATCGGATTGTCCCTATACGAATA
GCTTAGACAGCCGCTTAGCCGAATTGGATTACTTACTGAATAACGATCTG
GCCGATGTGGATTGCGAAAACTGGGAAGAGGACACTCCATTTAAAGATCC
GCGCGAGCTGTATGATTTTTTAAAGACGGAAAAGCCCGAAGAGGAACTTG
TCTTTTCCCACGGCGACCTGGGAGACAGCAACATCTTTGTGAAAGATGGC
AAAGTAAGTGGCTTTATTGATCTTGGGAGAAGCGGCAGGGCGGACAAGTG
GTATGACATTGCCTTCTGCGTCCGGTCGCTCAGGGAGGATATCGGGGAAG
AACAGTATGTCGAGCTATTTTTTGACTTACTGGGGATCAAGCCTGATTGG
GAGAAAATAAAATATTATATTTTACTGGATGAATTGTTTTAGGAGCCTGT
TTAATAAGATGATCTTCTTGAGATCGTTTTGGTCTGCGCGTAATCTCTTG
CTCTGAAAACGAAAAAACCGCCTTGCAGGGCGGTTTTTCGAAGGTTCTCT
GAGCTACCAACTCTTTGAACCGAGGTAACTGGCTTGGAGGAGCGCAGTCA
CCAAAACTTGTCCTTTCAGTTTAGCCTTAACCGGCGCATGACTTCAAGAC
TAACTCCTCTAAATCAATTACCAGTGGCTGCTGCCAGTGGTGCTTTTGCA
TGTCTTTCCGGGTTGGACTCAAGACGATAGTTACCGGATAAGGCGCAGCG
GTCGGACTGAACGGGGGGTTCGTGCATACAGTCCAGCTTGGAGCGAACTG
CCTACCCGGAACTGAGTGTCAGGCGTGGAATGAGACAAACGCGGCCATAA
CAGCGGAATGACACCGGTAAACCGAAAGGCAGGAACAGGAGAGCGCACGA
GGGAGCCGCCAGGGGGAAACGCCTGGTATCTTTATAGTCCTGTCGGGTTT
CGCCACCACTGATTTGAGCGTCAGATTTCGTGATGCTTGTCAGGGGGGCG
GAGCCTATGGAAAAACGGCTTTGCCGCGGCCCTCTCACTTCCCTGTTAAG
TATCTTCCTGGCATCTTCCAGGAAATCTCCGCCCCGTTCGTAAGCCATTT
CCGCTCGCCGCAGTCGAACGACCGAGCGTAGCGAGTCAGTGAGCGAGGAA
GCGGAATATATCCTGTATCACATATTCTGCTGACGCACCGGTGCAGCCTT
TTTTCTCCTGCCACATGAAGCACTTCACTGACACCCTCATCAGTGCCAAC
ATAGTAAGCCAGTATACACTCCGCTAGCGCGCAAGCGGCCGCA
>pMZLK_VR
(SEQ ID NO: 33)

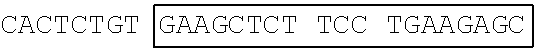

GGTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGACC

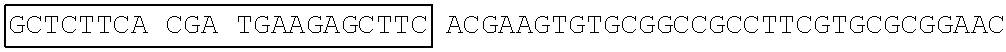

CCCTATTTGTTTATTTTTCTAAATACATTCAAATATGTATCCGCTCATGA
GACAATAACCCTGATAAATGCTTCAATAATATTGAAAAAGGAAGAGTATG
GCTAAAATGAGAATATCACCGGAATTGAAAAAACTGATCGAAAAATACCG
CTGCGTAAAAGATACGGAAGGAATGTCTCCTGCTAAGGTATATAAGCTGG
TGGGAGAAAATGAAAACCTATATTTAAAAATGACGGACAGCCGGTATAAA
GGGACCACCTATGATGTGGAACGGGAAAAGGACATGATGCTATGGCTGGA
AGGAAAGCTGCCTGTTCCAAAGGTCCTGCACTTTGAACGGCATGATGGCT
GGAGCAATCTGCTCATGAGTGAGGCCGATGGCGTCCTTTGCTCGGAAGAG
TATGAAGATGAACAAAGCCCTGAAAAGATTATCGAGCTGTATGCGGAGTG
CATCAGGCTCTTTCACTCCATCGACATATCGGATTGTCCCTATACGAATA
GCTTAGACAGCCGCTTAGCCGAATTGGATTACTTACTGAATAACGATCTG
GCCGATGTGGATTGCGAAAACTGGGAAGAGGACACTCCATTTAAAGATCC
GCGCGAGCTGTATGATTTTTTAAAGACGGAAAAGCCCGAAGAGGAACTTG
TCTTTTCCCACGGCGACCTGGGAGACAGCAACATCTTTGTGAAAGATGGC
AAAGTAAGTGGCTTTATTGATCTTGGGAGAAGCGGCAGGGCGGACAAGTG
GTATGACATTGCCTTCTGCGTCCGGTCGCTCAGGGAGGATATCGGGGAAG
AACAGTATGTCGAGCTATTTTTTGACTTACTGGGGATCAAGCCTGATTGG
GAGAAAATAAAATATTATATTTTACTGGATGAATTGTTTTAGGAGCCTGT
TTAATAAGATGATCTTCTTGAGATCGTTTTGGTCTGCGCGTAATCTCTTG
CTCTGAAAACGAAAAAACCGCCTTGCAGGGCGGTTTTTCGAAGGTTCTCT
GAGCTACCAACTCTTTGAACCGAGGTAACTGGCTTGGAGGAGCGCAGTCA
CCAAAACTTGTCCTTTCAGTTTAGCCTTAACCGGCGCATGACTTCAAGAC
TAACTCCTCTAAATCAATTACCAGTGGCTGCTGCCAGTGGTGCTTTTGCA
TGTCTTTCCGGGTTGGACTCAAGACGATAGTTACCGGATAAGGCGCAGCG
GTCGGACTGAACGGGGGGTTCGTGCATACAGTCCAGCTTGGAGCGAACTG
CCTACCCGGAACTGAGTGTCAGGCGTGGAATGAGACAAACGCGGCCATAA
CAGCGGAATGACACCGGTAAACCGAAAGGCAGGAACAGGAGAGCGCACGA
GGGAGCCGCCAGGGGGAAACGCCTGGTATCTTTATAGTCCTGTCGGGTTT
CGCCACCACTGATTTGAGCGTCAGATTTCGTGATGCTTGTCAGGGGGGCG
GAGCCTATGGAAAAACGGCTTTGCCGCGGCCCTCTCACTTCCCTGTTAAG
TATCTTCCTGGCATCTTCCAGGAAATCTCCGCCCCGTTCGTAAGCCATTT
CCGCTCGCCGCAGTCGAACGACCGAGCGTAGCGAGTCAGTGAGCGAGGAA
GCGGAATATATCCTGTATCACATATTCTGCTGACGCACCGGTGCAGCCTT
TTTTCTCCTGCCACATGAAGCACTTCACTGACACCCTCATCAGTGCCAAC
ATAGTAAGCCAGTATACACTCCGCTAGCGCGCAAGCGGCCGCA
>pMXLS_VL
(SEQ ID NO: 34)

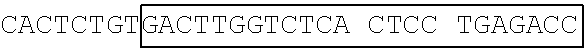

GTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGAC

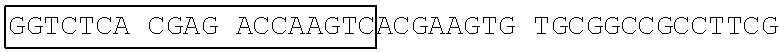

TGCGCGGAACCCCTATTTGTTTATTTTTCTAAATACATTCAAATATGTAT
CCGCTCATGAGACAATAACCCTGATAAATGCTTCAATAATATTGAAAAAG
GAAGAGTATGCGCTCACGCAACTGGTCCAGAACCTTGACCGAACGCAGCG
GTGGTAACGGCGCAGTGGCGGTTTTCATGGCTTGTTATGACTGTTTTTTTTT
GGGGTACAGTCTATGCCTCGGGCATCCAAGCAGCAAGCGCGTTACGCCGT
GGGTCGATGTTTGATGTTATGGAGCAGCAACGATGTTACGCAGCAGGGCA
GTCGCCCTAAAACAAAGTTAAACATCATGAGGGAAGCGGTGATCGCCGAA
GTATCGACTCAACTATCAGAGGTAGTTGGCGTCATCGAGCGCCATCTCGA
ACCGACGTTGCTGGCCGTACATTTGTACGGCTCCGCAGTGGATGGCGGCC
TGAAGCCACACAGCGATATTGATTTGCTGGTTACGGTGACCGTAAGGCTT
GATGAAACAACGCGGCGAGCTTTGATCAACGACCTTTTGGAAACTTCGGC
TTCCCCTGGAGAGAGCGAGATTCTCCGCGCTGTAGAAGTCACCATTGTTG
TGCACGACGACATCATTCCGTGGCGTTATCCAGCTAAGCGCGAACTGCAA
TTTGGAGAATGGCAGCGCAATGACATTCTTGCAGGTATCTTCGAGCCAGC
CACGATCGACATTGATCTGGCTATCTTGCTGACAAAAGCAAGAGAACATA
GCGTTGCCTTGGTAGGTCCAGCGGCGGAGGAACTCTTTGATCCGGTTCCT
GAACAGGATCTATTTGAGGCGCTAAATGAAACCTTAACGCTATGGAACTC
GCCGCCCGACTGGGCTGGCGATGAGCGAAATGTAGTGCTTACGTTGTCCC
GCATTTGGTACAGCGCAGTAACCGGCAAAATCGCGCCGAAGGATGTCGCT
GCCGACTGGGCAATGGAGCGCCTGCCGGCCCAGTATCAGCCCGTCATACT
TGAAGCTAGACAGGCTTATCTTGGACAAGAAGAAGATCGCTTGGCCTCGC
GCGCAGATCAGTTGGAAGAATTTGTCCATTACGTAAAAGGCGAGATCACC
AAGGTAGTCGGCAAATAAGAGCCTGTTTAATAAGATGATCTTCTTGAGAT
CGTTTTGGTCTGCGCGTAATCTCTTGCTCTGAAAACGAAAAAACCGCCTT
GCAGGGCGGTTTTTCGAAGGTTCTCTGAGCTACCAACTCTTTGAACCGAG
GTAACTGGCTTGGAGGAGCGCAGTCACCAAAACTTGTCCTTTCAGTTTAG
CCTTAACCGGCGCATGACTTCAAGACTAACTCCTCTAAATCAATTACCAG
TGGCTGCTGCCAGTGGTGCTTTTGCATGTCTTTCCGGGTTGGACTCAAGA
CGATAGTTACCGGATAAGGCGCAGCGGTCGGACTGAACGGGGGGTTCGTG
CATACAGTCCAGCTTGGAGCGAACTGCCTACCCGGAACTGAGTGTCAGGC
GTGGAATGAGACAAACGCGGCCATAACAGCGGAATGACACCGGTAAACCG
AAAGGCAGGAACAGGAGAGCGCACGAGGGAGCCGCCAGGGGGAAACGCCT
GGTATCTTTATAGTCCTGTCGGGTTTCGCCACCACTGATTTGAGCGTCAG
ATTTCGTGATGCTTGTCAGGGGGGCGGAGCCTATGGAAAAACGGCTTTGC
CGCGGCCCTCTCACTTCCCTGTTAAGTATCTTCCTGGCATCTTCCAGGAA
ATCTCCGCCCCGTTCGTAAGCCATTTCCGCTCGCCGCAGTCGAACGACCG
AGCGTAGCGAGTCAGTGAGCGAGGAAGCGGAATATATCCTGTATCACATA
TTCTGCTGACGCACCGGTGCAGCCTTTTTTCTCCTGCCACATGAAGCACT
TCACTGACACCCTCATCAGTGCCAACATAGTAAGCCAGTATACACTCCGC
TAGCGCGCAAGCGGCCGCA
>pMXLS_VM
(SEQ ID NO: 35)

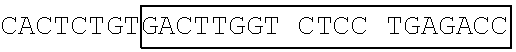

GTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGAC

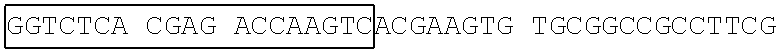

TGCGCGGAACCCCTATTTGTTTATTTTTCTAAATACATTCAAATATGTAT
CCGCTCATGAGACAATAACCCTGATAAATGCTTCAATAATATTGAAAAAG
GAAGAGTATGCGCTCACGCAACTGGTCCAGAACCTTGACCGAACGCAGCG
GTGGTAACGGCGCAGTGGCGGTTTTCATGGCTTGTTATGACTGTTTTTTTTT
GGGGTACAGTCTATGCCTCGGGCATCCAAGCAGCAAGCGCGTTACGCCGT
GGGTCGATGTTTGATGTTATGGAGCAGCAACGATGTTACGCAGCAGGGCA
GTCGCCCTAAAACAAAGTTAAACATCATGAGGGAAGCGGTGATCGCCGAA
GTATCGACTCAACTATCAGAGGTAGTTGGCGTCATCGAGCGCCATCTCGA
ACCGACGTTGCTGGCCGTACATTTGTACGGCTCCGCAGTGGATGGCGGCC
TGAAGCCACACAGCGATATTGATTTGCTGGTTACGGTGACCGTAAGGCTT
GATGAAACAACGCGGCGAGCTTTGATCAACGACCTTTTGGAAACTTCGGC
TTCCCCTGGAGAGAGCGAGATTCTCCGCGCTGTAGAAGTCACCATTGTTG
TGCACGACGACATCATTCCGTGGCGTTATCCAGCTAAGCGCGAACTGCAA
TTTGGAGAATGGCAGCGCAATGACATTCTTGCAGGTATCTTCGAGCCAGC
CACGATCGACATTGATCTGGCTATCTTGCTGACAAAAGCAAGAGAACATA
GCGTTGCCTTGGTAGGTCCAGCGGCGGAGGAACTCTTTGATCCGGTTCCT
GAACAGGATCTATTTGAGGCGCTAAATGAAACCTTAACGCTATGGAACTC
GCCGCCCGACTGGGCTGGCGATGAGCGAAATGTAGTGCTTACGTTGTCCC
GCATTTGGTACAGCGCAGTAACCGGCAAAATCGCGCCGAAGGATGTCGCT
GCCGACTGGGCAATGGAGCGCCTGCCGGCCCAGTATCAGCCCGTCATACT
TGAAGCTAGACAGGCTTATCTTGGACAAGAAGAAGATCGCTTGGCCTCGC
GCGCAGATCAGTTGGAAGAATTTGTCCATTACGTAAAAGGCGAGATCACC
AAGGTAGTCGGCAAATAAGAGCCTGTTTAATAAGATGATCTTCTTGAGAT
CGTTTTGGTCTGCGCGTAATCTCTTGCTCTGAAAACGAAAAAACCGCCTT
GCAGGGCGGTTTTTCGAAGGTTCTCTGAGCTACCAACTCTTTGAACCGAG
GTAACTGGCTTGGAGGAGCGCAGTCACCAAAACTTGTCCTTTCAGTTTAG
CCTTAACCGGCGCATGACTTCAAGACTAACTCCTCTAAATCAATTACCAG
TGGCTGCTGCCAGTGGTGCTTTTGCATGTCTTTCCGGGTTGGACTCAAGA
CGATAGTTACCGGATAAGGCGCAGCGGTCGGACTGAACGGGGGGTTCGTG
CATACAGTCCAGCTTGGAGCGAACTGCCTACCCGGAACTGAGTGTCAGGC
GTGGAATGAGACAAACGCGGCCATAACAGCGGAATGACACCGGTAAACCG
AAAGGCAGGAACAGGAGAGCGCACGAGGGAGCCGCCAGGGGGAAACGCCT
GGTATCTTTATAGTCCTGTCGGGTTTCGCCACCACTGATTTGAGCGTCAG
ATTTCGTGATGCTTGTCAGGGGGGCGGAGCCTATGGAAAAACGGCTTTGC
CGCGGCCCTCTCACTTCCCTGTTAAGTATCTTCCTGGCATCTTCCAGGAA
ATCTCCGCCCCGTTCGTAAGCCATTTCCGCTCGCCGCAGTCGAACGACCG
AGCGTAGCGAGTCAGTGAGCGAGGAAGCGGAATATATCCTGTATCACATA
TTCTGCTGACGCACCGGTGCAGCCTTTTTTCTCCTGCCACATGAAGCACT
TCACTGACACCCTCATCAGTGCCAACATAGTAAGCCAGTATACACTCCGC
TAGCGCGCAAGCGGCCGCA
>pMXLS_VR
(SEQ ID NO: 36)

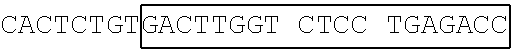

GTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGAC

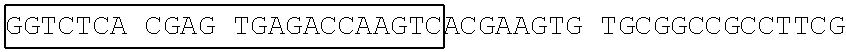

TGCGCGGAACCCCTATTTGTTTATTTTTCTAAATACATTCAAATATGTAT
CCGCTCATGAGACAATAACCCTGATAAATGCTTCAATAATATTGAAAAAG
GAAGAGTATGCGCTCACGCAACTGGTCCAGAACCTTGACCGAACGCAGCG
GTGGTAACGGCGCAGTGGCGGTTTTCATGGCTTGTTATGACTGTTTTTTTTT
GGGGTACAGTCTATGCCTCGGGCATCCAAGCAGCAAGCGCGTTACGCCGT
GGGTCGATGTTTGATGTTATGGAGCAGCAACGATGTTACGCAGCAGGGCA
GTCGCCCTAAAACAAAGTTAAACATCATGAGGGAAGCGGTGATCGCCGAA
GTATCGACTCAACTATCAGAGGTAGTTGGCGTCATCGAGCGCCATCTCGA
ACCGACGTTGCTGGCCGTACATTTGTACGGCTCCGCAGTGGATGGCGGCC
TGAAGCCACACAGCGATATTGATTTGCTGGTTACGGTGACCGTAAGGCTT
GATGAAACAACGCGGCGAGCTTTGATCAACGACCTTTTGGAAACTTCGGC
TTCCCCTGGAGAGAGCGAGATTCTCCGCGCTGTAGAAGTCACCATTGTTG
TGCACGACGACATCATTCCGTGGCGTTATCCAGCTAAGCGCGAACTGCAA
TTTGGAGAATGGCAGCGCAATGACATTCTTGCAGGTATCTTCGAGCCAGC
CACGATCGACATTGATCTGGCTATCTTGCTGACAAAAGCAAGAGAACATA
GCGTTGCCTTGGTAGGTCCAGCGGCGGAGGAACTCTTTGATCCGGTTCCT
GAACAGGATCTATTTGAGGCGCTAAATGAAACCTTAACGCTATGGAACTC
GCCGCCCGACTGGGCTGGCGATGAGCGAAATGTAGTGCTTACGTTGTCCC
GCATTTGGTACAGCGCAGTAACCGGCAAAATCGCGCCGAAGGATGTCGCT
GCCGACTGGGCAATGGAGCGCCTGCCGGCCCAGTATCAGCCCGTCATACT
TGAAGCTAGACAGGCTTATCTTGGACAAGAAGAAGATCGCTTGGCCTCGC
GCGCAGATCAGTTGGAAGAATTTGTCCATTACGTAAAAGGCGAGATCACC
AAGGTAGTCGGCAAATAAGAGCCTGTTTAATAAGATGATCTTCTTGAGAT
CGTTTTGGTCTGCGCGTAATCTCTTGCTCTGAAAACGAAAAAACCGCCTT
GCAGGGCGGTTTTTCGAAGGTTCTCTGAGCTACCAACTCTTTGAACCGAG
GTAACTGGCTTGGAGGAGCGCAGTCACCAAAACTTGTCCTTTCAGTTTAG
CCTTAACCGGCGCATGACTTCAAGACTAACTCCTCTAAATCAATTACCAG
TGGCTGCTGCCAGTGGTGCTTTTGCATGTCTTTCCGGGTTGGACTCAAGA
CGATAGTTACCGGATAAGGCGCAGCGGTCGGACTGAACGGGGGGTTCGTG
CATACAGTCCAGCTTGGAGCGAACTGCCTACCCGGAACTGAGTGTCAGGC
GTGGAATGAGACAAACGCGGCCATAACAGCGGAATGACACCGGTAAACCG
AAAGGCAGGAACAGGAGAGCGCACGAGGGAGCCGCCAGGGGGAAACGCCT
GGTATCTTTATAGTCCTGTCGGGTTTCGCCACCACTGATTTGAGCGTCAG
ATTTCGTGATGCTTGTCAGGGGGGCGGAGCCTATGGAAAAACGGCTTTGC
CGCGGCCCTCTCACTTCCCTGTTAAGTATCTTCCTGGCATCTTCCAGGAA
ATCTCCGCCCCGTTCGTAAGCCATTTCCGCTCGCCGCAGTCGAACGACCG
AGCGTAGCGAGTCAGTGAGCGAGGAAGCGGAATATATCCTGTATCACATA
TTCTGCTGACGCACCGGTGCAGCCTTTTTTCTCCTGCCACATGAAGCACT
TCACTGACACCCTCATCAGTGCCAACATAGTAAGCCAGTATACACTCCGC
TAGCGCGCAAGCGGCCGCA
>pMZLS_VL
(SEQ ID NO: 37)

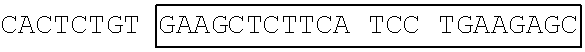

GGTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGACC

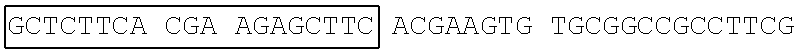

TGCGCGGAACCCCTATTTGTTTATTTTTCTAAATACATTCAAATATGTAT
CCGCTCATGAGACAATAACCCTGATAAATGCTTCAATAATATTGAAAAAG
GAAGAGTATGCGCTCACGCAACTGGTCCAGAACCTTGACCGAACGCAGCG
GTGGTAACGGCGCAGTGGCGGTTTTCATGGCTTGTTATGACTGTTTTTTTTT
GGGGTACAGTCTATGCCTCGGGCATCCAAGCAGCAAGCGCGTTACGCCGT
GGGTCGATGTTTGATGTTATGGAGCAGCAACGATGTTACGCAGCAGGGCA
GTCGCCCTAAAACAAAGTTAAACATCATGAGGGAAGCGGTGATCGCCGAA
GTATCGACTCAACTATCAGAGGTAGTTGGCGTCATCGAGCGCCATCTCGA
ACCGACGTTGCTGGCCGTACATTTGTACGGCTCCGCAGTGGATGGCGGCC
TGAAGCCACACAGCGATATTGATTTGCTGGTTACGGTGACCGTAAGGCTT
GATGAAACAACGCGGCGAGCTTTGATCAACGACCTTTTGGAAACTTCGGC
TTCCCCTGGAGAGAGCGAGATTCTCCGCGCTGTAGAAGTCACCATTGTTG
TGCACGACGACATCATTCCGTGGCGTTATCCAGCTAAGCGCGAACTGCAA
TTTGGAGAATGGCAGCGCAATGACATTCTTGCAGGTATCTTCGAGCCAGC
CACGATCGACATTGATCTGGCTATCTTGCTGACAAAAGCAAGAGAACATA
GCGTTGCCTTGGTAGGTCCAGCGGCGGAGGAACTCTTTGATCCGGTTCCT
GAACAGGATCTATTTGAGGCGCTAAATGAAACCTTAACGCTATGGAACTC
GCCGCCCGACTGGGCTGGCGATGAGCGAAATGTAGTGCTTACGTTGTCCC
GCATTTGGTACAGCGCAGTAACCGGCAAAATCGCGCCGAAGGATGTCGCT
GCCGACTGGGCAATGGAGCGCCTGCCGGCCCAGTATCAGCCCGTCATACT
TGAAGCTAGACAGGCTTATCTTGGACAAGAAGAAGATCGCTTGGCCTCGC
GCGCAGATCAGTTGGAAGAATTTGTCCATTACGTAAAAGGCGAGATCACC
AAGGTAGTCGGCAAATAAGAGCCTGTTTAATAAGATGATCTTCTTGAGAT
CGTTTTGGTCTGCGCGTAATCTCTTGCTCTGAAAACGAAAAAACCGCCTT
GCAGGGCGGTTTTTCGAAGGTTCTCTGAGCTACCAACTCTTTGAACCGAG
GTAACTGGCTTGGAGGAGCGCAGTCACCAAAACTTGTCCTTTCAGTTTAG
CCTTAACCGGCGCATGACTTCAAGACTAACTCCTCTAAATCAATTACCAG
TGGCTGCTGCCAGTGGTGCTTTTGCATGTCTTTCCGGGTTGGACTCAAGA
CGATAGTTACCGGATAAGGCGCAGCGGTCGGACTGAACGGGGGGTTCGTG
CATACAGTCCAGCTTGGAGCGAACTGCCTACCCGGAACTGAGTGTCAGGC
GTGGAATGAGACAAACGCGGCCATAACAGCGGAATGACACCGGTAAACCG
AAAGGCAGGAACAGGAGAGCGCACGAGGGAGCCGCCAGGGGGAAACGCCT
GGTATCTTTATAGTCCTGTCGGGTTTCGCCACCACTGATTTGAGCGTCAG
ATTTCGTGATGCTTGTCAGGGGGGCGGAGCCTATGGAAAAACGGCTTTGC
CGCGGCCCTCTCACTTCCCTGTTAAGTATCTTCCTGGCATCTTCCAGGAA
ATCTCCGCCCCGTTCGTAAGCCATTTCCGCTCGCCGCAGTCGAACGACCG
AGCGTAGCGAGTCAGTGAGCGAGGAAGCGGAATATATCCTGTATCACATA
TTCTGCTGACGCACCGGTGCAGCCTTTTTTCTCCTGCCACATGAAGCACT
TCACTGACACCCTCATCAGTGCCAACATAGTAAGCCAGTATACACTCCGC
TAGCGCGCAAGCGGCCGCA
>pMZLS_VM
(SEQ ID NO: 38)

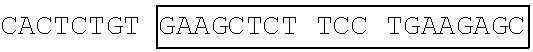

GGTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGACC

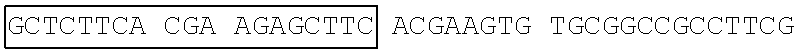

TGCGCGGAACCCCTATTTGTTTATTTTTCTAAATACATTCAAATATGTAT
CCGCTCATGAGACAATAACCCTGATAAATGCTTCAATAATATTGAAAAAG
GAAGAGTATGCGCTCACGCAACTGGTCCAGAACCTTGACCGAACGCAGCG
GTGGTAACGGCGCAGTGGCGGTTTTCATGGCTTGTTATGACTGTTTTTTTTT
GGGGTACAGTCTATGCCTCGGGCATCCAAGCAGCAAGCGCGTTACGCCGT
GGGTCGATGTTTGATGTTATGGAGCAGCAACGATGTTACGCAGCAGGGCA
GTCGCCCTAAAACAAAGTTAAACATCATGAGGGAAGCGGTGATCGCCGAA
GTATCGACTCAACTATCAGAGGTAGTTGGCGTCATCGAGCGCCATCTCGA
ACCGACGTTGCTGGCCGTACATTTGTACGGCTCCGCAGTGGATGGCGGCC
TGAAGCCACACAGCGATATTGATTTGCTGGTTACGGTGACCGTAAGGCTT
GATGAAACAACGCGGCGAGCTTTGATCAACGACCTTTTGGAAACTTCGGC
TTCCCCTGGAGAGAGCGAGATTCTCCGCGCTGTAGAAGTCACCATTGTTG
TGCACGACGACATCATTCCGTGGCGTTATCCAGCTAAGCGCGAACTGCAA
TTTGGAGAATGGCAGCGCAATGACATTCTTGCAGGTATCTTCGAGCCAGC
CACGATCGACATTGATCTGGCTATCTTGCTGACAAAAGCAAGAGAACATA
GCGTTGCCTTGGTAGGTCCAGCGGCGGAGGAACTCTTTGATCCGGTTCCT
GAACAGGATCTATTTGAGGCGCTAAATGAAACCTTAACGCTATGGAACTC
GCCGCCCGACTGGGCTGGCGATGAGCGAAATGTAGTGCTTACGTTGTCCC
GCATTTGGTACAGCGCAGTAACCGGCAAAATCGCGCCGAAGGATGTCGCT
GCCGACTGGGCAATGGAGCGCCTGCCGGCCCAGTATCAGCCCGTCATACT
TGAAGCTAGACAGGCTTATCTTGGACAAGAAGAAGATCGCTTGGCCTCGC
GCGCAGATCAGTTGGAAGAATTTGTCCATTACGTAAAAGGCGAGATCACC
AAGGTAGTCGGCAAATAAGAGCCTGTTTAATAAGATGATCTTCTTGAGAT
CGTTTTGGTCTGCGCGTAATCTCTTGCTCTGAAAACGAAAAAACCGCCTT
GCAGGGCGGTTTTTCGAAGGTTCTCTGAGCTACCAACTCTTTGAACCGAG
GTAACTGGCTTGGAGGAGCGCAGTCACCAAAACTTGTCCTTTCAGTTTAG
CCTTAACCGGCGCATGACTTCAAGACTAACTCCTCTAAATCAATTACCAG
TGGCTGCTGCCAGTGGTGCTTTTGCATGTCTTTCCGGGTTGGACTCAAGA
CGATAGTTACCGGATAAGGCGCAGCGGTCGGACTGAACGGGGGGTTCGTG
CATACAGTCCAGCTTGGAGCGAACTGCCTACCCGGAACTGAGTGTCAGGC
GTGGAATGAGACAAACGCGGCCATAACAGCGGAATGACACCGGTAAACCG
AAAGGCAGGAACAGGAGAGCGCACGAGGGAGCCGCCAGGGGGAAACGCCT
GGTATCTTTATAGTCCTGTCGGGTTTCGCCACCACTGATTTGAGCGTCAG
ATTTCGTGATGCTTGTCAGGGGGGCGGAGCCTATGGAAAAACGGCTTTGC
CGCGGCCCTCTCACTTCCCTGTTAAGTATCTTCCTGGCATCTTCCAGGAA
ATCTCCGCCCCGTTCGTAAGCCATTTCCGCTCGCCGCAGTCGAACGACCG
AGCGTAGCGAGTCAGTGAGCGAGGAAGCGGAATATATCCTGTATCACATA
TTCTGCTGACGCACCGGTGCAGCCTTTTTTCTCCTGCCACATGAAGCACT
TCACTGACACCCTCATCAGTGCCAACATAGTAAGCCAGTATACACTCCGC
TAGCGCGCAAGCGGCCGCA
>pMZLS_VR
(SEQ ID NO: 39)

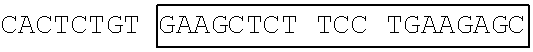

GGTCAGACCAAGTTTACTCAT
ATATACTTTAGATTGATTTAAAACTTCATTTTTAATTTAAAAGGATCTAG
GTGAAGATCCTTTTTGATAATCTCATGACCAAAATCCCTTAACGTGAGTT
TTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGATGAGCGCCCAATACGCAAACCGCCTCTCCCCGCG
CGTTGGCCGATTCATTAATGCAGCTGGCACGACAGGTTTCCCGACTGGAA
AGCGGGCAGTGAGCGCAACGCAATTAATGTGAGTTAGCTCACTCATTAGG
CACCCCAGGCTTTACACTTTATGCTTCCGGCTCGTATGTTGTGTGGAATT
GTGAGCGGATAACAATTTCACACAGGAAACAGCTATGACCATGATTACGC
CAAGCTTGCATGCCTGCAGGTCGACTCTAGAGGATCCCCGGGTACCGAGC
TCGAATTCACTGGCCGTCGTTTTACAACGTCGTGACTGGGAAAACCCTGG
CGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGC
CTGAATGGCGAATGGCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGACC

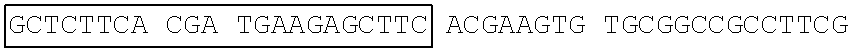

TGCGCGGAACCCCTATTTGTTTATTTTTCTAAATACATTCAAATATGTAT
CCGCTCATGAGACAATAACCCTGATAAATGCTTCAATAATATTGAAAAAG
GAAGAGTATGCGCTCACGCAACTGGTCCAGAACCTTGACCGAACGCAGCG
GTGGTAACGGCGCAGTGGCGGTTTTCATGGCTTGTTATGACTGTTTTTTTTT
GGGGTACAGTCTATGCCTCGGGCATCCAAGCAGCAAGCGCGTTACGCCGT
GGGTCGATGTTTGATGTTATGGAGCAGCAACGATGTTACGCAGCAGGGCA
GTCGCCCTAAAACAAAGTTAAACATCATGAGGGAAGCGGTGATCGCCGAA
GTATCGACTCAACTATCAGAGGTAGTTGGCGTCATCGAGCGCCATCTCGA
ACCGACGTTGCTGGCCGTACATTTGTACGGCTCCGCAGTGGATGGCGGCC
TGAAGCCACACAGCGATATTGATTTGCTGGTTACGGTGACCGTAAGGCTT
GATGAAACAACGCGGCGAGCTTTGATCAACGACCTTTTGGAAACTTCGGC
TTCCCCTGGAGAGAGCGAGATTCTCCGCGCTGTAGAAGTCACCATTGTTG
TGCACGACGACATCATTCCGTGGCGTTATCCAGCTAAGCGCGAACTGCAA
TTTGGAGAATGGCAGCGCAATGACATTCTTGCAGGTATCTTCGAGCCAGC
CACGATCGACATTGATCTGGCTATCTTGCTGACAAAAGCAAGAGAACATA
GCGTTGCCTTGGTAGGTCCAGCGGCGGAGGAACTCTTTGATCCGGTTCCT
GAACAGGATCTATTTGAGGCGCTAAATGAAACCTTAACGCTATGGAACTC
GCCGCCCGACTGGGCTGGCGATGAGCGAAATGTAGTGCTTACGTTGTCCC
GCATTTGGTACAGCGCAGTAACCGGCAAAATCGCGCCGAAGGATGTCGCT
GCCGACTGGGCAATGGAGCGCCTGCCGGCCCAGTATCAGCCCGTCATACT
TGAAGCTAGACAGGCTTATCTTGGACAAGAAGAAGATCGCTTGGCCTCGC
GCGCAGATCAGTTGGAAGAATTTGTCCATTACGTAAAAGGCGAGATCACC
AAGGTAGTCGGCAAATAAGAGCCTGTTTAATAAGATGATCTTCTTGAGAT
CGTTTTGGTCTGCGCGTAATCTCTTGCTCTGAAAACGAAAAAACCGCCTT
GCAGGGCGGTTTTTCGAAGGTTCTCTGAGCTACCAACTCTTTGAACCGAG
GTAACTGGCTTGGAGGAGCGCAGTCACCAAAACTTGTCCTTTCAGTTTAG
CCTTAACCGGCGCATGACTTCAAGACTAACTCCTCTAAATCAATTACCAG
TGGCTGCTGCCAGTGGTGCTTTTGCATGTCTTTCCGGGTTGGACTCAAGA
CGATAGTTACCGGATAAGGCGCAGCGGTCGGACTGAACGGGGGGTTCGTG
CATACAGTCCAGCTTGGAGCGAACTGCCTACCCGGAACTGAGTGTCAGGC
GTGGAATGAGACAAACGCGGCCATAACAGCGGAATGACACCGGTAAACCG
AAAGGCAGGAACAGGAGAGCGCACGAGGGAGCCGCCAGGGGGAAACGCCT
GGTATCTTTATAGTCCTGTCGGGTTTCGCCACCACTGATTTGAGCGTCAG
ATTTCGTGATGCTTGTCAGGGGGGCGGAGCCTATGGAAAAACGGCTTTGC
CGCGGCCCTCTCACTTCCCTGTTAAGTATCTTCCTGGCATCTTCCAGGAA
ATCTCCGCCCCGTTCGTAAGCCATTTCCGCTCGCCGCAGTCGAACGACCG
AGCGTAGCGAGTCAGTGAGCGAGGAAGCGGAATATATCCTGTATCACATA
TTCTGCTGACGCACCGGTGCAGCCTTTTTTCTCCTGCCACATGAAGCACT
TCACTGACACCCTCATCAGTGCCAACATAGTAAGCCAGTATACACTCCGC
TAGCGCGCAAGCGGCCGCA
>pMXP_A4E
(SEQ ID NO: 40)

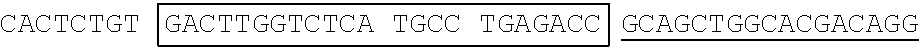

TTTCCCGACTGGAAAGCGGGCAGTGAGCGCAACGCAATTAATGTGAGTTA
GCTCACTCATTAGGCACCCCAGGCTTTACACTTTATGCTTCCGGCTCGTA
TGTTGTGTGGAATTGTGAGCGGATAACAATTTCACACAGGAAACAGCTAT
GACCATGATTACGCCAAGCTTGCATGCCTGCAGGTCGACTCTAGAGGATC
CCCGGGTACCGAGCTCGAATTCACTGGCCGTCGTTTTACAACGTCGTGAC
TGGGAAAACCCTGGCGTTACCCAACTTAATCGCCTTGCAGCACATCCCCC
TTTCGCCAGCTGGCGTAATAGCGAAGAGGCCCGCACCGATCGCCCTTCCC
AACAGTTGCGCAGCCTGAATGGCGAATGGCGCCTGATGCGGTATTTTCTC
CTTACGCATCTGTGCGGTATTTCACACCGCATATGGTGCACTCTCAGTAC
AATCTGCTCTGATGCCGCATAGTTAAGCCAGCCCCGACACCCGCCAACAC
CCGCTGACGCGCCCTGACGGGCTTGTCTGCTCCCGGCATCCGCTTACAGA

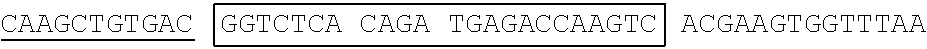

ACTATCAGTGTTTGACAGGATATATTGGCGGGTAAACCTAAGAGAAAAGA
GCGTTTATTAGAATAATCGGATATTTAAAAGGGCGTGAAAAGGTTTATCC
GTTCGTCCATTTGTATGTGCATGCCAACCACAGGGTTCCCCAGATCAGGC
GCTGGCTGCTGAACCCCCAGCCGGAACTGACCCCACAAGGCCCTAGCGTT
TGCAATGCACCAGGTCATCATTGACCCAGGCGTGTTCCACCAGGCCGCTG
CCTCGCAACTCTTCGCAGGCTTCGCCGACCTGCTCGCGCCACTTCTTCAC
GCGGGTGGAATCCGATCCGCACATGAGGCGGAAGGTTTCCAGCTTGAGCG
GGTACGGCTCCCGGTGCGAGCTGAAATAGTCGAACATCCGTCGGGCCGTC
GGCGACAGCTTGCGGTACTTCTCCCATATGAATTTCGTGTAGTGGTCGCC
AGCAAACAGCACGACGATTTCCTCGTCGATCAGGACCTGGCAACGGGACG
TTTTCTTGCCACGGTCCAGGACGCGGAAGCGGTGCAGCAGCGACACCGAT
TCCAGGTGCCCAACGCGGTCGGACGTGAAGCCCATCGCCGTCGCCTGTAG
GCGCGACAGGCATTCCTCGGCCTTCGTGTAATACCGGCCATTGATCGACC
AGCCCAGGTCCTGGCAAAGCTCGTAGAACGTGAAGGTGATCGGCTCGCCG
ATAGGGGTGCGCTTCGCGTACTCCAACACCTGCTGCCACACCAGTTCGTC
ATCGTCGGCCCGCAGCTCGACGCCGGTGTAGGTGATCTTCACGTCCTTGT
TGACGTGGAAAATGACCTTGTTTTGCAGCGCCTCGCGCGGGATTTTCTTG
TTGCGCGTGGTGAACAGGGCAGAGCGGGCCGTGTCGTTTGGCATCGCTCG
CATCGTGTCCGGCCACGGCGCAATATCGAACAAGGAAAGCTGCATTTCCT
TGATCTGCTGCTTCGTGTGTTTCAGCAACGCGGCCTGCTTGGCCTCGCTG
ACCTGTTTTGCCAGGTCCTCGCCGGCGGTTTTTCGCTTCTTGGTCGTCAT
AGTTCCTCGCGTGTCGATGGTCATCGACTTCGCCAAACCTGCCGCCTCCT
GTTCAAGACGACGCGAACGCTCCACGGCGGCCGATGGCGCGGGCAGGGCA
GGGGGAGCCAGTTGCACGCTGTCGCGCTCGATCTTGGCCGTAGCTTGCTG
GACCATCGAGCCGACGGACTGGAAGGTTTCGCGGGGCGCACGCATGACGG
TGCGGCTTGCGATGGTTTCGGCATCCTCGGCGGAAAACCCCGCGTCGATC
AGTTCTTGCCTGTATGCCTTCCGGTCAAACGTCCGATTCATTCACCCTCC
TTGCGGGATTGCCCCGACTCACGCCGGGGCAATGTGCCCTTATTCCTGAT
TTGACCCGCCTGGTGCCTTGGTGTCCAGATAATCCACCTTATCGGCAATG
AAGTCGGTCCCGTAGACCGTCTGGCCGTCCTTCTCGTACTTGGTATTCCG
AATCTTGCCCTGCACGAATACCAGCGACCCCTTGCCCAAATACTTGCCGT
GGGCCTCGGCCTGAGAGCCAAAACACTTGATGCGGAAGAAGTCGGTGCGC
TCCTGCTTGTCGCCGGCATCGTTGCGCCACATCTAGGATCTGCCAGGAAC
CGTAAAAAGGCCGCGTTGCTGGCGTTTTTCCATAGGCTCCGCCCCCCTGA
CGAGCATCACAAAAATCGACGCTCAAGTCAGAGGTGGCGAAACCCGACAG
GACTATAAAGATACCAGGCGTTTCCCCCTGGAAGCTCCCTCGTGCGCTCT
CCTGTTCCGACCCTGCCGCTTACCGGATACCTGTCCGCCTTTCTCCCTTC
GGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGT
AAGACACGACTTATCGCCACTGGCAGCAGCCACTGGTAACAGGATTAGCA
GAGCGAGGTATGTAGGCGGTGCTACAGAGTTCTTGAAGTGGTGGCCTAAC
TACGGCTACACTAGAAGGACAGTATTTGGTATCTGCGCTCTGCTGAAGCC
AGTTACCTTCGGAAAAAGAGTTGGTAGCTCTTGATCCGGCAAACAAACCA
CCGCTGGTAGCGGTGGTTTTTTTGTTTGCAAGCAGCAGATTACGCGCAGA
AAAAAAGGATCTCAAGAAGATCCTTTGATCTTTTCTACGGGGTCTGACGC
TCAGTGGAACGAAAACTCACGTTAAGGGATTTTGGTCATGAGATTATCAA
AAAGGATCTTCACCTAGATCCTTTTAAATTAAAAATGAAGTTTTAAATCA
ATCTAAAGTATATATGAGTAAACTTGGTCTGACAGTTACCAATGCTTAAT
CAGTGAGGCACCTATCTCAGCGATCTGTCTATTTCGTTCATCCATAGTTG
CCTGACTCCCCGTCGTGTAGATAACTACGATACGGGAGGGCTTACCATCT
GGCCCCAGTGCTGCAATGATACCGCGAGAACCACGCTCACCGGCTCCAGA
TTTATCAGCAATAAACCAGCCAGCCGGAAGGGCCGAGCGCAGAAGTGGTC
CTGCAACTTTATCCGCCTCCATCCAGTCTATTAATTGTTGCCGGGAAGCT
AGAGTAAGTAGTTCGCCAGTTAATAGTTTGCGCAACGTTGTTGCCATTGC
TACAGGCATCGTGGTGTCACGCTCGTCGTTTGGTATGGCTTCATTCAGCT
CCGGTTCCCAACGATCAAGGCGAGTTACATGATCCCCCATGTTGTGCAAA
AAAGCGGTTAGCTCCTTCGGTCCTCCGATCGTTGTCAGAAGTAAGTTGGC
CGCAGTGTTATCACTCATGGTTATGGCAGCACTGCATAATTCTCTTACTG
TCATGCCATCCGTAAGATGCTTTTCTGTGACTGGTGAGTACTCAACCAAG
TCATTCTGAGAATAGTGTATGCGGCGACCGAGTTGCTCTTGCCCGGCGTC
AATACGGGATAATACCGCGCCACATAGCAGAACTTTAAAAGTGCTCATCA
TTGGAAAACGTTCTTCGGGGCGAAAACTCTCAAGGATCTTACCGCTGTTG
AGATCCAGTTCGATGTAACCCACTCGTGCACCCAACTGATCTTCAGCATC
TTTTACTTTCACCAGCGTTTCTGGGTGAGCAAAAACAGGAAGGCAAAATG
CCGCAAAAAAGGGAATAAGGGCGACACGGAAATGTTGAATACTCATACTC
TTCCTTTTTCAATATTATTGAAGCATTTATCAGGGTTATTGTCTCATGAG
CGGATACATATTTGAATGTATTTAGAAAAATAAACAAATAGGGGTTCCGC
GCACGAATTGGCCAGCGCTGCCATTTTTGGGGTGAGGCCGTTCGCGGCCG
AGGGGCGCAGCCCCTGGGGGGATGGGAGGCCCGCGTTAGCGGGCCGGGAG
GGTTCGAGAAGGGGGGGCACCCCCCTTCGGCGTGCGCGGTCACGCGCACA
GGGCGCAGCCCTGGTTAAAAACAAGGTTTATAAATATTGGTTTAAAAGCA
GGTTAAAAGACAGGTTAGCGGTGGCCGAAAAACGGGCGGAAACCCTTGCA
AATGCTGGATTTTCTGCCTGTGGACAGCCCCTCAAATGTCAATAGGTGCG
CCCCTCATCTGTCAGCACTCTGCCCCTCAAGTGTCAAGGATCGCGCCCCT
CATCTGTCAGTAGTCGCGCCCCTCAAGTGTCAATACCGCAGGGCACTTAT
CCCCAGGCTTGTCCACATCATCTGTGGGAAACTCGCGTAAAATCAGGCGT
TTTCGCCGATTTGCGAGGCTGGCCAGCTCCACGTCGCCGGCCGAAATCGA
GCCTGCCCCTCATCTGTCAACGCCGCGCCGGGTGAGTCGGCCCCTCAAGT
GTCAACGTCCGCCCCTCATCTGTCAGTGAGGGCCAAGTTTTCCGCGAGGT
ATCCACAACGCCGGCGGCCGCGGTGTCTCGCACACGGCTTCGACGGCGTT
TCTGGCGCGTTTGCAGGGCCATAGACGGCCGCCAGCCCAGCGGCGAGGGC
AACCAGCCCGGTGAGCGTCGCAAAGGAGATCCTGATCTGACTGATGGGCT
GCCTGTATCGAGTGGTGATTTTGTGCCGAGCTGCCGGTCGGGGAGCTGTT
GGCTGGCTGGTGGCAGGATATATTGTGGTGTAAACAAATTGACGCTTAGA
CAACTTAATAACACATTGCGGACGTTTTTAATGTACTGGGGTGGATGCAG
TGGGCCC
>pMYP_A5E
(SEQ ID NO: 41)

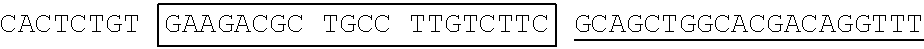

CCCGACTGGAAAGCGGGCAGTGAGCGCAACGCAATTAATGTGAGTTAGCT
CACTCATTAGGCACCCCAGGCTTTACACTTTATGCTTCCGGCTCGTATGT
TGTGTGGAATTGTGAGCGGATAACAATTTCACACAGGAAACAGCTATGAC
CATGATTACGCCAAGCTTGCATGCCTGCAGGTCGACTCTAGAGGATCCCC
GGGTACCGAGCTCGAATTCACTGGCCGTCGTTTTACAACGTCGTGACTGG
GAAAACCCTGGCGTTACCCAACTTAATCGCCTTGCAGCACATCCCCCTTT
CGCCAGCTGGCGTAATAGCGAAGAGGCCCGCACCGATCGCCCTTCCCAAC
AGTTGCGCAGCCTGAATGGCGAATGGCGCCTGATGCGGTATTTTCTCCTT
ACGCATCTGTGCGGTATTTCACACCGCATATGGTGCACTCTCAGTACAAT
CTGCTCTGATGCCGCATAGTTAAGCCAGCCCCGACACCCGCCAACACCCG
CTGACGCGCCCTGACGGGCTTGTCTGCTCCCGGCATCCGCTTACAGACAA

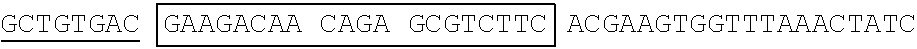

AGTGTTTGACAGGATATATTGGCGGGTAAACCTAAGAGAAAAGAGCGTTT
ATTAGAATAATCGGATATTTAAAAGGGCGTGAAAAGGTTTATCCGTTCGT
CCATTTGTATGTGCATGCCAACCACAGGGTTCCCCAGATCAGGCGCTGGC
TGCTGAACCCCCAGCCGGAACTGACCCCACAAGGCCCTAGCGTTTGCAAT
GCACCAGGTCATCATTGACCCAGGCGTGTTCCACCAGGCCGCTGCCTCGC
AACTCTTCGCAGGCTTCGCCGACCTGCTCGCGCCACTTCTTCACGCGGGT
GGAATCCGATCCGCACATGAGGCGGAAGGTTTCCAGCTTGAGCGGGTACG
GCTCCCGGTGCGAGCTGAAATAGTCGAACATCCGTCGGGCCGTCGGCGAC
AGCTTGCGGTACTTCTCCCATATGAATTTCGTGTAGTGGTCGCCAGCAAA
CAGCACGACGATTTCCTCGTCGATCAGGACCTGGCAACGGGACGTTTTCT
TGCCACGGTCCAGGACGCGGAAGCGGTGCAGCAGCGACACCGATTCCAGG
TGCCCAACGCGGTCGGACGTGAAGCCCATCGCCGTCGCCTGTAGGCGCGA
CAGGCATTCCTCGGCCTTCGTGTAATACCGGCCATTGATCGACCAGCCCA
GGTCCTGGCAAAGCTCGTAGAACGTGAAGGTGATCGGCTCGCCGATAGGG
GTGCGCTTCGCGTACTCCAACACCTGCTGCCACACCAGTTCGTCATCGTC
GGCCCGCAGCTCGACGCCGGTGTAGGTGATCTTCACGTCCTTGTTGACGT
GGAAAATGACCTTGTTTTGCAGCGCCTCGCGCGGGATTTTCTTGTTGCGC
GTGGTGAACAGGGCAGAGCGGGCCGTGTCGTTTGGCATCGCTCGCATCGT
GTCCGGCCACGGCGCAATATCGAACAAGGAAAGCTGCATTTCCTTGATCT
GCTGCTTCGTGTGTTTCAGCAACGCGGCCTGCTTGGCCTCGCTGACCTGT
TTTGCCAGGTCCTCGCCGGCGGTTTTTCGCTTCTTGGTCGTCATAGTTCC
TCGCGTGTCGATGGTCATCGACTTCGCCAAACCTGCCGCCTCCTGTTCAA
GACGACGCGAACGCTCCACGGCGGCCGATGGCGCGGGCAGGGCAGGGGGA
GCCAGTTGCACGCTGTCGCGCTCGATCTTGGCCGTAGCTTGCTGGACCAT
CGAGCCGACGGACTGGAAGGTTTCGCGGGGCGCACGCATGACGGTGCGGC
TTGCGATGGTTTCGGCATCCTCGGCGGAAAACCCCGCGTCGATCAGTTCT
TGCCTGTATGCCTTCCGGTCAAACGTCCGATTCATTCACCCTCCTTGCGG
GATTGCCCCGACTCACGCCGGGGCAATGTGCCCTTATTCCTGATTTGACC
CGCCTGGTGCCTTGGTGTCCAGATAATCCACCTTATCGGCAATGAAGTCG
GTCCCGTAGACCGTCTGGCCGTCCTTCTCGTACTTGGTATTCCGAATCTT
GCCCTGCACGAATACCAGCGACCCCTTGCCCAAATACTTGCCGTGGGCCT
CGGCCTGAGAGCCAAAACACTTGATGCGGAAGAAGTCGGTGCGCTCCTGC
TTGTCGCCGGCATCGTTGCGCCACATCTAGGATCTGCCAGGAACCGTAAA
AAGGCCGCGTTGCTGGCGTTTTTCCATAGGCTCCGCCCCCCTGACGAGCA
TCACAAAAATCGACGCTCAAGTCAGAGGTGGCGAAACCCGACAGGACTAT
AAAGATACCAGGCGTTTCCCCCTGGAAGCTCCCTCGTGCGCTCTCCTGTT
CCGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAGG
TCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACA
CGACTTATCGCCACTGGCAGCAGCCACTGGTAACAGGATTAGCAGAGCGA
GGTATGTAGGCGGTGCTACAGAGTTCTTGAAGTGGTGGCCTAACTACGGC
TACACTAGAAGGACAGTATTTGGTATCTGCGCTCTGCTGAAGCCAGTTAC
CTTCGGAAAAAGAGTTGGTAGCTCTTGATCCGGCAAACAAACCACCGCTG
GTAGCGGTGGTTTTTTTGTTTGCAAGCAGCAGATTACGCGCAGAAAAAAA
GGATCTCAAGAAGATCCTTTGATCTTTTCTACGGGGTCTGACGCTCAGTG
GAACGAAAACTCACGTTAAGGGATTTTGGTCATGAGATTATCAAAAAGGA
TCTTCACCTAGATCCTTTTAAATTAAAAATGAAGTTTTAAATCAATCTAA
AGTATATATGAGTAAACTTGGTCTGACAGTTACCAATGCTTAATCAGTGA
GGCACCTATCTCAGCGATCTGTCTATTTCGTTCATCCATAGTTGCCTGAC
TCCCCGTCGTGTAGATAACTACGATACGGGAGGGCTTACCATCTGGCCCC
AGTGCTGCAATGATACCGCGAGAACCACGCTCACCGGCTCCAGATTTATC
AGCAATAAACCAGCCAGCCGGAAGGGCCGAGCGCAGAAGTGGTCCTGCAA
CTTTATCCGCCTCCATCCAGTCTATTAATTGTTGCCGGGAAGCTAGAGTA
AGTAGTTCGCCAGTTAATAGTTTGCGCAACGTTGTTGCCATTGCTACAGG
CATCGTGGTGTCACGCTCGTCGTTTGGTATGGCTTCATTCAGCTCCGGTT
CCCAACGATCAAGGCGAGTTACATGATCCCCCATGTTGTGCAAAAAAGCG
GTTAGCTCCTTCGGTCCTCCGATCGTTGTCAGAAGTAAGTTGGCCGCAGT
GTTATCACTCATGGTTATGGCAGCACTGCATAATTCTCTTACTGTCATGC
CATCCGTAAGATGCTTTTCTGTGACTGGTGAGTACTCAACCAAGTCATTC
TGAGAATAGTGTATGCGGCGACCGAGTTGCTCTTGCCCGGCGTCAATACG
GGATAATACCGCGCCACATAGCAGAACTTTAAAAGTGCTCATCATTGGAA
AACGTTCTTCGGGGCGAAAACTCTCAAGGATCTTACCGCTGTTGAGATCC
AGTTCGATGTAACCCACTCGTGCACCCAACTGATCTTCAGCATCTTTTAC
TTTCACCAGCGTTTCTGGGTGAGCAAAAACAGGAAGGCAAAATGCCGCAA
AAAAGGGAATAAGGGCGACACGGAAATGTTGAATACTCATACTCTTCCTT
TTTCAATATTATTGAAGCATTTATCAGGGTTATTGTCTCATGAGCGGATA
CATATTTGAATGTATTTAGAAAAATAAACAAATAGGGGTTCCGCGCACGA
ATTGGCCAGCGCTGCCATTTTTGGGGTGAGGCCGTTCGCGGCCGAGGGGC
GCAGCCCCTGGGGGGATGGGAGGCCCGCGTTAGCGGGCCGGGAGGGTTCG
AGAAGGGGGGGCACCCCCCTTCGGCGTGCGCGGTCACGCGCACAGGGCGC
AGCCCTGGTTAAAAACAAGGTTTATAAATATTGGTTTAAAAGCAGGTTAA
AAGACAGGTTAGCGGTGGCCGAAAAACGGGCGGAAACCCTTGCAAATGCT
GGATTTTCTGCCTGTGGACAGCCCCTCAAATGTCAATAGGTGCGCCCCTC
ATCTGTCAGCACTCTGCCCCTCAAGTGTCAAGGATCGCGCCCCTCATCTG
TCAGTAGTCGCGCCCCTCAAGTGTCAATACCGCAGGGCACTTATCCCCAG
GCTTGTCCACATCATCTGTGGGAAACTCGCGTAAAATCAGGCGTTTTCGC
CGATTTGCGAGGCTGGCCAGCTCCACGTCGCCGGCCGAAATCGAGCCTGC
CCCTCATCTGTCAACGCCGCGCCGGGTGAGTCGGCCCCTCAAGTGTCAAC
GTCCGCCCCTCATCTGTCAGTGAGGGCCAAGTTTTCCGCGAGGTATCCAC
AACGCCGGCGGCCGCGGTGTCTCGCACACGGCTTCGACGGCGTTTCTGGC
GCGTTTGCAGGGCCATAGACGGCCGCCAGCCCAGCGGCGAGGGCAACCAG
CCCGGTGAGCGTCGCAAAGGAGATCCTGATCTGACTGATGGGCTGCCTGT
ATCGAGTGGTGATTTTGTGCCGAGCTGCCGGTCGGGGAGCTGTTGGCTGG
CTGGTGGCAGGATATATTGTGGTGTAAACAAATTGACGCTTAGACAACTT
AATAACACATTGCGGACGTTTTTAATGTACTGGGGTGGATGCAGTGGGCC
C
>pMZP_P6T
(SEQ ID NO: 42)

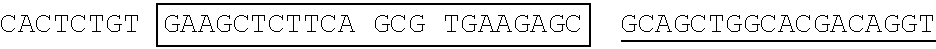

TTCCCGACTGGAAAGCGGGCAGTGAGCGCAACGCAATTAATGTGAGTTAG
CTCACTCATTAGGCACCCCAGGCTTTACACTTTATGCTTCCGGCTCGTAT
GTTGTGTGGAATTGTGAGCGGATAACAATTTCACACAGGAAACAGCTATG
ACCATGATTACGCCAAGCTTGCATGCCTGCAGGTCGACTCTAGAGGATCC
CCGGGTACCGAGCTCGAATTCACTGGCCGTCGTTTTACAACGTCGTGACT
GGGAAAACCCTGGCGTTACCCAACTTAATCGCCTTGCAGCACATCCCCCT
TTCGCCAGCTGGCGTAATAGCGAAGAGGCCCGCACCGATCGCCCTTCCCA
ACAGTTGCGCAGCCTGAATGGCGAATGGCGCCTGATGCGGTATTTTCTCC
TTACGCATCTGTGCGGTATTTCACACCGCATATGGTGCACTCTCAGTACA
ATCTGCTCTGATGCCGCATAGTTAAGCCAGCCCCGACACCCGCCAACACC
CGCTGACGCGCCCTGACGGGCTTGTCTGCTCCCGGCATCCGCTTACAGAC

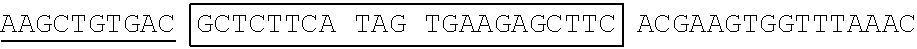

TATCAGTGTTTGACAGGATATATTGGCGGGTAAACCTAAGAGAAAAGAGC
GTTTATTAGAATAATCGGATATTTAAAAGGGCGTGAAAAGGTTTATCCGT
TCGTCCATTTGTATGTGCATGCCAACCACAGGGTTCCCCAGATCAGGCGC
TGGCTGCTGAACCCCCAGCCGGAACTGACCCCACAAGGCCCTAGCGTTTG
CAATGCACCAGGTCATCATTGACCCAGGCGTGTTCCACCAGGCCGCTGCC
TCGCAACTCTTCGCAGGCTTCGCCGACCTGCTCGCGCCACTTCTTCACGC
GGGTGGAATCCGATCCGCACATGAGGCGGAAGGTTTCCAGCTTGAGCGGG
TACGGCTCCCGGTGCGAGCTGAAATAGTCGAACATCCGTCGGGCCGTCGG
CGACAGCTTGCGGTACTTCTCCCATATGAATTTCGTGTAGTGGTCGCCAG
CAAACAGCACGACGATTTCCTCGTCGATCAGGACCTGGCAACGGGACGTT
TTCTTGCCACGGTCCAGGACGCGGAAGCGGTGCAGCAGCGACACCGATTC
CAGGTGCCCAACGCGGTCGGACGTGAAGCCCATCGCCGTCGCCTGTAGGC
GCGACAGGCATTCCTCGGCCTTCGTGTAATACCGGCCATTGATCGACCAG
CCCAGGTCCTGGCAAAGCTCGTAGAACGTGAAGGTGATCGGCTCGCCGAT
AGGGGTGCGCTTCGCGTACTCCAACACCTGCTGCCACACCAGTTCGTCAT
CGTCGGCCCGCAGCTCGACGCCGGTGTAGGTGATCTTCACGTCCTTGTTG
ACGTGGAAAATGACCTTGTTTTGCAGCGCCTCGCGCGGGATTTTCTTGTT
GCGCGTGGTGAACAGGGCAGAGCGGGCCGTGTCGTTTGGCATCGCTCGCA
TCGTGTCCGGCCACGGCGCAATATCGAACAAGGAAAGCTGCATTTCCTTG
ATCTGCTGCTTCGTGTGTTTCAGCAACGCGGCCTGCTTGGCCTCGCTGAC
CTGTTTTGCCAGGTCCTCGCCGGCGGTTTTTCGCTTCTTGGTCGTCATAG
TTCCTCGCGTGTCGATGGTCATCGACTTCGCCAAACCTGCCGCCTCCTGT
TCAAGACGACGCGAACGCTCCACGGCGGCCGATGGCGCGGGCAGGGCAGG
GGGAGCCAGTTGCACGCTGTCGCGCTCGATCTTGGCCGTAGCTTGCTGGA
CCATCGAGCCGACGGACTGGAAGGTTTCGCGGGGCGCACGCATGACGGTG
CGGCTTGCGATGGTTTCGGCATCCTCGGCGGAAAACCCCGCGTCGATCAG
TTCTTGCCTGTATGCCTTCCGGTCAAACGTCCGATTCATTCACCCTCCTT
GCGGGATTGCCCCGACTCACGCCGGGGCAATGTGCCCTTATTCCTGATTT
GACCCGCCTGGTGCCTTGGTGTCCAGATAATCCACCTTATCGGCAATGAA
GTCGGTCCCGTAGACCGTCTGGCCGTCCTTCTCGTACTTGGTATTCCGAA
TCTTGCCCTGCACGAATACCAGCGACCCCTTGCCCAAATACTTGCCGTGG
GCCTCGGCCTGAGAGCCAAAACACTTGATGCGGAAGAAGTCGGTGCGCTC
CTGCTTGTCGCCGGCATCGTTGCGCCACATCTAGGATCTGCCAGGAACCG
TAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCC
TGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCC
CGACCGCTGCGCCTTATCCGGTAACTATCGTCTTGAGTCCAACCCGGTAA
GACACGACTTATCGCCACTGGCAGCAGCCACTGGTAACAGGATTAGCAGA
GCGAGGTATGTAGGCGGTGCTACAGAGTTCTTGAAGTGGTGGCCTAACTA
CGGCTACACTAGAAGGACAGTATTTGGTATCTGCGCTCTGCTGAAGCCAG
TTACCTTCGGAAAAAGAGTTGGTAGCTCTTGATCCGGCAAACAAACCACC
GCTGGTAGCGGTGGTTTTTTTGTTTGCAAGCAGCAGATTACGCGCAGAAA
AAAAGGATCTCAAGAAGATCCTTTGATCTTTTCTACGGGGTCTGACGCTC
AGTGGAACGAAAACTCACGTTAAGGGATTTTGGTCATGAGATTATCAAAA
AGGATCTTCACCTAGATCCTTTTAAATTAAAAATGAAGTTTTAAATCAAT
CTAAAGTATATATGAGTAAACTTGGTCTGACAGTTACCAATGCTTAATCA
GTGAGGCACCTATCTCAGCGATCTGTCTATTTCGTTCATCCATAGTTGCC
TGACTCCCCGTCGTGTAGATAACTACGATACGGGAGGGCTTACCATCTGG
CCCCAGTGCTGCAATGATACCGCGAGAACCACGCTCACCGGCTCCAGATT
TATCAGCAATAAACCAGCCAGCCGGAAGGGCCGAGCGCAGAAGTGGTCCT
GCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCC
GGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCG
CAGTGTTATCACTCATGGTTATGGCAGCACTGCATAATTCTCTTACTGTC
ATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAA
TACGGGATAATACCGCGCCACATAGCAGAACTTTAAAAGTGCTCATCATT
GGAAAACGTTCTTCGGGGCGAAAACTCTCAAGGATCTTACCGCTGTTGAG
ATCCAGTTCGATGTAACCCACTCGTGCACCCAACTGATCTTCAGCATCTT
TTACTTTCACCAGCGTTTCTGGGTGAGCAAAAACAGGAAGGCAAAATGCC
GCAAAAAAGGGAATAAGGGCGACACGGAAATGTTGAATACTCATACTCTT
CCTTTTTCAATATTATTGAAGCATTTATCAGGGTTATTGTCTCATGAGCG
GATACATATTTGAATGTATTTAGAAAAATAAACAAATAGGGGTTCCGCGC
ACGAATTGGCCAGCGCTGCCATTTTTGGGGTGAGGCCGTTCGCGGCCGAG
GGGCGCAGCCCCTGGGGGGATGGGAGGCCCGCGTTAGCGGGCCGGGAGGG
TTCGAGAAGGGGGGGCACCCCCCTTCGGCGTGCGCGGTCACGCGCACAGG
GCGCAGCCCTGGTTAAAAACAAGGTTTATAAATATTGGTTTAAAAGCAGG
TTAAAAGACAGGTTAGCGGTGGCCGAAAAACGGGCGGAAACCCTTGCAAA
TGCTGGATTTTCTGCCTGTGGACAGCCCCTCAAATGTCAATAGGTGCGCC
CCTCATCTGTCAGCACTCTGCCCCTCAAGTGTCAAGGATCGCGCCCCTCA
TCTGTCAGTAGTCGCGCCCCTCAAGTGTCAATACCGCAGGGCACTTATCC
CCAGGCTTGTCCACATCATCTGTGGGAAACTCGCGTAAAATCAGGCGTTT
TCGCCGATTTGCGAGGCTGGCCAGCTCCACGTCGCCGGCCGAAATCGAGC
CTGCCCCTCATCTGTCAACGCCGCGCCGGGTGAGTCGGCCCCTCAAGTGT
CAACGTCCGCCCCTCATCTGTCAGTGAGGGCCAAGTTTTCCGCGAGGTAT
CCACAACGCCGGCGGCCGCGGTGTCTCGCACACGGCTTCGACGGCGTTTC
TGGCGCGTTTGCAGGGCCATAGACGGCCGCCAGCCCAGCGGCGAGGGCAA
CCAGCCCGGTGAGCGTCGCAAAGGAGATCCTGATCTGACTGATGGGCTGC
CTGTATCGAGTGGTGATTTTGTGCCGAGCTGCCGGTCGGGGAGCTGTTGG
CTGGCTGGTGGCAGGATATATTGTGGTGTAAACAAATTGACGCTTAGACA
ACTTAATAACACATTGCGGACGTTTTTAATGTACTGGGGTGGATGCAGTG
GGCCC
>pMXBG_A7I
(SEQ ID NO: 43)

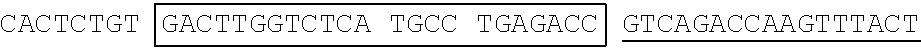

CATATATACTTTAGATTGATTTAAAACTTCATTTTTAATTTAAAAGGATC
TAGGTGAAGATCCTTTTTGATAATCTCATGACCAAAATCCCTTAACGTGA
GTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCT
TTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGG
CGATAAGTCGTGTCTTACCGGGTTGGACTCAAGACGATAGTTACCGGATA
AGGCGCAGCGGTCGGGCTGAACGGGGGGTTCGTGCACACAGCCCAGCTTG
GAGCGAACGACCTACACCGAACTGAGATACCTACAGCGTGAGCTATGAGA
AAGCGCCACGCTTCCCGAAGGGAGAAAGGCGGACAGGTATCCGGTAAGCG
GCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATG
TTCTTTCCTGCGTTATCCCCTGATTCTGTGGATAACCGTATTACCGCCTT
TGAGTGAGCTGATACCGCTCGCCGCAGCCGAACGACCGAGCGCAGCGAGT
CAGTGAGCGAGGAAGCGGATGAGCGCCCAATACGCAAACCGCCTCTCCCC
GCGCGTTGGCCGATTCATTAATGCAGCTGGCACGACAGGTTTCCCGACTG
GAAAGCGGGCAGTGAGCGCAACGCAATTAATGTGAGTTAGCTCACTCATT
AGGCACCCCAGGCTTTACACTTTATGCTTCCGGCTCGTATGTTGTGTGGA
ATTGTGAGCGGATAACAATTTCACACAGGAAACAGCTATGACCATGATTA
CGCCAAGCTTGCATGCCTGCAGGTCGACTCTAGAGGATCCCCGGGTACCG
AGCTCGAATTCACTGGCCGTCGTTTTACAACGTCGTGACTGGGAAAACCC
TGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGC
AGCCTGAATGGCGAATGGCGCCTGATGCGGTATTTTCTCCTTACGCATCT
GTGCGGTATTTCACACCGCATATGGTGCACTCTCAGTACAATCTGCTCTG
ATGCCGCATAGTTAAGCCAGCCCCGACACCCGCCAACACCCGCTGACGCG
CCCTGACGGGCTTGTCTGCTCCCGGCATCCGCTTACAGACAAGCTGTGAC

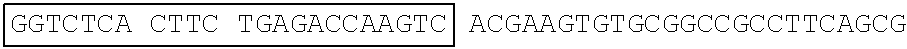

GCCGCACGAATCCATGTGGGAGTTTATTCTTGACACAGATATTTATGATA
TAATAACTGAGTAAGCTTAACATAAGGAGGAAAAACATATGTTACGCAGC
AGCAACGATGTTACGCAGCAGGGCAGTCGCCCTAAAACAAAGTTAGGTGG
CTCAAGTATGGGCATCATTCGCACATGTAGGCTCGGCCCTGACCAAGTCA
AATCCATGCGGGCTGCTCTTGATCTTTTCGGTCGTGAGTTCGGAGACGTA
GCCACCTACTCCCAACATCAGCCGGACTCCGATTACCTCGGGAACTTGCT
CCGTAGTAAGACATTCATCGCGCTTGCTGCCTTCGACCAAGAAGCGGTTG
TTGGCGCTCTCGCGGCTTACGTTCTGCCCAAGTTTGAGCAGCCGCGTAGT
GAGATCTATATCTATGATCTCGCAGTCTCCGGCGAGCACCGGAGGCAGGG
CATTGCCACCGCGCTCATCAATCTCCTCAAGCATGAGGCCAACGCGCTTG
GTGCTTATGTGATCTACGTGCAAGCAGATTACGGTGACGATCCCGCAGTG
GCTCTCTATACAAAGTTGGGCATACGGGAAGAAGTGATGCACTTTGATAT
CGACCCAAGTACCGCCACCTAAGAGCTGGAGCGGCGTAACCGTCGCACAG
GAAGGACAGAGAAAGCGCGGATCTGGGAAGTGACGGACAGAACGGTCAGG
ACCTGGATTGGGGAGGCGGTTGCCGCCGCTGCTGCTGACGGTGTGACGTT
CTCTGTTCCGGTCACACCACATACGTTCCGCCATTCCTATGCGATGCACA
TGCTGTATGCCGGTATACCGCTGAAAGTTCTGCAAAGCCTGATGGGACAT
AAGTCCATCAGTTCAACGGAAGTCTACACGAAGGTTTTTGCGCTGGATGT
GGCTGCCCGGCTCCGGGTGCAGTTTGCGATGCCGGAGTCTGATGCGGTTG
CGATGCTGAAACAATTATCCTGAGAATAAATGCCTTGGCCTTTATATGGA
AATGTGGAACTGAGTGGATATGCTGTTTTTGTCTGTTAAACAGAGAAGCT
GGCTGTTATCCACTGAGAAGCGAACGAAACAGTCGGGAAAATCTCCCATT
ATCGTAGAGATCCGCATTATTAATCTCAGGAGCCTGTGTAGCGTTTATAG
GAAGTAGTGTTCTGTCATGATGCCTGCAAGCGGTAACGAAAACGATTTGA
ATATGCCTTCAGGAACAATAGAAATCTTCGTGCGGTGTTACGTTGAAGTG
GAGCGGATTATGTCAGCAATGGACAGAACAACCTAATGAACACAGAACCA
TGATGTGGTCTGTCCTTTTACAGCCAGTAGTGCTCGCCGCAGTCGAGCGA
CAGGGCGAAGCCCTCGAGTGAGCGAGGAAGCACCAGGGAACAGCACTTAT
ATATTCTGCTTACACACGATGCCTGAAAAAACTTCCCTTGGGGTTATCCA
CTTATCCACGGGGATATTTTTATAATTATTTTTTTTATAGTTTTTAGATC
TTCTTTTTTAGAGCGCCTTGTAGGCCTTTATCCATGCTGGTTCTAGAGAA
GGTGTTGTGACAAATTGCCCTTTCAGTGTGACAAATCACCCTCAAATGAC
AGTCCTGTCTGTGACAAATTGCCCTTAACCCTGTGACAAATTGCCCTCAG
AAGAAGCTGTTTTTTCACAAAGTTATCCCTGCTTATTGACTCTTTTTTAT
TTAGTGTGACAATCTAAAAACTTGTCACACTTCACATGGATCTGTCATGG
CGGAAACAGCGGTTATCAATCACAAGAAACGTAAAAATAGCCCGCGAATC
GTCCAGTCAAACGACCTCACTGAGGCGGCATATAGTCTCTCCCGGGATCA
AAAACGTATGCTGTATCTGTTCGTTGACCAGATCAGAAAATCTGATGGCA
CCCTACAGGAACATGACGGTATCTGCGAGATCCATGTTGCTAAATATGCT
GAAATATTCGGATTGACCTCTGCGGAAGCCAGTAAGGATATACGGCAGGC
ATTGAAGAGTTTCGCGGGGAAGGAAGTGGTTTTTTATCGCCCTGAAGAGG
ATGCCGGCGATGAAAAAGGCTATGAATCTTTTCCTTGGTTTATCAAACGT
GCGCACAGTCCATCCAGAGGGCTTTACAGTGTACATATCAACCCATATCT
CATTCCCTTCTTTATCGGGTTACAGAACCGGTTTACGCAGTTTCGGCTTA
GTGAAACAAAAGAAATCACCAATCCGTATGCCATGCGTTTATACGAATCC
CTGTGTCAGTATCGTAAGCCGGATGGCTCAGGCATCGTGTCTCTGAAAAT
CGACTGGATCATAGAGCGTTACCAGCTGCCTCAAAGTTACCAGCGTATGC
CTGACTTCCGCCGCCGCTTCCTGCAGGTCTGTGTTAATGAGATCAACAGC
AGAACTCCAATGCGCCTCTCATACATTGAGAAAAAGAAAGGCCGCCAGAC
GACTCATATCGTATTTTCCTTCCGCGATATCACTTCCATGACGACAGGAT
AGTCTGAGGGTTATCTGTCACAGATTTGAGGGTGGTTCGTCACATTTGTT
CTGACCTACTGAGGGTAATTTGTCACAGTTTTGCTGTTTCCTTCAGCCTG
CATGGATTTTCTCATACTTTTTGAACTGTAATTTTTAAGGAAGCCAAATT
TGAGGGCAGTTTGTCACAGTTGATTTCCTTCTCTTTCCCTTCGTCATGTG
ACCTGATATCGGGGGTTAGTTCGTCATCATTGATGAGGGTTGATTATCAC
AGTTTATTACTCTGAATTGGCTATCCGCGTGTGTACCTCTACCTGGAGTT
TTTCCCACGGTGGATATTTCTTCTTGCGCTGAGCGTAAGAGCTATCTGAC
AGAACAGTTCTTCTTTGCTTCCTCGCCAGTTCGCTCGCTATGCTCGGTTA
CACGGCTGCGGCGAGCGCTAGTGATAATAAGTGACTGAGGTATGTGGTGT
TCTTATCTCCTTTTGTAGTGTTGCTCTTATTTTAAACAACTTTGCGGTTT
TTTGATGACTTTGCGATTTTGTTGTTGCTTTGCAGTAAATTGCAAGATTT
AATAAAAAAACGCAAAGCAATGATTAAAGGATGTTCAGAATGAAACTCAT
GGAAACACTTAACCAGTGCATAAACGCTGGTCATGAAATGACGAAGGCTA
TCGCCATTGCACAGTTTAATGATGACAGCCCGGAAGCGAGGAAAATAACC
CGGCGCTGGAGAATAGGTGAAGCAGCGGATTTAGTTGGGGTTTCTTCTCA
GGCTATCAGAGATGCCGAGAAAGCAGGGCGACTACCGCACCCGGATATGG
AAATTCGAGGACGGGTTGAGCAACGTGTTGGTTATACAATTGAACAAATT
AATCATATGCGTGATGTGTTTGGTACGCGATTGCGACGTGCTGAGGACGT
ATTTCCACCGGTGATCGGGGTTGCTGCCCATAAAGGTGGCGTTTACAAAA
CCTCAGTTTCTGTTCATCTTGCTCAGGATCTGGCTCTGAAGGGGCTACGT
GTTTTGCTCGTGGAAGGTAACGACCCCCAGGGAACAGCCTCAATGTATCA
CGGATGGGTACCAGATCTTCATATTCATGCAGAAGATACTCTCCTGCCTT
TCTATCTTGGGGAAAAGGACGATGTCACTTATGCAATAAAGCCCACTTGC
TGGCCGGGGCTTGACATTATTCCTTCCTGTCTGGCTCTGCACCGTATTGA
AACTGAGTTAATGGGCAAATTTGATGAAGGTAAACTGCCCACCGATCCAC
ACCTGATGCTCCGACTGGCCATTGAAACTGTTGCTCATGACTATGATGTC
ATAGTTATTGACAGCGCGCCTAACCTGGGTATCGGCACGATTAATGTCGT
ATGTGCTGCTGATGTGCTGATTGTTCCCACGCCTGCTGAGTIGTTTGACT
ACACCTCCGCACTGCAGTTTTTCGATATGCTTCGTGATCTGCTCAAGAAC
GTTGATCTTAAAGGGTTCGAGCCTGATGTACGTATTTTGCTTACCAAATA
CAGCAATAGTAATGGCTCTCAGTCCCCGTGGATGGAGGAGCAAATTCGGG
ATGCCTGGGGAAGCATGGTTCTAAAAAATGTTGTACGTGAAACGGATGAA
GTTGGTAAAGGTCAGATCCGGATGAGAACTGTTTTTGAACAGGCCATTGA
TCAACGCAGTTCAACTGGTGCCTGGAGAAATGCTCTTTCTATTTGGGAAC
CTGTCTGCAATGAAATTTTCGATCGTCTGATTAAACCACGCTGGGAGATT
AGATAATGAAGCGTGCGCCTGTTATTCCAAAACATACGCTCAATACTCAA
CCGGTTGAAGATACTTCGTTATCGACACCAGCTGCCCCGATGGTGGATTC
GTTAATTGCGCGCGTAGGAGTAATGGCTCGCGGTAATGCCATTACTTTGC
CTGTATGTGGTCGGGATGTGAAGTTTACTCTTGAAGTGCTCCGGGGTGAT
AGTGTTGAGAAAACCTCTCGGGTATGGTCAGGTAATGAACGTGACCAGGA
GCTGCTTACTGAGGACGCACTGGATGATCTCATCCCTTCTTTTCTACTGA
CTGGTCAACAGACACCGGCGTTCGGTCGAAGAGTATCTGGTGTCATAGAA
ATTGCCGATGGGAGTCGCCGTCGTAAAGCTGCTGCACTTACCGAAAGTGA
TTATCGTGTTCTGGTTGGCGAGCTGGATGATGAGCAGATGGCTGCATTAT
CCAGATTGGGTAACGATTATCGCCCAACAAGTGCTTATGAACGTGGTCAG
CGTTATGCAAGCCGATTGCAGAATGAATTTGCTGGAAATATTTCTGCGCT
GGCTGATGCGGAAAATATTTCACGTAAGATTATTACCCGCTGTATCAACA
CCGCCAAATTGCCTAAATCAGTTGTTGCTCTTTTTTCTCACCCCGGTGAA
CTATCTGCCCGGTCAGGTGATGCACTTCAAAAAGCCTTTACAGATAAAGA
GGAATTACTTAAGCAGCAGGCATCTAACCTTCATGAGCAGAAAAAAGCTG
GGGTGATATTTGAAGCTGAAGAAGTTATCACTCTTTTAACTTCTGTGCTT
AAAACGTCATCTGCATCAAGAACTAGTTTAAGCTCACGACATCAGTTTGC
TCCTGGAGCGACAGTATTGTATAAGGGCGATAAAATGGTGCTTAACCTGG
ACAGGTCACGTGTTCCAACTGAGTGTATAGAGAAAATTGAGGCCATTCTT
AAGGAACTTGAAAAGCCAGCACCCTGATGCGACCACGTTTTAGTCTACGT
TTATCTGTCTTTACTTAATGTCCTTTGTTACAGGCCAGAAAGCATAACTG
GCCTGAATATTCTCTCTGGGCCCACTGTTCCACTTGTATCGTCGGTCTGA
TAATCAGACTGGGACCACGGTCCCACTCGTATCGTCGGTCTGATTATTAG
TCTGGGACCACGGTCCCACTCGTATCGTCGGTCTGATTATTAGTCTGGGA
CCACGGTCCCACTCGTATCGTCGGTCTGATAATCAGACTGGGACCACGGT
CCCACTCGTATCGTCGGTCTGATTATTAGTCTGGGACCATGGTCCCACTC
GTATCGTCGGTCTGATTATTAGTCTGGGACCACGGTCCCACTCGTATCGT
CGGTCTGATTATTAGTCTGGAACCACGGTCCCACTCGTATCGTCGGTCTG
ATTATTAGTCTGGGACCACGGTCCCACTCGTATCGTCGGTCTGATTATTA
GTCTGGGACCACGATCCCACTCGTGTTGTCGGTCTGATTATCGGTCTGGG
ACCACGGTCCCACTTGTATTGTCGATCAGACTATCAGCGTGAGACTACGA
TTCCATCAATGCCTGTCAAGGGCAAGTATTGACATGTCGTCGTAACCTGT
AGAACGGAGTAACCTCGGTGTGCGGTTGTATGCCTGCTGTGGATTGCTGC
TGTGTCCTGCTTATCCACAACATTTTGCGCACGGTTATGTGGACAAAATA
CCTGGTTACCCAGGCCGTGCCGGCACGTTAACCGGGCTGCATCCGATGCA
AGTGTGTCGCTGTCGACGCAAGCGGCCGCA
>pMXBG_I7G
(SEQ ID NO: 44)

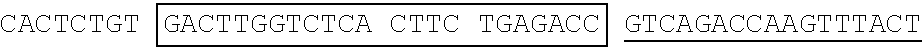

CATATATACTTTAGATTGATTTAAAACTTCATTTTTAATTTAAAAGGATC
TAGGTGAAGATCCTTTTTGATAATCTCATGACCAAAATCCCTTAACGTGA
GTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCT
TTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGG
CGATAAGTCGTGTCTTACCGGGTTGGACTCAAGACGATAGTTACCGGATA
AGGCGCAGCGGTCGGGCTGAACGGGGGGTTCGTGCACACAGCCCAGCTTG
GAGCGAACGACCTACACCGAACTGAGATACCTACAGCGTGAGCTATGAGA
AAGCGCCACGCTTCCCGAAGGGAGAAAGGCGGACAGGTATCCGGTAAGCG
GCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATG
TTCTTTCCTGCGTTATCCCCTGATTCTGTGGATAACCGTATTACCGCCTT
TGAGTGAGCTGATACCGCTCGCCGCAGCCGAACGACCGAGCGCAGCGAGT
CAGTGAGCGAGGAAGCGGATGAGCGCCCAATACGCAAACCGCCTCTCCCC
GCGCGTTGGCCGATTCATTAATGCAGCTGGCACGACAGGTTTCCCGACTG
GAAAGCGGGCAGTGAGCGCAACGCAATTAATGTGAGTTAGCTCACTCATT
AGGCACCCCAGGCTTTACACTTTATGCTTCCGGCTCGTATGTTGTGTGGA
ATTGTGAGCGGATAACAATTTCACACAGGAAACAGCTATGACCATGATTA
CGCCAAGCTTGCATGCCTGCAGGTCGACTCTAGAGGATCCCCGGGTACCG
AGCTCGAATTCACTGGCCGTCGTTTTACAACGTCGTGACTGGGAAAACCC
TGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGC
AGCCTGAATGGCGAATGGCGCCTGATGCGGTATTTTCTCCTTACGCATCT
GTGCGGTATTTCACACCGCATATGGTGCACTCTCAGTACAATCTGCTCTG
ATGCCGCATAGTTAAGCCAGCCCCGACACCCGCCAACACCCGCTGACGCG
CCCTGACGGGCTTGTCTGCTCCCGGCATCCGCTTACAGACAAGCTGTGAC

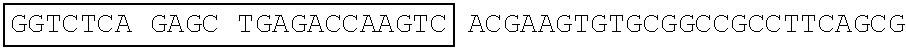

GCCGCACGAATCCATGTGGGAGTTTATTCTTGACACAGATATTTATGATA
TAATAACTGAGTAAGCTTAACATAAGGAGGAAAAACATATGTTACGCAGC
AGCAACGATGTTACGCAGCAGGGCAGTCGCCCTAAAACAAAGTTAGGTGG
CTCAAGTATGGGCATCATTCGCACATGTAGGCTCGGCCCTGACCAAGTCA
AATCCATGCGGGCTGCTCTTGATCTTTTCGGTCGTGAGTTCGGAGACGTA
GCCACCTACTCCCAACATCAGCCGGACTCCGATTACCTCGGGAACTTGCT
CCGTAGTAAGACATTCATCGCGCTTGCTGCCTTCGACCAAGAAGCGGTTG
TTGGCGCTCTCGCGGCTTACGTTCTGCCCAAGTTTGAGCAGCCGCGTAGT
GAGATCTATATCTATGATCTCGCAGTCTCCGGCGAGCACCGGAGGCAGGG
CATTGCCACCGCGCTCATCAATCTCCTCAAGCATGAGGCCAACGCGCTTG
GTGCTTATGTGATCTACGTGCAAGCAGATTACGGTGACGATCCCGCAGTG
GCTCTCTATACAAAGTTGGGCATACGGGAAGAAGTGATGCACTTTGATAT
CGACCCAAGTACCGCCACCTAAGAGCTGGAGCGGCGTAACCGTCGCACAG
GAAGGACAGAGAAAGCGCGGATCTGGGAAGTGACGGACAGAACGGTCAGG
ACCTGGATTGGGGAGGCGGTTGCCGCCGCTGCTGCTGACGGTGTGACGTT
CTCTGTTCCGGTCACACCACATACGTTCCGCCATTCCTATGCGATGCACA
TGCTGTATGCCGGTATACCGCTGAAAGTTCTGCAAAGCCTGATGGGACAT
AAGTCCATCAGTTCAACGGAAGTCTACACGAAGGTTTTTGCGCTGGATGT
GGCTGCCCGGCTCCGGGTGCAGTTTGCGATGCCGGAGTCTGATGCGGTTG
CGATGCTGAAACAATTATCCTGAGAATAAATGCCTTGGCCTTTATATGGA
AATGTGGAACTGAGTGGATATGCTGTTTTTGTCTGTTAAACAGAGAAGCT
GGCTGTTATCCACTGAGAAGCGAACGAAACAGTCGGGAAAATCTCCCATT
ATCGTAGAGATCCGCATTATTAATCTCAGGAGCCTGTGTAGCGTTTATAG
GAAGTAGTGTTCTGTCATGATGCCTGCAAGCGGTAACGAAAACGATTTGA
ATATGCCTTCAGGAACAATAGAAATCTTCGTGCGGTGTTACGTTGAAGTG
GAGCGGATTATGTCAGCAATGGACAGAACAACCTAATGAACACAGAACCA
TGATGTGGTCTGTCCTTTTACAGCCAGTAGTGCTCGCCGCAGTCGAGCGA
CAGGGCGAAGCCCTCGAGTGAGCGAGGAAGCACCAGGGAACAGCACTTAT
ATATTCTGCTTACACACGATGCCTGAAAAAACTTCCCTTGGGGTTATCCA
CTTATCCACGGGGATATTTTTATAATTATTTTTTTTATAGTTTTTAGATC
TTCTTTTTTAGAGCGCCTTGTAGGCCTTTATCCATGCTGGTTCTAGAGAA
GGTGTTGTGACAAATTGCCCTTTCAGTGTGACAAATCACCCTCAAATGAC
AGTCCTGTCTGTGACAAATTGCCCTTAACCCTGTGACAAATTGCCCTCAG
AAGAAGCTGTTTTTTCACAAAGTTATCCCTGCTTATTGACTCTTTTTTAT
TTAGTGTGACAATCTAAAAACTTGTCACACTTCACATGGATCTGTCATGG
CGGAAACAGCGGTTATCAATCACAAGAAACGTAAAAATAGCCCGCGAATC
GTCCAGTCAAACGACCTCACTGAGGCGGCATATAGTCTCTCCCGGGATCA
AAAACGTATGCTGTATCTGTTCGTTGACCAGATCAGAAAATCTGATGGCA
CCCTACAGGAACATGACGGTATCTGCGAGATCCATGTTGCTAAATATGCT
GAAATATTCGGATTGACCTCTGCGGAAGCCAGTAAGGATATACGGCAGGC
ATTGAAGAGTTTCGCGGGGAAGGAAGTGGTTTTTTATCGCCCTGAAGAGG
ATGCCGGCGATGAAAAAGGCTATGAATCTTTTCCTTGGTTTATCAAACGT
GCGCACAGTCCATCCAGAGGGCTTTACAGTGTACATATCAACCCATATCT
CATTCCCTTCTTTATCGGGTTACAGAACCGGTTTACGCAGTTTCGGCTTA
GTGAAACAAAAGAAATCACCAATCCGTATGCCATGCGTTTATACGAATCC
CTGTGTCAGTATCGTAAGCCGGATGGCTCAGGCATCGTGTCTCTGAAAAT
CGACTGGATCATAGAGCGTTACCAGCTGCCTCAAAGTTACCAGCGTATGC
CTGACTTCCGCCGCCGCTTCCTGCAGGTCTGTGTTAATGAGATCAACAGC
AGAACTCCAATGCGCCTCTCATACATTGAGAAAAAGAAAGGCCGCCAGAC
GACTCATATCGTATTTTCCTTCCGCGATATCACTTCCATGACGACAGGAT
AGTCTGAGGGTTATCTGTCACAGATTTGAGGGTGGTTCGTCACATTTGTT
CTGACCTACTGAGGGTAATTTGTCACAGTTTTGCTGTTTCCTTCAGCCTG
CATGGATTTTCTCATACTTTTTGAACTGTAATTTTTAAGGAAGCCAAATT
TGAGGGCAGTTTGTCACAGTTGATTTCCTTCTCTTTCCCTTCGTCATGTG
ACCTGATATCGGGGGTTAGTTCGTCATCATTGATGAGGGTTGATTATCAC
AGTTTATTACTCTGAATTGGCTATCCGCGTGTGTACCTCTACCTGGAGTT
TTTCCCACGGTGGATATTTCTTCTTGCGCTGAGCGTAAGAGCTATCTGAC
AGAACAGTTCTTCTTTGCTTCCTCGCCAGTTCGCTCGCTATGCTCGGTTA
CACGGCTGCGGCGAGCGCTAGTGATAATAAGTGACTGAGGTATGTGGTGT
TCTTATCTCCTTTTGTAGTGTTGCTCTTATTTTAAACAACTTTGCGGTTT
TTTGATGACTTTGCGATTTTGTTGTTGCTTTGCAGTAAATTGCAAGATTT
AATAAAAAAACGCAAAGCAATGATTAAAGGATGTTCAGAATGAAACTCAT
GGAAACACTTAACCAGTGCATAAACGCTGGTCATGAAATGACGAAGGCTA
TCGCCATTGCACAGTTTAATGATGACAGCCCGGAAGCGAGGAAAATAACC
CGGCGCTGGAGAATAGGTGAAGCAGCGGATTTAGTTGGGGTTTCTTCTCA
GGCTATCAGAGATGCCGAGAAAGCAGGGCGACTACCGCACCCGGATATGG
AAATTCGAGGACGGGTTGAGCAACGTGTTGGTTATACAATTGAACAAATT
AATCATATGCGTGATGTGTTTGGTACGCGATTGCGACGTGCTGAGGACGT
ATTTCCACCGGTGATCGGGGTTGCTGCCCATAAAGGTGGCGTTTACAAAA
CCTCAGTTTCTGTTCATCTTGCTCAGGATCTGGCTCTGAAGGGGCTACGT
GTTTTGCTCGTGGAAGGTAACGACCCCCAGGGAACAGCCTCAATGTATCA
CGGATGGGTACCAGATCTTCATATTCATGCAGAAGATACTCTCCTGCCTT
TCTATCTTGGGGAAAAGGACGATGTCACTTATGCAATAAAGCCCACTTGC
TGGCCGGGGCTTGACATTATTCCTTCCTGTCTGGCTCTGCACCGTATTGA
AACTGAGTTAATGGGCAAATTTGATGAAGGTAAACTGCCCACCGATCCAC
ACCTGATGCTCCGACTGGCCATTGAAACTGTTGCTCATGACTATGATGTC
ATAGTTATTGACAGCGCGCCTAACCTGGGTATCGGCACGATTAATGTCGT
ATGTGCTGCTGATGTGCTGATTGTTCCCACGCCTGCTGAGTIGTTTGACT
ACACCTCCGCACTGCAGTTTTTCGATATGCTTCGTGATCTGCTCAAGAAC
GTTGATCTTAAAGGGTTCGAGCCTGATGTACGTATTTTGCTTACCAAATA
CAGCAATAGTAATGGCTCTCAGTCCCCGTGGATGGAGGAGCAAATTCGGG
ATGCCTGGGGAAGCATGGTTCTAAAAAATGTTGTACGTGAAACGGATGAA
GTTGGTAAAGGTCAGATCCGGATGAGAACTGTTTTTGAACAGGCCATTGA
TCAACGCAGTTCAACTGGTGCCTGGAGAAATGCTCTTTCTATTTGGGAAC
CTGTCTGCAATGAAATTTTCGATCGTCTGATTAAACCACGCTGGGAGATT
AGATAATGAAGCGTGCGCCTGTTATTCCAAAACATACGCTCAATACTCAA
CCGGTTGAAGATACTTCGTTATCGACACCAGCTGCCCCGATGGTGGATTC
GTTAATTGCGCGCGTAGGAGTAATGGCTCGCGGTAATGCCATTACTTTGC
CTGTATGTGGTCGGGATGTGAAGTTTACTCTTGAAGTGCTCCGGGGTGAT
AGTGTTGAGAAAACCTCTCGGGTATGGTCAGGTAATGAACGTGACCAGGA
GCTGCTTACTGAGGACGCACTGGATGATCTCATCCCTTCTTTTCTACTGA
CTGGTCAACAGACACCGGCGTTCGGTCGAAGAGTATCTGGTGTCATAGAA
ATTGCCGATGGGAGTCGCCGTCGTAAAGCTGCTGCACTTACCGAAAGTGA
TTATCGTGTTCTGGTTGGCGAGCTGGATGATGAGCAGATGGCTGCATTAT
CCAGATTGGGTAACGATTATCGCCCAACAAGTGCTTATGAACGTGGTCAG
CGTTATGCAAGCCGATTGCAGAATGAATTTGCTGGAAATATTTCTGCGCT
GGCTGATGCGGAAAATATTTCACGTAAGATTATTACCCGCTGTATCAACA
CCGCCAAATTGCCTAAATCAGTTGTTGCTCTTTTTTCTCACCCCGGTGAA
CTATCTGCCCGGTCAGGTGATGCACTTCAAAAAGCCTTTACAGATAAAGA
GGAATTACTTAAGCAGCAGGCATCTAACCTTCATGAGCAGAAAAAAGCTG
GGGTGATATTTGAAGCTGAAGAAGTTATCACTCTTTTAACTTCTGTGCTT
AAAACGTCATCTGCATCAAGAACTAGTTTAAGCTCACGACATCAGTTTGC
TCCTGGAGCGACAGTATTGTATAAGGGCGATAAAATGGTGCTTAACCTGG
ACAGGTCACGTGTTCCAACTGAGTGTATAGAGAAAATTGAGGCCATTCTT
AAGGAACTTGAAAAGCCAGCACCCTGATGCGACCACGTTTTAGTCTACGT
TTATCTGTCTTTACTTAATGTCCTTTGTTACAGGCCAGAAAGCATAACTG
GCCTGAATATTCTCTCTGGGCCCACTGTTCCACTTGTATCGTCGGTCTGA
TAATCAGACTGGGACCACGGTCCCACTCGTATCGTCGGTCTGATTATTAG
TCTGGGACCACGGTCCCACTCGTATCGTCGGTCTGATTATTAGTCTGGGA
CCACGGTCCCACTCGTATCGTCGGTCTGATAATCAGACTGGGACCACGGT
CCCACTCGTATCGTCGGTCTGATTATTAGTCTGGGACCATGGTCCCACTC
GTATCGTCGGTCTGATTATTAGTCTGGGACCACGGTCCCACTCGTATCGT
CGGTCTGATTATTAGTCTGGAACCACGGTCCCACTCGTATCGTCGGTCTG
ATTATTAGTCTGGGACCACGGTCCCACTCGTATCGTCGGTCTGATTATTA
GTCTGGGACCACGATCCCACTCGTGTTGTCGGTCTGATTATCGGTCTGGG
ACCACGGTCCCACTTGTATTGTCGATCAGACTATCAGCGTGAGACTACGA
TTCCATCAATGCCTGTCAAGGGCAAGTATTGACATGTCGTCGTAACCTGT
AGAACGGAGTAACCTCGGTGTGCGGTTGTATGCCTGCTGTGGATTGCTGC
TGTGTCCTGCTTATCCACAACATTTTGCGCACGGTTATGTGGACAAAATA
CCTGGTTACCCAGGCCGTGCCGGCACGTTAACCGGGCTGCATCCGATGCA
AGTGTGTCGCTGTCGACGCAAGCGGCCGCA
>pMXBG_G7F
(SEQ ID NO: 45)

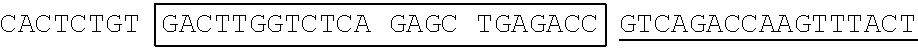

CATATATACTTTAGATTGATTTAAAACTTCATTTTTAATTTAAAAGGATC
TAGGTGAAGATCCTTTTTGATAATCTCATGACCAAAATCCCTTAACGTGA
GTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCT
TTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGG
CGATAAGTCGTGTCTTACCGGGTTGGACTCAAGACGATAGTTACCGGATA
AGGCGCAGCGGTCGGGCTGAACGGGGGGTTCGTGCACACAGCCCAGCTTG
GAGCGAACGACCTACACCGAACTGAGATACCTACAGCGTGAGCTATGAGA
AAGCGCCACGCTTCCCGAAGGGAGAAAGGCGGACAGGTATCCGGTAAGCG
GCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATG
TTCTTTCCTGCGTTATCCCCTGATTCTGTGGATAACCGTATTACCGCCTT
TGAGTGAGCTGATACCGCTCGCCGCAGCCGAACGACCGAGCGCAGCGAGT
CAGTGAGCGAGGAAGCGGATGAGCGCCCAATACGCAAACCGCCTCTCCCC
GCGCGTTGGCCGATTCATTAATGCAGCTGGCACGACAGGTTTCCCGACTG
GAAAGCGGGCAGTGAGCGCAACGCAATTAATGTGAGTTAGCTCACTCATT
AGGCACCCCAGGCTTTACACTTTATGCTTCCGGCTCGTATGTTGTGTGGA
ATTGTGAGCGGATAACAATTTCACACAGGAAACAGCTATGACCATGATTA
CGCCAAGCTTGCATGCCTGCAGGTCGACTCTAGAGGATCCCCGGGTACCG
AGCTCGAATTCACTGGCCGTCGTTTTACAACGTCGTGACTGGGAAAACCC
TGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGC
AGCCTGAATGGCGAATGGCGCCTGATGCGGTATTTTCTCCTTACGCATCT
GTGCGGTATTTCACACCGCATATGGTGCACTCTCAGTACAATCTGCTCTG
ATGCCGCATAGTTAAGCCAGCCCCGACACCCGCCAACACCCGCTGACGCG
CCCTGACGGGCTTGTCTGCTCCCGGCATCCGCTTACAGACAAGCTGTGAC

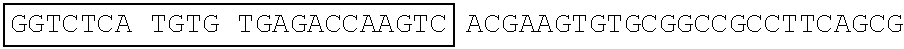

GCCGCACGAATCCATGTGGGAGTTTATTCTTGACACAGATATTTATGATA
TAATAACTGAGTAAGCTTAACATAAGGAGGAAAAACATATGTTACGCAGC
AGCAACGATGTTACGCAGCAGGGCAGTCGCCCTAAAACAAAGTTAGGTGG
CTCAAGTATGGGCATCATTCGCACATGTAGGCTCGGCCCTGACCAAGTCA
AATCCATGCGGGCTGCTCTTGATCTTTTCGGTCGTGAGTTCGGAGACGTA
GCCACCTACTCCCAACATCAGCCGGACTCCGATTACCTCGGGAACTTGCT
CCGTAGTAAGACATTCATCGCGCTTGCTGCCTTCGACCAAGAAGCGGTTG
TTGGCGCTCTCGCGGCTTACGTTCTGCCCAAGTTTGAGCAGCCGCGTAGT
GAGATCTATATCTATGATCTCGCAGTCTCCGGCGAGCACCGGAGGCAGGG
CATTGCCACCGCGCTCATCAATCTCCTCAAGCATGAGGCCAACGCGCTTG
GTGCTTATGTGATCTACGTGCAAGCAGATTACGGTGACGATCCCGCAGTG
GCTCTCTATACAAAGTTGGGCATACGGGAAGAAGTGATGCACTTTGATAT
CGACCCAAGTACCGCCACCTAAGAGCTGGAGCGGCGTAACCGTCGCACAG
GAAGGACAGAGAAAGCGCGGATCTGGGAAGTGACGGACAGAACGGTCAGG
ACCTGGATTGGGGAGGCGGTTGCCGCCGCTGCTGCTGACGGTGTGACGTT
CTCTGTTCCGGTCACACCACATACGTTCCGCCATTCCTATGCGATGCACA
TGCTGTATGCCGGTATACCGCTGAAAGTTCTGCAAAGCCTGATGGGACAT
AAGTCCATCAGTTCAACGGAAGTCTACACGAAGGTTTTTGCGCTGGATGT
GGCTGCCCGGCTCCGGGTGCAGTTTGCGATGCCGGAGTCTGATGCGGTTG
CGATGCTGAAACAATTATCCTGAGAATAAATGCCTTGGCCTTTATATGGA
AATGTGGAACTGAGTGGATATGCTGTTTTTGTCTGTTAAACAGAGAAGCT
GGCTGTTATCCACTGAGAAGCGAACGAAACAGTCGGGAAAATCTCCCATT
ATCGTAGAGATCCGCATTATTAATCTCAGGAGCCTGTGTAGCGTTTATAG
GAAGTAGTGTTCTGTCATGATGCCTGCAAGCGGTAACGAAAACGATTTGA
ATATGCCTTCAGGAACAATAGAAATCTTCGTGCGGTGTTACGTTGAAGTG
GAGCGGATTATGTCAGCAATGGACAGAACAACCTAATGAACACAGAACCA
TGATGTGGTCTGTCCTTTTACAGCCAGTAGTGCTCGCCGCAGTCGAGCGA
CAGGGCGAAGCCCTCGAGTGAGCGAGGAAGCACCAGGGAACAGCACTTAT
ATATTCTGCTTACACACGATGCCTGAAAAAACTTCCCTTGGGGTTATCCA
CTTATCCACGGGGATATTTTTATAATTATTTTTTTTATAGTTTTTAGATC
TTCTTTTTTAGAGCGCCTTGTAGGCCTTTATCCATGCTGGTTCTAGAGAA
GGTGTTGTGACAAATTGCCCTTTCAGTGTGACAAATCACCCTCAAATGAC
AGTCCTGTCTGTGACAAATTGCCCTTAACCCTGTGACAAATTGCCCTCAG
AAGAAGCTGTTTTTTCACAAAGTTATCCCTGCTTATTGACTCTTTTTTAT
TTAGTGTGACAATCTAAAAACTTGTCACACTTCACATGGATCTGTCATGG
CGGAAACAGCGGTTATCAATCACAAGAAACGTAAAAATAGCCCGCGAATC
GTCCAGTCAAACGACCTCACTGAGGCGGCATATAGTCTCTCCCGGGATCA
AAAACGTATGCTGTATCTGTTCGTTGACCAGATCAGAAAATCTGATGGCA
CCCTACAGGAACATGACGGTATCTGCGAGATCCATGTTGCTAAATATGCT
GAAATATTCGGATTGACCTCTGCGGAAGCCAGTAAGGATATACGGCAGGC
ATTGAAGAGTTTCGCGGGGAAGGAAGTGGTTTTTTATCGCCCTGAAGAGG
ATGCCGGCGATGAAAAAGGCTATGAATCTTTTCCTTGGTTTATCAAACGT
GCGCACAGTCCATCCAGAGGGCTTTACAGTGTACATATCAACCCATATCT
CATTCCCTTCTTTATCGGGTTACAGAACCGGTTTACGCAGTTTCGGCTTA
GTGAAACAAAAGAAATCACCAATCCGTATGCCATGCGTTTATACGAATCC
CTGTGTCAGTATCGTAAGCCGGATGGCTCAGGCATCGTGTCTCTGAAAAT
CGACTGGATCATAGAGCGTTACCAGCTGCCTCAAAGTTACCAGCGTATGC
CTGACTTCCGCCGCCGCTTCCTGCAGGTCTGTGTTAATGAGATCAACAGC
AGAACTCCAATGCGCCTCTCATACATTGAGAAAAAGAAAGGCCGCCAGAC
GACTCATATCGTATTTTCCTTCCGCGATATCACTTCCATGACGACAGGAT
AGTCTGAGGGTTATCTGTCACAGATTTGAGGGTGGTTCGTCACATTTGTT
CTGACCTACTGAGGGTAATTTGTCACAGTTTTGCTGTTTCCTTCAGCCTG
CATGGATTTTCTCATACTTTTTGAACTGTAATTTTTAAGGAAGCCAAATT
TGAGGGCAGTTTGTCACAGTTGATTTCCTTCTCTTTCCCTTCGTCATGTG
ACCTGATATCGGGGGTTAGTTCGTCATCATTGATGAGGGTTGATTATCAC
AGTTTATTACTCTGAATTGGCTATCCGCGTGTGTACCTCTACCTGGAGTT
TTTCCCACGGTGGATATTTCTTCTTGCGCTGAGCGTAAGAGCTATCTGAC
AGAACAGTTCTTCTTTGCTTCCTCGCCAGTTCGCTCGCTATGCTCGGTTA
CACGGCTGCGGCGAGCGCTAGTGATAATAAGTGACTGAGGTATGTGGTGT
TCTTATCTCCTTTTGTAGTGTTGCTCTTATTTTAAACAACTTTGCGGTTT
TTTGATGACTTTGCGATTTTGTTGTTGCTTTGCAGTAAATTGCAAGATTT
AATAAAAAAACGCAAAGCAATGATTAAAGGATGTTCAGAATGAAACTCAT
GGAAACACTTAACCAGTGCATAAACGCTGGTCATGAAATGACGAAGGCTA
TCGCCATTGCACAGTTTAATGATGACAGCCCGGAAGCGAGGAAAATAACC
CGGCGCTGGAGAATAGGTGAAGCAGCGGATTTAGTTGGGGTTTCTTCTCA
GGCTATCAGAGATGCCGAGAAAGCAGGGCGACTACCGCACCCGGATATGG
AAATTCGAGGACGGGTTGAGCAACGTGTTGGTTATACAATTGAACAAATT
AATCATATGCGTGATGTGTTTGGTACGCGATTGCGACGTGCTGAGGACGT
ATTTCCACCGGTGATCGGGGTTGCTGCCCATAAAGGTGGCGTTTACAAAA
CCTCAGTTTCTGTTCATCTTGCTCAGGATCTGGCTCTGAAGGGGCTACGT
GTTTTGCTCGTGGAAGGTAACGACCCCCAGGGAACAGCCTCAATGTATCA
CGGATGGGTACCAGATCTTCATATTCATGCAGAAGATACTCTCCTGCCTT
TCTATCTTGGGGAAAAGGACGATGTCACTTATGCAATAAAGCCCACTTGC
TGGCCGGGGCTTGACATTATTCCTTCCTGTCTGGCTCTGCACCGTATTGA
AACTGAGTTAATGGGCAAATTTGATGAAGGTAAACTGCCCACCGATCCAC
ACCTGATGCTCCGACTGGCCATTGAAACTGTTGCTCATGACTATGATGTC
ATAGTTATTGACAGCGCGCCTAACCTGGGTATCGGCACGATTAATGTCGT
ATGTGCTGCTGATGTGCTGATTGTTCCCACGCCTGCTGAGTIGTTTGACT
ACACCTCCGCACTGCAGTTTTTCGATATGCTTCGTGATCTGCTCAAGAAC
GTTGATCTTAAAGGGTTCGAGCCTGATGTACGTATTTTGCTTACCAAATA
CAGCAATAGTAATGGCTCTCAGTCCCCGTGGATGGAGGAGCAAATTCGGG
ATGCCTGGGGAAGCATGGTTCTAAAAAATGTTGTACGTGAAACGGATGAA
GTTGGTAAAGGTCAGATCCGGATGAGAACTGTTTTTGAACAGGCCATTGA
TCAACGCAGTTCAACTGGTGCCTGGAGAAATGCTCTTTCTATTTGGGAAC
CTGTCTGCAATGAAATTTTCGATCGTCTGATTAAACCACGCTGGGAGATT
AGATAATGAAGCGTGCGCCTGTTATTCCAAAACATACGCTCAATACTCAA
CCGGTTGAAGATACTTCGTTATCGACACCAGCTGCCCCGATGGTGGATTC
GTTAATTGCGCGCGTAGGAGTAATGGCTCGCGGTAATGCCATTACTTTGC
CTGTATGTGGTCGGGATGTGAAGTTTACTCTTGAAGTGCTCCGGGGTGAT
AGTGTTGAGAAAACCTCTCGGGTATGGTCAGGTAATGAACGTGACCAGGA
GCTGCTTACTGAGGACGCACTGGATGATCTCATCCCTTCTTTTCTACTGA
CTGGTCAACAGACACCGGCGTTCGGTCGAAGAGTATCTGGTGTCATAGAA
ATTGCCGATGGGAGTCGCCGTCGTAAAGCTGCTGCACTTACCGAAAGTGA
TTATCGTGTTCTGGTTGGCGAGCTGGATGATGAGCAGATGGCTGCATTAT
CCAGATTGGGTAACGATTATCGCCCAACAAGTGCTTATGAACGTGGTCAG
CGTTATGCAAGCCGATTGCAGAATGAATTTGCTGGAAATATTTCTGCGCT
GGCTGATGCGGAAAATATTTCACGTAAGATTATTACCCGCTGTATCAACA
CCGCCAAATTGCCTAAATCAGTTGTTGCTCTTTTTTCTCACCCCGGTGAA
CTATCTGCCCGGTCAGGTGATGCACTTCAAAAAGCCTTTACAGATAAAGA
GGAATTACTTAAGCAGCAGGCATCTAACCTTCATGAGCAGAAAAAAGCTG
GGGTGATATTTGAAGCTGAAGAAGTTATCACTCTTTTAACTTCTGTGCTT
AAAACGTCATCTGCATCAAGAACTAGTTTAAGCTCACGACATCAGTTTGC
TCCTGGAGCGACAGTATTGTATAAGGGCGATAAAATGGTGCTTAACCTGG
ACAGGTCACGTGTTCCAACTGAGTGTATAGAGAAAATTGAGGCCATTCTT
AAGGAACTTGAAAAGCCAGCACCCTGATGCGACCACGTTTTAGTCTACGT
TTATCTGTCTTTACTTAATGTCCTTTGTTACAGGCCAGAAAGCATAACTG
GCCTGAATATTCTCTCTGGGCCCACTGTTCCACTTGTATCGTCGGTCTGA
TAATCAGACTGGGACCACGGTCCCACTCGTATCGTCGGTCTGATTATTAG
TCTGGGACCACGGTCCCACTCGTATCGTCGGTCTGATTATTAGTCTGGGA
CCACGGTCCCACTCGTATCGTCGGTCTGATAATCAGACTGGGACCACGGT
CCCACTCGTATCGTCGGTCTGATTATTAGTCTGGGACCATGGTCCCACTC
GTATCGTCGGTCTGATTATTAGTCTGGGACCACGGTCCCACTCGTATCGT
CGGTCTGATTATTAGTCTGGAACCACGGTCCCACTCGTATCGTCGGTCTG
ATTATTAGTCTGGGACCACGGTCCCACTCGTATCGTCGGTCTGATTATTA
GTCTGGGACCACGATCCCACTCGTGTTGTCGGTCTGATTATCGGTCTGGG
ACCACGGTCCCACTTGTATTGTCGATCAGACTATCAGCGTGAGACTACGA
TTCCATCAATGCCTGTCAAGGGCAAGTATTGACATGTCGTCGTAACCTGT
AGAACGGAGTAACCTCGGTGTGCGGTTGTATGCCTGCTGTGGATTGCTGC
TGTGTCCTGCTTATCCACAACATTTTGCGCACGGTTATGTGGACAAAATA
CCTGGTTACCCAGGCCGTGCCGGCACGTTAACCGGGCTGCATCCGATGCA
AGTGTGTCGCTGTCGACGCAAGCGGCCGCA
>pMXBG_F7E
(SEQ ID NO: 46)

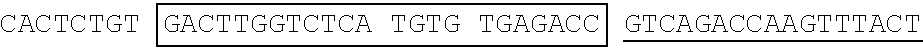

CATATATACTTTAGATTGATTTAAAACTTCATTTTTAATTTAAAAGGATC
TAGGTGAAGATCCTTTTTGATAATCTCATGACCAAAATCCCTTAACGTGA
GTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCT
TTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGG
CGATAAGTCGTGTCTTACCGGGTTGGACTCAAGACGATAGTTACCGGATA
AGGCGCAGCGGTCGGGCTGAACGGGGGGTTCGTGCACACAGCCCAGCTTG
GAGCGAACGACCTACACCGAACTGAGATACCTACAGCGTGAGCTATGAGA
AAGCGCCACGCTTCCCGAAGGGAGAAAGGCGGACAGGTATCCGGTAAGCG
GCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATG
TTCTTTCCTGCGTTATCCCCTGATTCTGTGGATAACCGTATTACCGCCTT
TGAGTGAGCTGATACCGCTCGCCGCAGCCGAACGACCGAGCGCAGCGAGT
CAGTGAGCGAGGAAGCGGATGAGCGCCCAATACGCAAACCGCCTCTCCCC
GCGCGTTGGCCGATTCATTAATGCAGCTGGCACGACAGGTTTCCCGACTG
GAAAGCGGGCAGTGAGCGCAACGCAATTAATGTGAGTTAGCTCACTCATT
AGGCACCCCAGGCTTTACACTTTATGCTTCCGGCTCGTATGTTGTGTGGA
ATTGTGAGCGGATAACAATTTCACACAGGAAACAGCTATGACCATGATTA
CGCCAAGCTTGCATGCCTGCAGGTCGACTCTAGAGGATCCCCGGGTACCG
AGCTCGAATTCACTGGCCGTCGTTTTACAACGTCGTGACTGGGAAAACCC
TGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGC
AGCCTGAATGGCGAATGGCGCCTGATGCGGTATTTTCTCCTTACGCATCT
GTGCGGTATTTCACACCGCATATGGTGCACTCTCAGTACAATCTGCTCTG
ATGCCGCATAGTTAAGCCAGCCCCGACACCCGCCAACACCCGCTGACGCG
CCCTGACGGGCTTGTCTGCTCCCGGCATCCGCTTACAGACAAGCTGTGAC

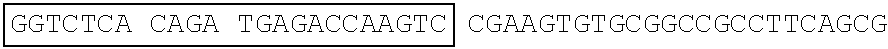

GCCGCACGAATCCATGTGGGAGTTTATTCTTGACACAGATATTTATGATA
TAATAACTGAGTAAGCTTAACATAAGGAGGAAAAACATATGTTACGCAGC
AGCAACGATGTTACGCAGCAGGGCAGTCGCCCTAAAACAAAGTTAGGTGG
CTCAAGTATGGGCATCATTCGCACATGTAGGCTCGGCCCTGACCAAGTCA
AATCCATGCGGGCTGCTCTTGATCTTTTCGGTCGTGAGTTCGGAGACGTA
GCCACCTACTCCCAACATCAGCCGGACTCCGATTACCTCGGGAACTTGCT
CCGTAGTAAGACATTCATCGCGCTTGCTGCCTTCGACCAAGAAGCGGTTG
TTGGCGCTCTCGCGGCTTACGTTCTGCCCAAGTTTGAGCAGCCGCGTAGT
GAGATCTATATCTATGATCTCGCAGTCTCCGGCGAGCACCGGAGGCAGGG
CATTGCCACCGCGCTCATCAATCTCCTCAAGCATGAGGCCAACGCGCTTG
GTGCTTATGTGATCTACGTGCAAGCAGATTACGGTGACGATCCCGCAGTG
GCTCTCTATACAAAGTTGGGCATACGGGAAGAAGTGATGCACTTTGATAT
CGACCCAAGTACCGCCACCTAAGAGCTGGAGCGGCGTAACCGTCGCACAG
GAAGGACAGAGAAAGCGCGGATCTGGGAAGTGACGGACAGAACGGTCAGG
ACCTGGATTGGGGAGGCGGTTGCCGCCGCTGCTGCTGACGGTGTGACGTT
CTCTGTTCCGGTCACACCACATACGTTCCGCCATTCCTATGCGATGCACA
TGCTGTATGCCGGTATACCGCTGAAAGTTCTGCAAAGCCTGATGGGACAT
AAGTCCATCAGTTCAACGGAAGTCTACACGAAGGTTTTTGCGCTGGATGT
GGCTGCCCGGCTCCGGGTGCAGTTTGCGATGCCGGAGTCTGATGCGGTTG
CGATGCTGAAACAATTATCCTGAGAATAAATGCCTTGGCCTTTATATGGA
AATGTGGAACTGAGTGGATATGCTGTTTTTGTCTGTTAAACAGAGAAGCT
GGCTGTTATCCACTGAGAAGCGAACGAAACAGTCGGGAAAATCTCCCATT
ATCGTAGAGATCCGCATTATTAATCTCAGGAGCCTGTGTAGCGTTTATAG
GAAGTAGTGTTCTGTCATGATGCCTGCAAGCGGTAACGAAAACGATTTGA
ATATGCCTTCAGGAACAATAGAAATCTTCGTGCGGTGTTACGTTGAAGTG
GAGCGGATTATGTCAGCAATGGACAGAACAACCTAATGAACACAGAACCA
TGATGTGGTCTGTCCTTTTACAGCCAGTAGTGCTCGCCGCAGTCGAGCGA
CAGGGCGAAGCCCTCGAGTGAGCGAGGAAGCACCAGGGAACAGCACTTAT
ATATTCTGCTTACACACGATGCCTGAAAAAACTTCCCTTGGGGTTATCCA
CTTATCCACGGGGATATTTTTATAATTATTTTTTTTATAGTTTTTAGATC
TTCTTTTTTAGAGCGCCTTGTAGGCCTTTATCCATGCTGGTTCTAGAGAA
GGTGTTGTGACAAATTGCCCTTTCAGTGTGACAAATCACCCTCAAATGAC
AGTCCTGTCTGTGACAAATTGCCCTTAACCCTGTGACAAATTGCCCTCAG
AAGAAGCTGTTTTTTCACAAAGTTATCCCTGCTTATTGACTCTTTTTTAT
TTAGTGTGACAATCTAAAAACTTGTCACACTTCACATGGATCTGTCATGG
CGGAAACAGCGGTTATCAATCACAAGAAACGTAAAAATAGCCCGCGAATC
GTCCAGTCAAACGACCTCACTGAGGCGGCATATAGTCTCTCCCGGGATCA
AAAACGTATGCTGTATCTGTTCGTTGACCAGATCAGAAAATCTGATGGCA
CCCTACAGGAACATGACGGTATCTGCGAGATCCATGTTGCTAAATATGCT
GAAATATTCGGATTGACCTCTGCGGAAGCCAGTAAGGATATACGGCAGGC
ATTGAAGAGTTTCGCGGGGAAGGAAGTGGTTTTTTATCGCCCTGAAGAGG
ATGCCGGCGATGAAAAAGGCTATGAATCTTTTCCTTGGTTTATCAAACGT
GCGCACAGTCCATCCAGAGGGCTTTACAGTGTACATATCAACCCATATCT
CATTCCCTTCTTTATCGGGTTACAGAACCGGTTTACGCAGTTTCGGCTTA
GTGAAACAAAAGAAATCACCAATCCGTATGCCATGCGTTTATACGAATCC
CTGTGTCAGTATCGTAAGCCGGATGGCTCAGGCATCGTGTCTCTGAAAAT
CGACTGGATCATAGAGCGTTACCAGCTGCCTCAAAGTTACCAGCGTATGC
CTGACTTCCGCCGCCGCTTCCTGCAGGTCTGTGTTAATGAGATCAACAGC
AGAACTCCAATGCGCCTCTCATACATTGAGAAAAAGAAAGGCCGCCAGAC
GACTCATATCGTATTTTCCTTCCGCGATATCACTTCCATGACGACAGGAT
AGTCTGAGGGTTATCTGTCACAGATTTGAGGGTGGTTCGTCACATTTGTT
CTGACCTACTGAGGGTAATTTGTCACAGTTTTGCTGTTTCCTTCAGCCTG
CATGGATTTTCTCATACTTTTTGAACTGTAATTTTTAAGGAAGCCAAATT
TGAGGGCAGTTTGTCACAGTTGATTTCCTTCTCTTTCCCTTCGTCATGTG
ACCTGATATCGGGGGTTAGTTCGTCATCATTGATGAGGGTTGATTATCAC
AGTTTATTACTCTGAATTGGCTATCCGCGTGTGTACCTCTACCTGGAGTT
TTTCCCACGGTGGATATTTCTTCTTGCGCTGAGCGTAAGAGCTATCTGAC
AGAACAGTTCTTCTTTGCTTCCTCGCCAGTTCGCTCGCTATGCTCGGTTA
CACGGCTGCGGCGAGCGCTAGTGATAATAAGTGACTGAGGTATGTGGTGT
TCTTATCTCCTTTTGTAGTGTTGCTCTTATTTTAAACAACTTTGCGGTTT
TTTGATGACTTTGCGATTTTGTTGTTGCTTTGCAGTAAATTGCAAGATTT
AATAAAAAAACGCAAAGCAATGATTAAAGGATGTTCAGAATGAAACTCAT
GGAAACACTTAACCAGTGCATAAACGCTGGTCATGAAATGACGAAGGCTA
TCGCCATTGCACAGTTTAATGATGACAGCCCGGAAGCGAGGAAAATAACC
CGGCGCTGGAGAATAGGTGAAGCAGCGGATTTAGTTGGGGTTTCTTCTCA
GGCTATCAGAGATGCCGAGAAAGCAGGGCGACTACCGCACCCGGATATGG
AAATTCGAGGACGGGTTGAGCAACGTGTTGGTTATACAATTGAACAAATT
AATCATATGCGTGATGTGTTTGGTACGCGATTGCGACGTGCTGAGGACGT
ATTTCCACCGGTGATCGGGGTTGCTGCCCATAAAGGTGGCGTTTACAAAA
CCTCAGTTTCTGTTCATCTTGCTCAGGATCTGGCTCTGAAGGGGCTACGT
GTTTTGCTCGTGGAAGGTAACGACCCCCAGGGAACAGCCTCAATGTATCA
CGGATGGGTACCAGATCTTCATATTCATGCAGAAGATACTCTCCTGCCTT
TCTATCTTGGGGAAAAGGACGATGTCACTTATGCAATAAAGCCCACTTGC
TGGCCGGGGCTTGACATTATTCCTTCCTGTCTGGCTCTGCACCGTATTGA
AACTGAGTTAATGGGCAAATTTGATGAAGGTAAACTGCCCACCGATCCAC
ACCTGATGCTCCGACTGGCCATTGAAACTGTTGCTCATGACTATGATGTC
ATAGTTATTGACAGCGCGCCTAACCTGGGTATCGGCACGATTAATGTCGT
ATGTGCTGCTGATGTGCTGATTGTTCCCACGCCTGCTGAGTTGTTTGACT
ACACCTCCGCACTGCAGTTTTTCGATATGCTTCGTGATCTGCTCAAGAAC
GTTGATCTTAAAGGGTTCGAGCCTGATGTACGTATTTTGCTTACCAAATA
CAGCAATAGTAATGGCTCTCAGTCCCCGTGGATGGAGGAGCAAATTCGGG
ATGCCTGGGGAAGCATGGTTCTAAAAAATGTTGTACGTGAAACGGATGAA
GTTGGTAAAGGTCAGATCCGGATGAGAACTGTTTTTGAACAGGCCATTGA
TCAACGCAGTTCAACTGGTGCCTGGAGAAATGCTCTTTCTATTTGGGAAC
CTGTCTGCAATGAAATTTTCGATCGTCTGATTAAACCACGCTGGGAGATT
AGATAATGAAGCGTGCGCCTGTTATTCCAAAACATACGCTCAATACTCAA
CCGGTTGAAGATACTTCGTTATCGACACCAGCTGCCCCGATGGTGGATTC
GTTAATTGCGCGCGTAGGAGTAATGGCTCGCGGTAATGCCATTACTTTGC
CTGTATGTGGTCGGGATGTGAAGTTTACTCTTGAAGTGCTCCGGGGTGAT
AGTGTTGAGAAAACCTCTCGGGTATGGTCAGGTAATGAACGTGACCAGGA
GCTGCTTACTGAGGACGCACTGGATGATCTCATCCCTTCTTTTCTACTGA
CTGGTCAACAGACACCGGCGTTCGGTCGAAGAGTATCTGGTGTCATAGAA
ATTGCCGATGGGAGTCGCCGTCGTAAAGCTGCTGCACTTACCGAAAGTGA
TTATCGTGTTCTGGTTGGCGAGCTGGATGATGAGCAGATGGCTGCATTAT
CCAGATTGGGTAACGATTATCGCCCAACAAGTGCTTATGAACGTGGTCAG
CGTTATGCAAGCCGATTGCAGAATGAATTTGCTGGAAATATTTCTGCGCT
GGCTGATGCGGAAAATATTTCACGTAAGATTATTACCCGCTGTATCAACA
CCGCCAAATTGCCTAAATCAGTTGTTGCTCTTTTTTCTCACCCCGGTGAA
CTATCTGCCCGGTCAGGTGATGCACTTCAAAAAGCCTTTACAGATAAAGA
GGAATTACTTAAGCAGCAGGCATCTAACCTTCATGAGCAGAAAAAAGCTG
GGGTGATATTTGAAGCTGAAGAAGTTATCACTCTTTTAACTTCTGTGCTT
AAAACGTCATCTGCATCAAGAACTAGTTTAAGCTCACGACATCAGTTTGC
TCCTGGAGCGACAGTATTGTATAAGGGCGATAAAATGGTGCTTAACCTGG
ACAGGTCACGTGTTCCAACTGAGTGTATAGAGAAAATTGAGGCCATTCTT
AAGGAACTTGAAAAGCCAGCACCCTGATGCGACCACGTTTTAGTCTACGT
TTATCTGTCTTTACTTAATGTCCTTTGTTACAGGCCAGAAAGCATAACTG
GCCTGAATATTCTCTCTGGGCCCACTGTTCCACTTGTATCGTCGGTCTGA
TAATCAGACTGGGACCACGGTCCCACTCGTATCGTCGGTCTGATTATTAG
TCTGGGACCACGGTCCCACTCGTATCGTCGGTCTGATTATTAGTCTGGGA
CCACGGTCCCACTCGTATCGTCGGTCTGATAATCAGACTGGGACCACGGT
CCCACTCGTATCGTCGGTCTGATTATTAGTCTGGGACCATGGTCCCACTC
GTATCGTCGGTCTGATTATTAGTCTGGGACCACGGTCCCACTCGTATCGT
CGGTCTGATTATTAGTCTGGAACCACGGTCCCACTCGTATCGTCGGTCTG
ATTATTAGTCTGGGACCACGGTCCCACTCGTATCGTCGGTCTGATTATTA
GTCTGGGACCACGATCCCACTCGTGTTGTCGGTCTGATTATCGGTCTGGG
ACCACGGTCCCACTTGTATTGTCGATCAGACTATCAGCGTGAGACTACGA
TTCCATCAATGCCTGTCAAGGGCAAGTATTGACATGTCGTCGTAACCTGT
AGAACGGAGTAACCTCGGTGTGCGGTTGTATGCCTGCTGTGGATTGCTGC
TGTGTCCTGCTTATCCACAACATTTTGCGCACGGTTATGTGGACAAAATA
CCTGGTTACCCAGGCCGTGCCGGCACGTTAACCGGGCTGCATCCGATGCA
AGTGTGTCGCTGTCGACGCAAGCGGCCGCA
>pMXBK_A7E
(SEQ ID NO: 47)

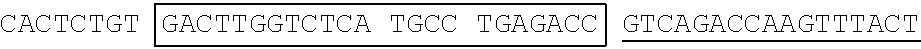

CATATATACTTTAGATTGATTTAAAACTTCATTTTTAATTTAAAAGGATC
TAGGTGAAGATCCTTTTTGATAATCTCATGACCAAAATCCCTTAACGTGA
GTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCT
TTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGG
CGATAAGTCGTGTCTTACCGGGTTGGACTCAAGACGATAGTTACCGGATA
AGGCGCAGCGGTCGGGCTGAACGGGGGGTTCGTGCACACAGCCCAGCTTG
GAGCGAACGACCTACACCGAACTGAGATACCTACAGCGTGAGCTATGAGA
AAGCGCCACGCTTCCCGAAGGGAGAAAGGCGGACAGGTATCCGGTAAGCG
GCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATG
TTCTTTCCTGCGTTATCCCCTGATTCTGTGGATAACCGTATTACCGCCTT
TGAGTGAGCTGATACCGCTCGCCGCAGCCGAACGACCGAGCGCAGCGAGT
CAGTGAGCGAGGAAGCGGATGAGCGCCCAATACGCAAACCGCCTCTCCCC
GCGCGTTGGCCGATTCATTAATGCAGCTGGCACGACAGGTTTCCCGACTG
GAAAGCGGGCAGTGAGCGCAACGCAATTAATGTGAGTTAGCTCACTCATT
AGGCACCCCAGGCTTTACACTTTATGCTTCCGGCTCGTATGTTGTGTGGA
ATTGTGAGCGGATAACAATTTCACACAGGAAACAGCTATGACCATGATTA
CGCCAAGCTTGCATGCCTGCAGGTCGACTCTAGAGGATCCCCGGGTACCG
AGCTCGAATTCACTGGCCGTCGTTTTACAACGTCGTGACTGGGAAAACCC
TGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGC
AGCCTGAATGGCGAATGGCGCCTGATGCGGTATTTTCTCCTTACGCATCT
GTGCGGTATTTCACACCGCATATGGTGCACTCTCAGTACAATCTGCTCTG
ATGCCGCATAGTTAAGCCAGCCCCGACACCCGCCAACACCCGCTGACGCG
CCCTGACGGGCTTGTCTGCTCCCGGCATCCGCTTACAGACAAGCTGTGAC

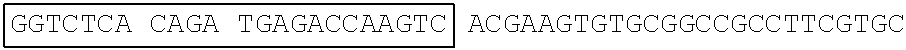

GCGGAACCCCTATTTGTTTATTTTTCTAAATACATTCAAATATGTATCCG
CTCATGAGACAATAACCCTGATAAATGCTTCAATAATATTGAAAAAGGAA
GAGTATGGCTAAAATGAGAATATCACCGGAATTGAAAAAACTGATCGAAA
AATACCGCTGCGTAAAAGATACGGAAGGAATGTCTCCTGCTAAGGTATAT
AAGCTGGTGGGAGAAAATGAAAACCTATATTTAAAAATGACGGACAGCCG
GTATAAAGGGACCACCTATGATGTGGAACGGGAAAAGGACATGATGCTAT
GGCTGGAAGGAAAGCTGCCTGTTCCAAAGGTCCTGCACTTTGAACGGCAT
GATGGCTGGAGCAATCTGCTCATGAGTGAGGCCGATGGCGTCCTTTGCTC
GGAAGAGTATGAAGATGAACAAAGCCCTGAAAAGATTATCGAGCTGTATG
CGGAGTGCATCAGGCTCTTTCACTCCATCGACATATCGGATTGTCCCTAT
ACGAATAGCTTAGACAGCCGCTTAGCCGAATTGGATTACTTACTGAATAA
CGATCTGGCCGATGTGGATTGCGAAAACTGGGAAGAGGACACTCCATTTA
AAGATCCGCGCGAGCTGTATGATTTTTTAAAGACGGAAAAGCCCGAAGAG
GAACTTGTCTTTTCCCACGGCGACCTGGGAGACAGCAACATCTTTGTGAA
AGATGGCAAAGTAAGTGGCTTTATTGATCTTGGGAGAAGCGGCAGGGCGG
ACAAGTGGTATGACATTGCCTTCTGCGTCCGGTCGCTCAGGGAGGATATC
GGGGAAGAACAGTATGTCGAGCTATTTTTTGACTTACTGGGGATCAAGCC
TGATTGGGAGAAAATAAAATATTATATTTTACTGGATGAATTGTTTTAGG
AGCTGGAGCGGCGTAACCGTCGCACAGGAAGGACAGAGAAAGCGCGGATC
TGGGAAGTGACGGACAGAACGGTCAGGACCTGGATTGGGGAGGCGGTTGC
CGCCGCTGCTGCTGACGGTGTGACGTTCTCTGTTCCGGTCACACCACATA
CGTTCCGCCATTCCTATGCGATGCACATGCTGTATGCCGGTATACCGCTG
AAAGTTCTGCAAAGCCTGATGGGACATAAGTCCATCAGTTCAACGGAAGT
CTACACGAAGGTTTTTGCGCTGGATGTGGCTGCCCGGCTCCGGGTGCAGT
TTGCGATGCCGGAGTCTGATGCGGTTGCGATGCTGAAACAATTATCCTGA
GAATAAATGCCTTGGCCTTTATATGGAAATGTGGAACTGAGTGGATATGC
TGTTTTTGTCTGTTAAACAGAGAAGCTGGCTGTTATCCACTGAGAAGCGA
ACGAAACAGTCGGGAAAATCTCCCATTATCGTAGAGATCCGCATTATTAA
TCTCAGGAGCCTGTGTAGCGTTTATAGGAAGTAGTGTTCTGTCATGATGC
CTGCAAGCGGTAACGAAAACGATTTGAATATGCCTTCAGGAACAATAGAA
ATCTTCGTGCGGTGTTACGTTGAAGTGGAGCGGATTATGTCAGCAATGGA
CAGAACAACCTAATGAACACAGAACCATGATGTGGTCTGTCCTTTTACAG
CCAGTAGTGCTCGCCGCAGTCGAGCGACAGGGCGAAGCCCTCGAGTGAGC
GAGGAAGCACCAGGGAACAGCACTTATATATTCTGCTTACACACGATGCC
TGAAAAAACTTCCCTTGGGGTTATCCACTTATCCACGGGGATATTTTTAT
AATTATTTTTTTTATAGTTTTTAGATCTTCTTTTTTAGAGCGCCTTGTAG
GCCTTTATCCATGCTGGTTCTAGAGAAGGTGTTGTGACAAATTGCCCTTT
CAGTGTGACAAATCACCCTCAAATGACAGTCCTGTCTGTGACAAATTGCC
CTTAACCCTGTGACAAATTGCCCTCAGAAGAAGCTGTTTTTTCACAAAGT
TATCCCTGCTTATTGACTCTTTTTTATTTAGTGTGACAATCTAAAAACTT
GTCACACTTCACATGGATCTGTCATGGCGGAAACAGCGGTTATCAATCAC
AAGAAACGTAAAAATAGCCCGCGAATCGTCCAGTCAAACGACCTCACTGA
GGCGGCATATAGTCTCTCCCGGGATCAAAAACGTATGCTGTATCTGTTCG
TTGACCAGATCAGAAAATCTGATGGCACCCTACAGGAACATGACGGTATC
TGCGAGATCCATGTTGCTAAATATGCTGAAATATTCGGATTGACCTCTGC
GGAAGCCAGTAAGGATATACGGCAGGCATTGAAGAGTTTCGCGGGGAAGG
AAGTGGTTTTTTATCGCCCTGAAGAGGATGCCGGCGATGAAAAAGGCTAT
GAATCTTTTCCTTGGTTTATCAAACGTGCGCACAGTCCATCCAGAGGGCT
TTACAGTGTACATATCAACCCATATCTCATTCCCTTCTTTATCGGGTTAC
AGAACCGGTTTACGCAGTTTCGGCTTAGTGAAACAAAAGAAATCACCAAT
CCGTATGCCATGCGTTTATACGAATCCCTGTGTCAGTATCGTAAGCCGGA
TGGCTCAGGCATCGTGTCTCTGAAAATCGACTGGATCATAGAGCGTTACC
AGCTGCCTCAAAGTTACCAGCGTATGCCTGACTTCCGCCGCCGCTTCCTG
CAGGTCTGTGTTAATGAGATCAACAGCAGAACTCCAATGCGCCTCTCATA
CATTGAGAAAAAGAAAGGCCGCCAGACGACTCATATCGTATTTTCCTTCC
GCGATATCACTTCCATGACGACAGGATAGTCTGAGGGTTATCTGTCACAG
ATTTGAGGGTGGTTCGTCACATTTGTTCTGACCTACTGAGGGTAATTTGT
CACAGTTTTGCTGTTTCCTTCAGCCTGCATGGATTTTCTCATACTTTTTG
AACTGTAATTTTTAAGGAAGCCAAATTTGAGGGCAGTTTGTCACAGTTGA
TTTCCTTCTCTTTCCCTTCGTCATGTGACCTGATATCGGGGGTTAGTTCG
TCATCATTGATGAGGGTTGATTATCACAGTTTATTACTCTGAATTGGCTA
TCCGCGTGTGTACCTCTACCTGGAGTTTTTCCCACGGTGGATATTTCTTC
TTGCGCTGAGCGTAAGAGCTATCTGACAGAACAGTTCTTCTTTGCTTCCT
CGCCAGTTCGCTCGCTATGCTCGGTTACACGGCTGCGGCGAGCGCTAGTG
ATAATAAGTGACTGAGGTATGTGGTGTTCTTATCTCCTTTTGTAGTGTTG
CTCTTATTTTAAACAACTTTGCGGTTTTTTGATGACTTTGCGATTTTGTT
GTTGCTTTGCAGTAAATTGCAAGATTTAATAAAAAAACGCAAAGCAATGA
TTAAAGGATGTTCAGAATGAAACTCATGGAAACACTTAACCAGTGCATAA
ACGCTGGTCATGAAATGACGAAGGCTATCGCCATTGCACAGTTTAATGAT
GACAGCCCGGAAGCGAGGAAAATAACCCGGCGCTGGAGAATAGGTGAAGC
AGCGGATTTAGTTGGGGTTTCTTCTCAGGCTATCAGAGATGCCGAGAAAG
CAGGGCGACTACCGCACCCGGATATGGAAATTCGAGGACGGGTTGAGCAA
CGTGTTGGTTATACAATTGAACAAATTAATCATATGCGTGATGTGTTTGG
TACGCGATTGCGACGTGCTGAGGACGTATTTCCACCGGTGATCGGGGTTG
CTGCCCATAAAGGTGGCGTTTACAAAACCTCAGTTTCTGTTCATCTTGCT
CAGGATCTGGCTCTGAAGGGGCTACGTGTTTTGCTCGTGGAAGGTAACGA
CCCCCAGGGAACAGCCTCAATGTATCACGGATGGGTACCAGATCTTCATA
TTCATGCAGAAGATACTCTCCTGCCTTTCTATCTTGGGGAAAAGGACGAT
GTCACTTATGCAATAAAGCCCACTTGCTGGCCGGGGCTTGACATTATTCC
TTCCTGTCTGGCTCTGCACCGTATTGAAACTGAGTTAATGGGCAAATTTG
ATGAAGGTAAACTGCCCACCGATCCACACCTGATGCTCCGACTGGCCATT
GAAACTGTTGCTCATGACTATGATGTCATAGTTATTGACAGCGCGCCTAA
CCTGGGTATCGGCACGATTAATGTCGTATGTGCTGCTGATGTGCTGATTG
TTCCCACGCCTGCTGAGTTGTTTGACTACACCTCCGCACTGCAGTTTTTC
GATATGCTTCGTGATCTGCTCAAGAACGTTGATCTTAAAGGGTTCGAGCC
TGATGTACGTATTTTGCTTACCAAATACAGCAATAGTAATGGCTCTCAGT
CCCCGTGGATGGAGGAGCAAATTCGGGATGCCTGGGGAAGCATGGTTCTA
AAAAATGTTGTACGTGAAACGGATGAAGTTGGTAAAGGTCAGATCCGGAT
GAGAACTGTTTTTGAACAGGCCATTGATCAACGCAGTTCAACTGGTGCCT
GGAGAAATGCTCTTTCTATTTGGGAACCTGTCTGCAATGAAATTTTCGAT
CGTCTGATTAAACCACGCTGGGAGATTAGATAATGAAGCGTGCGCCTGTT
ATTCCAAAACATACGCTCAATACTCAACCGGTTGAAGATACTTCGTTATC
GACACCAGCTGCCCCGATGGTGGATTCGTTAATTGCGCGCGTAGGAGTAA
TGGCTCGCGGTAATGCCATTACTTTGCCTGTATGTGGTCGGGATGTGAAG
TTTACTCTTGAAGTGCTCCGGGGTGATAGTGTTGAGAAAACCTCTCGGGT
ATGGTCAGGTAATGAACGTGACCAGGAGCTGCTTACTGAGGACGCACTGG
ATGATCTCATCCCTTCTTTTCTACTGACTGGTCAACAGACACCGGCGTTC
GGTCGAAGAGTATCTGGTGTCATAGAAATTGCCGATGGGAGTCGCCGTCG
TAAAGCTGCTGCACTTACCGAAAGTGATTATCGTGTTCTGGTTGGCGAGC
TGGATGATGAGCAGATGGCTGCATTATCCAGATTGGGTAACGATTATCGC
CCAACAAGTGCTTATGAACGTGGTCAGCGTTATGCAAGCCGATTGCAGAA
TGAATTTGCTGGAAATATTTCTGCGCTGGCTGATGCGGAAAATATTTCAC
GTAAGATTATTACCCGCTGTATCAACACCGCCAAATTGCCTAAATCAGTT
GTTGCTCTTTTTTCTCACCCCGGTGAACTATCTGCCCGGTCAGGTGATGC
ACTTCAAAAAGCCTTTACAGATAAAGAGGAATTACTTAAGCAGCAGGCAT
CTAACCTTCATGAGCAGAAAAAAGCTGGGGTGATATTTGAAGCTGAAGAA
GTTATCACTCTTTTAACTTCTGTGCTTAAAACGTCATCTGCATCAAGAAC
TAGTTTAAGCTCACGACATCAGTTTGCTCCTGGAGCGACAGTATTGTATA
AGGGCGATAAAATGGTGCTTAACCTGGACAGGTCACGTGTTCCAACTGAG
TGTATAGAGAAAATTGAGGCCATTCTTAAGGAACTTGAAAAGCCAGCACC
CTGATGCGACCACGTTTTAGTCTACGTTTATCTGTCTTTACTTAATGTCC
TTTGTTACAGGCCAGAAAGCATAACTGGCCTGAATATTCTCTCTGGGCCC
ACTGTTCCACTTGTATCGTCGGTCTGATAATCAGACTGGGACCACGGTCC
CACTCGTATCGTCGGTCTGATTATTAGTCTGGGACCACGGTCCCACTCGT
ATCGTCGGTCTGATTATTAGTCTGGGACCACGGTCCCACTCGTATCGTCG
GTCTGATAATCAGACTGGGACCACGGTCCCACTCGTATCGTCGGTCTGAT
TATTAGTCTGGGACCATGGTCCCACTCGTATCGTCGGTCTGATTATTAGT
CTGGGACCACGGTCCCACTCGTATCGTCGGTCTGATTATTAGTCTGGAAC
CACGGTCCCACTCGTATCGTCGGTCTGATTATTAGTCTGGGACCACGGTC
CCACTCGTATCGTCGGTCTGATTATTAGTCTGGGACCACGATCCCACTCG
TGTTGTCGGTCTGATTATCGGTCTGGGACCACGGTCCCACTTGTATTGTC
GATCAGACTATCAGCGTGAGACTACGATTCCATCAATGCCTGTCAAGGGC
AAGTATTGACATGTCGTCGTAACCTGTAGAACGGAGTAACCTCGGTGTGC
GGTTGTATGCCTGCTGTGGATTGCTGCTGTGTCCTGCTTATCCACAACAT
TTTGCGCACGGTTATGTGGACAAAATACCTGGTTACCCAGGCCGTGCCGG
CACGTTAACCGGGCTGCATCCGATGCAAGTGTGTCGCTGTCGACGCAAGC
GGCCGCA

1. A nucleic acid for use in DNA assembly, wherein the nucleic acid comprises at least one methylation-protectable restriction element, the methylation-protectable restriction element comprising:
a type IIS restriction enzyme recognition sequence; and
a DNA methylase recognition sequence,
wherein the type IIS restriction enzyme recognition sequence and the DNA methylase recognition sequence overlap such that the base modified by the DNA methylase lies within the type IIS restriction enzyme recognition sequence,
wherein the DNA methylase recognition sequence is not identical to or enclosed by the type IIS restriction enzyme recognition sequence, and
wherein the DNA methylase recognition sequence does not overlap with the sequence that would form the overhang end sequence generated by the type IIS restriction enzyme
wherein the nucleic acid further comprises an opposing type IS restriction enzyme recognition sequence provided on the opposing side of the cut site of the methylation-protectable restriction element thereby forming a truncated composite element,
wherein the opposing type IIS restriction enzyme recognition sequence of the truncating composite element is arranged to direct the type IIS restriction enzyme to cut the nucleic acid at a site that is within the recognition sequence of the type IIS restriction enzyme recognition sequence of the methylation-protectable restriction element, and/or within the nucleotides between the cut site and the recognition sequence of the type IIS restriction enzyme recognition sequence of the methylation-protectable restriction element.

2. (canceled)

3. The nucleic acid according to claim 1, wherein the nucleic acid comprises first and second methylation-protectable restriction elements wherein the, the first and second methylation-protectable restriction elements each comprise:
a type IIS restriction enzyme recognition sequence; and
a DNA methylase recognition sequence,
wherein the type IIS restriction enzyme recognition sequence and the DNA methylase recognition sequence overlap such that the base modified by the DNA methylase lies within the type IIS restriction enzyme recognition sequence,
wherein the DNA methylase recognition sequence is not identical to or enclosed by the type IIS restriction enzyme recognition sequence, and
wherein the DNA methylase recognition sequence does not overlap with the sequence that would form the overhang end sequence generated by the type IIS restriction enzyme,
wherein the nucleic acid further comprises an opposing type IIS restriction enzyme recognition sequences provided on the opposing side of the cut site of each of the first and second methylation-protectable restriction elements form:
A i) a maintained composite element or an insertional composite element, and ii) a truncated composite element; or
B) two truncated composite elements,
wherein the maintained composite element is an arrangement wherein the opposing type IIS restriction enzyme recognition sequence is arranged to direct the type IIS restriction enzyme to cut the nucleic acid at the same site as the type IIS restriction enzyme recognition sequence of the opposing methylation-protectable restriction element, such that the same overhang is produced,
wherein the insertional composite element is an arrangement comprising a functional sequence insert provided between the methylation-protectable restriction element and the opposing type IIS restriction enzyme recognition sequence, and
wherein the truncated composite element is an arrangement wherein the opposing type IS restriction enzyme recognition sequence of the truncating composite element is arranged to direct the type IIS restriction enzyme to cut the nucleic acid at a site that is within the recognition sequence of the type IIS restriction enzyme recognition sequence of the methylation-protectable restriction element, and/or within the nucleotides between the cut site and the recognition sequence of the type IIS restriction enzyme recognition sequence of the methylation-protectable restriction element.

4. The nucleic acid according to claim 3, wherein the nucleic acid comprises nucleic acid sequence between the cut sites of the two methylation-protectable restriction elements, which is a discard sequence; or
wherein the nucleic acid is linearized vector having a methylation-protectable restriction element at each end.

5-8. (canceled)

9. The nucleic acid according to claim 1, wherein the type IIS restriction enzyme recognition sequence of the methylation-protectable restriction element is recognized by the same type IIS restriction enzyme species as the opposing type IIS restriction enzyme recognition sequence.

10. (canceled)

11. The nucleic acid according to claim 1, wherein the nucleic acid further comprises a second methylation-protectable restriction element and an opposing type IIS restriction enzyme recognition sequence thereby forming a maintained composite element, wherein the opposing type IIS restriction enzyme recognition sequence is arranged to direct the type IIS restriction enzyme to cut the nucleic acid at the same site as the type IIS restriction enzyme recognition sequence of the methylation-protectable restriction element, such that the same overhang/sticky end would be produced.

12-14. (canceled)

15. The nucleic acid according to claim 1, wherein the nucleic acid further comprises a second methylation-protectable restriction element and an opposing type IIS restriction enzyme recognition sequence thereby forming an insertional composite element, wherein a functional sequence insert is provided between the methylation-protectable restriction element and the opposing type IIS restriction enzyme recognition sequence.

16. (canceled)

17. The nucleic acid according to claim 1,
wherein the nucleic acid comprises two truncating composite elements with a discard sequence therebetween, or a linearized version thereof with the discard sequence cut out.

18-19. (canceled)

20. The nucleic acid according to claim 2, wherein the DNA methylase recognition sequences of each methylation-protectable restriction element is recognised by the same methylase.

21. (canceled)

22. The nucleic acid according to claim 1, wherein the methylation-protectable restriction element comprises or consists of a sequence according to any one of the overlapping methylation/restriction sites identified in Table 3 herein.

23. The nucleic acid according to claim 1, wherein the methylation-protectable restriction element comprises or consists of the sequence GACNNGGTCTCNNNNN (BsaI/M.Osp807II—SEQ ID NO: 1) or GAAGACGCNNNNNN (BpiI/M2.NmeMC58II—SEQ ID NO: 2) or GAAGCTCTTCNNNN (LguI/M.XmnI—SEQ ID NO: 3).

24. The nucleic acid according to claim 1, wherein the methylation-protectable and/or opposing type IIS restriction enzyme recognition sequence comprises or consists of a sequence according to any one of the type IIS restriction enzyme recognition sequences identified in Table 3 herein.

25. The nucleic acid according to claim 1, wherein the methylation-protectable and/or opposing type IIS restriction enzyme recognition sequence comprises or consists of the sequence GGTCTC (BsaI—SEQ ID NO: 4) or GAAGAC (BpiI—SEQ ID NO: 5) or GCTCTTC (LguI—SEQ ID NO: 6).

26. (canceled)

27. The nucleic acid according to claim 1, wherein the DNA methylase recognition sequence comprises or consists of the sequence GACNNNGTC (M.Osp807II—SEQ ID NO: 7) or GACGC (M2.NmeMC58II—SEQ ID NO: 8) or (M.XmnI—SEQ ID NO: 9).

28-43. (canceled)

44. A method of scarless DNA assembly of DNA fragments comprising the steps of:
(A) providing a first linearised methylated nucleic acid by restriction enzyme cutting of a first nucleic acid,
wherein the first nucleic acid comprises first and second methylation-protectable restriction elements with a discard sequence therebetween, or providing a previously restriction enzyme cut version thereof that has the discard sequence excised,
wherein the first and second methylation-protectable restriction elements of the first nucleic acid each comprise:
a type IIS restriction enzyme recognition sequence; and
a DNA methylase recognition sequence,
wherein the type IIS restriction enzyme recognition sequence and the DNA methylase recognition sequence overlap such that the base modified by the DNA methylase lies within the type IIS restriction enzyme recognition sequence,
wherein the DNA methylase recognition sequence is not identical to or enclosed by the type IIS restriction enzyme recognition sequence, and
wherein the DNA methylase recognition sequence does not overlap with the sequence that would form the overhang end sequence generated by the type IIS restriction enzyme, and
wherein the first and second methylation-protectable restriction elements are methylated with the DNA methylase that recognises the DNA methylase recognition sequence; and
wherein the first nucleic acid further comprises opposing type IS restriction enzyme recognition sequences provided on the opposing side of the cut site of each of the first and second methylation-protectable restriction elements to provide i) a maintained composite element and ii) a truncated composite element,
wherein the maintained composite element is an arrangement wherein the opposing type IIS restriction enzyme recognition sequence is arranged to direct the type IIS restriction enzyme to cut the nucleic acid at the same site as the type IIS restriction enzyme recognition sequence of the opposing methylation-protectable restriction element, such that the same overhang is produced, and
wherein the truncating composite element is an arrangement wherein the opposing type IIS restriction enzyme recognition sequence of the truncating composite element is arranged to direct the type IIS restriction enzyme to cut the nucleic acid at a site that is within the recognition sequence of the type IIS restriction enzyme recognition sequence of the methylation-protectable restriction element, or within the nucleotides between the cut site and the recognition sequence of the type IIS restriction enzyme recognition sequence of the methylation-protectable restriction element;
wherein the first nucleic acid is restriction enzyme cut by a type IIS restriction enzyme that recognizes the opposing type IIS restriction enzyme recognition sequences thereby forming the first linearised methylated nucleic acid;
and wherein the maintained and truncating composite elements are methylated with the DNA methylase;
providing a first DNA fragment for assembly having overhang ends that are adapted to ligate to the overhang ends of the first linearised methylated nucleic acid;
ligating the first DNA fragment for assembly and first linearised methylated nucleic acid with a ligase to form a first methylated intermediate vector;
transforming the first methylated intermediate vector into a bacterial strain that does not express the DNA methylase(s) that recognises either of the DNA methylase recognition sequence(s) of the maintained and truncating composite elements, thereby forming a first intermediate vector that is not methylated;
isolating the first intermediate vector;
(B) providing a second linearised methylated nucleic acid by restriction enzyme cutting of a second nucleic acid,
wherein the second nucleic acid comprises first and second methylation-protectable restriction elements with a discard sequence therebetween, or providing a previously restriction enzyme cut version thereof that has the discard sequence excised,
wherein the first and second methylation-protectable restriction elements of the second nucleic acid each comprise:
a type IIS restriction enzyme recognition sequence; and
a DNA methylase recognition sequence,
wherein the type IIS restriction enzyme recognition sequence and the DNA methylase recognition sequence overlap such that the base modified by the DNA methylase lies within the type IIS restriction enzyme recognition sequence,
wherein the DNA methylase recognition sequence is not identical to or enclosed by the type IIS restriction enzyme recognition sequence, and
wherein the DNA methylase recognition sequence does not overlap with the sequence that would form the overhang end sequence generated by the type IIS restriction enzyme, and
wherein the first and second methylation-protectable restriction elements are methylated with the DNA methylase that recognises the DNA methylase recognition sequence; and
wherein the second nucleic acid further comprises opposing type IIS restriction enzyme recognition sequences provided on the opposing side of the cut site of each of the first and second methylation-protectable restriction elements to provide i) a maintained composite element and ii) a truncated composite element,
wherein the maintained composite element is an arrangement wherein the opposing type IIS restriction enzyme recognition sequence is arranged to direct the type IIS restriction enzyme to cut the nucleic acid at the same site as the type IIS restriction enzyme recognition sequence of the opposing methylation-protectable restriction element, such that the same overhang is produced, and
wherein the truncating composite element is an arrangement wherein the opposing type IIS restriction enzyme recognition sequence of the truncating composite element is arranged to direct the type IIS restriction enzyme to cut the nucleic acid at a site that is within the recognition sequence of the type IIS restriction enzyme recognition sequence of the methylation-protectable restriction element, and/or within the nucleotides between the cut site and the recognition sequence of the type IIS restriction enzyme recognition sequence of the methylation-protectable restriction element:
wherein the second nucleic acid is restriction enzyme cut by a type IIS restriction enzyme that recognizes the opposing type IIS restriction enzyme recognition sequences thereby forming the second linearised methylated nucleic acid;
and wherein the maintained and truncating composite elements are methylated with the DNA methylase, and wherein the overhang provided by the maintained composite element of the first linearised methylated nucleic acid is different in sequence to the overhang provided by the methylation-protectable restriction element of the second linearised methylated nucleic acid;
providing a second DNA fragment for assembly having overhang ends that are adapted to ligate to the overhang ends of the second linearised methylated nucleic acid;
ligating the second DNA fragment for assembly and second linearised methylated nucleic acid with a ligase to form a second methylated intermediate vector;
transforming the second methylated intermediate vector into a bacterial strain that does not express the DNA methylase(s) that recognises either of the DNA methylase recognition sequence(s) of the maintained and truncating composite elements, thereby forming a second intermediate vector that is not methylated;
isolating the second intermediate vector;
(C) cutting the first intermediate vector with a type IIS restriction enzyme that recognises the type IIS restriction enzyme recognition sequence of the maintained composite element and a type IIS restriction enzyme that recognises the type IIS restriction enzyme recognition sequence of the truncating composite element, thereby forming a first adapted DNA fragment insert that comprises a maintained-overhang sequence that is determined by the maintained composite element and an opposing native-overhang sequence that is determined by the native sequence of the first DNA fragment for assembly;
(D) cutting the second intermediate vector with a type IIS restriction enzyme that recognises the type IIS restriction enzyme recognition sequence of the maintained composite element and a type IIS restriction enzyme that recognises the type IIS restriction enzyme recognition sequence of the truncating composite element, thereby forming a second adapted DNA fragment insert that comprises a maintained-overhang sequence that is determined by the maintained composite element and an opposing native-overhang sequence that is determined by the native sequence of the second DNA fragment for assembly;
wherein
(i) the first and second adapted DNA fragments are end fragments wherein their native-overhang sequences are complementary, such that they are arranged to ligate together; or
(ii) one or more middle DNA fragments for assembly are provided wherein the first and second adapted DNA fragments are respective end fragments in the assembly, and the one or more middle DNA fragments are arranged to be ligated between the first and second adapted DNA fragments via complementary native-overhang sequences;
further comprising the step of ligating together, with a ligase, the first and second adapted DNA fragments, or the first and second adapted DNA fragments and one or more middle DNA fragments, to form an assembled DNA fragment having maintained-overhangs at each end.

45-46. (canceled)

47. The method according to claim 44, wherein the middle DNA fragment comprises a first native-overhang sequence that is complementary to the native-overhang of the first adapted DNA fragment and a second native-overhang sequence that is complementary to the native-overhang of the second adapted DNA fragment.

48. The method according to claim 44, wherein the method comprises two or more middle DNA fragments for assembly, the middle DNA fragments comprise native-overhang sequences that are complementary to the native-overhang of a neighbouring middle DNA fragment, such that they can be ligated together in a pre-determined order; and wherein the first and last middle DNA fragments in the sequence are arranged to ligate to the respective first adapted DNA fragment and second adapted DNA fragment via complementary native-overhang sequences.

49. The method according to claim 44, wherein a middle DNA fragment for assembly is provided by providing a further linearised methylated nucleic acid
by restriction enzyme cutting of a nucleic acid, wherein the nucleic acid comprises first and second methylation-protectable restriction elements with a discard sequence therebetween, or providing a previously restriction enzyme cut version thereof that has the discard sequence excised,
wherein the first and second methylation-protectable restriction elements of the nucleci acid each comprise:
a type IIS restriction enzyme recognition sequence; and
a DNA methylase recognition sequence,
wherein the type IIS restriction enzyme recognition sequence and the DNA methylase recognition sequence overlap such that the base modified by the DNA methylase lies within the type IIS restriction enzyme recognition sequence,
wherein the DNA methylase recognition sequence is not identical to or enclosed by the type IIS restriction enzyme recognition sequence, and
wherein the DNA methylase recognition sequence does not overlap with the sequence that would form the overhang end sequence generated by the type IIS restriction enzyme, and
wherein the first and second methylation-protectable restriction elements are methylated with the DNA methylase that recognises the DNA methylase recognition sequence; and
wherein the nucleic acid further comprises opposing type IIS restriction enzyme recognition sequences provided on the opposing side of the cut site of each of the first and second methylation-protectable restriction elements to provide two truncated composite elements,
wherein the truncating composite element is an arrangement wherein the opposing type IIS restriction enzyme recognition sequence of the truncating composite element is arranged to direct the type IIS restriction enzyme to cut the nucleic acid at a site that is within the recognition sequence of the type IIS restriction enzyme recognition sequence of the methylation-protectable restriction element, and/or within the nucleotides between the cut site and the recognition sequence of the type IIS restriction enzyme recognition sequence of the methylation-protectable restriction element;
wherein the nucleic acid is restriction enzyme cut by a type IIS restriction enzyme that recognizes the opposing type IS restriction enzyme recognition sequences thereby forming the further linearised methylated nucleic acid;
providing an adapted middle DNA fragment for assembly having overhang ends that are adapted to ligate to the overhang ends of the further linearised methylated nucleic acid;
ligating the adapted middle DNA fragment for assembly and further linearised methylated nucleic acid with a ligase to form a methylated intermediate vector;
transforming the methylated intermediate vector into a bacterial strain that does not express the DNA methylase(s) that recognises either of the DNA methylase recognition sequence(s) of the methylation-protectable restriction elements, thereby forming an intermediate vector that is not methylated;
isolating the intermediate vector;
cutting the intermediate vector with restriction enzymes that recognise the restriction enzyme recognition sequence of the methylation-protectable restriction elements, thereby forming a middle DNA fragment insert that comprises native-overhang sequences at each end that are determined by the native sequence of the middle DNA fragment for assembly.

50-51. (canceled)

52. The method according to claim 44, further comprise the step of providing a linearised destination vector for insertion of the assembled DNA fragments.

53. The method according to claim 52, wherein the linearised destination vector is provided by cutting a circular destination vector with the restriction enzyme(s) that recognise the type IIS restriction enzyme recognition sequences of the maintained and/or truncating composite element.

54-56. (canceled)

57. A kit comprising one or more nucleic acids according to claim 1.

58. The kit according to claim 56, further comprising a restriction enzyme and/or a ligase, such as T4 DNA ligase.

59. A host cell comprising nucleic acid according to claim 1.

60. (canceled)
